(19) United States
(12) Patent Application Publication
(10) Pub. No.: US 2022/0267420 A1
(43) Pub. Date: Aug. 25, 2022

(54) FOXP3 TARGETING AGENT COMPOSITIONS AND METHODS OF USE FOR ADOPTIVE CELL THERAPY

(71) Applicants: Memorial Sloan Kettering Cancer Center, New York, NY (US); Eureka Therapeutics, Inc., Emeryville, CA (US)

(72) Inventors: David A. SCHEINBERG, New York, NY (US); Cheng LIU, Emeryville, CA (US); Zhiyuan YANG, Albany, CA (US); Lianxing LIU, San Francisco, CA (US); Shaohua Xu, San Francisco, CA (US); Pei WANG, Albany, CA (US); Yiyang XU, Pleasanton, CA (US)

(73) Assignees: Memorial Sloan Kettering Cancer Center, New York, NY (US); Eureka Therapeutics, Inc., Emeryville, CA (US)

(21) Appl. No.: 16/970,332

(22) PCT Filed: Feb. 14, 2019
(86) PCT No.: PCT/US2019/018112

§ 371 Date: Aug. 14, 2020

Related U.S. Application Data
(60) Provisional application No. 62/631,465, filed on Feb. 15, 2018.

(51) Int. Cl. C07K 16/18; C07K 14/725; C07K 14/74; C07K 14/705; C12N 5/0783

(52) U.S. Cl. CPC C07K 16/18; C07K 14/7051; C07K 14/70539; C07K 14/70521; C12N 5/0636; A61K 38/00; C07K 2319/02; C07K 2317/622; C07K 2317/31; C07K 2317/565; C07K 2319/33

(57) ABSTRACT

Provided herein are compositions, kits, and methods for manufacturing cells for adoptive cell therapy comprising (a) an engineered receptor, vector encoding an engineered receptor, or engineered immune cell expressing such engineered receptor or comprising such vector; and (b) a Fox P3 targeting agent.

### CROSS-REFERENCE TO RELATED APPLICATION

This application is a National Stage Application of PCT/US2019/018112, filed Feb. 14, 2019, which claims priority to U.S. provisional application No. 62/631,465, filed Feb. 15, 2018, the entire contents of which are incorporated herein by reference.

### STATEMENT OF GOVERNMENT SUPPORT

This invention was made with government support under CA008748, CA055349 and CA023766 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Mar. 22, 2019, is named 115872-0531_SL.txt and is 259,290 bytes in size.

### BACKGROUND OF THE INVENTION

Adoptive and engineered T cell therapies, including chimeric antigen receptor (CAR) T cells, T cell receptor (TCR) engineered T cells, and antigen-adopted T cells, have emerged recently as important therapies for a variety of diseases, such as infectious diseases (e.g., HIV) and cancer. First generation CARs were designed by fusing the scFv to the intracellular signaling domain of the CD3-ζ chain, whereas subsequent generations of CARs were modified to include co-stimulatory molecules (e.g., CD28, CD80, and 4-1BB) and activation molecules (e.g., CD3ζ) for improved T cell activation and efficacy. However, immunosuppression by T regulatory cells (Tregs) and Treg-like cells presents a major obstacle for successful immunotherapy.

The transcription factor forkhead box p3 (Foxp3) is overexpressed in Tregs and Treg-like cells and plays a central role in the suppressive function of these cells. Cell samples used for adoptive cell therapies, including immune cell samples used to prepare engineered immune cells, contain mixtures of FoxP3 positive immunosuppressive cells (e.g., Tregs) and FoxP3 negative immune activator cells (e.g., effector cells). The presence of the immunosuppressive cells can negatively affect the production of cell populations for adoptive cell therapy and can impact their efficacy when administered to a patient. Accordingly, disclosed herein are compositions, kits, and methods for improving the manufacture of engineered immune cells and for increasing the efficacy of adoptive cell therapy.

### SUMMARY OF THE INVENTION

Provided herein, in certain embodiments are methods for manufacturing an engineered immune cell, comprising: contacting a sample comprising a plurality of immune cells with (a) a vector encoding an engineered receptor; and (b) a forkhead box P3 (FoxP3) targeting agent, thereby producing an engineered immune cell that comprises the vector. In some embodiments, the plurality of immune cells comprises one or more peripheral blood mononuclear cell (PBMC). In some embodiments, the one or more PBMC is a leukocyte. In some embodiments, the leukocyte is a lymphocyte. In some embodiments, the lymphocyte is a T cell. In some embodiments, the T cell is an effector T cell. In some embodiments, the effector T cell is a cytotoxic T cell. In some embodiments, the cytotoxic T cell is a cluster of differentiation 8 positive (CD8+) T cell. In some embodiments, the effector cell is a helper T cell. In some embodiments, the helper T cell is a cluster of differentiation 4 positive (CD4+) T cell. In some embodiments, the T cell is a regulatory T cell. In some embodiments, the plurality of immune cells comprises one or more FoxP3 expressing cells. In some embodiments, the plurality of immune cells comprises one or more cells that do not express FoxP3. In some embodiments, the plurality of immune cells comprises one or more FoxP3 expressing cells and one or more cells that do not express FoxP3. In some embodiments, at least one of the one or more FoxP3 expressing cells is lysed or killed. In some embodiments, at least one of the one or more FoxP3 expressing cells is separated from the cells that do not express FoxP3. In some embodiments, contacting the sample with the FoxP3 targeting agent comprises contacting the sample with two or more different FoxP3 targeting agents. In some embodiments, at least one of the one or more FoxP3 expressing cells is lysed or killed, and at least one of the one or more FoxP3 expressing cells is separated from the cells that do not express FoxP3. In some embodiments, the sample is contacted with the FoxP3 targeting agent prior to being contacted with the vector. In some embodiments, the sample is contacted with the FoxP3 targeting agent and the vector concurrently. In some embodiments, the sample is contacted with the FoxP3 targeting agent after being contacted with the vector.

In some embodiments, the engineered receptor of the engineered immune cell is selected from the group consisting of a chimeric antigen receptor (CAR), chimeric antibody-T cell receptor (caTCR), and engineered T cell receptor (eTCR). In some embodiments, the engineered receptor is a CAR. In some embodiments, the CAR comprises at least one extracellular antigen-binding domain. In some embodiments, the at least one extracellular antigen-binding domain comprises a single chain variable region fragment (scFv). In some embodiments, the CAR comprises at least one intracellular signaling domain. In some embodiments, the at least one intracellular signaling domain comprises a CD3zeta polypeptide or fragment thereof. In some embodiments, the engineered receptor is a caTCR. In some embodiments, the caTCR comprises at least one transmembrane domain. In some embodiments, the at least one transmembrane domain is derived from a transmembrane domain of a TCR. In some embodiments, the transmembrane domain of the TCR is the transmembrane domain of a gamma-delta TCR. In some embodiments, the caTCR comprises at least one constant region. In some embodiments, the at least one constant region comprises a heavy chain constant region or a fragment thereof. In some embodiments, the heavy chain constant region comprises one or more domains. In some embodiments, the heavy chain constant region comprises three domains. In some embodiments, the at least one constant region comprises a light chain constant region or fragment thereof. In some embodiments, the light chain constant region comprises at least one domain. In some embodiments, the at least one constant region is derived from a constant region of a TCR. In some embodiments, the constant region of the TCR is a constant region of a gamma-delta TCR.

In some embodiments, the caTCR comprises: (a) a first polypeptide chain comprising a first antigen-binding domain comprising a VH antibody domain and a first TCR domain (TCRD) comprising a first TCR transmembrane domain (TCR-TM); and (b) a second polypeptide chain comprising a second antigen-binding domain comprising a VL antibody domains and a second TCRD comprising a second TCR-TM, wherein the VH domain of the first antigen-binding domain and the VL domain of the second antigen-binding domain form an antigen-binding module that specifically binds to the target antigen, and wherein the first TCRD and the second TCRD form a TCR module (TCRM) that is capable of recruiting at least one TCR-associated signaling module. In some embodiments, the first TCR-TM is derived from one of the transmembrane domains of a first naturally occurring TCR and the second TCR-TM is derived from the other transmembrane domain of the first naturally occurring TCR. In some embodiments, the first naturally occurring TCR is a gamma-delta TCR. In some embodiments, the first polypeptide chain further comprises a first peptide linker between the first antigen-binding domain and the first TCRD and the second polypeptide chain further comprises a second peptide linker between the second antigen-binding domain and the second TCRD. In some embodiments, the first and/or second peptide linkers comprise, individually, a constant domain or fragment thereof from an immunoglobulin or TCR subunit. In some embodiments, the first and/or second peptide linkers comprise, individually, a CH1, CH2, CH3, CH4, or CL antibody domain, or a fragment thereof. In some embodiments, the first and/or second peptide linkers comprise, individually, a Cα, Cβ, Cγ, or Cδ TCR domain, or a fragment thereof.

In some embodiments, the engineered receptor is an eTCR. In some embodiments, the eTCR comprises an antigen/MHC-binding region. In some embodiments, the antigen/MHC-binding region is derived from an antigen/MHC-binding region of a naturally occurring TCR. In some embodiments, the engineered receptor binds to a cell surface antigen. In some embodiments, the cell surface antigen is selected from the group consisting of a protein, carbohydrate, and lipid. In some embodiments, the cell surface antigen is selected from the group consisting of cluster of differentiation 19 (CD19), CD20, CD47, glypican 3 (GPC-3), Receptor Tyrosine Kinase-Like Orphan Receptor 1 (ROR1), ROR2, B Cell Maturation Antigen (BCMA), G Protein-Coupled Receptor Class C Group 5 Member D (GPRC5D), and Fc Receptor Like 5 (FCRL5). In some embodiments, the cell surface antigen is CD19. In some embodiments, the engineered receptor binds to a complex comprising a peptide and a major histocompatibility complex (MHC) protein. In some embodiments, the peptide is derived from a protein selected from the group consisting of Wilms' tumor gene 1 (WT-1), alpha-fetoprotein (AFP), human papilloma virus 16 E7 protein (HPV16-E7), New York Esophageal Squamous Cell Carcinoma 1 (NY-ESO-1), preferentially expressed antigen of melanoma (PRAME), Epstein-Barr virus-latent membrane protein 2 alpha (EBV-LMP2A), human immunodeficiency virus 1 (HIV-1), KRAS, Histone H3.3, and prostate specific antigen (PSA). In some embodiments, the peptide is derived from AFP. In some embodiments, the peptide derived from AFP comprises the sequence of FMNKFIYEI (SEQ ID NO: 338). In some embodiments, the MHC protein is a MHC class I protein. In some embodiments, the MHC class I protein is the HLA-A*02:01 subtype of the HLA-A02 allele. In some embodiments, the engineered receptor is multispecific. In some embodiments, the engineered receptor is monospecific. In some embodiments, the vector encoding the engineered receptor is a mammalian expression vector. In some embodiments, the mammalian expression vector is a lentiviral vector or transposon vector.

In some embodiments, the FoxP3 targeting agent is an antibody, CAR, caTCR, or eTCR, or comprises antigen-binding fragment thereof. In some embodiments, the FoxP3 targeting agent is a TCR molecule or comprises an antigen-binding portion of a TCR molecule. In some embodiments, the FoxP3 targeting agent comprises an antigen-binding protein that binds to a complex comprising a FoxP3-derived peptide and an MHC protein. In some embodiments, the MHC protein is a MHC class I protein. In some embodiments, the MHC class I protein is a human leukocyte antigen (HLA) class I molecule. In some embodiments, the HLA class I molecule is HLA-A. In some embodiments, the HLA-A is HLA-A2. In some embodiments, the HLA-A2 is HLA-A*02:01. In some embodiments, the antigen-binding protein is an antibody, a CAR, or a caTCR. In some embodiments, the antigen-binding protein is monospecific. In some embodiments, the antigen-binding protein is a full-length antibody. In some embodiments, the antigen-binding protein is an IgG. In some embodiments, the antigen-binding protein is coupled to a solid support. In some embodiments, the solid support is selected from a group consisting of a bead, microwell, and planar glass surface. In some embodiments, the bead is selected from a group consisting of a magnetic bead, crosslinked polymer bead, and beaded agarose. In some embodiments, the antigen-binding protein is multispecific. In some embodiments, the antigen-binding protein is a bispecific antibody. In some embodiments, the bispecific antibody comprises: (a) an antigen-binding domain specific for the complex comprising the FoxP3 peptide and the MHC protein, and (b) an antigen-binding domain specific for cluster of differentiation 3 (CD3). In some embodiments, the antigen-binding protein is a chimeric antigen receptor (CAR). In some embodiments, the FoxP3 targeting agent is an anti-FoxP3 CAR-T cell. In some embodiments, the FoxP3-derived peptide fragment has a length of 8-12 amino acids. In some embodiments, the FoxP3-derived peptide fragment is selected from FoxP3-1 having the amino acid sequence set forth in SEQ ID NO: 2 or a portion thereof, FoxP3-2 having the amino acid sequence set forth in SEQ ID NO: 3 or a portion thereof, FoxP3-3 having the amino acid sequence set forth in SEQ ID NO: 4 or a portion thereof, FoxP3-4 having the amino acid sequence set forth in SEQ ID NO: 5 or a portion thereof, FoxP3-5 having the amino acid sequence set forth in SEQ ID NO: 6 or a portion thereof, FoxP3-6 having the amino acid sequence set forth in SEQ ID NO: 7 or a portion thereof; and FoxP3-7 having the amino acid sequence set forth in SEQ ID NO: 8 or a portion thereof. In some embodiments, the FoxP3-derived peptide fragment is FoxP3-7 having the amino acid sequence set forth in SEQ ID NO: 8 or a portion thereof. In some embodiments, the antigen-binding protein comprises: (a) a heavy chain variable region CDR1 comprising an amino acid sequence set forth in SEQ ID NO: 16; a heavy chain variable region CDR2 comprising an amino acid sequence set forth in SEQ ID NO: 17; a heavy chain variable region CDR3 comprising an amino acid sequence set forth in SEQ ID NO: 18; a light chain variable region CDR1 comprising an amino acid sequence set forth in SEQ ID NO: 19; a light chain variable region CDR2 comprising an amino acid sequence set forth in SEQ ID NO: 20; and a light chain variable region CDR3 comprising an amino acid sequence set forth in SEQ ID NO: 21; (b) a heavy chain variable region CDR1 comprising an amino acid sequence set forth in SEQ ID NO: 22; a heavy chain variable region CDR2 comprising an amino acid sequence set forth in SEQ ID NO: 23; a heavy chain variable region CDR3 comprising an amino acid sequence set forth in SEQ ID NO: 24; a light chain variable region CDR1 comprising an amino acid sequence set forth in SEQ ID NO: 25; a light chain variable region CDR2 comprising an amino acid sequence set forth in SEQ ID NO: 26; and a light chain variable region CDR3 comprising an amino acid sequence set forth in SEQ ID NO: 27; (c) a heavy chain variable region CDR1 comprising an amino acid sequence set forth in SEQ ID NO: 28; a heavy chain variable region CDR2 comprising an amino acid sequence set forth in SEQ ID NO: 29; a heavy chain variable region CDR3 comprising an amino acid sequence set forth in SEQ ID NO: 30; a light chain variable region CDR1 comprising an amino acid sequence set forth in SEQ ID NO: 31; a light chain variable region CDR2 comprising an amino acid sequence set forth in SEQ ID NO: 32; and a light chain variable region CDR3 comprising an amino acid sequence set forth in SEQ ID NO: 33; (d) a heavy chain variable region CDR1 comprising an amino acid sequence set forth in SEQ ID NO: 34; a heavy chain variable region CDR2 comprising an amino acid sequence set forth in SEQ ID NO: 35; a heavy chain variable region CDR3 comprising an amino acid sequence set forth in SEQ ID NO: 36; a light chain variable region CDR1 comprising an amino acid sequence set forth in SEQ ID NO: 37; a light chain variable region CDR2 comprising an amino acid sequence set forth in SEQ ID NO: 38; and a light chain variable region CDR3 comprising an amino acid sequence set forth in SEQ ID NO: 39; (e) a heavy chain variable region CDR1 comprising an amino acid sequence set forth in SEQ ID NO: 40; a heavy chain variable region CDR2 comprising an amino acid sequence set forth in SEQ ID NO: 41; a heavy chain variable region CDR3 comprising an amino acid sequence set forth in SEQ ID NO: 42; a light chain variable region CDR1 comprising an amino acid sequence set forth in SEQ ID NO: 43; a light chain variable region CDR2 comprising an amino acid sequence set forth in SEQ ID NO: 44; and a light chain variable region CDR3 comprising an amino acid sequence set forth in SEQ ID NO: 45; (f) a heavy chain variable region CDR1 comprising an amino acid sequence set forth in SEQ ID NO: 46; a heavy chain variable region CDR2 comprising an amino acid sequence set forth in SEQ ID NO: 47; a heavy chain variable region CDR3 comprising an amino acid sequence set forth in SEQ ID NO: 48; a light chain variable region CDR1 comprising an amino acid sequence set forth in SEQ ID NO: 49; a light chain variable region CDR2 comprising an amino acid sequence set forth in SEQ ID NO: 50; and a light chain variable region CDR3 comprising an amino acid sequence set forth in SEQ ID NO: 51; (g) a heavy chain variable region CDR1 comprising an amino acid sequence set forth in SEQ ID NO: 52; a heavy chain variable region CDR2 comprising an amino acid sequence set forth in SEQ ID NO: 53; a heavy chain variable region CDR3 comprising an amino acid sequence set forth in SEQ ID NO: 54; a light chain variable region CDR1 comprising an amino acid sequence set forth in SEQ ID NO: 55; a light chain variable region CDR2 comprising an amino acid sequence set forth in SEQ ID NO: 56; and a light chain variable region CDR3 comprising an amino acid sequence set forth in SEQ ID NO: 57; or (h) a heavy chain variable region CDR1 comprising an amino acid sequence set forth in SEQ ID NO: 58; a heavy chain variable region CDR2 comprising an amino acid sequence set forth in SEQ ID NO: 59; a heavy chain variable region CDR3 comprising an amino acid sequence set forth in SEQ ID NO: 60; a light chain variable region CDR1 comprising an amino acid sequence set forth in SEQ ID NO: 61; a light chain variable region CDR2 comprising an amino acid sequence set forth in SEQ ID NO: 62; and a light chain variable region CDR3 comprising an amino acid sequence set forth in SEQ ID NO: 63.

In some embodiments, the antigen-binding protein comprises a heavy chain variable region CDR1 comprising an amino acid sequence set forth in SEQ ID NO: 46; a heavy chain variable region CDR2 comprising an amino acid sequence set forth in SEQ ID NO: 47; a heavy chain variable region CDR3 comprising an amino acid sequence set forth in SEQ ID NO: 48; a light chain variable region CDR1 comprising an amino acid sequence set forth in SEQ ID NO: 49; a light chain variable region CDR2 comprising an amino acid sequence set forth in SEQ ID NO: 50; and a light chain variable region CDR3 comprising an amino acid sequence set forth in SEQ ID NO: 51.

In some embodiments, contacting the sample with the vector occurs at least 12, 24, 36, 48, 60, 72, 84, 96, 108, 120, 132, or 144 hours prior to contacting the sample with the FoxP3 targeting agent. In some embodiments, contacting the sample with the FoxP3 targeting agent occurs at least 4, 6, 8, 10, 12, 16, 20, 24, 36, or 48 hours prior to contacting the sample with the vector.

In some embodiments, contacting the sample with the FoxP3 targeting agent reduces the number of FoxP3 positive (FoxP3+) cells in the sample. In some embodiments, contacting the sample with the FoxP3 targeting agent reduces the number of FoxP3+ cells in the sample by at least about 30%, 40%, 50%, 60%, 70%, 80%, 90% or more as compared to the number of FoxP3+ cells in the sample prior to contact with the FoxP3 targeting agent. In some embodiments, contacting the sample with the FoxP3 targeting agent reduces the number of FoxP3+ cells in the sample by at least about 30%, 40%, 50%, 60%, 70%, 80%, 90% or more as compared to the number of FoxP3+ cells in a control sample that has not been contacted with the FoxP3 targeting agent.

In some embodiments, the at least one extracellular antigen binding domain or the antigen-binding module binds to CD19 and comprises: (a) (i) heavy chain CDR1, CDR2, and CDR3, respectively, comprising amino acid sequences that are at least 80%, at least 85%, at least 90%, or at least 95% identical to SEQ ID NOs: 105, 106, and 107; and/or (ii) light chain CDR1, CDR2, and CDR3, respectively, comprising amino acid sequences that are at least 80%, at least 85%, at least 90%, or at least 95% identical to SEQ ID NOs: 109, 110, or 111; (b)(i) heavy chain CDR1, CDR2, and CDR3, respectively, comprising amino acid sequences that are at least 80%, at least 85%, at least 90%, or at least 95% identical to SEQ ID NOs: 105, 106, and 108; and/or (ii) light chain CDR1, CDR2, and CDR3, respectively, comprising amino acid sequences that are at least 80%, at least 85%, at least 90%, or at least 95% identical to SEQ ID NOs: 109, 110, or 111; (c) (i) heavy chain CDR1, CDR2, and CDR3, respectively, comprising amino acid sequences that are at least 80%, at least 85%, at least 90%, or at least 95% identical to SEQ ID NOs: 105, 106, and 107; and/or (ii) light chain CDR1, CDR2, and CDR3, respectively, comprising amino acid sequences that are at least 80%, at least 85%, at least 90%, or at least 95% identical to SEQ ID NOs: 109, 110, or 112; or (d) (i) heavy chain CDR1, CDR2, and CDR3, respectively, comprising amino acid sequences that are at least 80%, at least 85%, at least 90%, or at least 95% identical to SEQ ID NOs: 105, 106, and 108; and/or (ii) light chain CDR1, CDR2, and CDR3, respectively, comprising amino acid sequences that are at least 80%, at least 85%, at least 90%, or at least 95% identical to SEQ ID NOs: 109, 110, or 112.

In some embodiments, the FoxP3 targeting agent is a chimeric antigen receptor (CAR) and wherein the CAR binds to a complex comprising a FoxP3 peptide and a major histocompatibility complex (MHC) protein. In some embodiments, the FoxP3 targeting CAR comprises an scFv that binds to complex comprising a FoxP3 peptide and a major histocompatibility complex (MHC) protein. In some embodiments, the FoxP3 targeting CAR further comprises a CD28-CD3zeta peptide that is fused to the scFv. In some embodiments, the FoxP3 targeting CAR comprises an scFv-CD28-CD3zeta fusion having an amino acid sequence that is at least 80%, at least 85%, at least 90%, or at least 95% identical to SEQ ID NO: 12. In some embodiments, the FoxP3 targeting CAR further comprises a 41BB-CD3zeta peptide that is fused to the scFv. In some embodiments, the FoxP3 targeting CAR comprises an scFv-41BB-CD3 zeta fusion having an amino acid sequence that is at least 80%, at least 85%, at least 90%, or at least 95% identical to SEQ ID NO: 13.

In some embodiments, the FoxP3 targeting agent is a chimeric antibody TCR (caTCR) and wherein the caTCR binds to a complex comprising a FoxP3 peptide and a major histocompatibility complex (MHC) protein. In some embodiments, the caTCR comprises a gamma chain of a TCR. In some embodiments, the caTCR further comprises a delta chain of a TCR. In some embodiments, the gamma chain of the TCR is fused to a light chain of an immunoglobulin molecule that binds to FoxP3. In some embodiments, the delta chain of the TCR is fused to a heavy chain of an immunoglobulin molecule that binds to FoxP3. In some embodiments, the FoxP3 targeting caTCR comprises: (a) a first polypeptide chain comprising a first antigen-binding domain comprising a VH antibody domain and a first TCR domain (TCRD) comprising a first TCR transmembrane domain (TCR-TM); and (b) a second polypeptide chain comprising a second antigen-binding domain comprising a VL antibody domains and a second TCRD comprising a second TCR-TM, wherein the VH domain of the first antigen-binding domain and the VL domain of the second antigen-binding domain form an antigen-binding module that specifically binds to the target antigen, and wherein the first TCRD and the second TCRD form a TCR module (TCRM) that is capable of recruiting at least one TCR-associated signaling module. In some embodiments, the first TCR-TM is derived from one of the transmembrane domains of a first naturally occurring TCR and the second TCR-TM is derived from the other transmembrane domain of the first naturally occurring TCR. In some embodiments, the first naturally occurring TCR is a gamma-delta TCR. In some embodiments, the caTCR comprises an anti-FoxP3 light chain/gamma chain fusion having an amino acid sequence that is at least 80%, at least 85%, at least 90%, or at least 95% identical to SEQ ID NO: 15. In some embodiments, the caTCR comprises an anti-FoxP3 heavy chain/delta chain fusion having an amino acid sequence that is at least 80%, at least 85%, at least 90%, or at least 95% identical to SEQ ID NO: 14.

Also provided herein, in certain embodiments are methods for depleting FoxP3 positive cells in a therapeutic composition comprising engineered immune cells expressing engineered receptor, the method comprising contacting the therapeutic composition with a FoxP3 targeting agent.

Also provided herein, in certain embodiments are methods for enriching for engineered-receptor-expressing cytotoxic T cells in a sample, comprising contacting the sample with a FoxP3 targeting agent.

Also provided herein, in certain embodiments are compositions comprising: (a) an engineered immune cell, wherein the engineered immune cell expresses an engineered receptor; and (b) a FoxP3 targeting agent.

Also provided herein, in certain embodiments are compositions comprising: (a) a vector encoding an engineered receptor; and (b) a FoxP3 targeting agent.

Provided herein, in certain embodiments, are compositions, kits, and methods for manufacturing an engineered immune cell.

Also provided herein, in certain embodiments, are compositions, kits, and methods for treating a disease in a subject in need thereof.

### BRIEF DESCRIPTION OF THE DRAWINGS

FIG. 1 illustrates Foxp3-TLI induces peptide-specific T cell response. (A). CD3 T cells from HLA-A0*201+Foxp3 donors were stimulated with Foxp3-TLI peptide for four rounds and T cell response was tested against TLI peptide or with irrelevant peptide EW by IFN-gamma Enzyme-Linked ImmunoSpot (ELISPOT) assay. CD14+APC serve as a negative control. (B). TLI-stimulated T cells also recognize MAC-1 and MAC-2A cells but not HLA-A0*201− cell line Jurkat (C) and (D). T cells from a HLA-A*02:01+ donor were stimulated for five rounds and the cytotoxicity was measured by ⁵¹Cr-release assay against the stimulating peptides pulsed onto T2 cells (C) or un-pulsed target cells (D) by ⁵¹Cr-release assay. HLA-A*02:01 negative AML cell line HL-60 were used as a negative control. Each data point represents average+/−SD from triplicate cultures. Data represent results from multiple similar experiments from multiple donors.

FIG. 2 illustrates binding properties of the bispecific antibodies. (A). Binding of the indicated bispecific mAb constructs to Foxp3+/HLA-A2+T lymphoma cell line MAC-2A and control cell line Jurkat. Since the bispecific mAb constructs were myc-tagged, the binding was tested by staining the cells with the bispecific mAbs, followed by a secondary mAb, mouse anti-myc conjugated to FITC. Controls include unstained cell (Line #1), control bispecific mAb clone NC-16 at 1 (Line #2) and 0.1 μg/ml (Line #3), or secondary mAb GA6×His (Line #4). Foxp3-#32 bispecific mAb was used at 1 μg (Line #5) or 0.1 μg/ml (Line #6). (B). Similarly, the binding of the mouse mAb Foxp3-#32 (Line #2) or its isotype control (Line #1) was used at 1 μg/ml. (C). HLA-A*02 expression was measured by staining the cells with anti-A2 mAb BB7 (Line #2) and its isotype control mouse IgG2b (Line #1), as indicated. Binding strength is shown by median fluorescent intensity.

FIG. 3 illustrates epitope specificity of the bispecific antibodies. (A). The Foxp3-TLI peptide sequence was substituted with alanine at positions 1, 2, 3, 4, 5, 7, 8, 9 or with glycine (G10) at position ten (sequences in Table 3) T2 cells were pulsed with indicated peptides at 50 μg/ml and the binding of Foxp3-#32-bispecific mAb was measured by flow cytometry. (B). Cells were simultaneously stained with anti-HLA-A2 mAb, clone BB7.2, to measure the relative binding of the peptides to HLA-A2 molecule. FIG. 3B discloses SEQ ID NOS 341-350, respectively, in order of appearance.

FIG. 4 illustrates specific binding of Foxp3-#32 mAb to natural Treg cells in PBMC in healthy donors. PBMCs were stained with mAbs specific for CD4, CD25 CD127 and Foxp3-#32 mouse IgG1. Data show that mAb Foxp3-#32 only bond to CD4+CD25highCD127low Tregs, not CD4+25highCD127high population (A), nor CD4+CD25highCD127low Tregs from a HLA-A0*201 negative donor (B). Data show representative results from 3 sets of different individuals.

FIG. 5 illustrates binding of Foxp3-#32 mAb to Treg cells generated in vitro from HLA-A*02:01+ donors. CD4+ T cells were FACS sorted and stimulated with either MAC-2A cells (A) or allo-PBMC (B) as both stimulator and feeder cells, in the presence of IL-2 (100 unit) and TGF-β (10 ng/ml) for a weekly stimulation. Cells were stained with mAbs to surface CD4, CD25, intracellular Foxp3 and mAb Foxp3-#32/APC. Mab Foxp3-#32 binding was determined by gating on the DAPI−, CD4 and CD25 double positive cells. The data show an overlay of Foxp3-#32 plus Foxp3 protein dual staining, and its isotype control mouse IgG1 and rat isotype control for mAb to Foxp3 (dual controls) and mAbs to Foxp3 protein plus isotype control mouse IgG1 for Foxp3-#32 mAb. (C). Cell lines MAC-2A and CSMJ transduced with HLA-A*02:01 were stained with mAbs to intracellular Foxp3 vs Foxp3-#32 mouse mAbs. Foxp3-#32 mAb and Foxp3 protein double positive cells, Foxp3 protein positive cells not bound by isotype for #32 mAb. and isotype controls for both intracellular Foxp3 protein and #32 mAb are shown (upper two panels). Histogram shows the HLA-A2 expression in respective cell lines (lower panels).

FIG. 6 illustrates Foxp3-Foxp3-#32-bispecific mAb-mediated T cell killing against Foxp3+/HLA-A02:01+ cells. PBMCs were incubated with TLI-pulsed T2 cells (A). Foxp3-#32 bispecific mAb against T2 alone (Line #1); control bispecific mAb against T2 alone (Line #2); Foxp3-#32 bispecific mAb against T2 pulsed with TLI peptide (Line #3); control bispecific mAb against T2 pulsed with TLI peptide (Line #4); Foxp3-#32 bispecific mAb against T2 pulsed with EW peptide (Line #5); control bispecific mAb against T2 pulsed with control peptide (Line #6); HL-60 (B), MAC-1 (C) or MAC-2A (D) target cells at an E:T ratio 50:1, with or without bispecific mAbs at the concentrations ranging from 1 μg/ml to 0.0003 μg/ml. Activated T cells were used as effector cells against MAC-2A (E), Jurkat (F), C5MJ/A2 (G) or C5MJ (H) at an E:T ratio 30:1. The cytotoxicity was measured by 5 hour ⁵¹Cr-release assay. The data represent the mean value of triplicate microwell cultures. Data represents results from multiple experiments.

FIG. 7 illustrates representative flow cytometry plots for Tregs in healthy donors and patient's samples. (A). Frequency of CD4+CD127 high or low population from a HLA-A*02:01+ donor after 2 days of culture was shown in left three columns. CD25+Foxp3 expression was shown in middle columns on CD4+CD127 low population and in right columns on CD4+CD127high population. (B). CD4+CD127 high (lower 3 panels) or low population (upper 3 panels) was further analyzed based on CD45RA vs Foxp3 expression from the same cells. Frequency of each fraction was indicated. (C). Similar gating strategy was used for the cells after 3 days of culture. Data show the CD4+Foxp3+ cells (middle 2 columns) or CD45RA vs Foxp3+ cells (right 2 columns) in the CD4+CD127low population (left 2 columns), from the same donor. Data represent one of three similar experiments. (D). Ascites cells from a HLA-A*02:01+ patient with ovarian cancer treated with Foxp3-#32 bispecific mAb for two days were stained with the above Treg markers. Cells were first gated on lymphocytes, excluding large tumor cells and monocyte population, on side scatters and forward scatters. Then CD4+ population were analyzed with two sets of Treg markers: CD25high vs intracellular Foxp3 or CD127 low vs intracellular Foxp3. Data represent one of two similar experiments from the same patient and total three patients.

FIG. 8 illustrates bispecific mAb-mediated cytotoxicity against normal PBMCs. Control cells or PBMCs from HLA-A*02:01 positive or negative donors were incubated in the presence or absence of 0.2 or 1 μg/ml Foxp3-#32 bispecific mAb or its control overnight. Cells were washed and stained with mAbs to human CD3, CD19 and CD33 to determine whether these cell lineages are killed by the bispecific mAbs. The percentage of remaining cells in each cell lineage after co-culture is shown. On the top of the Table, as controls, MAC-1 cells were incubated with HLA-A*02:01 negative PBMCs as effectors at an E:T ratio of 30:1, in the presence or absence of bispecific mAbs at 1 μg/ml. Cells were harvested and stained with mAb to HLA-A2 (BB7.2 clone). Since only MAC1 cells are HLA-A2 positive, the reduction or disappearance of HLA-A2+ population indicates the killing of MAC-1. The bottom of the table shows killing of HLA-A*02:01 positive PBMC (left) or HLA-A*02:01 negative PBMC (right). No significant killing was seen with either HLA type. The data represent one out of three similar experiments with different donors.

FIG. 9 illustrates Foxp3-#32 mAb does not bind to CD3+CD8+ T cells from HLA-A*02:01 positive donor. (A) Foxp3-#32 mAb was tested for its binding to CD3/CD8 double positive cells from HLA-A*02:01 positive healthy donor. No binding was observed compared to control mAb, shown by histogram overlay. Data represent one of flow cytometry data from multiple donors. (B). Percentage of lymphocytes from among all healthy PBMCs from one HLA-A0*2:01 positive donor treated with Foxp3-#32 bispecific mAb at 1 μg/ml for one to three days. Percentage lymphocytes was shown by gating on lymphocyte population in the plot of forward and side scatters. A slight reduction was observed in the Foxp3-#32 bispecific mAb treated group after two and three-day treatment. Each data point shows triplicate staining plus SD. Data represent one of two similar experiments.

FIG. 10A illustrates no Foxp3+Tregs were depleted in HLA-A*02:01 negative healthy donor. PBMCs from a healthy HLA-A*02:01 negative donor were treated with Foxp3-#32 bispecific mAb for two days, in the same experiments shown in FIG. 7 and Foxp3+ Treg depletion was measured by using Treg markers CD4, CD25, CD127, CD45RA surface staining and Foxp3 intracellular staining. Top three panels: untreated PBMCs; middle three panels: PBMCs treated with Foxp3-#32 bispecific mAb; lower three panels: PBMCs treated with control bispecific mAb. The data show representative data from two similar experiments. FIG. 10B illustrates Depletion of Foxp3+Tregs in ascites of a patient with ovarian cancer by Foxp3-#32 Fc-enhanced human IgG1. The ascites cells were treated with Foxp3-#32-Fc-enhanced mAb at a concentration of 10 μg/ml for two (upper panels) and three days (lower panels). Representative plot show CD45RA vs Foxp3 staining in the CD4+CD127low population. Data represent one of two similar experiments.

FIG. 11A illustrates Foxp3-#32 bispecific mAb-mediated T cell killing against in vitro-generated Tregs from HLA-A*02:01+ donors. Purified CD3+ T cells from a HLA-A2 negative donor were incubated with Treg lines generated from a HLA-A*02:01+ donor in the presence or absence of Foxp3-#32 or control bispecific mAb (1 μg/ml) at an E:T ratio 5:1, overnight. The percentage of Foxp3+ cells in HLA-A*02:01+ T cells was determined by flow cytometry. Reduction of the HLA-A2+Foxp3+ cells indicates the Foxp3-#32 bispecific mAb-mediated T cell killing. Upper left quadrant shows the culture of effector cells alone with Treg line and stained with mAbs to HLA-A2 versus intracellular Foxp3 protein; upper right quadrant shows the culture of effectors with Treg line in the presence of control bispecific mA, but X-axis is the staining with isotype control for intracellular Foxp3 protein to show a specific binding of the mAb to Foxp3 protein in other three panels. Lower two panels show the cultures of effectors with Treg line in the presence of Foxp3-#32-(left) or control bispecific mAb (right). Data show representative flow data from duplicate cultures. FIG. 11B provides a summary of similar results tested on two Treg lines, as described in 11A. FIG. 11C illustrates MAC-2A cells that have been transduced with GFP/luciferase were incubated with PBMCs from a HLA-A*02:01 negative donor at an E:T ratio 30:1, in the presence or absence of bispecific mAbs at 1 μg/ml for total 3 days. Luciferin 30 μg was added to each culture well before imaging. Total bioluminescence was measured in the indicated time points. Data represent average of three microwell cultures

FIG. 12 illustrates T2 cells pulsed with various HLA-A2-binding peptides derived from human proteins at 5 μg/ml and the binding of Foxp3-#32-mouse mAb was measured by flow cytometry, as described in the Materials and Methods. Foxp-3 #32 mAb bound to two peptides, peptide 11 and 14 (positions O11 and O14 on microwell plate), derived from minor histocompatibility antigens HA-1 and HA-8, in addition to binding to the Foxp3-TLI peptide. FIG. 12 discloses SEQ ID NOS 351-353, respectively, in order of appearance.

FIG. 13 provides a table of nucleic acid and amino acid sequences useful in the embodiments described herein.

### DETAILED DESCRIPTION OF THE INVENTION

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

### Definitions

Unless defined otherwise, all technical and scientific terms used herein have the meaning commonly understood by a person skilled in the art to which this disclosure belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the singular forms “a”, “an” and “the” are intended to include the plural forms as well, unless the context clearly indicates otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “administration” of an agent to a subject includes any route of introducing or delivering the agent to a subject to perform its intended function. Administration can be carried out by any suitable route, including, but not limited to, intravenously, intramuscularly, intraperitoneally, subcutaneously, and other suitable routes as described herein. Administration includes self-administration and the administration by another.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells, at least about 10,000 cells, at least about 100,000 cells, at least about 1×10⁶ cells, at least about 1×10⁷ cells, at least about 1×10⁸ cells, at least about 1×10⁹ cells, at least about 1×10¹⁰ cells, at least about 1×10¹¹ cells, at least about 1×10¹² cells, or more cells expressing similar or different phenotypes.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide is in the D form and a second plurality is in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

The terms “polypeptide,” “peptide,” and “protein” are used interchangeably herein to refer to a polymer of amino acid residues. The terms apply to naturally occurring amino acid polymers as well as amino acid polymers in which one or more amino acid residues is a non-naturally occurring amino acid, e.g., an amino acid analog. The terms encompass amino acid chains of any length, including full length proteins, wherein the amino acid residues are linked by covalent peptide bonds.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a composition known to exhibit the desired therapeutic effect) and a negative control (a subject or a sample that does not receive the therapy or receives a placebo) are typically employed.

As used herein, the term “effective amount” or “therapeutically effective amount” refers to a quantity of an agent sufficient to achieve a desired therapeutic effect. In the context of therapeutic applications, the amount of a therapeutic peptide administered to the subject can depend on the type and severity of the infection and on the characteristics of the individual, such as general health, age, sex, body weight and tolerance to drugs. It can also depend on the degree, severity and type of disease. The skilled artisan will be able to determine appropriate dosages depending on these and other factors.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of the compositions disclosed herein. The term “expression” also refers to one or more of the following events: (1) production of an RNA template from a DNA sequence (e.g., by transcription) within a cell; (2) processing of an RNA transcript (e.g., by splicing, editing, 5′ cap formation, and/or 3′ end formation) within a cell; (3) translation of an RNA sequence into a polypeptide or protein within a cell; (4) post-translational modification of a polypeptide or protein within a cell; (5) presentation of a polypeptide or protein on the cell surface; and (6) secretion or presentation or release of a polypeptide or protein from a cell.

The term “linker” refers to synthetic sequences (e.g., amino acid sequences) that connect or link two sequences, e.g., that link two polypeptide domains. In some embodiments, the linker contains 1, 2, 3, 4, 5, 6, 7, 8, 9, or 10 of amino acid sequences.

As used herein the term “immune cell” refers to any cell that plays a role in the immune response. Immune cells are of hematopoietic origin, and include lymphocytes, such as B cells and T cells; natural killer cells; myeloid cells, such as monocytes, macrophages, dendritic cells, eosinophils, neutrophils, mast cells, basophils, and granulocytes.

As used herein, the term “native immune cell” refers to an immune cell that naturally occurs in the immune system.

As used herein, the term “engineered immune cell” refers to an immune cell that is genetically modified.

The term “lymphocyte” refers to all immature, mature, undifferentiated and differentiated white lymphocyte populations including tissue specific and specialized varieties. It encompasses, by way of non-limiting example, B cells, T cells, NKT cells, and NK cells. In some embodiments, lymphocytes include all B cell lineages including pre-B cells, progenitor B cells, early pro-B cells, late pro-B cells, large pre-B cells, small pre-B cells, immature B cells, mature B cells, plasma B cells, memory B cells, B-1 cells, B-2 cells and anergic AN1/T3 cell populations.

As used herein, the term “T-cell” includes naïve T cells, CD4+ T cells, CD8+ T cells, memory T cells, activated T cells, anergic T cells, tolerant T cells, chimeric B cells, and antigen-specific T cells.

As used herein “adoptive cell therapeutic composition” refers to any composition comprising cells suitable for adoptive cell transfer. In exemplary embodiments, the adoptive cell therapeutic composition comprises a cell type selected from a group consisting of a tumor infiltrating lymphocyte (TIL), TCR (i.e. heterologous T-cell receptor) modified lymphocytes (e.g., eTCR T cells and caTCR T cells) and CAR (i.e. chimeric antigen receptor) modified lymphocytes (e.g., CAR T cells). In another embodiment, the adoptive cell therapeutic composition comprises a cell type selected from a group consisting of T-cells, CD8+ cells, CD4+ cells, NK-cells, delta-gamma T-cells, regulatory T-cells and peripheral blood mononuclear cells. In another embodiment, TILs, T-cells, CD8+ cells, CD4+ cells, NK-cells, delta-gamma T-cells, regulatory T-cells or peripheral blood mononuclear cells form the adoptive cell therapeutic composition. In one embodiment, the adoptive cell therapeutic composition comprises T cells.

As used herein “tumor-infiltrating lymphocytes” or TILs refer to white blood cells that have left the bloodstream and migrated into a tumor.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)2, and Fab. F(ab′)2, and Fab fragments that lack the Fc fragment of intact antibody, clear more rapidly from the circulation, and may have less non-specific tissue binding of an intact antibody (Wahl et al. (1983) J. Nucl. Med. 24:316-325). The antibodies of the invention comprise whole native antibodies, monoclonal antibodies, human antibodies, humanized antibodies, camelised antibodies, multispecific antibodies, bispecific antibodies, chimeric antibodies, Fab, Fab′, single chain V region fragments (scFv), single domain antibodies (e.g., nanobodies and single domain camelid antibodies), VNAR fragments, bispecific T-cell engager antibodies, minibodies, disulfide-linked Fvs (sdFv), and anti-idiotypic (anti-Id) antibodies, intrabodies, fusion polypeptides, unconventional antibodies and antigen-binding fragments of any of the above. In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Cl q) of the classical complement system. As used herein interchangeably, the terms “antigen-binding portion”, “antigen-binding fragment”, or “antigen-binding region” of an antibody, refer to the region or portion of an antibody that binds to the antigen and which confers antigen specificity to the antibody; fragments of antigen-binding proteins, for example, antibodies include one or more fragments of an antibody that retain the ability to specifically bind to an antigen (e.g., a peptide/HLA complex). It has been shown that the antigen-binding function of an antibody can be performed by fragments of a full-length antibody. Examples of antigen-binding portions encompassed within the term “antibody fragments” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al. (1989) Nature 341: 544-546), which consists of a V_{H} domain; and an isolated complementarity determining region (CDR).

Antibodies and antibody fragments can be wholly or partially derived from mammals (e.g., humans, non-human primates, goats, guinea pigs, hamsters, horses, mice, rats, rabbits and sheep) or non-mammalian antibody producing animals (e.g., chickens, ducks, geese, snakes, urodele amphibians). The antibodies and antibody fragments can be produced in animals or produced outside of animals, such as from yeast or phage (e.g., as a single antibody or antibody fragment or as part of an antibody library). As used herein, the phrase “derived from” includes antibodies and fragments thereof generated from a wild-type (i.e., native) sequence of an antibody or variants/mutants and homologs thereof.

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al. (1988) Science 242:423-426; and Huston et al. (1988) Proc. Natl. Acad. Sci. 85: 5879-5883. These antibody fragments are obtained using conventional techniques known to those of ordinary skill in the art, and the fragments are screened for utility in the same manner as are intact antibodies.

An “isolated antibody” or “isolated antigen-binding protein” is one which has been identified and separated and/or recovered from a component of its natural environment. “Synthetic antibodies” or “recombinant antibodies” are generally generated using recombinant technology or using peptide synthetic techniques known to those of skill in the art.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}:V_{L} heterodimer. The heavy (V_{H}) and light chains (V_{L}) are either joined directly or joined by a peptide-encoding linker (e.g., about 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain.

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston et al. (1988) Proc. Nat. Acad. Sci. USA, 85:5879-5883. See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al. (2008) Hybridoma (Larchmt) 27(6):455-51; Peter et al. J Cachexia Sarcopenia Muscle (2012); Shieh et al. (2009) J Imunol 183(4):2277-85; Giomarelli et al. (2007) Thromb Haemost 97(6):955-63; Fife et al. (2006) J Clin Invst 116(8):2252-61; Brocks et al. (1997) Immunotechnology 3(3): 173-84; Moosmayer et al. (1995) Ther Immunol 2(10):31-40). Agonistic scFvs having stimulatory activity have been described (see, e.g., Peter et al. (2003) J Biol Chem 25278(38):36740-7; Xie et al. (1997) Nat Biotech 15(8):768-71; Ledbetter et al. (1997) Crit Rev Immunol 17(5-6):427-55; Ho et al. (2003) Bio Chim Biophys Acta 1638(3):257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab¹) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. Sequences of Proteins of Immunological Interest, Fifth Edition, U.S. Department of Health and Human Services, NIH Publication No. 91-3242(1991)).

As used herein, the term “affinity” is meant a measure of binding strength. Without being bound to theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes (e.g., either monovalent or multivalent). Methods for calculating the affinity of an antibody for an antigen are known in the art, comprising use of binding experiments to calculate affinity. Antibody activity in functional assays (e.g., flow cytometry assay) is also reflective of antibody affinity. Antibodies and affinities can be phenotypically characterized and compared using functional assays (e.g., flow cytometry assay). Nucleic acid molecules useful in the presently disclosed subject matter include any nucleic acid molecule that encodes a polypeptide or a fragment thereof. In certain embodiments, nucleic acid molecules useful in the presently disclosed subject matter include nucleic acid molecules that encode an antibody or an antigen-binding portion thereof. Such nucleic acid molecules need not be 100% identical with an endogenous nucleic acid sequence, but will typically exhibit substantial identity. Polynucleotides having “substantial homology” or “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger, Methods Enzymol. 152:399 (1987); Kimmel, A. R. Methods Enzymol. 152:507 (1987)).

The terms “substantially homologous” or “substantially identical” mean a polypeptide or nucleic acid molecule that exhibits at least 50% or greater homology or identity to a reference amino acid sequence (for example, any one of the amino acid sequences described herein) or nucleic acid sequence (for example, any one of the nucleic acid sequences described herein). For example, such a sequence is at least about 60%, about 65%, about 70%, about 75%, about 80%, about 85%, about 90%, about 95% or about 99% homologous or identical at the amino acid level or nucleic acid to the sequence used for comparison (e.g., a wild-type, or native, sequence). In some embodiments, a substantially homologous or substantially identical polypeptide contains one or more amino acid amino acid substitutions, insertions, or deletions relative to the sequence used for comparison. In some embodiments, a substantially homologous or substantially identical polypeptide contains one or more non-natural amino acids or amino acid analogs, including, D-amino acids and retroinverso amino, to replace homologous sequences.

Sequence homology or sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program can be used, with a probability score between e⁻³ and e⁻¹⁰⁰ indicating a closely related sequence.

As used herein, the term “analog” refers to a structurally related polypeptide or nucleic acid molecule having the function of a reference polypeptide or nucleic acid molecule.

As used herein, the term “a conservative sequence modification” refers to an amino acid modification that does not significantly affect or alter the binding characteristics of the presently disclosed engineered receptor (e.g., the extracellular antigen-binding domain of the engineered receptor) comprising the amino acid sequence. Conservative modifications can include amino acid substitutions, additions and deletions. Modifications can be introduced into the human scFv of the presently disclosed engineered receptor by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

As used herein, the term “ligand” refers to a molecule that binds to a receptor. In particular, the ligand binds a receptor on another cell, allowing for cell-to-cell recognition and/or interaction.

As used herein, the term, “co-stimulatory signaling domain,” or “co-stimulatory domain”, refers to the portion of the engineered receptor comprising the intracellular domain of a co-stimulatory molecule. Co-stimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1BB (CD137), OX40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domains derived from CD28 and 4-1BB, other costimulatory domains are contemplated for use with the engineered receptors described herein. The inclusion of one or more co-stimulatory signaling domains can enhance the efficacy and expansion of T cells expressing engineered receptors. The intracellular signaling and co-stimulatory signaling domains can be linked in any order in tandem to the carboxyl terminus of the transmembrane domain.

As used herein, the term “chimeric co-stimulatory receptor” or “CCR” refers to a chimeric receptor that binds to an antigen and provides co-stimulatory signals, but does not provide a T-cell activation signal.

As used herein, regulatory region of a nucleic acid molecule means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, but are not limited to, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, “operably linked” with reference to nucleic acid sequences, regions, elements or domains means that the nucleic acid regions are functionally related to each other. For example, nucleic acid encoding a leader peptide can be operably linked to nucleic acid encoding a polypeptide, whereby the nucleic acids can be transcribed and translated to express a functional fusion protein, wherein the leader peptide effects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods. As used herein, production by recombinant means by using recombinant DNA methods means the use of the well-known methods of molecular biology for expressing proteins encoded by cloned DNA.

As used herein, “expression” refers to the process by which polypeptides are produced by transcription and translation of polynucleotides. The level of expression of a polypeptide can be assessed using any method known in art, including, for example, methods of determining the amount of the polypeptide produced from the host cell. Such methods can include, but are not limited to, quantitation of the polypeptide in the cell lysate by ELISA, Coomassie blue staining following gel electrophoresis, Lowry protein assay and Bradford protein assay.

As used herein, a “host cell” is a cell that is used in to receive, maintain, reproduce and amplify a vector. A host cell also can be used to express the polypeptide encoded by the vector. The nucleic acid contained in the vector is replicated when the host cell divides, thereby amplifying the nucleic acids.

As used herein, a “vector” is a replicable nucleic acid from which one or more heterologous proteins can be expressed when the vector is transformed into an appropriate host cell. Reference to a vector includes those vectors into which a nucleic acid encoding a polypeptide or fragment thereof can be introduced, typically by restriction digest and ligation. Reference to a vector also includes those vectors that contain nucleic acid encoding a polypeptide. The vector is used to introduce the nucleic acid encoding the polypeptide into the host cell for amplification of the nucleic acid or for expression/display of the polypeptide encoded by the nucleic acid. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

As used herein, a vector also includes “virus vectors” or “viral vectors.” Viral vectors are engineered viruses that are operatively linked to exogenous genes to transfer (as vehicles or shuttles) the exogenous genes into cells.

As used herein, an “expression vector” includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, the term “disease” refers to any condition or disorder that damages or interferes with the normal function of a cell, tissue, or organ. Examples of diseases include neoplasia or pathogen infection of cell.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease (e.g., a neoplasia), or otherwise reduce the pathological consequences of the disease (e.g., a neoplasia). The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the engineered immune cells administered.

As used herein, the term “neoplasia” refers to a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasia can affect a variety of cell types, tissues, or organs, including but not limited to an organ selected from the group consisting of bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells).

As used herein, the term “heterologous nucleic acid molecule or polypeptide” refers to a nucleic acid molecule (e.g., a cDNA, DNA or RNA molecule) or polypeptide that is not normally present in a cell or sample obtained from a cell. This nucleic acid can be from another organism, or it can be, for example, an mRNA molecule that is not normally expressed in a cell or sample.

As used herein, the term “immunoresponsive cell” refers to a cell that functions in an immune response or a progenitor, or progeny thereof.

As used herein, the term “modulate” refers positively or negatively alter. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

As used herein, the term “isolated cell” refers to a cell that is separated from the molecular and/or cellular components that naturally accompany the cell.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “secreted” is meant a polypeptide that is released from a cell via the secretory pathway through the endoplasmic reticulum, Golgi apparatus, and as a vesicle that transiently fuses at the cell plasma membrane, releasing the proteins outside of the cell. Small molecules, such as drugs, can also be secreted by diffusion through the membrane to the outside of cell.

As used herein, the term “specifically binds” or “specifically binds to” or “specifically target” is meant a polypeptide or fragment thereof that recognizes and binds a biological molecule of interest (e.g., a polypeptide), but which does not substantially recognize and bind other molecules in a sample, for example, a biological sample, which includes or expresses a tumor antigen.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “subject” refers to any animal (e.g., a mammal), including, but not limited to, humans, non-human primates, rodents, and the like (e.g., which is to be the recipient of a particular treatment, or from whom cells are harvested).

### Overview

Adoptive transfer of engineered T cells has been shown to be an effective therapy for various diseases, such as cancer and infectious diseases. However, immunosuppression by Tregs and Treg-like cells presents a major obstacle for successful immunotherapy. While greater potency and mechanisms are needed to defeat the immunosuppressive disease microenvironment, improved protocols are also needed for decreasing immunosuppressive effects of Tregs present during manufacturing of engineered immune cells ex vivo prior to adoptive transfer to the patient. Given the role of Foxp3 in the immunosuppressive functions of Tregs, it is a selective and ideal target for eliminating Tregs and Treg-like cells. Accordingly, the addition of FoxP3 targeting agents to the manufacturing process for engineered immune cells can deplete the number of FoxP3 positive immunosuppressive cells in the sample, thereby enriching for FoxP3 negative immune activator cells. Provided herein are compositions comprising engineered immune cells and FoxP3 targeting agents and methods of use thereof, that address these issues.

In addition, provided herein are compositions comprising engineered receptors (e.g., vectors comprising polynucleotides encoding an engineered receptor, polynucleotides encoding an engineered receptor, engineered immune cells expressing an engineered receptor) and FoxP3 targeting agents and methods of using such compositions for the manufacture of an engineered immune cell. Without intending to be bound by theory, the use of a FoxP3 targeting agent in the process of producing an engineered immune cell is expected to increase the yield of engineered immune cells that are immune activator cells and/or reduce the yield of engineered immune cells that are FoxP3⁺ immunosuppressant cells. Because samples for producing an engineered immune cell often contain mixtures of immune activator cells and immunosuppressant cells, the resulting engineered immune cells are also mixtures of immune activator cells and immunosuppressant cells. By treating the sample with a FoxP3 targeting agent, the FoxP3+ immunosuppressant cells are depleted from the sample, which results in a higher yield of engineered immune cells that are immune activator cells and/or reduced yield of engineered immune cells that are immunosuppressant cells.

In some embodiments, the engineered immune cells provided herein express a T-cell receptor (TCR) or other cell-surface ligand that binds to a target antigen, such as a tumor antigen or viral protein. In some embodiments, the T cell receptor is a wild-type, or native, T-cell receptor. In some embodiments, the TCR is an engineered receptor. In some embodiments, the engineered receptor is an engineered TCR (eTCR). In some embodiments, the engineered receptor is a chimeric antibody TCR (caTCR). In some embodiments, the engineered receptor is a chimeric antigen receptor (CAR).

In exemplary embodiments, the engineered immune cells provided herein express an engineered receptor (e.g., a CAR, caTCR, or eTCR) or other cell-surface ligand that binds to a Wilms' tumor protein 1 (WT1) tumor antigen. In some embodiments, the engineered immune cells provided herein express an engineered receptor (e.g., a CAR, caTCR, or eTCR) or other cell-surface ligand that binds to a WT1 tumor antigen presented in the context of an WIC molecule. In some embodiments, the engineered immune cells provided herein express an engineered receptor (e.g., a CAR, caTCR, or eTCR) or other cell-surface ligand that binds to a WT1 tumor antigen presented in the context of an HLA-A2 molecule. WT1 is an important, validated, and NCI-top ranked, cancer target antigen. WT1 is a zinc finger transcription factor essential to the embryonal development of the urogenital system. WT1 is highly expressed in most leukemias including AML, CML, ALL and MDS as well as in myeloma and several solid tumors, particularly ovarian carcinoma and mesothelioma. WT1 vaccines have advanced into clinical trials for patients with a variety of cancers. WT1 is distinguished by its importance to the survival of clonogenic leukemic cells, and the ability to treat tumors with T-cells specific for WT1 peptides in xenografted NOD/SCID mice, without adversely affecting normal hematopoiesis. WT1 peptide vaccination has been associated with complete or partial remissions of disease and prolonged survival.

In exemplary embodiments, the engineered immune cells provided herein express an engineered receptor (e.g., a CAR, caTCR, or eTCR) or other cell-surface ligand that binds to the Receptor tyrosine kinase-like Orphan Receptor 2 (ROR2). In some embodiments, the engineered immune cells provided herein express an engineered receptor (e.g., a CAR, caTCR, or eTCR) or other cell-surface ligand that binds to ROR2 presented in the context of an MHC molecule. In some embodiments, the engineered immune cells provided herein express an engineered receptor (e.g., a CAR, caTCR, or eTCR) or other cell-surface ligand that binds to ROR2 presented in the context of an HLA-A2 molecule. ROR2 is a Type-I transmembrane receptor tyrosine kinase important in developmental biology. The extracellular region of ROR2 contains an immunoglobulin (Ig) domain, a cysteine-rich domain (CRD), also called a Frizzled domain, and a Kringle (Kr) domain. All three domains are involved in protein-protein interactions. Intracellularly, ROR2 possesses a tyrosine kinase (TK) domain and a proline-rich domain (PRD) straddled by two serine/threonine-rich domains. ROR2 is normally expressed at high levels during development, playing a key role in skeletal and neural organogenesis, but then expression is suppressed in adult tissues. ROR2 has been shown to play a role in establishing cellular polarity and in tumor-like behavior, such as cell migration and cell invasiveness. ROR2 is highly expressed in several types of human cancer tissues, such as OS, renal cell carcinoma, gastric cancer, malignant melanoma, oral squamous cell carcinoma, prostate cancer, leimyosarcoma, Gastrointestinal Stromal Tumor (GIST), and NB. ROR2 is transactivated in a majority of OS, and knockdown of ROR2 in OS cell lines results in significantly inhibited cell proliferation, migration and invasion. Evidence links Wnt5a and ROR2 within OS, where ROR2 has an additional role in the degradation of the extracellular matrix and invadopodia formation. Research has also shown that expression of ROR2 tends to increase as the degree of malignancy rises in oral squamous cell carcinoma and in metastatic nodules of melanoma. In a xenograft metastasis model, silencing ROR2 significantly decreased lung metastasis of melanoma cells. Like its mouse counterpart, human ROR2 expression cannot be detected in normal adult tissues, except for low levels in stomach and thyroid. Overexpression of ROR2 appears to strongly correlate with poor survival in patients with NB. This differential expression of ROR2 between human cancers and normal tissues makes it an excellent therapeutic target.

In exemplary embodiments, the engineered immune cells provided herein express an engineered receptor (e.g., a CAR, caTCR, or eTCR) or other cell-surface ligand that binds to the cluster of differentiation 19 (CD19). Exemplary engineered receptors that bind to CD19 are described in International Publication No. WO2017070608, which is incorporated by reference in its entirety.

In exemplary embodiments, the engineered immune cells provided herein express an engineered receptor (e.g., a CAR, caTCR, or eTCR) or other cell-surface ligand that binds to the alpha-fetoprotein (AFP). In some embodiments, the engineered immune cells provided herein express an engineered receptor (e.g., a CAR, caTCR, or eTCR) or other cell-surface ligand that binds to AFP presented in the context of an MEW molecule. In some embodiments, the engineered immune cells provided herein express an engineered receptor (e.g., a CAR, caTCR, or eTCR) or other cell-surface ligand that binds to AFP presented in the context of an HLA-A2 molecule. Exemplary engineered receptors that bind to AFP are described in International Publication No. WO2016161390, which is incorporated by reference in its entirety.

In exemplary embodiments, the FoxP3 targeting agents provided herein are antigen-binding proteins, including antibodies, chimeric antigen receptors (CARs), chimeric antibody TCRs (caTCRs), and engineered TCRs (eTCRS) specific for a FoxP3 polypeptide. In some embodiments, the FoxP3 targeting agent is specific for an epitope of the FoxP3 polypeptide. In some embodiments, the FoxP3 targeting agent binds to FoxP3 presented in the context of an MHC molecule (e.g., FoxP3/MHC complex). In some embodiments, the FoxP3 targeting agent binds to FoxP3 presented in the context of an HLA-A molecule (e.g., FoxP3/HLA-A complex). In some embodiments, the FoxP3 targeting agent binds to FoxP3 presented in the context of an HLA-A2 molecule (e.g., FoxP3/HLA-A2 complex). In some embodiments, the FoxP3 targeting agent binds to FoxP3 presented in the context of an HLA-A*02:01 molecule (e.g., FoxP3/HLA-A*02:01 complex).

In exemplary embodiments, the FoxP3 targeting agents provided herein are bispecific antibodies. In some embodiments, the bispecific antibody binds to a FoxP3 polypeptide, or fragment thereof, and a cell surface protein. In some embodiments, cell surface protein is CD3 or CD16.

In exemplary embodiments, the FoxP3 targeting agents are engineered immune cells that express an engineered receptor (e.g., a CAR, caTCR, or eTCR) or other cell-surface ligand that binds to FoxP3. In some embodiments, the FoxP3 targeting agents are engineered immune cells that express an engineered receptor (e.g., a CAR, caTCR, or eTCR) or other cell-surface ligand that binds to FoxP3 presented in the context of an MHC molecule. In some embodiments, the FoxP3 targeting agents are engineered immune cells that express an engineered receptor (e.g., a CAR, caTCR, or eTCR) or other cell-surface ligand that binds to FoxP3 presented in the context of an HLA-A2 molecule.

### Targeting Ligands and Target Antigens of Engineered Immune Cells

In some embodiments, the engineered immune cells provided herein express a T-cell receptor (TCR) or other cell-surface ligand that binds to a target antigen (i.e., cell surface antigen), such as a tumor antigen or viral protein. The cell-surface ligand can be any molecule that directs an immune cell to a target site (e.g., a tumor site). Exemplary cell surface ligands include, for example endogenous receptors, engineered receptors, or other specific ligands to achieve targeting of the immune cell to a target site. In some embodiments, the receptor is a T cell receptor. In some embodiments, the T cell receptor is a wild-type, or native, T-cell receptor that binds to a target antigen. In some embodiments, the receptor, e.g. a T cell receptor, is non-native receptor (e.g., not endogenous to the immune cells). In some embodiments, the TCR is an engineered receptor. In some embodiments, the engineered receptor is an engineered TCR (eTCR). In some embodiments, the engineered receptor is a chimeric antibody TCR (caTCR). In some embodiments, the engineered receptor is a chimeric antigen receptor (CAR).

In some embodiments, the target antigen (i.e., cell surface antigen) cell surface antigen is selected from the group consisting of protein, carbohydrate, and lipid. In some embodiments, the target antigen (i.e., cell surface antigen) is expressed by a tumor cell. In some embodiments, the target antigen is expressed on the surface of a tumor cell. In some embodiments, the target antigen is a cell surface receptor. In some embodiments, the target antigen is a cell surface glycoprotein. In some embodiments, the target antigen is secreted by a tumor cell. In some embodiments, the target antigen is localized to the tumor microenvironment. In some embodiments, the target antigen is localized to the extracellular matrix or stroma of the tumor microenvironment. In some embodiments, the target antigen is expressed by one or more cells located within the extracellular matrix or stroma of the tumor microenvironment.

In some embodiments, the target antigen (i.e., cell surface antigen) is selected from among 5T4, alpha 5β1-integrin, 707-AP, A33, AFP, ART-4, B7H4, BAGE, Bcl-2, β-catenin, Bcr-Abl, MN/C IX antibody, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, KRAS, LAGE, LDLR/FUT, LRP, LMP2, MAGE, MART, MART-1/melan-A, MART-2/Ski, MC1R, mesothelin, MUC, MUM-1-B, myc, MUM-2, MUM-3, NA88-A, NYESO-1, NY-Eso-B, p53, PD1, proteinase-3, p190 minor bcr-abl, Pml/RARa, PRAME, progesterone receptor, PSA, PSM, PSMA, ras, RAGE, RU1 or RU2, RORI, ROR2, SART-1 or SART-3, survivin, TEL/AML1, TGFβ, TPI/m, TRP-1, TRP-2, TRP-2/INT2, tenascin, TSTA tyrosinase, VEGF, and WT1. In certain embodiments, the target antigen is selected from among ROR2, WT1, preferentially expressed antigen of melanoma (PRAME), Kirsten rat sarcoma viral oncogene (KRAS), programmed cell death 1 (PD1), latent membrane protein 2 (LMP2), and alpha-fetoprotein (AFP). In some embodiments, the target antigen (i.e., cell surface antigen) is selected from the group consisting of CD19, CD20, CD47, GPC-3, ROR1, ROR2, BCMA, GPRC5D, and FCRL5). In some embodiments, the target antigen in CD19. In some embodiments, the target antigen (i.e., cell surface antigen) comprises a peptide and a major histocompatibility complex (MHC) protein. In some embodiments, the peptide is derived from a protein selected from the group consisting of WT-1, AFP, HPV16-E7, NY-ESO-1, PRAME, EBV-LMP2A, HIV-1, KRAS, Histone H3.3, and PSA. In some embodiments, the peptide is derived from.

Exemplary target antigens and epitopes within the target antigens that can be bound by a TCR or other cell-surface ligand expressed on an engineered immune cell are described in, e.g., WO2015/070061, WO2016/142768, WO2015/011450, WO2017/070608, WO2017/066136, WO2016/191246, WO2016/165047, WO2016/210129, WO2016/201124, WO2016/161390, which are incorporated by reference in their entirety, including the sequence listings provided therein.

In some embodiments, the target antigen is ROR2. The DNA sequence encoding one embodiment of human ROR2 is provided herein as SEQ ID NO: 328, as follows:

[SEQ ID NO: 328]
ATGGCCCGGGGCTCGGCGCTCCCGCGGCGGCCGCTGCTGTGCATCCCGGCCGTCT
GGGCGGCCGCCGCGCTTCTGCTCTCAGTGTCCCGGACTTCAGGTGAAGTGGAGGT
TCTGGATCCGAACGACCCTTTAGGACCCCTTGATGGGCAGGACGGCCCGATTCCA
ACTCTGAAAGGTTACTTTCTGAATTTTCTGGAGCCAGTAAACAATATCACCATTG
TCCAAGGCCAGACGGCAATTCTGCACTGCAAGGTGGCAGGAAACCCACCCCCTA
ACGTGCGGTGGCTAAAGAATGATGCCCCGGTGGTGCAGGAGCCGCGGCGGATCA
TCATCCGGAAGACAGAATATGGTTCACGACTGCGAATCCAGGACCTGGACACGA
CAGACACTGGCTACTACCAGTGCGTGGCCACCAACGGGATGAAGACCATTACCG
CCACTGGCGTCCTGTTTGTGCGGCTGGGTCCAACGCACAGCCCAAATCATAACTT
TCAGGATGATTACCACGAGGATGGGTTCTGCCAGCCTTACCGGGGAATTGCCTGT
GCACGCTTCATTGGCAACCGGACCATTTATGTGGACTCGCTTCAGATGCAGGGGG
AGATTGAAAACCGAATCACAGCGGCCTTCACCATGATCGGCACGTCTACGCACC
TGTCGGACCAGTGCTCACAGTTCGCCATCCCATCCTTCTGCCACTTCGTGTTTCCT
CTGTGCGACGCGCGCTCCCGGACACCCAAGCCGCGTGAGCTGTGCCGCGACGAG
TGCGAGGTGCTGGAGAGCGACCTGTGCCGCCAGGAGTACACCATCGCCCGCTCC
AACCCGCTCATCCTCATGCGGCTTCAGCTGCCCAAGTGTGAGGCGCTGCCCATGC
CTGAGAGCCCCGACGCTGCCAACTGCATGCGCATTGGCATCCCAGCCGAGAGGC
TGGGCCGCTACCATCAGTGCTATAACGGCTCAGGCATGGATTACAGAGGAACGG
CAAGCACCACCAAGTCAGGCCACCAGTGCCAGCCGTGGGCCCTGCAGCACCCCC
ACAGCCACCACCTGTCCAGCACAGACTTCCCTGAGCTTGGAGGGGGGCACGCCT
ACTGCCGGAACCCCGGAGGCCAGATGGAGGGCCCCTGGTGCTTTACGCAGAATA
AAAACGTACGCATGGAACTGTGTGACGTACCCTCGTGTAGTCCCCGAGACAGCA
GCAAGATGGGGATTCTGTACATCTTGGTCCCCAGCATCGCAATTCCACTGGTCAT
CGCTTGCCTTTTCTTCTTGGTTTGCATGTGCCGGAATAAGCAGAAGGCATCTGCG
TCCACACCGCAGCGGCGACAGCTGATGGCCTCGCCCAGCCAAGACATGGAAATG
CCCCTCATTAACCAGCACAAACAGGCCAAACTCAAAGAGATCAGCCTGTCTGCG
GTGAGGTTCATGGAGGAGCTGGGAGAGGACCGGTTTGGGAAAGTCTACAAAGGT
CACCTGTTCGGCCCTGCCCCGGGGGAGCAGACCCAGGCTGTGGCCATCAAAACG
CTGAAGGACAAAGCGGAGGGGCCCCTGCGGGAGGAGTTCCGGCATGAGGCTATG
CTGCGAGCACGGCTGCAACACCCCAACGTCGTCTGCCTGCTGGGCGTGGTGACC
AAGGACCAGCCCCTGAGCATGATCTTCAGCTACTGTTCGCACGGCGACCTCCACG
AATTCCTGGTCATGCGCTCGCCGCACTCGGACGTGGGCAGCACCGATGATGACC
GCACGGTGAAGTCCGCCCTGGAGCCCCCCGACTTCGTGCACCTTGTGGCACAGAT
CGCGGCGGGGATGGAGTACCTATCCAGCCACCACGTGGTTCACAAGGACCTGGC
CACCCGCAATGTGCTAGTGTACGACAAGCTGAACGTGAAGATCTCAGACTTGGG
CCTCTTCCGAGAGGTGTATGCCGCCGATTACTACAAGCTGCTGGGGAACTCGCTG
CTGCCTATCCGCTGGATGGCCCCAGAGGCCATCATGTACGGCAAGTTCTCCATCG
ACTCAGACATCTGGTCCTACGGTGTGGTCCTGTGGGAGGTCTTCAGCTACGGCCT
GCAGCCCTACTGCGGGTACTCCAACCAGGATGTGGTGGAGATGATCCGGAACCG
GCAGGTGCTGCCTTGCCCCGATGACTGTCCCGCCTGGGTGTATGCCCTCATGATC
GAGTGCTGGAACGAGTTCCCCAGCCGGCGGCCCCGCTTCAAGGACATCCACAGC
CGGCTCCGAGCCTGGGGCAACCTTTCCAACTACAACAGCTCGGCGCAGACCTCG
GGGGCCAGCAACACCACGCAGACCAGCTCCCTGAGCACCAGCCCAGTGAGCAAT
GTGAGCAACGCCCGCTACGTGGGGCCCAAGCAGAAGGCCCCGCCCTTCCCACAG
CCCCAGTTCATCCCCATGAAGGGCCAGATCAGACCCATGGTGCCCCCGCCGCAG
CTCTACGTCCCCGTCAACGGCTACCAGCCGGTGCCGGCCTATGGGGCCTACCTGC
CCAACTTCTACCCGGTGCAGATCCCAATGCAGATGGCCCCGCAGCAGGTGCCTCC
TCAGATGGTCCCCAAGCCCAGCTCACACCACAGTGGCAGTGGCTCCACCAGCAC
AGGCTACGTCACCACGGCCCCCTCCAACACATCCATGGCAGACAGGGCAGCCCT
GCTCTCAGAGGGCGCTGATGACACACAGAACGCCCCAGAAGATGGGGCCCAGAG
CACCGTGCAGGAAGCAGAGGAGGAGGAGGAAGGCTCTGTCCCAGAGACTGAGC
TGCTGGGGGACTGTGACACTCTGCAGGTGGACGAGGCCCAAGTCCAGCTGGAAG
CTTGA.

The polypeptide sequence of one embodiment of human ROR2 is provided herein as SEQ ID NO: 329, as follows:

[SEQ ID NO: 329]
MARGSALPRRPLLCIPAVWAAAALLLSVSRTSGEVEVLDPNDPLGPLDGQDGPIPTL
KGYFLNFLEPVNNITIVQGQTAILHCKVAGNPPPNVRWLKNDAPVVQEPRRIIIRKTE
YGSRLRIQDLDTTDTGYYQCVATNGMKTITATGVLFVRLGPTHSPNHNFQDDYHED
GFCQPYRGIACARFIGNRTIYVDSLQMQGEIENRITAAFTMIGTSTHLSDQCSQFAIPSF
CHFVFPLCDARSRTPKPRELCRDECEVLESDLCRQEYTIARSNPLILMRLQLPKCEALP
MPESPDAANCMRIGIPAERLGRYHQCYNGSGMDYRGTASTTKSGHQCQPWALQHP
HSHHLSSTDFPELGGGHAYCRNPGGQMEGPWCFTQNKNVRMELCDVPSCSPRDSSK
MGILYILVPSIAIPLVIACLFFLVCMCRNKQKASASTPQRRQLMASPSQDMEMPLINQ
HKQAKLKEISLSAVRFMEELGEDRFGKVYKGHLFGPAPGEQTQAVAIKTLKDKAEG
PLREEFRHEAMLRARLQHPNVVCLLGVVTKDQPLSMIFSYCSHGDLHEFLVMRSPHS
DVGSTDDDRTVKSALEPPDFVHLVAQIAAGMEYLSSHHVVHKDLATRNVLVYDKL
NVKISDLGLFREVYAADYYKLLGNSLLPIRWMAPEAIMYGKFSIDSDIWSYGVVLWE
VFSYGLQPYCGYSNQDVVEMIRNRQVLPCPDDCPAWVYALMIECWNEFPSRRPRFK
DIHSRLRAWGNLSNYNSSAQTSGASNTTQTSSLSTSPVSNVSNARYVGPKQKAPPFP
QPQFIPMKGQIRPMVPPPQLYVPVNGYQPVPAYGAYLPNFYPVQIPMQMAPQQVPPQ
MVPKPSSUESGSGSTSTGYVTTAPSNTSMADRAALLSEGADDTQNAPEDGAQSTVQ
EAEEEEEGSVPETELLGDCDTLQVDEAQVQLEA.

In some embodiments, the target antigen is an epitope of ROR2. In some embodiments, the epitope of ROR2 has an amino acid sequence selected from KTITATGVLFVRLGP (SEQ ID NO: 330), TGYYQCVATNGMKTI (SEQ ID NO: 331), RGIACARFIGNRTIY (SEQ ID NO: 332), CQPYRGIACARFIGNRTIY (SEQ ID NO: 333), QCSQFAIPSFCHFVFPLCD (SEQ ID NO: 334), ELCRDECEVLESDLC (SEQ ID NO: 335), and ANCMRIGIPAERLGR (SEQ ID NO: 336). In some embodiments, the epitope is KTITATGVLFVRLGP (SEQ ID NO: 330).

In some embodiments, the target antigen is an extracellular domain of ROR2 or a fragment thereof. In one embodiment, the amino acid sequence of the extracellular domain of ROR2 is described herein as SEQ ID NO: 337, as follows:

(SEQ ID NO: 337)
EVEVLDPNDPLGPLDGQDGPIPTLKGYFLNFLEPVNNITIVQGQTAILHC
KVAGNPPPNVRWLKNDAPVVQEPRRIIIRKTEYGSRLRIQDLDTTDTGYY
QCVATNGMKTITATGVLFVRLGPTHSPNHNFQDDYHEDGFCQPYRGIACA
RFIGNRTIYVDSLQMQGEIENRITAAFTMIGTSTHLSDQCSQFAIPSFCH
FVFPLCDARSRTPKPRELCRDECEVLESDLCRQEYTIARSNPLILMRLQL
PKCEALPMPESPDAANCMRIGIPAERLGRYHQCYNGSGMDYRGTASTTKS
GHQCQPWALQHPHSHHLSSTDFPELGGGHAYCRNPGGQMEGPWCFTQNKN
VRMELCDVPSCSPRDSSKMG.

In some embodiments, the target antigen is WT1. In some embodiments, the target antigen is an epitope of WT1. In some embodiments, the epitope of WT1 has the amino acid sequence of RMFPNAPYL (SEQ ID NO: 190).

In some embodiments, the target antigen-associated disease is cancer. In some embodiments, the cancer is selected from among acute lymphoblastic leukemia (ALL), acute myeloid/myelogenous leukemia (AML), adrenocortical carcinoma, bladder cancer, brain tumor, breast cancer, cervical cancer, cholangiocarcinoma, chronic myelocytic leukemia (CML), chronic osteosarcoma, colorectal cancers, esophageal cancer, gastrointestinal cancer, glioblastoma, glioma, hepatocellular carcinoma, head and neck cancer, kidney cancer, lymphoma, leukemia, lung cancer, melanoma, mesothelioma, multiple myeloma (MM), myelodysplastic syndrome (MDS), neuroblastoma, oral squamous cell carcinoma, osteosarcoma, ovarian cancer, pancreatic cancer, pheochromocytoma, plasmacytoma, prostate cancer, renal cancer, sarcoma, stomach cancer, thyroid cancer, and uterine cancer.

In some embodiments, the target antigen-associated disease is viral infection. In some embodiments, the viral infection is caused by a virus selected from the group consisting of Cytomegalovirus (CMV), Epstein-Barr Virus (EBV), Hepatitis B Virus (HBV), Kaposi's Sarcoma associated herpesvirus (KSHV), Human papillomavirus (HPV), Molluscum contagiosum virus (MCV), Human T cell leukemia virus 1 (HTLV-1), HIV (Human immunodeficiency virus), and Hepatitis C Virus (HCV).

Examples of CD19 positive cancers include, but are not limited to, B-cell lymphoma. Examples of B-cell lymphomas include Hodgkin's lymphomas and non-Hodgkin's lymphomas. Examples of non-Hodgkin's lymphomas include diffuse large B-cell lymphoma (DLBCL), follicular lymphoma, marginal zone B-cell lymphoma (MZL) or Mucosa-Associated Lymphatic Tissue lymphoma (MALT), small lymphocytic lymphoma (also known as chronic lymphocytic leukemia, CLL), and mantle cell lymphoma (MCL).

Examples of AFP positive cancers include, but are not limited to, liver cancer and nonseminomatous germ cell tumors of the ovary and testis. Examples of liver cancer include hepatocellular carcinoma and hepatoblastoma. Examples of nonseminomatous germ cell tumors of the ovary and testis include yolk sac and embryonal carcinoma.

Examples of ROR2 positive cancers include, but are not limited to, chronic OS, renal cell carcinoma, gastric cancer, malignant melanoma, oral squamous cell carcinoma, prostate cancer, osteosarcoma, and neuroblastoma.

Examples of WT1 positive cancers include, but are not limited to, chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma.

Typical therapeutic anti-cancer mAb, like those that bind to CD19, recognize cell surface proteins, which constitute only a tiny fraction of the cellular protein content. Most mutated or oncogenic tumor associated proteins are typically nuclear or cytoplasmic. In certain instances, these intracellular proteins can be degraded in the proteasome, processed and presented on the cell surface by WIC class I molecules as T cell epitopes that are recognized by T cell receptors (TCRs). The development of mAb that mimic TCR function, “TCR mimic (TCRm)” or “TCR-like”; (i.e., that recognize peptide antigens of key intracellular proteins in the context of MHC on the cell surface) greatly extends the potential repertoire of tumor targets addressable by potent mAb. TCRm Fab, or scFv, and mouse IgG specific for the melanoma Ags, NY-ESO-1, hTERT, MART 1, gp100, and PR1, among others, have been developed. The antigen binding portions of such antibodies can be incorporated into the engineered receptors provided herein. HLA-A2 is the most common HLA haplotype in the USA and EU (about 40% of the population). Therefore, potent TCRm mAb and native TCRs against tumor antigens presented in the context of HLA-A2 are useful in the treatment of a large populations.

Accordingly, in some embodiments, a target antigen is a tumor antigen presented in the context of an MHC molecule. In some embodiments, the MHC protein is a MHC class I protein. In some embodiments, the MHC Class I protein is an HLA-A, HLA-B, or HLA-C molecules. In some embodiments, target antigen is a tumor antigen presented in the context of an HLA-A2 molecule. mAbs for intracellular WT1 and ROR2 antigens presented in the context of surface HLA-A2 molecules have previously been developed. IgG1, afucosylated Fc forms, bispecific antibodies and engineered T cell formats have been made that exhibit potent therapeutic activity in multiple preclinical animal models. Such antibodies or portion thereof can be employed as described herein for the recognition of target antigens present on the surface of a target cell (e.g., a tumor cell) in the context of an MHC molecule.

### Engineered Receptors

In some embodiments, the engineered immune cells provided herein express at least one engineered receptor (e.g., CAR, caTCR, eTCR). In some embodiments, the engineered receptor grafts or confers a specificity of interest onto an immune effector cell. For example, engineered receptors can be used to graft the specificity of a monoclonal antibody onto an immune cell, such as a T cell. In some embodiments, transfer of the coding sequence of the engineered is facilitated by nucleic acid vector, such as a retroviral vector.

In some embodiments, the engineered receptor is a CAR. There are currently three generations of CARs. In some embodiments, the engineered immune cells provided herein express a “first generation” CAR. “First generation” CARs are typically composed of an extracellular antigen binding domain (e.g., a single-chain variable fragment (scFv)) fused to a transmembrane domain fused to cytoplasmic/intracellular domain of the T cell receptor (TCR) chain. “First generation” CARs typically have the intracellular domain from the CD3ζ chain, which is the primary transmitter of signals from endogenous TCRs. “First generation” CARs can provide de novo antigen recognition and cause activation of both CD4⁺ and CD8⁺ T cells through their CD3ζ chain signaling domain in a single fusion molecule, independent of HLA-mediated antigen presentation.

In some embodiments, the engineered immune cells provided herein express a “second generation” CAR. “Second generation” CARs add intracellular domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-IBB) and activation (e.g., CD3). Preclinical studies have indicated that “Second Generation” CARs can improve the antitumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL).

In some embodiments, the engineered immune cells provided herein express a “third generation” CAR. “Third generation” CARs comprise those that provide multiple co-stimulation (e.g., CD28 and 4-1BB) and activation (e.g., CD3).

In accordance with the presently disclosed subject matter, the CARs of the engineered immune cells provided herein comprise an extracellular antigen-binding domain, a transmembrane domain and an intracellular domain.

In some embodiments, the engineered receptor is a caTCR. In some embodiments, the caTCR does not in itself comprise a TCR-associated signaling molecule (such as CD3δc, CD3γε, and/or CD3ζζ), at least not a functional one or a functional fragment of one. In some embodiments, the caTCR comprises an antigen-binding module (i.e., extracellular antigen binding domain) that provides the antigen specificity and a T cell receptor module (TCRM) that allows for CD3 recruitment and signaling. The antigen-binding module (i.e., extracellular antigen binding domain) is not a naturally occurring T cell receptor antigen-binding moiety. In some embodiments, the antigen-binding module (i.e., extracellular antigen binding domain) is linked to the amino terminus of a polypeptide chain in the TCRM. In some embodiments, the antigen-binding module (i.e., extracellular antigen binding domain) is an antibody moiety. In some embodiments, the antibody moiety is a Fab, a Fab′, a (Fab′)2, an Fv, or a single chain Fv (scFv). The TCRM comprises a transmembrane module derived from the transmembrane domains of one or more TCRs (TCR-TMs), such as an aβ and/or γδ TCR, and optionally further comprises one or both of the connecting peptides or fragments thereof of a TCR and/or one or more TCR intracellular domains or fragments thereof. In some embodiments, the TCRM comprises two polypeptide chains, each polypeptide chain comprising, from amino terminus to carboxy terminus, a connecting peptide, a transmembrane domain, and optionally a TCR intracellular domain. In some embodiments, the TCRM comprises one or more non-naturally occurring TCR domains. For example, in some embodiments, the TCRM comprises one or two non-naturally occurring TCR transmembrane domains. A non-naturally occurring TCR domain can be a corresponding domain of a naturally occurring TCR modified by substitution of one or more amino acids, and/or by replacement of a portion of the corresponding domain with a portion of an analogous domain from another TCR. The caTCR can comprise a first polypeptide chain and a second polypeptide chain, wherein the first and second polypeptide chains together form the antigen-binding module and the TCRM. In some embodiments, the first and second polypeptide chains are separate polypeptide chains, and the caTCR is a multimer, such as a dimer. In some embodiments, the first and second polypeptide chains are covalently linked, such as by a peptide linkage, or by another chemical linkage, such as a disulfide linkage. In some embodiments, the first polypeptide chain and the second polypeptide chain are linked by at least one disulfide bond. In some embodiments, the caTCR further comprises one or more T cell co-stimulatory signaling sequences. Examples of caTCRs are described in, for example, International Publication No. WO2017/070608 and U.S. Provisional Application No. 62/490,576, filed Apr. 26, 2017, both of which are incorporated by reference in their entireties.

In some embodiments, the engineered receptor is an eTCR. In some embodiments, an eTCR differs from a naturally occurring TCR in that the antigen/WIC-binding region of the naturally occurring TCR is modified. In some embodiments, an eTCR comprises an alpha chain TRAC constant domain sequence and/or a beta chain TRBC1 or TRBC2 constant domain sequence. In some embodiments, the alpha and beta chain constant domain sequences are modified by truncation or substitution to delete the native disulfide bond between Cys4 of exon 2 of TRAC and Cys2 of exon 2 of TRBC1 or TRBC2. The alpha and/or beta chain constant domain sequence(s) can also be modified by substitution of cysteine residues for Thr 48 of TRAC and Ser 57 of TRBC1 or TRBC2, the said cysteines forming a disulfide bond between the alpha and beta constant domains of the TCR. eTCRs can be in single chain format, for example see WO 2004/033685. Single chain formats include β TCR polypeptides of the V-L-vβ, vβ-L-V, V-C-L-vβ, Va-L-Vb-Cb, V-C-L-Vb-Cb types, wherein Va and Vb are TCR alpha and beta variable regions respectively, Ca and Cb are TCR alpha and beta constant regions respectively, and L is a linker sequence. In certain embodiments single chain eTCRs can have an introduced disulfide bond between residues of the respective constant domains, as described in WO 2004/033685. Examples of eTCRs are described, for example, in International Publication No. WO2015/011450, which is incorporated by reference in its entirety.

### Extracellular Antigen-Binding Domain of an Engineered Receptor

In some embodiments, the extracellular antigen-binding domain of an engineered receptor (e.g., CAR, caTCR, eTCR) binds to a target antigen (i.e., cell surface antigen). In certain embodiments, the extracellular antigen-binding domain of an engineered receptor specifically binds a tumor antigen. In certain embodiments, the extracellular antigen-binding domain is derived from a monoclonal antibody (mAb) that binds to a target antigen (i.e., cell surface antigen, such as tumor antigen or viral protein). In some embodiments, the extracellular antigen-binding domain comprises an scFv. In some embodiments, the extracellular antigen-binding domain comprises a Fab, which is optionally crosslinked. In some embodiments, the extracellular binding domain comprises a F(ab)₂. In some embodiments, any of the foregoing molecules are comprised in a fusion protein with a heterologous sequence to form the extracellular antigen-binding domain. In certain embodiments, the extracellular antigen-binding domain comprises a human scFv that binds specifically to a tumor antigen. In certain embodiments, the scFv is identified by screening scFv phage library with tumor antigen-Fc fusion protein.

In certain embodiments, the extracellular antigen-binding domain of a presently disclosed engineered receptor has a high binding specificity and high binding affinity to a tumor antigen (e.g., a mammalian tumor antigen, such as a human tumor antigen). For example, in some embodiments, the extracellular antigen-binding domain of the engineered receptor (embodied, for example, in a human scFv or an analog thereof) binds to a particular tumor antigen with a dissociation constant (K_{d}) of about 1×10⁻⁵ M or less. In certain embodiments, the K_{d} is about 5×10⁻⁶ M or less, about 1×10⁻⁶ M or less, about 5×10⁻⁷ M or less, about 1×10⁻⁷ M or less, about 5×10⁻⁸ M or less, about 1×10⁻⁸ M or less, about 5×10⁻⁹ or less, about 4×10⁻⁹ or less, about 3×10⁻⁹ or less, about 2×10⁻⁹ or less, about 1×10⁻⁹ M or less, about 1×10⁻¹⁰ or less, about 1×10⁻¹¹ or less, about 1×10⁻¹² or less, about 1×10⁻¹³ or less, about 1×10⁻¹⁴ or less, or about 1×10⁻¹⁵ or less. In certain non-limiting embodiments, the K_{d} is from about 5×10⁻⁷ M or less. In certain non-limiting embodiments, the K_{d} is from about 3×10⁻⁹ M or less. In certain non-limiting embodiments, the K_{d} is from about 1×10⁻¹³ M or less. In certain non-limiting embodiments, the K_{d} is from about 1×10⁻¹³ M to about 5×10⁻⁷ M. In certain non-limiting embodiments, the K_{d} is from about 3×10⁻⁹ to about 2×10⁻⁷.

Binding of the extracellular antigen-binding domain (embodiment, for example, in a human scFv or an analog thereof) of a presently disclosed tumor antigen-targeted engineered receptor can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or a scFv) specific for the complex of interest. For example, the scFv can be radioactively labeled and used in a radioimmunoassay (MA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the tumor antigen-targeted engineered receptor is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet). In certain embodiments, the human scFv of a presently disclosed tumor antigen-targeted engineered receptor is labeled with GFP.

In some embodiments, the extracellular antigen-binding domain of the expressed engineered receptor (e.g., a CAR, caTCR, or eTCR) binds to tumor antigen that is expressed by a tumor cell. In some embodiments, the extracellular antigen-binding domain of the expressed engineered receptor (e.g., a CAR, caTCR, or eTCR) binds to tumor antigen that is expressed on the surface of a tumor cell. In some embodiments, the extracellular antigen-binding domain of the expressed engineered receptor (e.g., a CAR, caTCR, or eTCR) binds to tumor antigen that is expressed on the surface of a tumor cell in combination with an MHC protein. In some embodiments, the MHC protein is a MEW class I protein. In some embodiments, the MEW Class I protein is an HLA-A, HLA-B, or HLA-C molecules. In some embodiments, the extracellular antigen-binding domain of the expressed engineered receptor (e.g., a CAR, caTCR, or eTCR) binds to a target antigen (i.e., cell surface antigen, such as a tumor antigen or viral protein) that is expressed on the surface of a tumor cell not in combination with an MHC protein.

In some embodiments, the extracellular antigen-binding domain of the expressed engineered receptor (e.g., a CAR, caTCR, or eTCR) binds to a protein selected from among 5T4, alpha 5β1-integrin, 707-AP, A33, AFP, ART-4, B7H4, BAGE, Bcl-2, β-catenin, Bcr-Abl, MN/C IX antibody, CA125, CA19-9, CAMEL, CAP-1, CASP-8, CD3, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, KRAS, LAGE, LDLR/FUT, LRP, LMP2, MAGE, MART, MART-1/melan-A, MART-2/Ski, MC1R, mesothelin, MUC, MUM-1-B, myc, MUM-2, MUM-3, NA88-A, NYESO-1, NY-Eso-B, p53, PD1, proteinase-3, p190 minor bcr-abl, Pml/RARα, PRAME, progesterone receptor, PSA, PSM, PSMA, ras, RAGE, RU1 or RU2, RORI, ROR2, SART-1 or SART-3, survivin, TEL/AML1, TGFβ, TPI/m, TRP-1, TRP-2, TRP-2/INT2, tenascin, TSTA tyrosinase, VEGF, and WT1. In certain embodiments, the extracellular antigen-binding domain of the expressed engineered receptor (e.g., a CAR, caTCR, or eTCR) binds to protein selected from among ROR2, WT1, PRAME, KRAS, PD1, LMP2, and AFP, or a fragment thereof. In certain embodiments, the extracellular antigen-binding domain of the expressed engineered receptor (e.g., a CAR, caTCR, or eTCR) binds to ROR2 or a fragment thereof. In certain embodiments, the extracellular antigen-binding domain of the expressed engineered receptor (e.g., a CAR, caTCR, or eTCR) binds to WT1 or a fragment thereof.

In certain embodiments, the TCR or cell-surface ligand binds to two or more target antigens. In some embodiments, the TCR or cell-surface ligand comprises two or more extracellular antigen-binding domains. In some embodiments, the TCR or cell-surface ligand comprises an extracellular antigen-binding domain that is a bispecific antibody. In some embodiments, the bispecific antibody is a trifunctional antibody, chemically linked Fab, or bi-specific T cell engager. In some embodiments, the TCR or cell-surface ligand comprises a first extracellular antigen-binding domain that binds to protein selected from among ROR2, WT1, PRAME, KRAS, PD1, LMP2, AFP, HPV16-E7, NY-ESO-1, EBV-LMP2A, HIV-1, KRAS, Histone H3.3, PSA, CD19, CD20, CD47, GPC-3, ROR1, ROR2, BCMA, GPRC5D, and FCRL5, or a fragment thereof. In some embodiments, the TCR or cell-surface ligand comprises a second extracellular antigen-binding domain that binds to a second target antigen. In some embodiments, the second target antigen is a cell surface protein (e.g., CD3).

Exemplary extracellular antigen-binding domains and methods of generating such domains and associated CARs are described in, e.g., WO2015/070061, WO2016/142768, WO2015/011450, WO2017/070608, WO2016/191246, WO2016/165047, WO2016/210129, WO2016/201124, WO2016/161390, WO2016/191246, WO2017/023859, WO2015/188141, WO2015/070061, WO2012/135854, WO2014/055668, which are incorporated by reference in their entirety, including the sequence listings provided therein.

Extracellular Antigen Binding Domain of an Engineered Receptor that Binds to CD19

In some embodiments, extracellular antigen-binding domain of the expressed engineered receptor (e.g., a CAR, caTCR, or eTCR) binds to CD19.

In certain embodiments, the extracellular antigen-binding domain binds to CD19 or a fragment thereof. In some embodiments, the extracellular antigen binding domain comprises a heavy chain variable region comprising amino acids having a sequence of SEQ ID NO: 101, or a functional fragment or variant thereof. In some embodiments, the extracellular antigen-binding domain (e.g., human scFv) comprises a light chain variable region comprising amino acids having a sequence of SEQ ID NO: 102, or a functional fragment or variant thereof. In some embodiments, the extracellular antigen-binding domain is a human scFv, which comprises a heavy chain variable region comprising amino acids having the sequence set forth SEQ ID NO: 101, or a functional fragment or variant thereof and a light chain variable region comprising amino acids having the sequence set forth in SEQ ID NO: 102, or a functional fragment or variant thereof, optionally with (iii) a linker sequence, for example a linker peptide, between the heavy chain variable region and the light chain variable region. In certain embodiments, the linker comprises amino acids having the sequence set forth in SEQ ID NO: 118 (SRGGGGSGGGGSGGGGSLEMA). In certain embodiments, the extracellular antigen-binding domain is a human scFv-Fc fusion protein or full length human IgG with V_{H} and V_{L} regions.

In certain embodiments, the extracellular antigen-binding domain comprises a V_{H} comprising an amino acid sequence that is at least 80%, at least 85%, at least 90%, or at least 95% identical to SEQ ID NO: 101. For example, the extracellular antigen-binding domain comprises a V_{H} comprising an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 101. In certain embodiments, the extracellular antigen-binding domain comprises a V_{H} comprising amino acids having the sequence set forth in SEQ ID NO: 101. In certain embodiments, the extracellular antigen-binding domain comprises a V_{L} comprising an amino acid sequence that is at least 80%, at least 85%, at least 90%, or at least 95% identical to SEQ ID NO: 101. For example, the extracellular antigen-binding domain comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NO: 102. In certain embodiments, the extracellular antigen-binding domain comprises a V_{L} comprising amino acids having the sequence set forth in SEQ ID NO: 102.

In some embodiments, the V_{H} and/or V_{L} amino acid sequences having at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology to the specified sequences (e.g., SEQ ID NOs: 101 and 102) contain substitutions (e.g., conservative substitutions), insertions, or deletions relative to the specified sequence(s), but retain the ability to bind to the respective target antigen. In certain embodiments, a total of 1 to 10 amino acids are substituted, inserted and/or deleted in SEQ ID NOs: 101 and 102. In certain embodiments, substitutions, insertions, or deletions occur in regions outside the CDRs (e.g., in the framework regions (FRs)) of the extracellular antigen-binding domain. In certain embodiments, the extracellular antigen-binding domain comprises a V_{H} and/or V_{L} sequence selected from the group consisting of SEQ ID NOs: 101 and 102, including post-translational modifications of that sequence.

In some embodiments, the engineered receptor is a caTCR that binds to CD19. In some embodiments, the caTCR comprises a TCR delta chain comprising an amino acid sequence that is at least 80%, at least 85%, at least 90%, or at least 95% identical to SEQ ID NO: 103. In some embodiments, the caTCR comprises a TCR gamma chain comprising an amino acid sequence that is at least 80%, at least 85%, at least 90%, or at least 95% identical to SEQ ID NO: 104.

In some embodiments, the engineered receptor comprises (a) a heavy chain CDR1 comprising an amino acid sequence that is at least 80%, at least 85%, at least 90%, or at least 95% identical to SEQ ID NO: 105, (b) a heavy chain CDR2 comprising an amino acid sequence that is at least 80%, at least 85%, at least 90%, or at least 95% identical to SEQ ID NO: 106, and (c) a heavy chain CDR3 comprising an amino acid sequence that is at least 80%, at least 85%, at least 90%, or at least 95% identical to SEQ ID NOs: 107 or 108. In some embodiments, the heavy chain CDR3 comprises an amino acid sequence that is at least 80%, at least 85%, at least 90%, or at least 95% identical to SEQ ID NO: 107. In some embodiments, the engineered receptor comprises (a) a light chain CDR1 comprising an amino acid sequence that is at least 80%, at least 85%, at least 90%, or at least 95% identical to SEQ ID NO: 109, (b) a light chain CDR2 comprising an amino acid sequence that is at least 80%, at least 85%, at least 90%, or at least 95% identical to SEQ ID NO: 110, and (c) a light chain CDR3 comprising an amino acid sequence that is at least 80%, at least 85%, at least 90%, or at least 95% identical to SEQ ID NOs: 111 or 112. In some embodiments, the light chain CDR3 comprises an amino acid sequence that is at least 80%, at least 85%, at least 90%, or at least 95% identical to SEQ ID NO: 111.

Additional extracellular antigen-binding domains that bind to CD19, including scFv and CDR amino acid and nucleotide sequences are described in WO2017070608, which is incorporated by reference in its entirety, including the sequence listings provided therein. Extracellular Antigen Binding Domain of an Engineered Receptor that Binds to AFP

In some embodiments, extracellular antigen-binding domain of the expressed engineered receptor (e.g., a CAR, caTCR, or eTCR) binds to AFP. In some embodiments, the extracellular antigen-binding domain of the expressed engineered receptor (e.g., a CAR, caTCR, or eTCR) binds to AFP presented in the context of an MHC molecule. In some embodiments, the extracellular antigen-binding domain binds to AFP presented in the context of an HLA-A2 molecule.

In certain embodiments, the extracellular antigen-binding domain binds to AFP or a fragment thereof. In some embodiments, the extracellular antigen binding domain comprises a scFv comprising amino acids having a sequence of SEQ ID NO: 98, or a functional fragment or variant thereof. In some embodiments, the extracellular antigen-binding domain is a human scFv comprising amino acids having the sequence set forth SEQ ID NO: 98, or a functional fragment or variant thereof, optionally with (iii) a linker sequence, for example a linker peptide, between the heavy chain variable region and the light chain variable region. In certain embodiments, the linker comprises amino acids having the sequence set forth in SEQ ID NO: 118 (SRGGGGSGGGGSGGGGSLEMA). In certain embodiments, the extracellular antigen-binding domain is a human scFv-Fc fusion protein or full length human IgG with V_{H} and V_{L} regions. In certain embodiments, the scFv is fused to a CD28-CD3zeta peptide. In some embodiments, the CD28-CD3zeta peptide comprises amino acids having the sequence set forth in SEQ ID NO: 99. In some embodiments, the scFv is fused to a 41BB-CD3zeta peptide. In some embodiments, the 41BB-CD3zeta peptide has the following sequence:

(SEQ ID NO: 100)
TGTTTPAPRPPTPAPTIASQPLSLRPEACRPAAGGAVHTRGLDFACDIYI
WAPLAGTCGVLLLSLVITLYCKRGRKKLLYIFKQPFMRPVQTTQEEDGCS
CRFPEEEEGGCELRVKFSRSADAPAYQQGQNQLYNELNLGRREEYDVLDK
RRGRDPEMGGKPRRKNPQEGLYNELQKDKMAEAYSEIGMKGERRRGKGHD
GLYQGLSTATKDTYDALHMQALPPR.

In certain embodiments, the extracellular antigen-binding domain comprises (a) a scFv comprising an amino acid sequence that is at least 80%, at least 85%, at least 90%, or at least 95% identical to SEQ ID NO: 98; and (b) a CD28-CD3zeta peptide comprising an amino acid sequence that is at least 80%, at least 85%, at least 90%, or at least 95% identical to SEQ ID NO: 99. For example, the extracellular antigen-binding domain comprises (a) a scFv comprising an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 98; and (b) a CD28-CD3zeta peptide comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 99.

In certain embodiments, the extracellular antigen-binding domain comprises (a) a scFv comprising an amino acid sequence that is at least 80%, at least 85%, at least 90%, or at least 95% identical to SEQ ID NO: 98; and (b) a CD28-CD3zeta peptide comprising an amino acid sequence that is at least 80%, at least 85%, at least 90%, or at least 95% identical to SEQ ID NO: 100. For example, the extracellular antigen-binding domain comprises (a) a scFv comprising an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 98; and (b) a CD28-CD3zeta peptide comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 100.

In some embodiments, the engineered receptor comprises (a) a heavy chain CDR1 comprising an amino acid sequence that is at least 80%, at least 85%, at least 90%, or at least 95% identical to SEQ ID NO: 92, (b) a heavy chain CDR2 comprising an amino acid sequence that is at least 80%, at least 85%, at least 90%, or at least 95% identical to SEQ ID NO: 93, and (c) a heavy chain CDR3 comprising an amino acid sequence that is at least 80%, at least 85%, at least 90%, or at least 95% identical to SEQ ID NO: 94. In some embodiments, the engineered receptor comprises (a) a light chain CDR1 comprising an amino acid sequence that is at least 80%, at least 85%, at least 90%, or at least 95% identical to SEQ ID NO: 95, (b) a light chain CDR2 comprising an amino acid sequence that is at least 80%, at least 85%, at least 90%, or at least 95% identical to SEQ ID NO: 96, and (c) a light chain CDR3 comprising an amino acid sequence that is at least 80%, at least 85%, at least 90%, or at least 95% identical to SEQ ID NO: 97.

Additional extracellular antigen-binding domains that bind to AFP, including scFv and CDR amino acid and nucleotide sequences are described in WO2016161390, which is incorporated by reference in its entirety, including the sequence listings provided therein.

Extracellular Antigen Binding Domain of an Engineered Receptor that Binds to WT1

In some embodiments, extracellular antigen-binding domain of the expressed engineered receptor (e.g., a CAR, caTCR, or eTCR) binds to a WT1 tumor antigen. In some embodiments, the extracellular antigen-binding domain of the expressed engineered receptor (e.g., a CAR, caTCR, or eTCR) binds to a WT1 tumor antigen presented in the context of an MEW molecule. In some embodiments, the extracellular antigen-binding domain binds to a WT1 tumor antigen presented in the context of an HLA-A2 molecule.

In certain embodiments, the extracellular antigen-binding domain binds to WT1 tumor antigen or a fragment thereof. In some embodiments, the extracellular antigen binding domain comprises a heavy chain variable region comprising amino acids having a sequence selected from SEQ ID NOs: 134-140, or a functional fragment or variant thereof. In some embodiments, the extracellular antigen-binding domain (e.g., human scFv) comprises a light chain variable region comprising amino acids having a sequence selected from SEQ ID NOs: 141-147, or a functional fragment or variant thereof. In some embodiments, the extracellular antigen-binding domain is a human scFv, which comprises a heavy chain variable region comprising amino acids having the sequence selected from SEQ ID NOs: 134-140, or a functional fragment or variant thereof and a light chain variable region comprising amino acids having the sequence selected from SEQ ID NOs: 141-147, or a functional fragment or variant thereof, optionally with (iii) a linker sequence, for example a linker peptide, between the heavy chain variable region and the light chain variable region. In certain embodiments, the linker comprises amino acids having the sequence set forth in SEQ ID NO: 118 (SRGGGGSGGGGSGGGGSLEMA). In certain embodiments, the extracellular antigen-binding domain is a human scFv-Fc fusion protein or full length human IgG with V_{H} and V_{L} regions

In certain embodiments, the extracellular antigen-binding domain comprises a V_{H} comprising an amino acid sequence that is at least 80%, at least 85%, at least 90%, or at least 95% identical to a sequence selected from SEQ ID NOs: 134-140. For example, the extracellular antigen-binding domain comprises a V_{H} comprising an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to a sequence selected from SEQ ID NOs: 134-140. In certain embodiments, the extracellular antigen-binding domain comprises a V_{H} comprising amino acids having the sequence selected from SEQ ID NOs: 134-140. In certain embodiments, the extracellular antigen-binding domain comprises a V_{L} comprising an amino acid sequence that is at least 80%, at least 85%, at least 90%, or at least 95% identical to a sequence selected from SEQ ID NOs: 141-147. For example, the extracellular antigen-binding domain comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to a sequence selected from SEQ ID NOs: 141-147. In certain embodiments, the extracellular antigen-binding domain comprises a V_{L} comprising amino acids having the sequence selected from SEQ ID NOs: 141-147.

In some embodiments, the V_{H} and/or V_{L} amino acid sequences having at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology to the specified sequences (e.g., SEQ ID NOs: 141-147) contain substitutions (e.g., conservative substitutions), insertions, or deletions relative to the specified sequence(s), but retain the ability to bind to the respective target antigen. In certain embodiments, a total of 1 to 10 amino acids are substituted, inserted and/or deleted in SEQ ID NOs: 141-147. In certain embodiments, substitutions, insertions, or deletions occur in regions outside the CDRs (e.g., in the framework regions (FRs)) of the extracellular antigen-binding domain. In certain embodiments, the extracellular antigen-binding domain comprises a V_{H} and/or V_{L} sequence selected from the group consisting of SEQ ID NOs: 141-147, including post-translational modifications of that sequence.

In some embodiments, the engineered receptor comprises (A) (i) a heavy chain (HC) variable region comprising HC-CDR1, HC-CDR2 and HC-CDR3 and HC-CDR3 respectively, comprising amino acid sequences that is at least 80%, at least 85%, at least 90%, or at least 95% identical to SEQ ID NOs: 148, 149, and 150; and a light chain (LC) variable region comprising LC-CDR1, LC-CDR2 and LC-CDR3 respectively, comprising amino acid sequences that is at least 80%, at least 85%, at least 90%, or at least 95% identical to SEQ ID NOs: 151, 152, and 153; (ii) a heavy chain (HC) variable region comprising HC-CDR1, HC-CDR2 and HC-CDR3 respectively, comprising amino acid sequences that is at least 80%, at least 85%, at least 90%, or at least 95% identical to SEQ ID NOs: 154, 155, and 156; and a light chain (LC) variable region comprising LC-CDR1, LC-CDR2 and LC-CDR3 respectively, comprising amino acid sequences that is at least 80%, at least 85%, at least 90%, or at least 95% identical to SEQ ID NOs: 157, 158, and 159; (iii) a heavy chain (HC) variable region comprising HC-CDR1, HC-CDR2 and HC-CDR3 respectively, comprising amino acid sequences that is at least 80%, at least 85%, at least 90%, or at least 95% identical to SEQ ID NOs: 160, 161, and 162; and a light chain (LC) variable region comprising LC-CDR1, LC-CDR2 and LC-CDR3 respectively, comprising amino acid sequences that is at least 80%, at least 85%, at least 90%, or at least 95% identical to SEQ ID NOs: 163, 164, and 165; (iv) a heavy chain (HC) variable region comprising HC-CDR1, HC-CDR2 and HC-CDR3 respectively, comprising amino acid sequences that is at least 80%, at least 85%, at least 90%, or at least 95% identical to SEQ ID NOs: 166, 167, and 168; and a light chain (LC) variable region comprising LC-CDR1, LC-CDR2 and LC-CDR3 respectively, comprising amino acid sequences that are at least 80%, at least 85%, at least 90%, or at least 95% identical to SEQ ID NOs: 169, 170, and 171; (v) a heavy chain (HC) variable region comprising HC-CDR1, HC-CDR2 and HC-CDR3 respectively, comprising amino acid sequences that are at least 80%, at least 85%, at least 90%, or at least 95% identical to SEQ ID NOs: 172, 173, and 174; and a light chain (LC) variable region comprising LC-CDR1, LC-CDR2 and LC-CDR3 respectively, comprising amino acid sequences that are at least 80%, at least 85%, at least 90%, or at least 95% identical to SEQ ID NOs: 175, 176, and 177; or (vi) a heavy chain (HC) variable region comprising HC-CDR1, HC-CDR2 and HC-CDR3 respectively, comprising amino acid sequences that are at least 80%, at least 85%, at least 90%, or at least 95% identical to SEQ ID NOs: 178, 179, and 180; and a light chain (LC) variable region comprising LC-CDR1, LC-CDR2 and LC-CDR3 respectively, comprising amino acid sequences that are at least 80%, at least 85%, at least 90%, or at least 95% identical to SEQ ID NOs: 181, 182, and 183; or (B) a V_{H} and V_{L} comprising first and second amino acid sequences, respectively, selected from SEQ ID NOs: 134 and 141; 135 and 142; 136 and 143; 137 and 144; 138 and 145; or 139 and 146; or (C) an amino acid sequence selected from SEQ ID NOs: 184-189.

Additional extracellular antigen-binding domains that bind to WT1, including anti-WT1 antibodies, scFv and CDR amino acid and nucleotide sequences are described in WO2015/070061, which is incorporated by reference in its entirety, including the sequence listings provided therein, can be employed in any of the methods provided herein.

Extracellular Antigen Binding Domain of an Engineered Receptor that Binds to ROR2

In some embodiments, extracellular antigen-binding domain of the expressed engineered receptor (e.g., a CAR, caTCR, or eTCR) binds to a ROR2 protein. In some embodiments, the extracellular antigen-binding domain of the expressed engineered receptor (e.g., a CAR, caTCR, or eTCR) binds to a ROR2 protein presented in the context of an MEW molecule. In some embodiments, the extracellular antigen-binding domain binds to a ROR2 protein presented in the context of an HLA-A2 molecule.

In certain embodiments, the extracellular antigen-binding domain binds to a ROR2 protein or a fragment thereof. In some embodiments, the extracellular antigen binding domain comprises a heavy chain variable region comprising amino acids having a sequence of SEQ ID NOs: 191-203, or a functional fragment or variant thereof. In some embodiments, the extracellular antigen-binding domain (e.g., human scFv) comprises a light chain variable region comprising amino acids having a sequence of SEQ ID NOs: 204-216, or a functional fragment or variant thereof. In some embodiments, the extracellular antigen-binding domain is a human scFv, which comprises a heavy chain variable region comprising amino acids having the sequence set forth SEQ ID NOs: 191-203, or a functional fragment or variant thereof and a light chain variable region comprising amino acids having the sequence set forth in SEQ ID NOs: 204-216, or a functional fragment or variant thereof, optionally with (iii) a linker sequence, for example a linker peptide, between the heavy chain variable region and the light chain variable region. In certain embodiments, the linker comprises amino acids having the sequence set forth in SEQ ID NO: 118 (SRGGGGSGGGGSGGGGSLEMA). In certain embodiments, the extracellular antigen-binding domain is a human scFv-Fc fusion protein or full length human IgG with V_{H} and V_{L} regions.

In certain embodiments, the extracellular antigen-binding domain comprises a V_{H} comprising an amino acid sequence that is at least 80%, at least 85%, at least 90%, or at least 95% identical to SEQ ID NOs: 191-203. For example, the extracellular antigen-binding domain comprises a V_{H} comprising an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NOs: 191-203. In certain embodiments, the extracellular antigen-binding domain comprises a V_{H} comprising amino acids having the sequence set forth in SEQ ID NOs: 191-203. In certain embodiments, the extracellular antigen-binding domain comprises a V_{L} comprising an amino acid sequence that is at least 80%, at least 85%, at least 90%, or at least 95% identical to SEQ ID NOs: 204-216. For example, the extracellular antigen-binding domain comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NOs: 204-216. In certain embodiments, the extracellular antigen-binding domain comprises a V_{L} comprising amino acids having the sequence set forth in SEQ ID NOs: 204-216.

In some embodiments, V_{H} and/or V_{L} amino acid sequences having at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology to the specified sequences (e.g., SEQ ID NOs: 191-216) contain substitutions (e.g., conservative substitutions), insertions, or deletions relative to the specified sequence(s), but retain the ability to bind to the respective target antigen. In certain embodiments, a total of 1 to 10 amino acids are substituted, inserted and/or deleted in a sequence selected from SEQ ID NOs: 191-216. In certain embodiments, substitutions, insertions, or deletions occur in regions outside the CDRs (e.g., in the framework regions (FRs)) of the extracellular antigen-binding domain. In certain embodiments, the extracellular antigen-binding domain comprises a V_{H} and/or V_{L} sequence selected from the group consisting of SEQ ID NOs: 191-216, including post-translational modifications of that sequence.

In some embodiments, the extracellular antigen-binding domain comprises a V_{H} having an amino acid sequence of SEQ ID NO: 203. In some embodiments, the extracellular antigen-binding domain comprises a V_{H} encoded by the nucleotide sequence of SEQ ID NO: 242. In some embodiments, the extracellular antigen-binding domain comprises a V_{L} having an amino acid sequence of SEQ ID NO: 216. In some embodiments, the extracellular antigen-binding domain comprises a V_{L} encoded by the nucleotide sequence of SEQ ID NO: 241. In some embodiments, the V_{H} and V_{L} chains are linked by a linker having the amino acid sequence of (GGGGS)ₙ (SEQ ID NO: 120), wherein n=3.

In some embodiments, the engineered receptor comprises (i) a heavy chain (HC) variable region comprising HC-CDR1, HC-CDR2 and HC-CDR3 and HC-CDR3 respectively, comprising amino acid sequences that is at least 80%, at least 85%, at least 90%, or at least 95% identical to SEQ ID NOs: 243-245; and a light chain (LC) variable region comprising LC-CDR1, LC-CDR2 and LC-CDR3 respectively, comprising amino acid sequences that is at least 80%, at least 85%, at least 90%, or at least 95% identical to SEQ ID NOs: 246-248; (ii) a heavy chain (HC) variable region comprising HC-CDR1, HC-CDR2 and HC-CDR3 respectively, comprising amino acid sequences that is at least 80%, at least 85%, at least 90%, or at least 95% identical to SEQ ID NOs: 249-251; and a light chain (LC) variable region comprising LC-CDR1, LC-CDR2 and LC-CDR3 respectively, comprising amino acid sequences that is at least 80%, at least 85%, at least 90%, or at least 95% identical to SEQ ID NOs: 252-254; (iii) a heavy chain (HC) variable region comprising HC-CDR1, HC-CDR2 and HC-CDR3 respectively, comprising amino acid sequences that is at least 80%, at least 85%, at least 90%, or at least 95% identical to SEQ ID NOs: 255-257; and a light chain (LC) variable region comprising LC-CDR1, LC-CDR2 and LC-CDR3 respectively, comprising amino acid sequences that is at least 80%, at least 85%, at least 90%, or at least 95% identical to SEQ ID NOs: 258-260; (iv) a heavy chain (HC) variable region comprising HC-CDR1, HC-CDR2 and HC-CDR3 respectively, comprising amino acid sequences that is at least 80%, at least 85%, at least 90%, or at least 95% identical to SEQ ID NOs: 261-263; and a light chain (LC) variable region comprising LC-CDR1, LC-CDR2 and LC-CDR3 respectively, comprising amino acid sequences that are at least 80%, at least 85%, at least 90%, or at least 95% identical to SEQ ID NOs: 264-266; (v) a heavy chain (HC) variable region comprising HC-CDR1, HC-CDR2 and HC-CDR3 respectively, comprising amino acid sequences that are at least 80%, at least 85%, at least 90%, or at least 95% identical to SEQ ID NOs: 267-269; and a light chain (LC) variable region comprising LC-CDR1, LC-CDR2 and LC-CDR3 respectively, comprising amino acid sequences that are at least 80%, at least 85%, at least 90%, or at least 95% identical to SEQ ID NOs: 270-272; (vi) a heavy chain (HC) variable region comprising HC-CDR1, HC-CDR2 and HC-CDR3 respectively, comprising amino acid sequences that are at least 80%, at least 85%, at least 90%, or at least 95% identical to SEQ ID NOs: 273-275; and a light chain (LC) variable region comprising LC-CDR1, LC-CDR2 and LC-CDR3 respectively, comprising amino acid sequences that are at least 80%, at least 85%, at least 90%, or at least 95% identical to SEQ ID NOs: 276-278; (vii) a heavy chain (HC) variable region comprising HC-CDR1, HC-CDR2 and HC-CDR3 respectively, comprising amino acid sequences that are at least 80%, at least 85%, at least 90%, or at least 95% identical to SEQ ID NOs: 279-281; and a light chain (LC) variable region comprising LC-CDR1, LC-CDR2 and LC-CDR3 respectively, comprising amino acid sequences that are at least 80%, at least 85%, at least 90%, or at least 95% identical to SEQ ID NOs: 282-284; (viii) a heavy chain (HC) variable region comprising HC-CDR1, HC-CDR2 and HC-CDR3 respectively, comprising amino acid sequences that are at least 80%, at least 85%, at least 90%, or at least 95% identical to SEQ ID NOs: 285-287; and a light chain (LC) variable region comprising LC-CDR1, LC-CDR2 and LC-CDR3 respectively, comprising amino acid sequences that are at least 80%, at least 85%, at least 90%, or at least 95% identical to SEQ ID NOs: 288-290; (ix) a heavy chain (HC) variable region comprising HC-CDR1, HC-CDR2 and HC-CDR3 respectively, comprising amino acid sequences that are at least 80%, at least 85%, at least 90%, or at least 95% identical to SEQ ID NOs: 291-293; and a light chain (LC) variable region comprising LC-CDR1, LC-CDR2 and LC-CDR3 respectively, comprising amino acid sequences that are at least 80%, at least 85%, at least 90%, or at least 95% identical to SEQ ID NOs: 294-296; (x) a heavy chain (HC) variable region comprising HC-CDR1, HC-CDR2 and HC-CDR3 respectively, comprising amino acid sequences that are at least 80%, at least 85%, at least 90%, or at least 95% identical to SEQ ID NOs: 297-299; and a light chain (LC) variable region comprising LC-CDR1, LC-CDR2 and LC-CDR3 respectively, comprising amino acid sequences that are at least 80%, at least 85%, at least 90%, or at least 95% identical to SEQ ID NOs: 300-302; (xi) a heavy chain (HC) variable region comprising HC-CDR1, HC-CDR2 and HC-CDR3 respectively, comprising amino acid sequences that are at least 80%, at least 85%, at least 90%, or at least 95% identical to SEQ ID NOs: 303-305; and a light chain (LC) variable region comprising LC-CDR1, LC-CDR2 and LC-CDR3 respectively, comprising amino acid sequences that are at least 80%, at least 85%, at least 90%, or at least 95% identical to SEQ ID NOs: 306-308; (xii) a heavy chain (HC) variable region comprising HC-CDR1, HC-CDR2 and HC-CDR3 respectively, comprising amino acid sequences that are at least 80%, at least 85%, at least 90%, or at least 95% identical to SEQ ID NOs: 309-311; and a light chain (LC) variable region comprising LC-CDR1, LC-CDR2 and LC-CDR3 respectively, comprising amino acid sequences that are at least 80%, at least 85%, at least 90%, or at least 95% identical to SEQ ID NOs: 312-314; or (xiii) a heavy chain (HC) variable region comprising HC-CDR1, HC-CDR2 and HC-CDR3 respectively, comprising amino acid sequences that are at least 80%, at least 85%, at least 90%, or at least 95% identical to SEQ ID NOs: 315-317; and a light chain (LC) variable region comprising LC-CDR1, LC-CDR2 and LC-CDR3 respectively, comprising amino acid sequences that are at least 80%, at least 85%, at least 90%, or at least 95% identical to SEQ ID NOs: 318-320. 101.661 Additional extracellular antigen-binding domains that bind to ROR2, including scFv and CDR amino acid and nucleotide sequences are described in WO2016/142768, which is incorporated by reference in its entirety, including the sequence listings provided therein.

Extracellular Antigen Binding Domain that Binds to CD3

In some embodiments, the TCR expresses an extracellular antigen-binding domain that binds to CD3. In some embodiments, the extracellular antigen-binding comprises a scFv that binds to CD3 (e.g., anti-CD3 scFv). In some embodiments, the extracellular antigen-binding domain comprises a scFv having an amino acid sequence of SEQ ID NO: 113, or a functional fragment or variant thereof.

In certain embodiments, the extracellular antigen-binding domain comprises a scFv comprising an amino acid sequence that is at least 80%, at least 85%, at least 90%, or at least 95% identical to SEQ ID NO: 113. For example, the extracellular antigen-binding domain comprises a scFv comprising an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 113. In certain embodiments, the extracellular antigen-binding domain comprises a scFv comprising amino acids having the sequence set forth in SEQ ID NO: 113. In certain embodiments, the extracellular antigen-binding domain comprises a scFv encoded by a polynucleotide sequence that is at least 80%, at least 85%, at least 90%, or at least 95% identical to SEQ ID NO: 114. For example, the extracellular antigen-binding domain comprises a scFv encoded by a polynucleotide sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NO: 114. In certain embodiments, the extracellular antigen-binding domain comprises a scFv encoded by a polynucleotide sequence having the sequence set forth in SEQ ID NO: 114.

In some embodiments, the scFv amino acid sequences having at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology to the specified sequences (e.g., SEQ ID NO: 113) contain substitutions (e.g., conservative substitutions), insertions, or deletions relative to the specified sequence(s), but retain the ability to bind to the respective target antigen. In certain embodiments, a total of 1 to 10 amino acids are substituted, inserted and/or deleted in SEQ ID NO: 113. In certain embodiments, substitutions, insertions, or deletions occur in regions outside the CDRs (e.g., in the framework regions (FRs)) of the extracellular antigen-binding domain. In certain embodiments, the extracellular antigen-binding domain comprises a scFv sequence of SEQ ID NO: 113, including post-translational modifications of that sequence.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4: 1 1-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally, or alternatively, the amino acids sequences of the presently disclosed subject matter can further be used as a “query sequence” to perform a search against public databases to, for example, identify related sequences. Such searches can be performed using the XBLAST program (version 2.0) of Altschul et al. (1990) J Mol. Biol. 215:403-10. BLAST protein searches can be performed with the XBLAST program, score=50, wordlength=3 to obtain amino acid sequences homologous to the specified sequences disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

In certain non-limiting embodiments, an extracellular antigen-binding domain of the presently disclosed engineered receptor comprises a linker connecting the heavy chain variable region and light chain variable region of the extracellular antigen-binding domain. As used herein, the term “linker” refers to a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises amino acids having the sequence set forth in SEQ ID NO: 118 (SRGGGGSGGGGSGGGGSLEMA). In certain embodiments, the nucleotide sequence encoding the amino acid sequence of SEQ ID NO: 118 (SRGGGGSGGGGSGGGGSLEMA) is set forth in SEQ ID NO: 119 (ctagaggtggtggtggtagcggcggcggcggctctggtggtggtggatcc).

In addition, the extracellular antigen-binding domain can comprise a leader or a signal peptide that directs the nascent protein into the endoplasmic reticulum. Signal peptide or leader can be essential if the engineered receptor is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the N-terminus of the extracellular antigen-binding domain. In certain embodiments, the signal peptide comprises a CD8 signal polypeptide comprising amino acids having the sequence set forth in SEQ ID NO: 122 as provided below.

(SEQ ID NO: 122)
MALPVTALLLPLALLLHAARP.

The nucleotide sequence encoding the amino acid sequence of SEQ ID NO: 123 is set forth in SEQ ID NO: 123, which is provided below:

(SEQ ID NO: 123)
atggccagccagtaacggctctgctgctgccacttgactgacctccatgc
agccaggcct.

### Bispecific Engineered Receptor

In some embodiments, the engineered receptor (e.g., CAR, caTCR, eTCR) or other cell-surface ligand is bispecific. In some embodiments, the bispecific TCR or cell-surface ligand comprises (a) an antibody moiety that specific binds to a target antigen (i.e., cell surface antigen); and (b) a TCR module (TCRM) that is capable of recruiting a TCR-associated signaling module. Examples of such bispecific TCRs or cell-surface ligands are described in WO2017/070608, which is incorporated by reference in its entirety, including the sequence listings provided therein.

In some embodiments, the bispecific engineered receptor or cell-surface ligand comprises (a) a first extracellular antigen-binding domain that binds to a first target antigen or a fragment thereof; and (b) a second extracellular antigen-binding domain that binds to a second target antigen or fragment thereof. In some embodiments, the first target antigen is CD19, AFP1, ROR2 or WT1. In some embodiments, the second target antigen is a cell surface protein. In some embodiments, the cell surface protein is CD3.

In some embodiments, the bispecific TCR or cell-surface ligand comprises (a) a first extracellular antigen-binding domain that binds to ROR2; and (b) a second extracellular antigen-binding domain that binds to CD3. In some embodiments, the bispecific TCR or cell-surface antigen has an amino acid sequence of SEQ ID NO: 321. In some embodiments, the extracellular antigen-binding domain that binds to ROR2 comprises a light chain variable region (V_{L}) (e.g., anti-ROR2 V_{L}) encoded by the polynucleotide sequence of SEQ ID NO: 241. In some embodiments, the extracellular antigen-binding domain that binds to ROR2 comprises a V_{L} having the amino acid sequence of SEQ ID NO: 216. In some embodiments, the extracellular antigen-binding domain that binds to ROR2 comprises a heavy chain variable region (V_{H}) (e.g., anti-ROR2 V_{H}) encoded by the polynucleotide sequence of SEQ ID NO: 242. In some embodiments, the extracellular antigen-binding domain that binds to ROR2 comprises a V_{H} having the amino acid sequence of SEQ ID NO: 203. In some embodiments, the extracellular antigen-binding domain that binds to CD3 comprises a scFv (e.g., anti-CD3 scFv) encoded by the polynucleotide sequence of SEQ ID NO: 114. In some embodiments, the extracellular antigen-binding domain that binds to CD3 comprises a scFv having the amino acid sequence of SEQ ID NO: 113. In some embodiments, the anti-ROR2 V_{L} is attached to the anti-ROR2 V_{H} via a linker. In some embodiments, the linker connecting the anti-ROR2 V_{L} and anti-ROR2 V_{H} has is encoded by the polynucleotide sequence of SEQ ID NO: 119. In some embodiments, the linker connecting the anti-ROR2 V_{L} and anti-ROR2 V_{H} has the amino acid sequence of SEQ ID NO: 118. In some embodiments, the anti-ROR2 V_{H} is attached to the anti-CD3 scFv via a linker. In some embodiments, the linker connecting the anti-ROR2 V_{H} and the anti-CD3 scFv is encoded by the polynucleotide sequence of SEQ ID NO: 121. In some embodiments, the linker connecting the anti-ROR2 V_{H} and the anti-CD3 scFv has the amino acid sequence of SEQ ID NO: 120.

### Transmembrane Domain of an Engineered Receptor

In certain non-limiting embodiments, the transmembrane domain of the engineered receptor (e.g., CAR, caTCR, eTCR) comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the transmembrane domain of the engineered receptor comprises a CD8 polypeptide, a CD28 polypeptide, a CD3ζ polypeptide, a CD4 polypeptide, a 4-IBB polypeptide, an OX40 polypeptide, an SEQ ID NO: 129, a CTLA-4 polypeptide, a PD-1 polypeptide, a LAG-3 polypeptide, a 2B4 polypeptide, a BTLA polypeptide, a synthetic peptide (e.g., a transmembrane peptide not based on a protein associated with the immune response), or a combination thereof.

In certain embodiments, the transmembrane domain of a presently disclosed engineered receptor comprises a CD28 polypeptide. The CD28 polypeptide can have an amino acid sequence that is at least about 85%, about 90%, about 95%, about 96%, about 97%, about 98%, about 99% or 100% homologous to the sequence having a NCBI Reference No: PI0747 or NP006130 (SEQ ID NO: 125), or fragments thereof, and/or can optionally comprise up to one or up to two or up to three conservative amino acid substitutions. In certain embodiments, the CD28 polypeptide can have an amino acid sequence that is a consecutive portion of SEQ ID NO: 125 which is at least 20, or at least 30, or at least 40, or at least 50, and up to 220 amino acids in length. Alternatively, or additionally, in non-limiting various embodiments, the CD28 polypeptide has an amino acid sequence of amino acids 1 to 220, 1 to 50, 50 to 100, 100 to 150, 114 to 220, 150 to 200, or 200 to 220 of SEQ ID NO: 125. In certain embodiments, the engineered receptor of the presently disclosed comprises a transmembrane domain comprising a CD28 polypeptide, and an intracellular domain comprising a co-stimulatory signaling region that comprises a CD28 polypeptide. In certain embodiments, the CD28 polypeptide comprised in the transmembrane domain and the intracellular domain has an amino acid sequence of amino acids 114 to 220 of SEQ ID NO: 125.

SEQ ID NO: 125 is provided below:

(SEQ ID NO: 125)
MLRLLLALNLFPSIQVTGNKILVKQSPMLVAYDNALSCKYSYNLFSREFR
ASLHKGLDSAVEVCWYGNYSQQLQVYSKTGFNCDGKLGNESVTFYLQNLY
QTDIYFCKIEVMYPPPYLDNEKSNGTIIHVKGKHLCPSPLFPGPSKPFWV
LVWGGVLACYSLLVTVAFIIFWVRSKRSRLLHSDYMNMTPRRPGPTRKHY
QPYAPPRDFAAYRS

In accordance with the presently disclosed subject matter, a “CD28 nucleic acid molecule” refers to a polynucleotide encoding a CD28 polypeptide. In certain embodiments, the CD28 nucleic acid molecule encoding the CD28 polypeptide comprised in the transmembrane domain and the intracellular domain (e.g., the co-stimulatory signaling region) of the presently disclosed engineered receptor (amino acids 114 to 220 of SEQ ID NO: 125) comprises nucleic acids having the sequence set forth in SEQ ID NO: 126 as provided below.

(SEQ ID NO: 126)
attgaagttatgtatcctcctccttacctagacaatgagaagagcaatgg
aaccattatccatgtgaaagggaaacacctttgtccaagtcccctatttc
ccggaccttctaagcccttttgggtgctggtggtggttggtggagtcctg
gcttgctatagcttgctagtaacagtggcctttattattttctgggtgag
gagtaagaggagcaggctcctgcacagtgactacatgaacatgactcccc
gccgccccgggcccacccgcaagcattaccagccctatgccccaccacgc
gacttcgcagcctatcgctcc

In certain embodiments, the transmembrane domain comprises a CD8 polypeptide. The CD8 polypeptide can have an amino acid sequence that is at least about 85%, about 90%, about 95%, about 96%, about 97%, about 98%, about 99% or about 100%) homologous to SEQ ID NO: 124 (homology herein can be determined using standard software such as BLAST or FASTA) as provided below, or fragments thereof, and/or can optionally comprise up to one or up to two or up to three conservative amino acid substitutions. In certain embodiments, the CD8 polypeptide can have an amino acid sequence that is a consecutive portion of SEQ ID NO: 124 which is at least 20, or at least 30, or at least 40, or at least 50, and up to 235 amino acids in length. Alternatively, or additionally, in non-limiting various embodiments, the CD8 polypeptide has an amino acid sequence of amino acids 1 to 235, 1 to 50, 50 to 100, 100 to 150, 150 to 200, or 200 to 235 of SEQ ID NO: 124.

SEQ ID NO: 124 is provided below:

(SEQ ID NO: 124)
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPWKSGDKPSLSARYV

In accordance with the presently disclosed subject matter, a “CD8 nucleic acid molecule” refers to a polynucleotide encoding a CD8 polypeptide.

In certain non-limiting embodiments, an engineered receptor also comprises a spacer region that links the extracellular antigen-binding domain to the transmembrane domain. The spacer region can be flexible enough to allow the antigen-binding domain to orient in different directions to facilitate antigen recognition while preserving the activating activity of the engineered receptor (e.g., a CAR, caTCR, or eTCR). In certain non-limiting embodiments, the spacer region can be the hinge region from IgG1, the CH₂CH₃ region of immunoglobulin and portions of CD3, a portion of a CD28 polypeptide (e.g., SEQ ID NO: 125), a portion of a CD8 polypeptide (e.g., SEQ ID NO: 124), a variation of any of the foregoing which is at least about 80%, at least about 85%>, at least about 90%, or at least about 95% homologous thereto, or a synthetic spacer sequence. In certain non-limiting embodiments, the spacer region can have a length between about 1-50 (e.g., 5-25, 10-30, or 30-50) amino acids.

### Intracellular Domain of an Engineered Receptor

In certain non-limiting embodiments, an intracellular domain of the CAR can comprise a CD3 polypeptide, which can activate or stimulate a cell (e.g., a cell of the lymphoid lineage, e.g., a T cell). CD3 comprises 3 ITAMs, and transmits an activation signal to the cell (e.g., a cell of the lymphoid lineage, e.g., a T cell) after antigen is bound. The CD3 polypeptide can have an amino acid sequence that is at least about 85%, about 90%, about 95%, about 96%, about 97%, about 98%, about 99% or about 100% homologous to the sequence having a NCBI Reference No: NP_932170 (SEQ ID NO: 115), or fragments thereof, and/or can optionally comprise up to one or up to two or up to three conservative amino acid substitutions. In non-limiting certain embodiments, the CD3 polypeptide can have an amino acid sequence that is a consecutive portion of SEQ ID NO: 115 which is at least 20, or at least 30, or at least 40, or at least 50, and up to 164 amino acids in length. Alternatively, or additionally, in non-limiting various embodiments, the CD3ζ polypeptide has an amino acid sequence of amino acids 1 to 164, 1 to 50, 50 to 100, 100 to 150, or 150 to 164 of SEQ ID NO: 115. In certain embodiments, the CD3ζ polypeptide has an amino acid sequence of amino acids 52 to 164 of SEQ ID NO: 115.

SEQ ID NO: 115 is provided below:

(SEQ ID NO: 115)
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR

In certain embodiments, the CD3 polypeptide has the amino acid sequence set forth in SEQ ID NO: 116, which is provided below:

(SEQ ID NO: 116)
RVKFSRSAEPPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

In accordance with the presently disclosed subject matter, a “CD3ζ nucleic acid molecule” refers to a polynucleotide encoding a CD3ζ polypeptide. In certain embodiments, the CD3ζ nucleic acid molecule encoding the CD3ζ polypeptide (SEQ ID NO: 117) comprised in the intracellular domain of the presently disclosed engineered receptor (e.g., a CAR, caTCR, or eTCR) comprises a nucleotide sequence as set forth in SEQ ID NO: 117 as provided below.

(SEQ ID NO: 117)
agagtgaagttcagcaggagcgcagagccccccgcgtaccagcagggccag
aaccagctctataacgagctcaatctaggacgaagagaggagtacgatgtt
ttggacaagagacgtggccgggaccctgagatggggggaaagccgagaagg
aagaaccctcaggaaggcctgtacaatgaactgcagaaagataagatggcg
gaggcctacagtgagattgggatgaaaggcgagcgccggaggggcaagggg
cacgatggcctttaccagggtctcagtacagccaccaaggacacctacgac
gcccttcacatgcaggccctgccccctcgcg

In certain non-limiting embodiments, an intracellular domain of the engineered receptor (e.g., a CAR, caTCR, or eTCR) further comprises at least one signaling region. The at least one signaling region can include. for example, a CD28 polypeptide, a 4-IBB polypeptide, an OX40 polypeptide, an SEQ ID NO: 129, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof.

In certain embodiments, the signaling region is a co-stimulatory signaling region. In certain embodiments, the co-stimulatory signaling region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-IBB polypeptide, an OX40 polypeptide, an SEQ ID NO: 129, a DAP-10 polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to the extracellular antigen binding domain of an engineered receptor. Co-stimulatory ligands, include, but are not limited to CD80, CD86, CD70, OX40L, 4-1BBL, CD48, and TNFRSF14. As one example, a 4-1BB ligand (i.e., 4-1BBL) can bind to 4-1BB (also known as “CD 137”) for providing an intracellular signal that in combination with an extracellular signal induces an effector cell function of the engineered T cell. Engineered receptors comprising an intracellular domain that comprises a co-stimulatory signaling region comprising 4-1BB, ICOS or DAP-10 are disclosed in U.S. Pat. No. 7,446,190, which is herein incorporated by reference in its entirety. In certain embodiments, the intracellular domain of the engineered receptor comprises a co-stimulatory signaling region that comprises a CD28 polypeptide. In certain embodiments, the intracellular domain of the engineered receptor comprises a co-stimulatory signaling region that comprises two co-stimulatory molecules: CD28 and 4-1BB or CD28 and OX40.

4-IBB can act as a tumor necrosis factor (TNF) ligand and have stimulatory activity. The 4-IBB polypeptide can have an amino acid sequence that is at least about 85%, about 90%, about 95%, about 96%, about 97%, about 98%, about 99% or 100% homologous to the sequence having a NCBI Reference No: P41273 or NP_001552 (SEQ ID NO: 127) or fragments thereof, and/or can optionally comprise up to one or up to two or up to three conservative amino acid substitutions.

SEQ ID NO: 127 is provided below:

(SEQ ID NO: 127)
MGNSCYNIVATLLLVLNFERTRSLQDPCSNCPAGTFCDNNRNQICSPCPPN
SFSSAGGQRTCDICRQCKGVFRTRKECSSTSNAECDCTPGFHCLGAGCSMC
EQDCKQGQELTKKGCKDCCFGTFNDQKRGICRPWTNCSLDGKSVLGTKERD
WCGPSPADLSPGASSVTPPAPAREPGHSPQIISFFLALTSTALLFLLFFLT
LRFSWKRGRKKLLYIFKQPFMRPVQTTQEEDGCSCRFPEEEEGGCEL.

In accordance with the presently disclosed subject matter, a “4-IBB nucleic acid molecule” refers to a polynucleotide encoding a 4-IBB polypeptide.

An OX40 polypeptide can have an amino acid sequence that is at least about 85%, about 90%, about 95%, about 96%, about 97%, about 98%, about 99% or 100% homologous to the sequence having a NCBI Reference No: P43489 or NP_003318 (SEQ ID NO: 128), or fragments thereof, and/or can optionally comprise up to one or up to two or up to three conservative amino acid substitutions.

SEQ ID NO: 128 is provided below:

(SEQ ID NO: 128)
MCVGARRLGRGPCAALLLLGLGLSTVTGLHCVGDTYPSNDRCCHECRPGNG
MVSRCSRSQNTVCRPCGPGFYNDWSSKPCKPCTWCNLRSGSERKQLCTATQ
DTVCRCRAGTQPLDSYKPGVDCAPCPPGHFSPGDNQACKPWTNCTLAGKHT
LQPASNSSDAICEDRDPPATQPQETQGPPARPITVQPTEAWPRTSQGPSTR
PVEVPGGRAVAAILGLGLVLGLLGPLAILLALYLLRRDQRLPPDAHKPPGG
GSFRTPIQEEQADAHSTLAKI.

In accordance with the presently disclosed subject matter, an “OX40 nucleic acid molecule” refers to a polynucleotide encoding an OX40 polypeptide.

An ICOS polypeptide can have an amino acid sequence that is at least about 85%, about 90%, about 95%, about 96%, about 97%, about 98%, about 99% or 100% homologous to the sequence having a NCBI Reference No: NP_036224 (SEQ ID NO: 129) or fragments thereof, and/or can optionally comprise up to one or up to two or up to three conservative amino acid substitutions.

SEQ ID NO: 129 is provided below:

(SEQ ID NO: 129)
MKSGLWYFFLFCLRIKVLTGEINGSANYEMFIFHNGGVQILCKYPDIVQQF
KMQLLKGGQILCDLTKTKGSGNTVSIKSLKFCHSQLSNNSVSFFLYNLDHS
HANYYFCNLSIFDPPPFKVTLTGGYLHIYESQLCCQLKFWLPIGCAAFVWC
ILGCILICWLTKKKYSSSVHDPNGEYMFMRATAKKSRLTDVTL.

In accordance with the presently disclosed subject matter, an “ICOS nucleic acid molecule” refers to a polynucleotide encoding an SEQ ID NO: 129.

CTLA-4 is an inhibitory receptor expressed by activated T cells, which when engaged by its corresponding ligands (CD80 and CD86; B7-1 and B7-2, respectively), mediates activated T cell inhibition or anergy. In both preclinical and clinical studies, CTLA-4 blockade by systemic antibody infusion, enhanced the endogenous anti-tumor response albeit, in the clinical setting, with significant unforeseen toxicities.

CTLA-4 contains an extracellular V domain, a transmembrane domain, and a cytoplasmic tail. Alternate splice variants, encoding different isoforms, have been characterized. The membrane-bound isoform functions as a homodimer interconnected by a disulfide bond, while the soluble isoform functions as a monomer. The intracellular domain is similar to that of CD28, in that it has no intrinsic catalytic activity and contains one YVKM motif able to bind PI3K, PP2A and SHP-2 and one proline-rich motif able to bind SH3 containing proteins. One role of CTLA-4 in inhibiting T cell responses seem to be directly via SHP-2 and PP2A dephosphorylation of TCR-proximal signaling proteins such as CD3 and LAT. CTLA-4 can also affect signaling indirectly via competing with CD28 for CD80/86 binding. CTLA-4 has also been shown to bind and/or interact with PI3K, CD80, AP2M1, and PPP2R5A.

In accordance with the presently disclosed subject matter, a CTLA-4 polypeptide can have an amino acid sequence that is at least about 85%, about 90%, about 95%, about 96%, about 97%, about 98%, about 99% or about 100% homologous to UniProtKB/Swiss-Prot Ref. No.: P16410.3 (SEQ ID NO: 130) (homology herein can be determined using standard software such as BLAST or FASTA) or fragments thereof, and/or can optionally comprise up to one or up to two or up to three conservative amino acid substitutions.

SEQ ID NO: 130 is provided below:

(SEQ ID NO: 130)
MACLGFQRHKAQLNLATRTWPCTLLFFLLFIPVFCKAMHVAQPAWLASSRG
IASFVCEYASPGKATEVRVTVLRQADSQVTEVCAATYMMGNELTFLDDSIC
TGTSSGNQLTIQGLRAMDTGLYICKVELMYPPPYYLGIGNGTQIYVIDPEP
CPDSDFLLWILAAVSSGLFFYSFLLTAVSLSKMLKKRSPLTTGVYVKMPPT
EPECEKQFQPYFIPIN.

In accordance with the presently disclosed subject matter, a “CTLA-4 nucleic acid molecule” refers to a polynucleotide encoding a CTLA-4 polypeptide.

Lymphocyte-activation protein 3 (LAG-3) is a negative immune regulator of immune cells. LAG-3 belongs to the immunoglobulin (Ig) superfamily and contains 4 extracellular Ig-like domains. The LAG3 gene contains 8 exons. The sequence data, exon/intron organization, and chromosomal localization all indicate a close relationship of LAG3 to CD4. LAG3 has also been designated CD223 (cluster of differentiation 223).

In accordance with the presently disclosed subject matter, a LAG-3 polypeptide can have an amino acid sequence that is at least about 85%, about 90%, about 95%, about 96%, about 97%, about 98%, about 99% or about 100% homologous to UniProtKB/Swiss-Prot Ref. No.: P18627.5 (SEQ ID NO: 131) or fragments thereof, and/or can optionally comprise up to one or up to two or up to three conservative amino acid substitutions.

SEQ ID NO: 131 is provided below:

(SEQ ID NO: 131)
MWEAQFLGLLFLQPLWVAPVKPLQPGAEVPWWAQEGAPAQLPCSPTIPLQD
LSLLRRAGVTWQHQPDSGPPAAAPGHPLAPGPHPAAPSSWGPRPRRYTVLS
VGPGGLRSGRLPLQPRVQLDERGRQRGDFSLWLRPARRADAGEYRAAVHLR
DRALSCRLRLRLGQASMTASPPGSLRASDWVILNCSFSRPDRPASVHWFRN
RGQGRVPVRESPHHHLAESFLFLPQVSPMDSGPWGCILTYRDGFNVSIIVI
YNLTVLGLEPPTPLTVYAGAGSRVGLPCRLPAGVGTRSFLTAKWTPPGGGP
DLLVTGDNGDFTLRLEDVSQAQAGTYTCHIHLQEQQLNATVTLAIITVTPK
SFGSPGSLGKLLCEVTPVSGQERFVWSSLDTPSQRSFSGPWLEAQEAQLLS
QPWQCQLYQGERLLGAAVYFTELSSPGAQRSGRAPGALPAGHLLLFLILGV
LSLLLLVTGAFGFHLWRRQWRPRRFSALEQGIHPPQAQSKIEELEQEPEPE
PEPEPEPEPEPEPEQL.

In accordance with the presently disclosed subject matter, a “LAG-3 nucleic acid molecule” refers to a polynucleotide encoding a LAG-3 polypeptide. Natural Killer Cell Receptor 2B4 (2B4) mediates non-MHC restricted cell killing on NK cells and subsets of T cells. To date, the function of 2B4 is still under investigation, with the 2B4-S isoform believed to be an activating receptor, and the 2B4-L isoform believed to be a negative immune regulator of immune cells. 2B4 becomes engaged upon binding its high-affinity ligand, CD48. 2B4 contains a tyrosine-based switch motif, a molecular switch that allows the protein to associate with various phosphatases. 2B4 has also been designated CD244 (cluster of differentiation 244).

In accordance with the presently disclosed subject matter, a 2B4 polypeptide can have an amino acid sequence that is at least about 85%, about 90%, about 95%, about 96%, about 97%, about 98%, about 99% or about 100% homologous to UniProtKB/Swiss-Prot Ref. No.: Q9BZW8.2 (SEQ ID NO: 132) or fragments thereof, and/or can optionally comprise up to one or up to two or up to three conservative amino acid substitutions.

SEQ ID NO: 132 is provided below:

(SEQ ID NO: 132)
MLGQWTLILLLLLKVYQGKGCQGSADHWSISGVPLQLQPNSIQTKVDSIAW
KKLLPSQNGFHHILKWENGSLPSNTSNDRFSFIVKNLSLLIKAAQQQDSGL
YCLEVTSISGKVQTATFQVFVFESLLPDKVEKPRLQGQGKILDRGRCQVAL
SCLVSRDGNVSYAWYRGSKLIQTAGNLTYLDEEVDINGTHTYTCNVSNPVS
WESHTLNLTQDCQNAHQEFRFWPFLVIIVILSALFLGTLACFCVWRRKRKE
KQSETSPKEFLTIYEDVKDLKTRRNHEQEQTFPGGGSTIYSMIQSQSSAPT
SQEPAYTLYSLIQPSRKSGSRKRNHSPSFNSTIYEVIGKSQPKAQNPARLS
RKELENFDVYS.

In accordance with the presently disclosed subject matter, a “2B4 nucleic acid molecule” refers to a polynucleotide encoding a 2B4 polypeptide.

B- and T-lymphocyte attenuator (BTLA) expression is induced during activation of T cells, and BTLA remains expressed on Th1 cells but not Th2 cells. Like PD1 and CTLA4, BTLA interacts with a B7 homolog, B7H4. However, unlike PD-1 and CTLA-4, BTLA displays T-Cell inhibition via interaction with tumor necrosis family receptors (TNF-R), not just the B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses. BTLA activation has been shown to inhibit the function of human CD8⁺ cancer-specific T cells. BTLA has also been designated as CD272 (cluster of differentiation 272).

In accordance with the presently disclosed subject matter, a BTLA polypeptide can have an amino acid sequence that is at least about 85%>, about 90%, about 95%, about 96%, about 97%, about 98%, about 99% or about 100% homologous to UniProtKB/Swiss-Prot Ref. No.: Q7Z6A9.3 (SEQ ID NO: 133) or fragments thereof, and/or can optionally comprise up to one or up to two or up to three conservative amino acid substitutions.

SEQ ID NO: 133 is provided below:

(SEQ ID NO: 133)
MKTLPAMLGTGKLFWVFFLIPYLDIWNIHGKESCDVQLYIKRQSEHSILAG
DPFELECPVKYCANRPHVTWCKLNGTTCVKLEDRQTSWKEEKNISFFILHF
EPVLPNDNGSYRCSANFQSNLIESHSTTLYVTDVKSASERPSKDEMASRPW
LLYRLLPLGGLPLLITTCFCLFCCLRRHQGKQNELSDTAGREINLVDAHLK
SEQTEASTRQNSQVLLSETGIYDNDPDLCFRMQEGSEVYSNPCLEENKPGI
VYASLNHSVIGPNSRLARNVKEAPTEYASICVRS.

In accordance with the presently disclosed subject matter, a “BTLA nucleic acid molecule” refers to a polynucleotide encoding a BTLA polypeptide.

### Immune Cells

The presently disclosed subject matter provides engineered immune cells expressing an engineered receptor (e.g., a CAR, caTCR, or eTCR) or other ligand that comprises an extracellular antigen-binding domain, a transmembrane domain and an intracellular domain, where the extracellular antigen-binding domain specifically binds a tumor antigen, including a tumor receptor or ligand, as described above. In certain embodiments immune cells can be transduced with a presently disclosed vectors encoding an engineered receptor such that the cells express the engineered receptor. The presently disclosed subject matter also provides methods of using such cells for the treatment of a tumor. The engineered immune cells of the presently disclosed subject matter can be cells of the lymphoid lineage or myeloid lineage. The lymphoid lineage, comprising B, T, and natural killer (NK) cells, provides for the production of antibodies, regulation of the cellular immune system, detection of foreign agents in the blood, detection of cells foreign to the host, and the like. Non-limiting examples of immune cells of the lymphoid lineage include T cells, Natural Killer (NK) cells, embryonic stem cells, and pluripotent stem cells (e.g., those from which lymphoid cells can be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, T helper cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells. In certain embodiments, the engineered T cells express Foxp3 to achieve and maintain a T regulatory phenotype.

Natural killer (NK) cells can be lymphocytes that are part of cell-mediated immunity and act during the innate immune response. NK cells do not require prior activation in order to perform their cytotoxic effect on target cells.

The engineered immune cells of the presently disclosed subject matter can express an extracellular antigen-binding domain (e.g., a human scFV, a Fab that is optionally crosslinked, or a F(ab)₂) that specifically binds to a tumor antigen, for the treatment of cancer, e.g., for treatment of solid tumor. Such engineered immune cells can be administered to a subject (e.g., a human subject) in need thereof for the treatment of cancer. In some embodiments, the immune cell is a lymphocyte, such as a T cell, a B cell or a natural killer (NK) cell. In certain embodiments, the engineered immune cell is a T cell. The T cell can be a CD4⁺ T cell or a CD8⁺ T cell. In certain embodiments, the T cell is a CD4⁺ T cell. In certain embodiments, the T cell is a CD8⁺ T cell.

A presently disclosed engineered immune cells can further include at least one recombinant or exogenous co-stimulatory ligand. For example, a presently disclosed engineered immune cells can be further transduced with at least one co-stimulatory ligand, such that the engineered immune cells co-expresses or is induced to co-express the tumor antigen-targeted engineered receptor and the at least one co-stimulatory ligand. The interaction between the tumor antigen-targeted engineered receptor and at least one co-stimulatory ligand provides a non-antigen-specific signal important for full activation of an immune cell (e.g., T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD 154, CD137L/4-1BBL, TNF-a, CD134L/OX40L/CD252, CD27L/CD70, Fas ligand (FasL), CD30L/CD153, tumor necrosis factor beta (TNFP)/lymphotoxin-alpha (LTa), lymphotoxin-beta O-Tβ), CD257/B cell-activating factor (B AFF)/Bly s/THANK/Tall-1, glucocorticoid-induced TNF Receptor ligand (GITRL), and T F-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the engineered immune cell comprises one recombinant co-stimulatory ligand that is 4-1BBL. In certain embodiments, the engineered immune cell comprises two recombinant co-stimulatory ligands that are 4-1BBL and CD80. Engineered receptors comprising at least one co-stimulatory ligand are described in U.S. Pat. No. 8,389,282, which is incorporated by reference in its entirety.

Furthermore, a presently disclosed engineered immune cells can further comprise at least one exogenous cytokine. For example, a presently disclosed engineered immune cell can be further transduced with at least one cytokine, such that the engineered immune cells secrete the at least one cytokine as well as expresses the tumor antigen-targeted engineered receptor. In certain embodiments, the at least one cytokine is selected from the group consisting of IL-2, IL-3, IL-6, IL-7, IL-11, IL-12, IL-15, IL-17, and IL-21. In certain embodiments, the cytokine is IL-12.

The engineered immune cells can be generated from peripheral donor lymphocytes, e.g., those disclosed in Sadelain, M., et al., Nat Rev Cancer 3:35-45 (2003) (disclosing peripheral donor lymphocytes genetically modified to express CARs), in Morgan, R. A. et al. (2006) Science 314: 126-129 (disclosing peripheral donor lymphocytes genetically modified to express a full-length tumor antigen-recognizing T cell receptor complex comprising the α and β heterodimer), in Panelli et al. (2000) J Immunol 164:495-504; Panelli et al. (2000) J Immunol 164:4382-4392 (2000) (disclosing lymphocyte cultures derived from tumor infiltrating lymphocytes (TILs) in tumor biopsies), and in Dupont et al. (2005) Cancer Res 65:5417-5427; Papanicolaou et al. (2003) Blood 102:2498-2505 (disclosing selectively in v/Yro-expanded antigen-specific peripheral blood leukocytes employing artificial antigen-presenting cells (AAPCs) or pulsed dendritic cells). The engineered immune cells (e.g., T cells) can be autologous, non-autologous (e.g., allogeneic), or derived in vitro from engineered progenitor or stem cells.

In certain embodiments, a presently disclosed engineered immune cells (e.g., T cells) expresses from about 1 to about 5, from about 1 to about 4, from about 2 to about 5, from about 2 to about 4, from about 3 to about 5, from about 3 to about 4, from about 4 to about 5, from about 1 to about 2, from about 2 to about 3, from about 3 to about 4, or from about 4 to about 5 vector copy numbers per cell of a presently disclosed tumor antigen-targeted engineered receptor.

For example, the higher the engineered receptor expression level in an engineered immune cell, the greater cytotoxicity and cytokine production the engineered immune cell exhibits. An engineered immune cell (e.g., T cell) having a high tumor antigen-targeted engineered receptor expression level can induce antigen-specific cytokine production or secretion and/or exhibit cytotoxicity to a tissue or a cell having a low expression level of tumor antigen-targeted engineered receptor, e.g., about 2,000 or less, about 1,000 or less, about 900 or less, about 800 or less, about 700 or less, about 600 or less, about 500 or less, about 400 or less, about 300 or less, about 200 or less, about 100 or less of tumor antigen binding sites/cell. Additionally, or alternatively, the cytotoxicity and cytokine production of a presently disclosed engineered immune cell (e.g., T cell) are proportional to the expression level of tumor antigen in a target tissue or a target cell. For example, the higher the expression level of human tumor antigen in the target, the greater cytotoxicity and cytokine production the engineered immune cell exhibits.

As described herein, the use of a FoxP3 targeting agent increases the cytotoxic effect in the engineered immune cells by depleting the disease microenvironment of FoxP3+ immunosuppressant cells (e.g., Tregs and Treg-like cells). In certain embodiments, an engineered immune cells of the present disclosure exhibits a cytotoxic effect against tumor antigen-expressing cells that is at least about 2-times, about 3-times, about 4-times, about 5-times, about 6-times, about 7-times, about 8-times, about 9-times, about 10-times, about 20-times, about 30-times, about 40-times, about 50-times, about 60-times, about 70-times, about 80-times, about 90-times, or about 100-times more than the cytotoxic effect of the engineered immune cell in the absence of the FoxP3 targeting agent.

The unpurified source of immune cells can be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-immune cell initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. In some embodiments, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). In some embodiments, the cells are collected in a medium comprising 2% fetal calf serum (FCS) or 0.2% bovine serum albumin (BSA) or any other suitable, preferably sterile, isotonic medium.

Alternatively, or in addition to separation or removal of irrelevant cells, a FoxP3 target agent can be used in the manufacture of an engineered immune cell. In some embodiments, the FoxP3 targeting agent is administered to the cell sample prior to transduction or transfection with a vector encoding an engineered receptor. In other embodiments, the FoxP3 targeting agent is administered to the cell sample during transduction or transfection with a vector encoding an engineered receptor. In other embodiments, the FoxP3 targeting agent is administered to the cell sample after transduction or transfection with a vector encoding an engineered receptor.

The use of a FoxP3 targeting agent in the manufacture of the engineered immune cell can increase the yield of engineered immune cells that are effector cells by depleting the FoxP3+ immunosuppressant cells (e.g., Tregs and Treg-like cells) from the cell sample. In certain embodiments, a composition comprising engineered immune cells manufactured in the presence of a FoxP3 targeting agent contains at least about 2-times, about 3-times, about 4-times, about 5-times, about 6-times, about 7-times, about 8-times, about 9-times, about 10-times, about 20-times, about 30-times, about 40-times, about 50-times, about 60-times, about 70-times, about 80-times, about 90-times, or about 100-times more effector cells than the number of effector cells produced in the absence of the FoxP3 targeting agent.

In some embodiments, the engineered immune cells comprise one or more additional modifications. For example, in some embodiments, the engineered immune cells comprise and express (is transduced to express) an antigen recognizing receptor that binds to a second antigen that is different from the tumor antigen. The inclusion of an antigen recognizing receptor in addition to a presently disclosed engineered receptor on the engineered immune cell can increase the avidity of the engineered receptor or the engineered immune cell comprising thereof on a targeted cell, especially, the engineered receptor is one that has a low binding affinity to a particular tumor antigen, e.g., a K_{d} of about 2×10⁻⁸ M or more, about 5×10⁻⁸ M or more, about 8×10⁻⁸ M or more, about 9×10⁻⁸ M or more, about 1×10⁻⁷ M or more, about 2×10⁻⁷ M or more, or about 5×10⁻⁷ M or more.

In certain embodiments, the antigen recognizing receptor is a chimeric co-stimulatory receptor (CCR). CCR is described in Krause et al. (1998) J Exp. Med. 188(4):619-626, and US20020018783, the contents of which are incorporated by reference in their entireties. CCRs mimic co-stimulatory signals, but unlike, engineered receptors, do not provide a T-cell activation signal, e.g., CCRs lack a CD3ζ polypeptide. CCRs provide co-stimulation, e.g., a CD28-like signal, in the absence of the natural co-stimulatory ligand on the antigen-presenting cell. A combinatorial antigen recognition, i.e., use of a CCR in combination with an engineered receptor, can augment T-cell reactivity against the dual-antigen expressing T cells, thereby improving selective tumor targeting. Kloss et al., describe a strategy that integrates combinatorial antigen recognition, split signaling, and, critically, balanced strength of T-cell activation and costimulation to generate T cells that eliminate target cells that express a combination of antigens while sparing cells that express each antigen individually (Kloss et al. (2013) Nature Biotechnology 31(1):71-75). With this approach, T-cell activation requires engineered receptor-mediated recognition of one antigen, whereas costimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen. In certain embodiments, the CCR comprises an extracellular antigen-binding domain that binds to an antigen different than selected tumor antigen, a transmembrane domain, and a co-stimulatory signaling region that comprises at least one co-stimulatory molecule, including, but not limited to, CD28, 4-1BB, OX40, ICOS, PD-1, CTLA-4, LAG-3, 2B4, and BTLA. In certain embodiments, the co-stimulatory signaling region of the CCR comprises one co-stimulatory signaling molecule. In certain embodiments, the one co-stimulatory signaling molecule is CD28. In certain embodiments, the one co-stimulatory signaling molecule is 4-IBB. In certain embodiments, the co-stimulatory signaling region of the CCR comprises two co-stimulatory signaling molecules. In certain embodiments, the two co-stimulatory signaling molecules are CD28 and 4-IBB. A second antigen is selected so that expression of both selected tumor antigen and the second antigen is restricted to the targeted cells (e.g., cancerous tissue or cancerous cells). Similar to an engineered receptor, the extracellular antigen-binding domain can be a scFv, a Fab, a F(ab)₂; or a fusion protein with a heterologous sequence to form the extracellular antigen-binding domain. In certain embodiments, the CCR comprises a scFv that binds to CD 138, transmembrane domain comprising a CD28 polypeptide, and a co-stimulatory signaling region comprising two co-stimulatory signaling molecules that are CD28 and 4-IBB.

In certain embodiments, the antigen recognizing receptor is a truncated CAR. A “truncated CAR” is different from a CAR by lacking an intracellular signaling domain. For example, a truncated CAR comprises an extracellular antigen-binding domain and a transmembrane domain, and lacks an intracellular signaling domain. In accordance with the presently disclosed subject matter, the truncated CAR has a high binding affinity to the second antigen expressed on the targeted cells, e.g., myeloma cells. The truncated CAR functions as an adhesion molecule that enhances the avidity of a presently disclosed engineered receptor, especially, one that has a low binding affinity to tumor antigen, thereby improving the efficacy of the presently disclosed engineered receptor or engineered immune cell (e.g., T cell) comprising thereof. In certain embodiments, the truncated CAR comprises an extracellular antigen-binding domain that binds to CD 138, a transmembrane domain comprising a CD8 polypeptide. A presently disclosed T cell comprises or is transduced to express a presently disclosed engineered receptor targeting tumor antigen and a truncated CAR targeting CD138. In certain embodiments, the targeted cells are solid tumor cells.

In some embodiments, the engineered immune cells are further modified to suppress expression of one or more genes. In some embodiments, the engineered immune cells are further modified via genome editing. Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, for example, U.S. Pat. Nos. 7,888,121; 7,972,854; 7,914,796; 7,951,925; 8,110,379; 8,409,861; 8,586,526; U.S. Patent Publications 20030232410; 20050208489; 20050026157; 20050064474; 20060063231; 201000218264; 20120017290; 20110265198; 20130137104; 20130122591; 20130177983 and 20130177960, the disclosures of which are incorporated by reference in their entireties. These methods often involve the use of engineered cleavage systems to induce a double strand break (DSB) or a nick in a target DNA sequence such that repair of the break by an error born process such as non-homologous end joining (NHEJ) or repair using a repair template (homology directed repair or HDR) can result in the knock out of a gene or the insertion of a sequence of interest (targeted integration). Cleavage can occur through the use of specific nucleases such as engineered zinc finger nucleases (ZFN), transcription-activator like effector nucleases (TALENs), or using the CRISPR/Cas system with an engineered crRNA/tracr RNA (‘single guide RNA’) to guide specific cleavage. In some embodiments, the engineered immune cells are modified to disrupt or reduce expression of an endogenous T-cell receptor gene (see, e.g. WO 2014153470, which is incorporated by reference in its entirety). In some embodiments, the engineered immune cells are modified to result in disruption or inhibition of PD1, PDL-1 or CTLA-4 (see, e.g. U.S. Patent Publication 20140120622), or other immunosuppressive factors known in the art (Wu et al. (2015) Oncoimmunology 4(7): e1016700, Mahoney et al. (2015) Nature Reviews Drug Discovery 14, 561-584).

### FoxP3 Targeting Agents

In some embodiments, provided herein are FoxP3 targeting agents for use in enhancing the efficacy of an engineered immune cell expressing a T-cell receptor (TCR) or other cell-surface ligand that binds to a target antigen, such as a tumor antigen or viral protein. Also provided herein are FoxP3 targeting agents for use in the manufacture of an engineered immune cell expressing a T-cell receptor (TCR) or other cell-surface ligand that binds to a target antigen, such as a tumor antigen or viral protein.

In some embodiments, the FoxP3 targeting agents are antigen-binding proteins, including antibodies, chimeric antigen receptors (CARs), chimeric antibody TCRs (caTCRS), and/or engineered TCRs (eTCRs) specific for a FoxP3 polypeptide of FoxP3-derived peptide fragment. In some embodiments, the FoxP3 polypeptide comprises the amino acid sequence set forth in SEQ ID NO: 1.

In some embodiments, FoxP3-derived peptide fragment has a length of 8-12 amino acids. In some embodiments, the FoxP3-derived peptide fragment is selected from FoxP3-1 having the amino acid sequence set forth in SEQ ID NO: 2 or a portion thereof, FoxP3-2 having the amino acid sequence set forth in SEQ ID NO: 3 or a portion thereof, FoxP3-3 having the amino acid sequence set forth in SEQ ID NO: 4 or a portion thereof, FoxP3-4 having the amino acid sequence set forth in SEQ ID NO: 5 or a portion thereof, FoxP3-5 having the amino acid sequence set forth in SEQ ID NO: 6 or a portion thereof, FoxP3-6 having the amino acid sequence set forth in SEQ ID NO: 7 or a portion thereof; and FoxP3-7 having the amino acid sequence set forth in SEQ ID NO: 8 or a portion thereof. In some embodiments, the FoxP3-derived peptide fragment is FoxP3-7 having the amino acid sequence set forth in SEQ ID NO: 8 or a portion thereof.

In some embodiments, the FoxP3 targeting agent binds to FoxP3 presented in the context of an MHC molecule (e.g., FoxP3/MHC complex). In some embodiments, the FoxP3 targeting agent binds to FoxP3 presented in the context of an HLA-A molecule (e.g., FoxP3/HLA-A complex). In some embodiments, the FoxP3 targeting agent binds to FoxP3 presented in the context of an HLA-A2 molecule (e.g., FoxP3/HLA-A2 complex). In some embodiments, the FoxP3 targeting agent binds to FoxP3 presented in the context of an HLA-A*02:01 molecule (e.g., FoxP3/HLA-A*02:01 complex).

In exemplary embodiments, the FoxP3 targeting agents provided herein are bispecific antibodies. In some embodiments, the bispecific antibody binds to a FoxP3 polypeptide, or fragment thereof, and a cell surface protein. In some embodiments, cell surface protein is CD3 or CD16.

In exemplary embodiments, the FoxP3 targeting agents are engineered immune cells that express an engineered receptor (e.g., a CAR, caTCR, or eTCR) or other cell-surface ligand that binds to FoxP3. In some embodiments, the FoxP3 targeting agents are engineered immune cells that express an engineered receptor (e.g., a CAR, caTCR, or eTCR) or other cell-surface ligand that binds to FoxP3 presented in the context of an MEW molecule. In some embodiments, the FoxP3 targeting agents are engineered immune cells that express an engineered receptor (e.g., a CAR, caTCR, or eTCR) or other cell-surface ligand that binds to FoxP3 presented in the context of an HLA-A2 molecule. In some embodiments, the TCR or other cell-surface ligand that binds to FoxP3 comprises a transmembrane domain of an engineered receptor, intracellular domain of an engineered receptor, and/or linker of an engineered receptor as described above. In some embodiments, the engineered immune cell that expresses a TCR (i.e., engineered receptor) or other cell-surface ligand that binds to FoxP3 is an immune cell as described above. In some embodiments, the engineered immune cell that expresses a TCR or other cell-surface ligand that binds to FoxP3 comprises one or more features of an engineered immune cell that expresses a TCR or other cell-surface ligand that binds to a target antigen as described above.

In exemplary embodiments, the engineered immune cells express a single type of engineered receptor (e.g., a CAR, caTCR, or eTCR) or other cell-surface ligand that binds to FoxP3 presented in the context of an MEW molecule. In some embodiments, the engineered immune cells express two or more engineered receptors (e.g., a CAR, caTCR, or eTCR) or other cell-surface ligand that binds to FoxP3 presented in the context of an MHC molecule. In some embodiments, the engineered immune cells express one or more engineered receptors (e.g., a CAR, caTCR, or eTCR) or other cell-surface ligand that binds to FoxP3 presented in the context of an MHC molecule and also express one or more additional engineered receptors (e.g., a CAR, caTCR, or eTCR) or other cell-surface ligand that binds to a different cell-surface receptor (e.g., CD19).

In some embodiments, the antigen-binding proteins specific for FoxP3 comprise a heavy chain variable region CDR1 comprising an amino acid sequence set forth in SEQ ID NO: 16; a heavy chain variable region CDR2 comprising an amino acid sequence set forth in SEQ ID NO: 17; a heavy chain variable region CDR3 comprising an amino acid sequence set forth in SEQ ID NO: 18; a light chain variable region CDR1 comprising an amino acid sequence set forth in SEQ ID NO: 19; a light chain variable region CDR2 comprising an amino acid sequence set forth in SEQ ID NO: 20; and a light chain variable region CDR3 comprising an amino acid sequence set forth in SEQ ID NO: 21. In some embodiments, the antigen-binding proteins specific for FoxP3 comprise a heavy chain variable region CDR1 comprising an amino acid sequence set forth in SEQ ID NO: 22; a heavy chain variable region CDR2 comprising an amino acid sequence set forth in SEQ ID NO: 23; a heavy chain variable region CDR3 comprising an amino acid sequence set forth in SEQ ID NO: 24; a light chain variable region CDR1 comprising an amino acid sequence set forth in SEQ ID NO: 25; a light chain variable region CDR2 comprising an amino acid sequence set forth in SEQ ID NO: 26; and a light chain variable region CDR3 comprising an amino acid sequence set forth in SEQ ID NO: 27. In some embodiments, the antigen-binding proteins specific for FoxP3 comprise a heavy chain variable region CDR1 comprising an amino acid sequence set forth in SEQ ID NO: 28; a heavy chain variable region CDR2 comprising an amino acid sequence set forth in SEQ ID NO: 29; a heavy chain variable region CDR3 comprising an amino acid sequence set forth in SEQ ID NO: 30; a light chain variable region CDR1 comprising an amino acid sequence set forth in SEQ ID NO: 31; a light chain variable region CDR2 comprising an amino acid sequence set forth in SEQ ID NO: 32; and a light chain variable region CDR3 comprising an amino acid sequence set forth in SEQ ID NO: 33. In some embodiments, the antigen-binding proteins specific for FoxP3 comprise a heavy chain variable region CDR1 comprising an amino acid sequence set forth in SEQ ID NO: 34; a heavy chain variable region CDR2 comprising an amino acid sequence set forth in SEQ ID NO: 35; a heavy chain variable region CDR3 comprising an amino acid sequence set forth in SEQ ID NO: 36; a light chain variable region CDR1 comprising an amino acid sequence set forth in SEQ ID NO: 37; a light chain variable region CDR2 comprising an amino acid sequence set forth in SEQ ID NO: 38; and a light chain variable region CDR3 comprising an amino acid sequence set forth in SEQ ID NO: 39. In some embodiments, the antigen-binding proteins specific for FoxP3 comprise a heavy chain variable region CDR1 comprising an amino acid sequence set forth in SEQ ID NO: 40; a heavy chain variable region CDR2 comprising an amino acid sequence set forth in SEQ ID NO: 41; a heavy chain variable region CDR3 comprising an amino acid sequence set forth in SEQ ID NO: 42; a light chain variable region CDR1 comprising an amino acid sequence set forth in SEQ ID NO: 43; a light chain variable region CDR2 comprising an amino acid sequence set forth in SEQ ID NO: 44; and a light chain variable region CDR3 comprising an amino acid sequence set forth in SEQ ID NO: 45. In some embodiments, the antigen-binding proteins specific for FoxP3 comprise a heavy chain variable region CDR1 comprising an amino acid sequence set forth in SEQ ID NO: 46; a heavy chain variable region CDR2 comprising an amino acid sequence set forth in SEQ ID NO: 47; a heavy chain variable region CDR3 comprising an amino acid sequence set forth in SEQ ID NO: 48; a light chain variable region CDR1 comprising an amino acid sequence set forth in SEQ ID NO: 49; a light chain variable region CDR2 comprising an amino acid sequence set forth in SEQ ID NO: 50; and a light chain variable region CDR3 comprising an amino acid sequence set forth in SEQ ID NO: 51. In some embodiments, the antigen-binding proteins specific for FoxP3 comprise a heavy chain variable region CDR1 comprising an amino acid sequence set forth in SEQ ID NO: 52; a heavy chain variable region CDR2 comprising an amino acid sequence set forth in SEQ ID NO: 53; a heavy chain variable region CDR3 comprising an amino acid sequence set forth in SEQ ID NO: 54; a light chain variable region CDR1 comprising an amino acid sequence set forth in SEQ ID NO: 55; a light chain variable region CDR2 comprising an amino acid sequence set forth in SEQ ID NO: 56; and a light chain variable region CDR3 comprising an amino acid sequence set forth in SEQ ID NO: 57. In some embodiments, the antigen-binding proteins specific for FoxP3 comprise a heavy chain variable region CDR1 comprising an amino acid sequence set forth in SEQ ID NO: 58; a heavy chain variable region CDR2 comprising an amino acid sequence set forth in SEQ ID NO: 59; a heavy chain variable region CDR3 comprising an amino acid sequence set forth in SEQ ID NO: 60; a light chain variable region CDR1 comprising an amino acid sequence set forth in SEQ ID NO: 61; a light chain variable region CDR2 comprising an amino acid sequence set forth in SEQ ID NO: 62; and a light chain variable region CDR3 comprising an amino acid sequence set forth in SEQ ID NO: 63.

In some embodiments, the antigen-binding protein specific for FoxP3 comprises a heavy chain variable region comprising amino acids having a sequence of SEQ ID NOs: 64-77, or a functional fragment or variant thereof. In some embodiments, the extracellular antigen-binding domain (e.g., human scFv) comprises a light chain variable region comprising amino acids having a sequence of SEQ ID NOs: 78-91, or a functional fragment or variant thereof. In some embodiments, the extracellular antigen-binding domain is a human scFv, which comprises a heavy chain variable region comprising amino acids having the sequence set forth SEQ ID NOs: 64-77, or a functional fragment or variant thereof and a light chain variable region comprising amino acids having the sequence set forth in SEQ ID NOs: 78-91, or a functional fragment or variant thereof, optionally with (iii) a linker sequence, for example a linker peptide, between the heavy chain variable region and the light chain variable region. In certain embodiments, the linker comprises amino acids having the sequence set forth in SEQ ID NO: 118 (SRGGGGSGGGGSGGGGSLEMA). In certain embodiments, the extracellular antigen-binding domain is a human scFv-Fc fusion protein or full length human IgG with V_{H} and V_{L} regions.

In certain embodiments, the extracellular antigen-binding domain comprises a V_{H} comprising an amino acid sequence that is at least 80%, at least 85%, at least 90%, or at least 95% identical to SEQ ID NO: 64-77. For example, the extracellular antigen-binding domain comprises a V_{H} comprising an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NOs: 64-77. In certain embodiments, the extracellular antigen-binding domain comprises a V_{H} comprising amino acids having the sequence set forth in SEQ ID NOs: 64-77. In certain embodiments, the extracellular antigen-binding domain comprises a V_{L} comprising an amino acid sequence that is at least 80%, at least 85%, at least 90%, or at least 95% identical to SEQ ID NOs: 78-91. For example, the extracellular antigen-binding domain comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to SEQ ID NOs: 78-91. In certain embodiments, the extracellular antigen-binding domain comprises a V_{L} comprising amino acids having the sequence set forth in SEQ ID NOs: 78-91.

In some embodiments, the V_{H} and/or V_{L} amino acid sequences having at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology to the specified sequences (e.g., SEQ ID NOs: 64-91) contain substitutions (e.g., conservative substitutions), insertions, or deletions relative to the specified sequence(s), but retain the ability to bind to the respective target antigen. In certain embodiments, a total of 1 to 10 amino acids are substituted, inserted and/or deleted in SEQ ID NOs: 64-91. In certain embodiments, substitutions, insertions, or deletions occur in regions outside the CDRs (e.g., in the framework regions (FRs)) of the extracellular antigen-binding domain. In certain embodiments, the extracellular antigen-binding domain comprises a V_{H} and/or V_{L} sequence selected from the group consisting of SEQ ID NOs: 64-91, including post-translational modifications of that sequence.

In some embodiments, the antigen-binding proteins specific for FoxP3 comprise a heavy chain variable region comprising an amino acid sequence set forth in SEQ ID NO: 69, and a light chain variable region that comprising an amino acid sequence set forth in SEQ ID NO: 83.

In some embodiments, the antigen-binding proteins specific for FoxP3 comprise a heavy chain variable region comprising an amino acid sequence set forth in WO2017/124001, which is incorporated by reference in its entirety.

In some embodiments, the antigen-binding proteins specific for FoxP3 comprise a heavy chain variable region CDR1 having an amino acid sequence set forth in WO2017/124001, which is incorporated by reference in its entirety.

In some embodiments, the antigen-binding proteins specific for FoxP3 comprise a heavy chain variable region CDR2 having an amino acid sequence set forth in WO2017/124001, which is incorporated by reference in its entirety.

In some embodiments, the antigen-binding proteins specific for FoxP3 comprise a heavy chain variable region CDR3 having an amino acid sequence set forth in WO2017/124001, which is incorporated by reference in its entirety.

In some embodiments, the antigen-binding proteins specific for FoxP3 comprise a light chain variable region CDR1 having an amino acid sequence set forth in WO2017/124001, which is incorporated by reference in its entirety.

In some embodiments, the antigen-binding proteins specific for FoxP3 comprise a light chain variable region CDR2 having an amino acid sequence set forth in WO2017/124001, which is incorporated by reference in its entirety.

In some embodiments, the antigen-binding proteins specific for FoxP3 comprise a light chain variable region CDR3 having an amino acid sequence set forth in WO2017/124001, which is incorporated by reference in its entirety.

In some embodiments, the antigen-binding proteins specific for FoxP3 comprise a light chain variable region comprising an amino acid sequence set forth in WO2017/124001, which is incorporated by reference in its entirety.

In some embodiments, the antigen-binding proteins specific for FoxP3 comprise a scFv having an amino acid sequence set forth in WO2017/124001, which is incorporated by reference in its entirety.

All FoxP3 scFv, antibody, and CARs sequences as described in WO2017/124001 are incorporated by reference in their entirety, including the amino acid and nucleotide sequences provided therein. These sequences include, without limitation, the amino acid and nucleotide sequences of Tables 1, 2, and 3, and Appendices A, B C, D, E, F, and G of WO2017/124001, which include amino acid and nucleotide sequences for selected FoxP3 antibody scFV, light chain, heavy chain, and CDR sequences. Any of the above sequences can be incorporated as part of the FoxP3 targeting agents described herein.

### Vectors

Many expression vectors are available and known to those of skill in the art and can be used for expression of polypeptides provided herein. The choice of expression vector will be influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector in the cells.

Vectors also can contain additional nucleotide sequences operably linked to the ligated nucleic acid molecule, such as, for example, an epitope tag such as for localization, e.g. a hexa-his tag (SEQ ID NO: 354) or a myc tag, hemagglutinin tag or a tag for purification, for example, a GST fusion, and a sequence for directing protein secretion and/or membrane association.

Expression of the antibodies or antigen-binding fragments thereof can be controlled by any promoter/enhancer known in the art. Suitable bacterial promoters are well known in the art and described herein below. Other suitable promoters for mammalian cells, yeast cells and insect cells are well known in the art and some are exemplified below. Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bernoist and Chambon, Nature 290:304-310(1981)), the promoter contained in the 3′ long terminal repeat of Rous sarcoma virus (Yamamoto et al. (1980) Cell 22:787-797), the herpes thymidine kinase promoter (Wagner et al. (1981) Proc. Natl. Acad. Sci. USA 75: 1441-1445), the regulatory sequences of the metallothionein gene (Brinster et al. (1982) Nature 296:39-42); prokaryotic expression vectors such as the β-lactamase promoter (Jay et al. (1981) Proc. Natl. Acad. Sci. USA 75:5543) or the tac promoter (DeBoer et al. (1983) Proc. Natl. Acad. Sci. USA 50:21-25); see also “Useful Proteins from Recombinant Bacteria” (1980) in Scientific American 242:79-94); plant expression vectors containing the nopaline synthetase promoter (Herrera-Estrella et al. (1984) Nature 505:209-213) or the cauliflower mosaic virus 35S RNA promoter (Gardner et al. (1981) Nucleic Acids Res. 9:2871), and the promoter of the photosynthetic enzyme ribulose bisphosphate carboxylase (Herrera-Estrella et al. (1984) Nature 510: 1 15-120); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al. (1984) Cell 55:639-646; Ornitz et al. (1986) Cold Spring Harbor Symp. Quant. Biol. 50:399-409; MacDonald (1987) Hepatology 7:425-515); insulin gene control region which is active in pancreatic beta cells (Hanahan et al. (1985) Nature 515: 115-122), immunoglobulin gene control region which is active in lymphoid cells (Grosschedl et al. (1984) Cell 55:647-658; Adams et al. (1985) Nature 515:533-538; Alexander et al. (1987) Mol. Cell Biol. 7: 1436-1444), mouse mammary tumor virus control region which is active in testicular, breast, lymphoid and mast cells (Leder et al. (1986) Cell 15:485-495), albumin gene control region which is active in liver (Pinckert et al. (1987) Genes and Devel. 1:268-276), alpha-fetoprotein gene control region which is active in liver (Krumlauf et al. (1985) Mol. Cell. Biol. 5:1639-403); Hammer et al. (1987) Science 255:53-58), alpha-1 antitrypsin gene control region which is active in liver (Kelsey et al. (1987) Genes and Devel. 7:161-171), beta globin gene control region which is active in myeloid cells (Magram et al. (1985) Nature 515:338-340); Kollias et al. (1986) Cell 5:89-94), myelin basic protein gene control region which is active in oligodendrocyte cells of the brain (Readhead et al. (1987) Cell 15:703-712), myosin light chain-2 gene control region which is active in skeletal muscle (Shani (1985) Nature 514:283-286), and gonadotrophic releasing hormone gene control region which is active in gonadotrophs of the hypothalamus (Mason et al. (1986) Science 254: 1372-1378).

In addition to the promoter, the expression vector typically contains a transcription unit or expression cassette that contains all the additional elements required for the expression of the antibody, or portion thereof, in host cells. A typical expression cassette contains a promoter operably linked to the nucleic acid sequence encoding the antibody chain and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a germline antibody chain under the direction of the polyhedron promoter or other strong baculovirus promoter.

Any methods known to those of skill in the art for the insertion of DNA fragments into a vector can be used to construct expression vectors containing a nucleic acid encoding any of the polypeptides provided herein. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

Exemplary plasmid vectors useful to produce the polypeptides provided herein contain a strong promoter, such as the HCMV immediate early enhancer/promoter or the MHC class I promoter, an intron to enhance processing of the transcript, such as the HCMV immediate early gene intron A, and a polyadenylation (poly A) signal, such as the late SV40 polyA signal.

Genetic modification of engineered immune cells (e.g., T cells, NK cells) can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding the tumor antigen-targeted engineered receptor and the FoxP3 targeting agent can be cloned into a retroviral vector and expression can be driven from its endogenous promoter, from the retroviral long terminal repeat, or from an alternative internal promoter.

Non-viral vectors or RNA can be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide tumor antigen-targeted engineered receptor and/or the FoxP3 targeting agent expressing cells or to produce FoxP3 targeting agents, a retroviral vector can be employed for transduction. However, any other suitable viral vector or non-viral delivery system can be used for genetic modification of cells. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller et al. (1985) Mol. Cell. Biol. 5:431-437); PA317 (Miller et al. (1986) Mol. Cell. Biol. 6:2895-2902); and CRIP (Danos et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni et al. (1992) Blood 80: 1418-1422, or culturing with viral supernatant alone or concentrated vector stocks with or without appropriate growth factors and polycations, e.g., by the method of Xu et al. (1994) Exp. Hemat. 22:223-230; and Hughes et al. (1992) J Clin. Invest. 89: 1817.

Transducing viral vectors can be used to express a co-stimulatory ligand and/or secretes a cytokine (e.g., 4-1BBL and/or IL-12) in an engineered immune cell. In some embodiments, the chosen vector exhibits high efficiency of infection and stable integration and expression (see, e.g., Cayouette et al. (1997) Human Gene Therapy 8:423-430; Kido et al. (1996) Current Eye Research 15:833-844; Bloomer et al. (1997) Journal of Virology 71:6641-6649; Naldini et al. (1996) Science 272:263 267; and Miyoshi et al. (1997) Proc. Natl. Acad. Sci. U.S.A. 94: 10319). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller (1990) Human Gene Therapy 15-14, Friedman (1989) Science 244: 1275-1281; Eglitis et al. (1988) BioTechniques 6:608-614; Tolstoshev et al. (1990) Current Opinion in Biotechnology 1:55-61; Sharp (1991) The Lancet 337: 1277-1278; Cornetta et al. (1987) Nucleic Acid Research and Molecular Biology 36:311-322; Anderson (1984) Science 226:401-409; Moen (1991) Blood Cells 17:407-416; Miller et al. (1989) Biotechnology 7:980-990; Le Gal La Salle et al. (1993) Science 259:988-990; and Johnson (1995) Chest 107:77S-83S). Retroviral vectors are particularly well developed and have been used in clinical settings (Rosenberg et al. (1990) N. Engl. J. Med 323:370; Anderson et al., U.S. Pat. No. 5,399,346).

In certain non-limiting embodiments, the vector expressing a presently disclosed tumor antigen-targeted engineered receptor is a retroviral vector, e.g., an oncoretroviral vector. In some instances, the retroviral vector is a SFG retroviral vector or murine stem cell virus (MSCV) retroviral vector. In certain non-limiting embodiments, the vector expressing a presently disclosed tumor antigen-targeted engineered receptor is a lentiviral vector. In certain non-limiting embodiments, the vector expressing a presently disclosed tumor antigen-targeted engineered receptor is a transposon vector.

Non-viral approaches can also be employed for the expression of a protein in cell. For example, a nucleic acid molecule can be introduced into a cell by administering the nucleic acid in the presence of lipofection (Feigner et al. (1987) Proc. Nat'l. Acad. Sci. U.S.A. 84:7413; Ono et al. (1990) Neuroscience Letters 17:259; Brigham et al. (1989)Am. J Med. Sci. 298:278; Staubinger et al. (1983) Methods in Enzymology 101:512), asialoorosomucoid-polylysine conjugation (Wu et al. (1988) Journal of Biological Chemistry 263: 14621; Wu et al. (1989) Journal of Biological Chemistry 264: 16985), or by micro-injection under surgical conditions (Wolff et al. (1990) Science 247: 1465). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALE nucleases). Transient expression can be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g., the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes. VI. Polypeptides and Analogs and Polynucleotides

Also included in the presently disclosed subject matter are extracellular antigen-binding domains that specifically binds to a tumor antigen (e.g., human tumor antigen) (e.g., an scFv (e.g., a human scFv), a Fab, or a (Fab)₂), CD3ζ, CD8, CD28, etc. polypeptides or fragments thereof, and polynucleotides encoding thereof that are modified in ways that enhance their anti-tumor activity when expressed in an engineered immune cell. The presently disclosed subject matter provides methods for optimizing an amino acid sequence or a nucleic acid sequence by producing an alteration in the sequence. Such alterations can comprise certain mutations, deletions, insertions, or post-translational modifications. The presently disclosed subject matter further comprises analogs of any naturally-occurring polypeptide of the presently disclosed subject matter. Analogs can differ from a naturally-occurring polypeptide of the presently disclosed subject matter by amino acid sequence differences, by post-translational modifications, or by both. Analogs of the presently disclosed subject matter can generally exhibit at least about 85%, about 90%, about 91%, about 92%, about 93%, about 94%, about 95%, about 96%, about 97%), about 98%, about 99% or more identity or homology with all or part of a naturally-occurring amino, acid sequence of the presently disclosed subject matter. The length of sequence comparison is at least about 5, about 10, about 15, about 20, about 25, about 50, about 75, about 100 or more amino acid residues. Again, in an exemplary approach to determining the degree of identity, a BLAST program can be used, with a probability score between e⁻³ and e⁻¹⁰⁰ indicating a closely related sequence. Modifications comprise in vivo and in vitro chemical derivatization of polypeptides, e.g., acetylation, carboxylation, phosphorylation, or glycosylation; such modifications can occur during polypeptide synthesis or processing or following treatment with isolated modifying enzymes. Analogs can also differ from the naturally-occurring polypeptides of the presently disclosed subject matter by alterations in primary sequence. These include genetic variants, both natural and induced (for example, resulting from random mutagenesis by irradiation or exposure to ethanemethyl sulfate or by site-specific mutagenesis as described in Sambrook, Fritsch and Maniatis, Molecular Cloning: A Laboratory Manual (2nd ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., beta ((3) or gamma (γ) amino acids.

In addition to full-length polypeptides, the presently disclosed subject matter also provides fragments of any one of the polypeptides or peptide domains of the presently disclosed subject matter. A fragment can be at least about 5, about 10, about 13, or about 15 amino acids. In some embodiments, a fragment is at least about 20 contiguous amino acids, at least about 30 contiguous amino acids, or at least about 50 contiguous amino acids. In some embodiments, a fragment is at least about 60 to about 80, about 100, about 200, about 300 or more contiguous amino acids. Fragments of the presently disclosed subject matter can be generated by methods known to those of ordinary skill in the art or can result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein of the invention. Such analogs are administered according to methods of the presently disclosed subject matter. Such analogs can exceed the physiological activity of the original polypeptide. Methods of analog design are well known in the art, and synthesis of analogs can be carried out according to such methods by modifying the chemical structures such that the resultant analogs increase the antineoplastic activity of the original polypeptide when expressed in an engineered immune cell. These chemical modifications include, but are not limited to, substituting alternative R groups and varying the degree of saturation at specific carbon atoms of a reference polypeptide. The protein analogs can be relatively resistant to in vivo degradation, resulting in a more prolonged therapeutic effect upon administration. Assays for measuring functional activity include, but are not limited to, those described in the Examples below.

In accordance with the presently disclosed subject matter, the polynucleotides encoding an extracellular antigen-binding domain that specifically binds to tumor antigen (e.g., human tumor antigen) (e.g., an scFv (e.g., a human scFv), a Fab, or a (Fab)₂), CD3, CD8, CD28) can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotides of the presently disclosed subject matter, including, but not limited to, OptimumGene™, Encor optimization, and Blue Heron.

### Administration

Engineered immune cells expressing the tumor antigen-targeted engineered receptor and a FoxP3 targeting agent of the presently disclosed subject matter can be provided systemically or directly to a subject for treating or preventing a disease, such as neoplasia or viral infection. In certain embodiments, engineered immune cells and/or FoxP3 targeting agent are directly injected into an organ of interest (e.g., an organ affected by a neoplasia). Alternatively, or additionally, the engineered immune cells and/or FoxP3 targeting agent are provided indirectly to the organ of interest, for example, by administration into the circulatory system (e.g., the tumor vasculature). Expansion and differentiation agents can be provided prior to, during or after administration of the engineered immune cells and/or FoxP3 targeting agent.

Engineered immune cells and/or FoxP3 targeting agents of the presently disclosed subject matter can be administered in any physiologically acceptable vehicle, systemically or regionally, normally intravascularly, intraperitoneally, intrathecally, or intrapleurally, although they can also be introduced into bone or other convenient site where the cells can find an appropriate site for regeneration and differentiation (e.g., thymus). In certain embodiments, at least 1×10⁵ cells can be administered, eventually reaching 1×10¹⁰ or more. In certain embodiments, at least 1×10⁶ cells can be administered. A cell population comprising engineered immune cells can comprise a purified population of cells. Those skilled in the art can readily determine the percentage of engineered immune cells in a cell population using various well-known methods, such as fluorescence activated cell sorting (FACS). The ranges of purity in cell populations comprising engineered immune cells can be from about 50% to about 55%, from about 55% to about 60%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity can require an increase in dosage). The engineered immune cells and/or FoxP3 targeting agents can be introduced by injection, catheter, or the like. If desired, factors can also be included, including, but not limited to, interleukins, e.g., IL-2, IL-3, IL 6, IL-11, IL-7, IL-12, IL-15, IL-21, as well as the other interleukins, the colony stimulating factors, such as G-, M- and GM-CSF, interferons, e.g., γ-interferon.

In certain embodiments, compositions of the presently disclosed subject matter comprise pharmaceutical compositions comprising engineered immune cells expressing a tumor antigen-targeted engineered receptor and a FoxP3 targeting agent with a pharmaceutically acceptable carrier. Administration can be autologous or non-autologous. For example, engineered immune cells expressing a tumor antigen-targeted engineered receptor and a FoxP3 targeting agent and compositions comprising thereof can be obtained from one subject, and administered to the same subject or a different, compatible subject. Peripheral blood derived T cells of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition of the presently disclosed subject matter (e.g., a pharmaceutical composition comprising engineered immune cells expressing a tumor antigen-targeted engineered receptor and a FoxP3 targeting agent), it can be formulated in a unit dosage injectable form (solution, suspension, emulsion).

### Formulations

Engineered immune cells expressing a tumor antigen-targeted engineered receptor and a FoxP3 targeting agent and compositions comprising thereof can be conveniently provided as sterile liquid preparations, e.g., isotonic aqueous solutions, suspensions, emulsions, dispersions, or viscous compositions, which can be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions of the presently disclosed subject matter, e.g., a composition comprising engineered immune cells, in the required amount of the appropriate solvent with various amounts of the other ingredients, as desired. Such compositions can be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, can be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the engineered immune cells and FoxP3 targeting agents of the presently disclosed subject matter.

The compositions can be isotonic, i.e., they can have the same osmotic pressure as blood and lacrimal fluid. The desired isotonicity of the compositions of the presently disclosed subject matter can be accomplished using sodium chloride, or other pharmaceutically acceptable agents such as dextrose, boric acid, sodium tartrate, propylene glycol or other inorganic or organic solutes. Sodium chloride is preferred, in some embodiments, particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the engineered immune cells as described in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the engineered immune cells (including in some instances FoxP3 targeting agents that are engineered immune cells) of the presently disclosed subject matter is the quantity of cells necessary to achieve an optimal effect. The quantity of cells to be administered will vary for the subject being treated. In certain embodiments, from about 10² to about 10¹², from about 10³ to about 10¹¹, from about 10⁴ to about 10¹⁰, from about 10⁵ to about 10⁹, or from about 10⁶ to about 10⁸ engineered immune cells of the presently disclosed subject matter are administered to a subject. More effective cells can be administered in even smaller numbers. In some embodiments, at least about 1×10⁸, about 2×10⁸, about 3×10⁸, about 4×10⁸, about 5×10⁸, about 1×10⁹, about 5×10⁹, about 1×10¹⁰, about 5×10¹⁰, about 1×10¹¹, about 5×10¹¹, about 1×10¹² or more engineered immune cells of the presently disclosed subject matter are administered to a human subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation

### Methods for Therapy

For treatment, the amount of the engineered immune cells provided herein administered is an amount effective in producing the desired effect, for example, treatment of a cancer or infectious disease or one or more symptoms of a cancer or infectious disease. An effective amount can be provided in one or a series of administrations of the engineered immune cells and/or FoxP3 targeting agents provided herein. An effective amount can be provided in a bolus or by continuous perfusion. For adoptive immunotherapy using antigen-specific T cells, cell doses in the range of about 10⁶ to about 10¹⁰ are typically infused. The engineered immune cells of the presently disclosed subject matter can be administered by any methods known in the art, including, but not limited to, pleural administration, intravenous administration, subcutaneous administration, intranodal administration, intratumoral administration, intrathecal administration, intrapleural administration, intraperitoneal administration, and direct administration to the thymus. In certain embodiments, the engineered immune cells and the compositions comprising thereof are intravenously administered to the subject in need. Methods for administering cells for adoptive cell therapies, including, for example, donor lymphocyte infusion and engineered T cell therapies, and regimens for administration are known in the art and can be employed for administration of the engineered immune cells provided herein.

The presently disclosed subject matter provides various methods of using the engineered immune cells (e.g., T cells) provided herein, expressing a tumor antigen-targeted engineered receptor (e.g., a CAR, caTCR, or eTCR). For example, the presently disclosed subject matter provides methods of reducing tumor burden in a subject. In one non-limiting example, the method of reducing tumor burden comprises administering an effective amount of the presently disclosed engineered immune cells to the subject, thereby inducing tumor cell death in the subject.

The presently disclosed engineered immune cells can reduce the number of tumor cells, reduce tumor size, and/or eradicate the tumor in the subject. In certain embodiments, the method of reducing tumor burden comprises administering an effective amount of engineered immune cells to the subject, thereby inducing tumor cell death in the subject. Non-limiting examples of suitable tumors include adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, acute and chronic leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof. In some embodiments, the cancer is a relapsed or refractory cancer. In some embodiments, the cancer is resistant to one or more cancer therapies, e.g., one or more chemotherapeutic drugs.

The presently disclosed subject matter also provides methods of increasing or lengthening survival of a subject having a neoplasia (e.g., a tumor). In one non-limiting example, the method of increasing or lengthening survival of a subject having neoplasia (e.g., a tumor) comprises administering an effective amount of the presently disclosed engineered immune cell to the subject, thereby increasing or lengthening survival of the subject. The presently disclosed subject matter further provides methods for treating or preventing a neoplasia (e.g., a tumor) in a subject, comprising administering the presently disclosed engineered immune cells to the subject.

Cancers whose growth can be inhibited using the engineered immune cells of the presently disclosed subject matter comprise cancers typically responsive to immunotherapy. Non-limiting examples of cancers for treatment include multiple myeloma, neuroblastoma, glioma, acute myeloid leukemia, colon cancer, pancreatic cancer, thyroid cancer, small cell lung cancer, and NK cell lymphoma. In certain embodiments, the cancer is multiple myeloma.

Additionally, the presently disclosed subject matter provides methods of increasing immune-activating cytokine production in response to a cancer cell or virally infected cell in a subject. In one non-limiting example, the method comprises administering the presently disclosed engineered immune cell and FoxP3 targeting agent to the subject. The immune-activating cytokine can be granulocyte macrophage colony stimulating factor (GM-CSF), IFNα, IFN-γ, TNF-α, IL-2, IL-3, IL-6, IL-11, IL-7, IL-12, IL-15, IL-21, interferon regulatory factor 7 (IRF7), and combinations thereof. In certain embodiments, the engineered immune cells including a tumor antigen-specific engineered receptor of the presently disclosed subject matter increase the production of GM-CSF, IFN-γ, and/or TNF-α.

Suitable human subjects for therapy typically comprise two treatment groups that can be distinguished by clinical criteria. Subjects with “advanced disease” or “high tumor burden” are those who bear a clinically measurable tumor (e.g., multiple myeloma). A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in the presently disclosed subject matter is administered to these subjects to elicit an anti-tumor response, with the objective of palliating their condition. Ideally, reduction in tumor mass occurs as a result, but any clinical improvement constitutes a benefit. Clinical improvement comprises decreased risk or rate of progression or reduction in pathological consequences of the tumor (e.g., multiple myeloma).

A second group of suitable subjects is known in the art as the “adjuvant group.” These are individuals who have had a history of neoplasia (e.g., multiple myeloma), but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor (e.g., multiple myeloma) has invaded neighboring tissues, or who show involvement of lymph nodes. Another group has a genetic predisposition to neoplasia (e.g., multiple myeloma) but has not yet evidenced clinical signs of neoplasia (e.g., multiple myeloma). For instance, women testing positive for a genetic mutation associated with breast cancer, but still of childbearing age, can wish to receive one or more of the compositions described herein in treatment prophylactically to prevent the occurrence of neoplasia until it is suitable to perform preventive surgery.

The subjects can have an advanced form of disease (e.g., multiple myeloma), in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the tumor antigen-targeted engineered receptor-expressing engineered immune cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. Modification of the engineered immune cells can include engineering a suicide gene into the tumor antigen-targeted engineered receptor-expressing T cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the C-terminus of the intracellular domain of the tumor antigen-targeted engineered receptor. The suicide gene can be included within the vector comprising nucleic acids encoding the presently disclosed tumor antigen-targeted engineered receptors. A presently disclosed engineered immune cell (e.g., a T cell) incorporated with a suicide gene can be pre-emptively eliminated at a given time point post CAR T cell infusion, or eradicated at the earliest signs of toxicity.

### Method for Manufacturing Engineered Immune Cells

In some embodiments, the engineered immune cell that expresses T-cell receptor (TCR) or other cell-surface ligand that binds to a target antigen is manufactured in the absence of a FoxP3 targeting agent. In such cases, the engineered immune cell is manufactured by any method known in the art. Exemplary methods for manufacturing engineered immune cells in the absence of a FoxP3 targeting agent are described, for example, in WO2016/191246, WO2015/011450, WO2017/070608, and WO2017/124001, which are incorporated by reference in their entireties. In some embodiments, the engineered immune cells that are manufactured in the absence of a FoxP3 targeting agent are co-administered to a subject with a FoxP3 targeting agent.

In other embodiments, the engineered immune cell that expresses T-cell receptor (TCR) or other cell-surface ligand that binds to a target antigen is manufactured in the presence of a FoxP3 targeting agent. In some embodiments, methods for manufacturing an engineered immune cell comprise (a) contacting a cell with a vector encoding an engineered receptor, wherein the vector comprises a nucleotide sequence that encodes for an extracellular antigen-binding domain that binds a target antigen (i.e., cell surface antigen); and (b) contacting the cell with a FoxP3 targeting agent. In some embodiments, the cell is contacted with a vector encoding an engineered receptor prior to contact with the FoxP3 targeting agent. In other embodiments, the cell is contacted with the FoxP3 targeting agent prior to contact with the vector encoding an engineered receptor. In other embodiments, the cell is contacted with the vector encoding an engineered receptor and FoxP3 targeting agent simultaneously.

In some embodiments, the method further comprises stimulating and expanding the cell prior to contact with the vector encoding an engineered receptor. In some embodiments, stimulating and expanding the cell comprises contacting the cell with CD3 and/or CD28 beads. In some embodiments, stimulating and expanding the cell occurs in the presence of interleukin-2 (IL-2). In some embodiments, stimulating and expanding the cell occurs in the presence of a FoxP3 targeting agent.

In some embodiments, the cell is in a sample comprising a plurality of cells. In some embodiments, contacting the cell with the FoxP3 targeting agent results in depletion of FoxP3⁺ cells from the sample. In some embodiments, depletion of FoxP3⁺ cells from the sample results in at least a 5%, 10%, 20%, 30%, 40%, 50%, 60%, 70%, 80%, 90%, 100%, 150%, 200%, 250%, or 300% reduction in the number of the FoxP3⁺ cells in the sample, as compared to a sample that has not been contacted with a FoxP3 targeting agent. In some embodiments, contacting the cell with the FoxP3 targeting agent results in enrichment of FoxP3⁻ cells in the sample. In some embodiments, enrichment of FoxP3⁻ cells in the sample results in at least a 25%, 30%, 40%, 50%, 60%, 70%, 80%, 90%, 100%, 150%, 200%, 250%, or 300% increase in the number of cells in the sample that are FoxP3⁻, as compared to a sample that has not been contacted with a FoxP3 targeting agent.

### Articles of Manufacture and Kits

The presently disclosed subject matter provides kits for the treatment or prevention of a disease, such as neoplasia (e.g., solid tumor) or infectious diseases. In certain embodiments, the kit comprises a therapeutic or prophylactic composition containing an effective amount of an engineered immune cell comprising a tumor antigen-targeted engineered receptor (e.g., a CAR, caTCR, or eTCR). In particular embodiments, the cells further expresses at least one co-stimulatory ligand.

If desired, the engineered immune cell can be provided together with instructions for administering the engineered immune cell to a subject having or at risk of developing a neoplasia (e.g., solid tumor). The instructions will generally include information about the use of the composition for the treatment or prevention of a neoplasia (e.g., solid tumor). In other embodiments, the instructions include at least one of the following: description of the therapeutic agent; dosage schedule and administration for treatment or prevention of a neoplasia (e.g., solid tumor) or symptoms thereof; precautions; warnings; indications; counter-indications; overdose information; adverse reactions; animal pharmacology; clinical studies; and/or references. The instructions can be printed directly on the container (when present), or as a label applied to the container, or as a separate sheet, pamphlet, card, or folder supplied in or with the container.

Also provided herein are kits for use in the manufacture of an engineered immune cell that expresses T-cell receptor (TCR) or other cell-surface ligand that binds to a target antigen, such as a tumor antigen or viral protein. In certain embodiments, the kit comprises (a) a vector encoding an engineered receptor; and (b) a FoxP3 targeting agent.

In some embodiments, the kits provided herein comprise a sterile container, such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In some embodiments, the sterile container contains a therapeutic or prophylactic vaccine.

### EXAMPLES

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, can be considered in making and practicing the invention. Particularly useful techniques for particular embodiments will be discussed in the sections that follow.

The following examples are put forth so as to provide those of ordinary skill in the art with a complete disclosure and description of how to make and use the compositions, and assay, screening, and therapeutic methods of the invention, and are not intended to limit the scope of what the inventors regard as their invention.

### Example 1. Synthesis of Anti-FoxP3 Antibodies

This example describes the synthesis of exemplary FoxP3 targeting agents, such as TCR-mimic monoclonal antibodies specific for FoxP3-derived epitopes (e.g., scFv specific for FoxP3-derived epitopes and FoxP3-BsAb) and chimeric antigen receptor (CAR) T cells targeting FoxP3.

scFv clones targeting FoxP3 were previously identified and describe in International Publication Number WO2017124001, which is incorporated by reference in its entirety. The complementary determining regions (CDRs) of the heavy and light chain of non-limiting examples of FoxP3 targeting scFv clones are shown in the table below. These scFv clones are engineered into full-length human IgG1, bispecific antibody (BsAb), and/or chimeric antigen receptor (CAR) T cells.

Examples of FoxP3 scFv1 Heavy chain CDRs (HCDRs) and Light
chain CDRs (LCDRs)

| clones | HCDR1 | HCDR2 | HCDR3 | LCDR1 | LCDR2 | LCDR3 |
|---|---|---|---|---|---|---|
| EXT017- | GDTFSRYA | IIPIFGTP | ARSIYRYSEYDH | SSNIGAGYD | GNS | QSYDSSLSGYV |
| 17 | (SEQ ID NO: | (SEQ ID | (SEQ ID NO: 18) | (SEQ ID NO: | (SEQ ID | (SEQ ID NO: 21) |
| | 16) | NO: 17) | | 19) | NO: 20) | |
| EXT017- | GYTFSNYY | INPSVGTT | ARDWWGQMMY | SSNIGSNT | SNN | AAWDDSLNGQG |
| 18 | (SEQ ID NO: | (SEQ ID | DG | (SEQ ID NO: | (SEQ ID | V |
| | 22) | NO: 23) | (SEQ ID NO: 24) | 25) | NO: 26) | (SEQ ID NO: 27) |
| EXT017- | GGTFSSYA | IIPIFGTA | ARYSYKYGELDT | SSNIGAGYD | GNS | QSYDSSLSGSV |
| 20 | (SEQ ID NO: | (SEQ ID | (SEQ ID NO: 30) | (SEQ ID NO: | (SEQ ID | (SEQ ID NO: 33) |
| | 28) | NO: 29) | | 31) | NO: 32) | |
| EXT017- | GYTFTNYY | IRPSGGIT | ARSWDYFASNDF | NIGSES | DDD | QVVVDRSSDHWF |
| 27 | (SEQ ID NO: | (SEQ ID | (SEQ ID NO: 36) | (SEQ ID NO: | (SEQ ID | (SEQ ID NO: 39) |
| | 34) | NO: 35) | | 37) | NO: 38) | |
| EXT017- | GGTFSTYA | IIPIFGTA | ARAEYVYGEYD | SSNIGAGYD | GNS | QSYDSSLSGYV |
| 28 | (SEQ ID NO: | (SEQ ID | Q | (SEQ ID NO: | (SEQ ID | (SEQ ID NO: 45) |
| | 40) | NO: 41) | (SEQ ID NO: 42) | 43) | NO: 44) | |
| EXT017- | GFTFNNHA | ISFDGDDK | SRDPYHFASGSY | NIGSKS | YDS | QVWDSSSDHYV |
| 32 | (SEQ ID NO: | (SEQ ID | SYFDY | (SEQ ID NO: | (SEQ ID | (SEQ ID NO: 51) |
| | 46) | NO: 47) | (SEQ ID NO: 48) | 49) | NO: 50) | |
| EXT017- | GYTFTNYY | IRPSGGNT | ARSWNSRDVDS | SGSIASHY | ENN | QSYDRSNHVV |
| 53 | (SEQ ID NO: | (SEQ ID | (SEQ ID NO: 54) | (SEQ ID NO: | (SEQ ID | (SEQ ID NO: 57) |
| | 52) | NO: 53) | | 55) | NO: 56) | |
| EXT017- | GGTFSSYA | IIPIFGTA | ARPSYYSIKSAW | TSNIGKNG | NDH | ATWDDTLDLPL |
| 54 | (SEQ ID NO: | (SEQ ID | DH | (SEQ ID NO: | (SEQ ID | (SEQ ID NO: 63) |
| | 58) | NO: 59) | (SEQ ID NO: 60) | 61) | NO: 62) | |

Construction of Full Length Human IgG1 Using the Selected scFv Fragments

Full-length human IgG1 of the selected phage clones were produced in HEK293 and Chinese hamster ovary (CHO) cell lines. In brief, antibody variable regions were subcloned into mammalian expression vectors, with matching Lambda or Kappa light chain constant sequences and IgG1 subclass Fc. Molecular weights of the purified full length IgG antibodies were measured under both reducing and non-reducing conditions by electrophoresis.

The heavy chain sequence of a full length IgG1 of clone EXT017-32 is shown below:

(SEQ ID NO: 9)
EVQLVESGGGVVQPGRSLRLSCAASGFTFNNHAMHWVRQAPGKGLEWVAVI
SFDGDDKFYADSVKGRFTISRDNSRNTLFLQMNNLRPEDTAVYYCSRDPYH
FASGSYSYFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDSD
GSFFLYSKLTVDKRWQQGNVFSCSVMHEALHNHYTQKSLSLSPGK.

The light chain sequence of a full length IgG1 of clone EXT017-32 is shown below:

(SEQ ID NO: 10)
QSVLTQPPSVSVAPGKTARITCGGNNIGSKSVHWYQQKPGQAPVLVIYYDS
DRPSGIPERFSGSNSGNTATLTISRVEAGDEADYYCQVWDSSSDHYVFGTG
TKVTVLGQPKANPTVTLFPPSSEELQANKATLVCLISDFYPGAVTVAWKAD
GSPVKAGVETTKPSKQSNNKYAASSYLSLTPEQWKSHRSYSCQVTHEGSTV
EKTVAPTECS.

Construction, expression and purification of FoxP3-BsAb

FoxP3-#32 BsAb was engineered as previously described (Dao et al. (2015) Nat Biotechnol. 33(10):1079-86. N-terminal end of mAb #32 scFv was linked to the C-terminal end of an anti-human CD3c scFv of a mouse monoclonal antibody by a flexible linker. The DNA fragments encoding for the scFv of two mAbs were synthesized by GeneArt (InVitrogen) and subcloned into Eureka's mammalian expression vector pGSN-Hyg using standard DNA technology. A hexahistidine (His) tag (SEQ ID NO: 354) was inserted downstream of the #32 BsAb at the C-terminal end for the detection and purification of the BsAb.

Chinese hamster ovary (CHO) cells were transfected with the FoxP3-#32BsAb expression vector and stable expression was achieved by standard drug selection with methionine sulfoximine (MSX), a glutamine synthetase (GS)-based method. CHO cell supernatants containing secreted FoxP3-#32 BsAb molecules were collected. FoxP3-#32 BsAb was purified using HisTrap HP column (GE healthcare) by FPLC AKTA system. Briefly, CHO cell culture was clarified and loaded onto the column with low imidazole concentration (20 mM), and then an isocratic high imidazole concentration elution buffer (500 mM) was used to elute the bound FoxP3-#32 BsAb. A negative control BsAb, was constructed from an irrelevant human IgG1 antibody (Cat #ET901, Eureka Therapeutics) replacing Fox3-#32 scFv.

The sequence of the FoxP3-#32 BsAb is provided below:

(SEQ ID NO: 11)
QSVLTQPPSVSVAPGKTARITCGGNNIGSKSVHWYQQKPGQAPVLVIYYDS
DRPSGIPERFSGSNSGNTATLTISRVEAGDEADYYCQVWDSSSDHYVFGTG
TKVTVLGSRGGGGSGGGGSGGGGSLEMAEVQLVESGGGVVQPGRSLRLSCA
ASGFTFNNHAMHWVRQAPGKGLEWVAVISFDGDDKFYADSVKGRFTISRDN
SRNTLFLQMNNLRPEDTAVYYCSRDPYHFASGSYSYFDYWGQGTLVTVSST
SGGGGSDVQLVQSGAEVKKPGASVKVSCKASGYTFTRYTIVIHWVRQAPGQ
GLEWIGYINPSRGYTNYADSVKGRFTITTDKSTSTAYMELSSLRSEDTATY
YCARYYDDHYCLDYWGQGTTVTVSSGEGTSTGSGGSGGSGGADDIVLTQSP
ATLSLSPGERATLSCRASQSVSYMNWYQQKPGKAPKRWIYDTSKVASGVPA
RFSGSGSGTDYSLTINSLEAEDAATYYCQQWSSNPLTFGGGTKVEIKHHHH
HH.

Construction of CAR T Cells Targeting FoxP3

A FoxP3 scFv sequence is used to generate a second generation CAR targeting FoxP3. The variable heavy and light chains (connected with a (Gly₄Ser)₃ linker (SEQ ID NO: 120)) and a c-myc tag are added to allow detection of CAR expression by flow cytometry. The CAR is optimized to include a spacer domain upstream of the CD28 transmembrane domain if required. This is cloned into the SFG retroviral vector containing the CD28 and CD3 zeta or 4-1BB or other similar signaling CAR forms that are well known in the art, e.g., Park (2016). Stable 293 viral producing cell lines are generated, and used to transduce primary human T cells as described previously (Rafiq (2017)). Following transduction, CAR expression is verified by flow cytometry, staining for the c-myc tag incorporated into the FoxP3-CAR. Retroviral transduction of primary human T cells has been previously described (Koneru (2015)).

The sequence of FoxP3 scFv-CD28-CD3zeta in the CAR vector is shown below

(SEQ ID NO: 12)
QSVLTQPPSVSVAPGKTARITCGGNNIGSKSVHWYQQKPGQAPVLVIYYDS
DRPSGIPERFSGSNSGNTATLTISRVEAGDEADYYCQVWDSSSDHYVFGTG
TKVTVLGSRGGGGSGGGGSGGGGSLEMAEVQLVESGGGVVQPGRSLRLSCA
ASGFTFNNHAMHWVRQAPGKGLEWVAVISFDGDDKFYADSVKGRFTISRDN
SRNTLFLQMNNLRPEDTAVYYCSRDPYHFASGSYSYFDYWGQGTLVTVSSA
AAIEVMYPPPYLDNEKSNGTIIHVKGKHLCPSPLFPGPSKPFWVLVVVGGV
LACYSLLVTVAFIIFWVRSKRSRLLHSDYMNMTPRRPGPTRKHYQPYAPPR
DFAAYRSRVKFSRSADAPAYQQGQNQLYNELNLGRREEYDVLDKRRGRDPE
MGGKPRRKNPQEGLYNELQKDKMAEAYSEIGMKGERRRGKGHDGLYQGLST
ATKDTYDALHMQALPPR.

The sequence of the FoxP3 scFv-41BB-CD3zeta in the CAR vector is shown below:

(SEQ ID NO: 13)
QSVLTQPPSVSVAPGKTARITCGGNNIGSKSVHWYQQKPGQAPVLVIYYDS
DRPSGIPERFSGSNSGNTATLTISRVEAGDEADYYCQVWDSSSDHYVFGTG
TKVTVLGSRGGGGSGGGGSGGGGSLEMAEVQLVESGGGVVQPGRSLRLSCA
ASGFTFNNHAMHWVRQAPGKGLEWVAVISFDGDDKFYADSVKGRFTISRDN
SRNTLFLQMNNLRPEDTAVYYCSRDPYHFASGSYSYFDYWGQGTLVTVSST
GTTTPAPRPPTPAPTIASQPLSLRPEACRPAAGGAVHTRGLDFACDIYIWA
PLAGTCGVLLLSLVITLYCKRGRKKLLYIFKQPFMRPVQTTQEEDGCSCRF
PEEEEGGCELRVKFSRSADAPAYQQGQNQLYNELNLGRREEYDVLDKRRGR
DPEMGGKPRRKNPQEGLYNELQKDKMAEAYSEIGMKGERRRGKGHDGLYQG
LSTATKDTYDALHMQALPPR.

Construction of a Chimeric Antibody/T Cell Receptor (caTCR) Targeting FoxP3

A caTCR targeting FoxP3 is produced as described in International Publication No. WO2017/070608, which is incorporated by reference in its entirety. Briefly, the constant and variable regions of the IgG1 heavy chain targeting FoxP3 is attached to the delta chain of a T cell receptor (TCR) to produce a heavy chain of the caTCR. The constant and variable regions of the IgG1 light chain targeting FoxP3 is attached to the gamma chain of a T cell receptor (TCR) to produce a heavy chain of the caTCR. The polynucleotide encoding these proteins are cloned into a vector. T cells are transduced with the vector to express the caTCR, thereby producing anti-FoxP3 caTCR T-cells.

The heavy chain sequence of a caTCR targeting FoxP3 is shown below:

(SEQ ID NO: 14)
METDTLLLWVLLLWVPGSTGEVQLVESGGGVVQPGRSLRLSCAASGFTFNN
HAMHWVRQAPGKGLEWVAVISFDGDDKFYADSVKGRFTISRDNSRNTLFLQ
MNNLRPEDTAVYYCSRDPYHFASGSYSYFDYWGQGTLVTVSSASTKGPSVF
PLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCEVKTDSTD
HVKPKETENTKQPSKSCHKPKAIVHTEKVNMMSLTVLGLRMLFAKTVAVNF
LLTAKLFFL.

The light chain sequence of a caTCR targeting FoxP3 is shown below:

(SEQ ID NO: 15)
METDTLLLWVLLLWVPGSTGQSVLTQPPSVSVAPGKTARITCGGNNIGSKS
VHWYQQKPGQAPVLVIYYDSDRPSGIPERFSGSNSGNTATLTISRVEAGDE
ADYYCQVWDSSSDHYVFGTGTKVTVLGQPKANPTVTLFPPSSEELQANKAT
LVCLISDFYPGAVTVAWKADGSPVKAGVETTKPSKQSNNKYAASSYLSLTP
EQWKSHRSYSCQVTHEGSTVEKTVAPTECSPIKTDVITMDPKDNCSKDAND
TLLLQLTNTSAYYMYLLLLLKSVVYFAIITCCLLRRTAFCCNGEKS.

### Example 2. Use of FoxP3 Targeting Agents in the Manufacture of an Anti-CD19 caTCR-T Cell Population

Examples 2a-2f evaluate the effect of various FoxP3 targeting agents in improving the manufacture of an anti-CD19 caTCR-T cell population. In some examples, the FoxP3 targeting agent is added to the cell sample after contact with a vector encoding an engineered receptor that binds to CD19. In other examples, the FoxP3 targeting agent is added to the cell sample prior to contact with a vector encoding an engineered receptor that binds to CD19.

### Example 2a: Generation of an Anti-CD19 caTCR-T Cell Population in the Presence of a FoxP3-Targeting Bi-Specific Antibody (BsAb)

In this example, the ability of anti-FoxP3 BsAb to improve the manufacturing efficiency or efficacy of anti-CD19 caTCR-T cells is investigated. A representative anti-FoxP3 BsAb as described in Example 1 (SEQ ID NO: 11) and a lentiviral vector encoding a representative anti-CD19 caTCR construct are used in this example. The caTCR construct has an anti-CD19 IgV_{H}-TCR delta chain and an anti-CD19 IgV_{L}-TCR gamma chain.

The sequence of the anti-CD19 IgV_{H}-TCR delta chain is shown below:

(SEQ ID NO: 103)
EVQLVQSGAEVKKPGESLKISCKGSGYSFTSYWIGWVRQMPGKGLEWMGII
YPGDSDTRYSPSFQGQVTISADKSISTAYLQWSSLKASDTAMYYCARQVWG
WQGGMYPRSNWWYNLDSWGQGTLVTVSSASTKGPSVFPLAPSSKSTSGGTA
ALGCLVKDYFPEPVTVSWNSGALTSGVHTFPAVLQSSGLYSLSSVVTVPSS
SLGTQTYICNVNHKPSNTKVDKRVEPKSCEVKTDSTDHVKPKETENTKQPS
KSCHKPKAIVHTEKVNMMSLTVLGLRMLFAKTVAVNFLLTAKLFFL.

The sequence of the anti-CD19 IgV_{L}-TCR gamma chain is shown below:

(SEQ ID NO: 104)
LPVLTQPPSVSVAPGKTARITCGGNNIGSKSVHWYQQKPGQAPVLVVYDDS
DRPSGIPERFSGSNSGNTATLTISRVEAGDEADYYCQVWDSSSDYVVFGGG
TKLTVLGQPKANPTVTLFPPSSEELQANKATLVCLISDFYPGAVTVAWKAD
GSPVKAGVETTKPSKQSNNKYAASSYLSLTPEQWKSHRSYSCQVTHEGSTV
EKTVAPTECSPIKTDVITMDPKDNCSKDANDTLLLQLTNTSAYYMYLLLLL
KSVVYFAIITCCLLRRTAFCCNGEKS.

PBMCs are obtained from patients and treated with CD3/CD28 beads to isolate and stimulate T cells on Day 0. On Day 1, the stimulated/activated T cells are separated into six groups: Group 1 (no anti-CD19 caTCR-encoding vector or anti-FoxP3 BsAb is added throughout the process), Groups 2-6 all have the anti-CD19 caTCR-encoding vector added on Day 1. Group 2 has no anti-FoxP3 BsAb added throughout the process, while Groups 3, 4, 5, and 6 have anti-FoxP3 BsAb added on Days 1, 2, 3, and 4, respectively. CD3/CD28 beads and the anti-CD19 caTCR viral vector are removed on Day 5 and the T cells are expanded for three or four days. The anti-FoxP3 BsAb is washed away on Day 5 or before T cell harvesting around Day 8 or Day 9.

The efficacy of depleting immunosuppressive Tregs is evaluated by antibody staining (e.g. CD4, CD25 and FoxP3 antibodies) and Flow Cytometry analysis. The improved manufacturing efficiency or efficacy of the anti-CD19 caTCR-T cells is determined by higher proliferation capacity and increased LDH killing activity. The proliferation assay and LDH killing assay are performed as described in International Publication No. WO2017070608, which is incorporated by reference in its entirety. Briefly, for proliferation assay, the anti-CD19 caTCR-T cells are labeled with Carboxyfluorescein succinimidyl ester dye (CFSE) and incubated with target cancer cells (e.g. NALM6 or Raji) and the proliferation capacity of the caTCR-T cells is presented by CFSE FACS signal. Higher proliferation capacity correlates with improved function of the engineered anti-CD19 caTCR-T cells. For LDH killing assay, anti-CD19 caTCR-T cells are incubated with target cancer cells (e.g. NALM6 or Raji) and the killing activity of the supernatant is determined by LDH assay. In addition, in vivo cancer cell killing efficacy of the anti-CD19 caTCR-T cells are tested in CD19 positive human lymphoma xenograft model in NOD SCID gamma (NSG) mice.

### Example 2b: Generation of Anti-CD19 caTCR-T Cell Population with Treatment of a FoxP3-Targeting IgG Antibody

In this example, the ability of anti-FoxP3 IgG antibody to improve the manufacturing efficiency or efficacy of anti-CD19 caTCR-T cells is investigated. A representative anti-FoxP3 IgG1 as described in Example 1 (SEQ ID NO: 9 and SEQ ID NO: 10) and a lentiviral vector encoding the same representative anti-CD19 caTCR construct as described in Example 2a are used in this example.

PBMCs are obtained from patients and treated with the anti-FoxP3 IgG1 without CD3/CD28 beads in order to kill Treg cells in the presence of NK cells within the PBMCs. A portion of the PBMCs are not treated with anti-FoxP3 IgG1 to serve as a negative control. After a period of 4 hours to 2 days of anti-FoxP3 IgG1 treatment (e.g., 4 h, 6 h, 8 h, 10 h, 12 h, 14 h, 16 h, 20 h, 24 h, 36 h, or 48 h), the IgG1 is washed away and the PBMCs are treated with CD3/CD28 beads to isolate and activate T cells. This day is considered as Day 0. Activated T cells are then transduced with anti-CD19 caTCR-encoding lentiviral vector starting on Day 1 in the presence of CD3/CD28 beads for 3-5 days. The CD3/CD28 beads and the anti-CD19 caTCR viral vector are removed on Days 4-6 and the T cells are expanded for three or four days. Anti-CD19 caTCR T cells are harvested around Day 8 or Day 9.

The efficacy of depleting immunosuppressive Tregs is evaluated by antibody staining and Flow Cytometry analysis prior to T cell activation and confirmed when transduced T cells are harvested. The improved manufacturing efficiency or efficacy of the anti-CD19 caTCR-T cells is determined by higher proliferation capacity and increased LDH killing activity in vitro and higher antitumor activity in vivo as described in Example 2a.

### Example 2c: Generation of Anti-CD19 caTCR-T Cell Population with Treatment of FoxP3-Targeting CAR-T Cells

In this example, the ability of anti-FoxP3 CAR-T cells to improve the manufacturing efficiency or efficacy of anti-CD19 caTCR-T cells is investigated. A lentiviral vector encoding a representative anti-FoxP3 CAR as described in Example 1 (e.g., SEQ ID NO: 12 or SEQ ID NO: 13) and a lentiviral vector encoding the same representative anti-CD19 caTCR construct as described in Example 2a (e.g., SEQ ID NO: 103 and SEQ ID NO: 104) are used in this example.

PBMCs are obtained from patients and treated with CD3/CD28 beads on Day 0 to isolate and stimulate/activate T cells. On Day 1 the activated T cells are split into three groups of cells. Group 1 is transduced with anti-CD19 caTCR-encoding vector, Group 2 is transduced with anti-FoxP3 CAR-encoding vector, while Group 3 is mock transduced (not with either vector). After four, five, or six days of transduction, the viral vectors are washed away and CD3/CD28 beads are removed from Group 1 and Group 2. Group 1 cells (anti-CD19 caTCR-transduced T cells) are split into two groups: Group 1a cells are mixed with Group 2 cells for anti-FoxP3 CAR T cells to kill the Treg cells, while Group 1b cells are mixed with Group 3 cells as a control. After 2, 3, 4, or 5 days of incubating the cell mixtures, anti-FoxP3 CAR T cells are removed either with methods such as those described in Lim and June (2017) Cell 168:724-740, Wang et al. (2011) Blood 118:1255-1263, and Stasi et al. (2011) N Engl J Med 365:1673-1683 (e.g., with iCasp9 or Expression of extracellular domain of EGFR), each of which are incorporated by reference in their entireties, or by positive selection of anti-CD19 caTCR T cells, e.g., using anti-idiotype antibodies.

Anti-CD19 caTCR T cells are harvested around Day 8 or Day 9. The efficacy of depleting immunosuppressive Tregs is evaluated by antibody staining and Flow Cytometry analysis as described in Example 2a. The improved manufacturing efficiency or efficacy of the anti-CD19 caTCR-T cells is determined by higher proliferation capacity and increased LDH killing activity in vitro and higher antitumor activity in vivo as described in Example 2a.

### Example 2d: Generation of Anti-CD19 caTCR-T Cell Population with Treatment of FoxP3-Targeting caTCR-T Cells

In this example, the ability of anti-FoxP3 caTCR-T cells to improve the manufacturing efficiency or efficacy of anti-CD19 caTCR-T cells is investigated. A lentiviral vector encoding a representative anti-FoxP3 caTCR as described in Example 1 (e.g., SEQ ID NO: 14 and SEQ ID NO: 15) and a lentiviral vector encoding the same representative anti-CD19 caTCR construct as described in Example 2a (e.g., SEQ ID NO: 103 and SEQ ID NO: 104) are used in this example.

The experiment is done in the same way as described in Example 2c except that the lentiviral vector encoding anti-FoxP3 caTCR is used in place of the vector encoding anti-FoxP3 CAR.

Anti-CD19 caTCR T cells are harvested around Day 8 or Day 9. The efficacy of depleting immunosuppressive Tregs is evaluated by antibody staining and flow Cytometry analysis as described in Example 2a. The improved manufacturing efficiency or efficacy of the anti-CD19 caTCR-T cells is determined by higher proliferation capacity and increased LDH killing activity in vitro and higher antitumor activity in vivo as described in Example 2a.

### Example 2e: Generation of Anti-CD19 caTCR-T Cell Population with Treatment of Anti-FoxP3 Microbeads

In this example, the ability of anti-FoxP3 microbeads to improve the manufacturing efficiency or efficacy of anti-CD19 caTCR-T cells is investigated. Anti-FoxP3 antibody (IgG, IgA, IgD, IgM, or IgE, full-length antibodies or antibody fragments comprising antigen-binding moieties) is coupled to magnetic beads (e.g., CliniMACS Anti-Biotin MicroBeads [Miltenyl Biotec Cat #130-019-201], Dynabeads® Biotin Binder [Thermofisher Scientific Cat #11047] according to the manufacturers' instructions.

On Day 0, PBMCs are obtained from patients and are split into two groups. A test group is treated with anti-FoxP3 magnetic beads to deplete FoxP3 positive immunosuppressive Tregs while a control group is not. The PBMCs are then treated with CD3/CD28 beads to isolate and stimulate T cells. On Day 1, the T cells are transduced by anti-CD19 caTCR-encoding lentiviral vector for 4-6 days. Anti-CD19 caTCR T cells are harvested around Day 8 or Day 9.

The efficacy of depleting immunosuppressive Tregs is evaluated by antibody staining and flow cytometry analysis prior to T cell activation and confirmed when transduced T cells are harvested. The improved manufacturing efficiency or efficacy of the anti-CD19 caTCR-T cells is determined by higher proliferation capacity and increased LDH killing activity in vitro and higher antitumor activity in vivo as described in Example 2a.

### Example 2f: Generation of Anti-CD19 caTCR-T Cell Population with Treatment of a Combination of Anti-FoxP3 Microbeads (to Physically Separate Tregs) and Anti-FoxP3 BsAb/CAR-T/caTCR-T (to Induce Killing of Tregs by T Cells) or a Free IgG (to Induce Killing of Tregs by NK Cells)

In this example, the ability of anti-FoxP3 microbeads, anti-FoxP3 BsAB, anti-FoxP3 CAR-T cells, and anti-FoxP3 caTCR-T cells to improve the manufacturing efficiency or efficacy of anti-CD19 caTCR-T cells is investigated. Anti-FoxP3 microbeads are generated as described in Example 2e, anti-FoxP3 BsAB and anti-FoxP3 IgG1 are generated as described in Example 1, anti-FoxP3 CAR-T cells are generated as described in Example 2c, and anti-FoxP3 caTCR-T cells are generated as described in Example 2d. In addition, a lentiviral vector encoding the same representative anti-CD19 caTCR construct as described in Example 2a (e.g., SEQ ID NO: 103 and SEQ ID NO: 104) is used in this example.

On Day 0, PBMCs are obtained from patients and are split into two groups (Groups 1 and 2). Group 1 is treated with anti-FoxP3 magnetic beads to deplete FoxP3 positive immunosuppressive Tregs while Group 2 is not. The PBMCs from Groups 1 and 2 are then treated with CD3/CD28 beads to isolate and stimulate T cells. On Day 1, the T cells are transduced by anti-CD19 caTCR-encoding lentiviral vector for 4-6 days. Anti-CD19 caTCR T cells are harvested around Day 8 or Day 9.

The anti-CD19 caTCR T cells from Groups 1 and 2 are further split into subgroups as shown below:

Group

| | | |
|---|---|---|
| Subgroup | Group 1 | Group 2 |

| | |
|---|---|
| A | No additional anti-FoxP3 targeting agent |
| B | anti-FoxP3 BsAB |
| C | anti-FoxP3 IgG1 |
| D | anti-FoxP3 CAR-T cells |
| E | anti-FoxP3 caTCR-T cells |

As shown in the table above, Groups 1 and 2 are divided into 5 subgroups. Subgroup A is not mixed with IgG1 or any additional anti-FoxP3 targeting agent. Subgroup B is mixed with anti-FoxP3 BsAb as described in Example 2a. Subgroup C is mixed with anti-FoxP3 IgG1 as described in Example 2b. Subgroup D is mixed with anti-FoxP3 CAR-T cells as described in Example 2c. Subgroup E is mixed with anti-FoxP3 caTCR-T cells as described in Example 2d.

The efficacy of depleting immunosuppressive Tregs is evaluated by antibody staining and flow cytometry in Examples 2a and e. The improved manufacturing efficiency or efficacy of the anti-CD19 caTCR-T cells is determined by higher proliferation capacity and increased LDH killing activity in vitro and higher antitumor activity in vivo as described in Example 2a.

### Example 3. Use of FoxP3 Targeting Agents in the Manufacture of an Anti-AFP caTCR-T Cell Population

Examples 3a-3f evaluate the effect of various FoxP3 targeting agents in improving the manufacture of an anti-AFP caTCR-T cell population. In some examples, the FoxP3 targeting agent is added to the cell sample after contact with a vector encoding an engineered receptor that binds to AFP. In other examples, the FoxP3 targeting agent is added to the cell sample prior to contact with a vector encoding an engineered receptor that binds to AFP.

### Example 3a: Generation of Anti-AFP CAR-T Cell Population in the Presence of a FoxP3-Targeting Bi-Specific Antibody (BsAb)

In this example, the ability of anti-FoxP3 BsAb to improve the manufacturing efficiency or efficacy of anti-alpha fetal protein (AFP) CAR-T cells is investigated. The representative anti-FoxP3 BsAb as used in Example 2a and a lentiviral vector encoding a representative anti-AFP CAR construct are used in this example. The anti-AFP CAR construct has an scFv that specifically binds a complex comprising an AFP peptide and an MHC class I protein, but does not bind the AFP peptide or the MHC alone. The anti-AFP CAR construct has a fragment of CD28 and CD3zeta fused to the scFv fragment.

The sequence of the anti-AFP scFv is shown below:

(SEQ ID NO: 98)
QSVLTQPASVSGSPGQSITISCTGTSSDVGGYNYVSWYQQHPGKAPKLMIY
DVNNRPSEVSNRFSGSKSGNTASLTISGLQAEDEADYYCSSYTTGSRAVFG
GGTKLTVLGSRGGGGSGGGGSGGGGSLEMAEVQLVQSGAEVKKPGESLTIS
CKASGYSFPNYWITWVRQMSGGGLEWMGRIDPGDSYTTYNPSFQGHVTISI
DKSTNTAYLHWNSLKASDTAMYYCARYYVSLVDIWGQGTLVTVSS

The sequence of the CD28-CD3zeta fragment that is fused to the anti-AFP scFv is shown below:

(SEQ ID NO: 99)
AAAIEVMYPPPYLDNEKSNGTIIHVKGKHLCPSPLFPGPSKPFWVLVVVGG
VLACYSLLVTVAFIIFWVRSKRSRLLHSDYMNMTPRRPGPTRKHYQPYAPP
RDFAAYRSRVKFSRSADAPAYQQGQNQLYNELNLGRREEYDVLDKRRGRDP
EMGGKPRRKNPQEGLYNELQKDKMAEAYSEIGMKGERRRGKGHDGLYQGLS
TATKDTYDALHMQALPPR.

PBMCs are obtained from patients and treated with CD3/CD28 beads to isolate and stimulate T cells on Day 0. On Day 1, the stimulated/activated T cells are separated into five groups: Group 1 (no anti-AFP CAR-encoding vector or anti-FoxP3 BsAb is added throughout the process), Groups 2-5 all have the anti-AFP CAR-encoding vector added on Day 1, Group 2 has no anti-FoxP3 BsAb added throughout the process, while Groups 3, 4, and 5 have anti-FoxP3 BsAb added on Days 1, 3, and 5, respectively. The anti-FoxP3 BsAb is washed away before T cell harvesting around Day 8 or Day 9.

The efficacy of depleting immunosuppressive Tregs is evaluated by antibody staining (e.g. CD4, CD25 and FoxP3 antibodies) and Flow Cytometry analysis. The improved manufacturing efficiency or efficacy of the anti-AFP CAR-T cells is determined by higher proliferation capacity and increased LDH killing activity. For proliferation assay, the anti-AFP CAR-T cells are labeled with Carboxyfluorescein succinimidyl ester dye (CF SE) and incubated with target cancer cells (e.g., HEPG2 and SK-HEP1-MiniG, a SK-HEP1 cell line transfected with an AFP158 minigene cassette) and the proliferation capacity of the caTCR-T cells is presented by CFSE FACS signal. Higher proliferation capacity correlates with improved function of the engineered anti-AFP CAR-T cells. For LDH killing assay, anti-AFP CAR-T cells are incubated with target cancer cells (e.g., HEPG2 and SK-HEP1-MiniG, a SK-HEP1 cell line transfected with an AFP158 minigene cassette) and the killing activity of the supernatant is determined by LDH assay. In addition, in vivo cancer cell killing efficacy of the anti-AFP CAR-T cells are tested in AFP positive human hepatocellular carcinoma xenograft model in NOD SCID gamma (NSG) mice.

### Example 3b: Generation of Anti-AFP CAR-T Cell Population with Treatment of a FoxP3-Targeting IgG Antibody

In this example, the ability of anti-FoxP3 IgG antibody to improve the manufacturing efficiency or efficacy of anti-AFP CAR-T cells is investigated. In this example, the generation of anti-AFP CAR-T cells are performed in an almost identical manner to Example 2b, with the exception that anti-AFP CAR-T cells are produced using an anti-AFP CAR-encoding lentiviral vector (as described in Example 3a) instead of producing anti-CD19 CAR T cells using an anti-CD19 CAR-encoding lentiviral vector.

The efficacy of depleting immunosuppressive Tregs is evaluated by antibody staining and Flow Cytometry analysis prior to T cell activation and confirmed when transduced T cells are harvested. The improved manufacturing efficiency or efficacy of the anti-AFP CAR-T cells is determined by higher proliferation capacity and increased LDH killing activity in vitro and higher antitumor activity in vivo as described in Example 3a.

### Example 3c: Generation of Anti-AFP CAR-T Cell Population with Treatment of FoxP3-Targeting CAR-T Cells

In this example, the ability of anti-FoxP3 CAR-T cells to improve the manufacturing efficiency or efficacy of anti-AFP CAR-T cells is investigated. In this example, the generation of anti-AFP CAR-T cells are performed in an almost identical manner to Example 2c, with the exception that anti-AFP CAR-T cells are produced using an anti-AFP CAR-encoding lentiviral vector (as described in Example 3a) instead of producing anti-CD19 CAR T cells using an anti-CD19 CAR-encoding lentiviral vector.

Anti-AFP CAR T cells are harvested around Day 8 or Day 9. The efficacy of depleting immunosuppressive Tregs is evaluated by antibody staining and Flow Cytometry analysis as described in Example 3a. The improved manufacturing efficiency or efficacy of the anti-AFP CAR-T cells is determined by higher proliferation capacity and increased LDH killing activity in vitro and higher antitumor activity in vivo as described in Example 3a.

### Example 3d: Generation of Anti-AFP CAR-T Cell Population with Treatment of FoxP3-Targeting caTCR-T Cells

In this example, the ability of anti-FoxP3 caTCR-T cells to improve the manufacturing efficiency or efficacy of anti-AFP CAR-T cells is investigated. In this example, the generation of anti-AFP CAR-T cells are performed in an almost identical manner to Example 2d, with the exception that anti-AFP CAR-T cells are produced using an anti-AFP CAR-encoding lentiviral vector (as described in Example 3a) instead of producing anti-CD19 CAR T cells using an anti-CD19 CAR-encoding lentiviral vector.

The efficacy of depleting immunosuppressive Tregs is evaluated by antibody staining and flow cytometry analysis as described in Example 3a. The improved manufacturing efficiency or efficacy of the anti-AFP CAR-T cells is determined by higher proliferation capacity and increased LDH killing activity in vitro and higher antitumor activity in vivo as described in Example 3a.

### Example 3e. Generation of Anti-AFP caTCR-T Cell Population with Treatment of Anti-FoxP3 Microbeads

In this example, the ability of anti-FoxP3 microbeads to improve the manufacturing efficiency or efficacy of anti-AFP caTCR-T cells is investigated. In this example, the generation of anti-AFP CAR-T cells are performed in an almost identical manner to Example 2e, with the exception that anti-AFP CAR-T cells are produced using an anti-AFP CAR-encoding lentiviral vector (as described in Example 3a) instead of producing anti-CD19 CAR T cells using an anti-CD19 CAR-encoding lentiviral vector.

The efficacy of depleting immunosuppressive Tregs is evaluated by antibody staining and flow cytometry analysis prior to T cell activation and confirmed when transduced T cells are harvested. The improved manufacturing efficiency or efficacy of the anti-AFP CAR-T cells is determined by higher proliferation capacity and increased LDH killing activity in vitro and higher antitumor activity in vivo as described in Example 3a.

### Example 3f. Generation of Anti-AFP caTCR-T Cell Population with Treatment of a Combination of Anti-FoxP3 Microbeads (to Physically Separate Tregs) and Anti-FoxP3 BsAb/CAR-T/caTCR-T (to Induce Killing of Tregs by T Cells) or a Free IgG (to Induce Killing of Tregs by NK Cells)

In this example, the ability of anti-FoxP3 microbeads, anti-FoxP3 BsAB, anti-FoxP3 CAR-T cells, and anti-FoxP3 caTCR-T cells to improve the manufacturing efficiency or efficacy of anti-AFP caTCR-T cells is investigated. Anti-FoxP3 microbeads are generated as described in Example 2e, anti-FoxP3 BsAB and anti-FoxP3 IgG1 are generated as described in Example 1, anti-FoxP3 CAR-T cells are generated as described in Example 2c, and anti-FoxP3 caTCR-T cells are generated as described in Example 2d. In addition, a lentiviral vector encoding the same representative anti-AFP caTCR construct as described in Example 3a (e.g., SEQ ID NO: 98 and SEQ ID NO: 99) is used in this example.

In this example, the generation of anti-AFP CAR-T cells are performed in an almost identical manner to Example 2f, with the exception that anti-AFP CAR-T cells are produced using an anti-AFP CAR-encoding lentiviral vector (as described in Example 3a) instead of producing anti-CD19 CAR T cells using an anti-CD19 CAR-encoding lentiviral vector.

The efficacy of depleting immunosuppressive Tregs is evaluated by antibody staining and flow cytometry analysis as described in Examples 3a and e. The improved manufacturing efficiency or efficacy of the anti-AFP CAR-T cells is determined by higher proliferation capacity and increased LDH killing activity in vitro and higher antitumor activity in vivo as described in Example 3a.

### Example 4. Synthesis of CAR T Cells Expressing scFvs Targeting ROR2 Using a FoxP3 Targeting Agent

In some embodiments, an engineered immune cell expresses a CAR that targets ROR2. In this example, a method of generating an engineered immune cell expressing a CAR comprising a scFv that targets ROR2 is described.

Sequences for CARs Targeting ROR 2

In some embodiments, the CAR comprises an anti-ROR2 antibody or an antigen binding fragment thereof. For each antibody, the information is organized as following:
1. Name of antibody;
2. Light chain variable region (LCVR) DNA sequence;
3. Light chain variable region (LCVR) protein sequence;
4. Heavy chain variable region (HCVR) DNA sequence; and
5. Heavy chain variable region (HCVR) protein sequence.
   The CARs disclosed herein can comprise a LCVR and/or HCVR having the protein or DNA sequence of the LCVRs and/or HCVRs of the anti-ROR2 antibodies described below. Alternatively, or additionally, the CARs described herein can comprise a LCVR and/or HCVR having the protein or DNA sequence of the light chain complementarity determining region (LCDR) or heavy chain CDR (HCDR) of the anti-ROR2 antibodies described below (see also Tables 5 and 6 of WO2016142768A1, which is incorporated by reference in its entirety).

1) Antibody ROR2 clone #016
016-Lambda light chain variable region (DNA sequence)
[SEQ ID NO: 217]
tcttctgagctgactcaggaccctgctgtgtctgtggccttgggacagacagtcaggatca
catgccaaggagacagcctcagaagctattatgcaagctggtaccagcagaagccaggaca
ggcccctgtacttgtcatctatggtaaaaacaaccggccctcagggatcccagaccgattc
tctggctccagctcaggaaacacagcttccttgaccatcactggggctcaggcggaagatg
aggctgactattactgtaactcccgggacagcagtggtaaccatctggtattcggcggagg
gaccaagctgaccgtcctagg
016-Lambda light chain variable region (amino acid sequence)
[SEQ ID NO: 204]
SSELTQDPAVSVALGQTVRITCQGDSLRSYYASWYQQKPGQAPVLVIYGKNNRPSGIPDRF
SGSSSGNTASLTITGAQAEDEADYYCNSRDSSGNHLVFGGGTKLTVLG
016-Heavy chain variable region (DNA sequence)
[SEQ ID NO: 218]
gaggtccagctggtacagtctggggctgaggtgaagaagcctggggcctcagtgaaggtct
cctgcaaggcttctggatacaccttcaccgactactatatacactgggtgcggcaggcccc
tggacaagggctggagtggatgggatggatgaaccctaacagtgggaactcagtctctgca
cagaagttccagggcagagtcaccatgaccagggatacctccataaacacagcctacatgg
agctgagcagcctgacatctgacgacacggccgtgtattactgtgcgcgcaactctgaatg
gcatccgtggggttactacgattactggggtcaaggtactctggtgaccgtctcctca
016-Heavy chain variable region (amino acid sequence)
[SEQ ID NO: 191]
EVQLVQSGAEVKKPGASVKVSCKASGYTFTDYYIHWVRQAPGQGLEWMGWMNPNSGNSVSA
QKFQGRVTMTRDTSINTAYMELSSLTSDDTAVYYCARNSEWHPWGYYDYWGQGTLVTVSS
2) Antibody ROR2 clone #023
023-Kappalight chain variable region (DNA sequence)
[SEQ ID NO: 219]
gaaacgacactcacgcagtctccaggcaccctgtctgtgtctccaggggaaagagccaccc
tctcctgcagggccagtcagagtgttagcagcaacttagcctggtaccagcagaaacgtgg
ccaggctcccaggctcctcatctatggtgcgtctacccgggccactggtatcccagtcagg
ttcagtggcagtgggtctgggacagagttcactctcaccatcagcagattggagcctgaag
attttgcagtgtattactgtcagcagtatggtaggtcaccgctcactttcggcggagggac
caaagtggatatcaaacgt
023-Kappa light chain variable region (amino acid sequence)
[SEQ ID NO: 205]
ETTLTQSPGTLSVSPGERATLSCRASQSVSSNLAWYQQKRGQAPRLLIYGASTRATGIPVR
FSGSGSGTEFTLTISRLEPEDFAVYYCQQYGRSPLTFGGGTKVDIKR
023-Heavy chain variable region (DNA sequence
[SEQ ID NO: 220]
gaagtgcagctggtgcagtctggagcagaggtgaaaaagcccggggagtctctgaagatct
cctgtcagggttctggatacaggttcagcaagtactggatcggctgggtgcgccagatgcc
cgggaaaggcctggagtggatggggatcatctatcctggtgactctgataccagatacagc
ccgtccttccaaggccaggtcaccatctcagccgacaagtccatcagcaccgcctacctgc
agtggagcagcctgaaggcctcggacaccgccatgtattactgtgcgcgctctttctcttc
tttcatctacgattactggggtcaaggtactctggtgaccgtctcctca
023-Heavy chain variable region (amino acid sequence)
[SEQ ID NO: 192]
EVQLVQSGAEVKKPGESLKISCQGSGYRFSKYWIGWVRQMPGKGLEWMGIIYPGDSDTRYS
PSFQGQVTISADKSISTAYLQWSSLKASDTAMYYCARSFSSFIYDYWGQGTLVTVS
3) Antibody ROR2 clone #024
024-Kappalight chain variable region (DNA sequence)
[SEQ ID NO: 221]
gaaattgtgatgacacagtctccagccaccctgtctgtgtctccaggggaaagtgccaccc
tctcctgcagggccagtcagggtgttggcatcaacttagcctggtaccagcagagacctgg
ccagcctcccaggctcctcatctatgatgcatccaacagggccactggcatcccagccagg
ttcagtggcagtgggtctgggacagatttcactctcaccatcagcagcctgcaggctgaag
atgtggcagtctattactgtcagcaatactatagttttccgtggacgttcggccaggggac
caaggtggaaatcaaacgt
024-Kappa light chain variable region (amino acid sequence)
[SEQ ID NO: 206]
EIVMTQSPATLSVSPGESATLSCRASQGVGINLAWYQQRPGQPPRLLIYDASNRATGIPAR
FSGSGSGTDFTLTISSLQAEDVAVYYCQQYYSFPWTFGQGTKVEIKR
024-Heavy chain variable region (DNA sequence)
[SEQ ID NO: 222]
gaggtgcagctggtgcagtctggggcagaggtgaaaaagcccggggagtctctgaaaatct
cctgtaaggcttctggatacagctttagcaactactggatcggctgggtgcgccagatgcc
cgggaaaggcctggagtggatggggatcatctatcctgatgactctgataccagatacagc
ccgtccgtccaaggccaggtcaccatctcagccgacaagtccatcagcaccgcctacctgc
agtggtacagcctgaaggtcgcggacaccgccaaatattactgtgtgcgccctaggggggc
ttttgatatctggggccaagggaccacggtcaccgtctcctca
024-Heavy chain variable region (amino acid sequence)
[SEQ ID NO: 193]
EVQLVQSGAEVKKPGESLKISCKASGYSFSNYWIGWVRQMPGKGLEWMGIIYPDDSDTRYS
PSVQGQVTISADKSISTAYLQWYSLKVADTAKYYCVRPRGAFDIWGQGTTVTVSS
4) Antibody ROR2 clone #027
027-Light chain variable region (DNAsequence)
[SEQ ID NO: 223]
cagtctgtgctgacgcagccgccctcagtgtctggggccccagggcagagggtcacgatct
cctgcactgggagtagctccaacatcggggcaggtcatgctgtacactggtaccagcaact
tccaggaacagcccccaaactcctcatctatgataacgccaatcggccctcaggggtccct
gaccgattctctggctcccagtctggcacttcagcctccctggccatcaccggactccaga
ctggggacgaggccgattattactgcggaacatgggatgacagcccgagtgcttatgtctt
cggaactgggaccaaggtcaccgtcctaggt
027-Light chain variable region (amino acid sequence)
[SEQ ID NO: 207]
QSVLTQPPSVSGAPGQRVTISCTGSSSNIGAGHAVHWYQQLPGTAPKLLIYDNANRPSGVP
DRFSGSQSGTSASLAITGLQTGDEADYYCGTWDDSPSAYVFGTGTKVTVLG
027-Heavy chain variable region (DNA sequence)
[SEQ ID NO: 224]
caggtgcagctggtggagtctggggcagaggtgaaaaagcccggggagtctctgaaaatct
cctgtaaggcttctggatacagctttagcaactactggatcggctgggtgcgccagatgcc
cgggaaaggcctggagtggatggggatcatctatcctgatgactctgataccagatacagc
ccgtccttccaaggccaggtcaccatctcagccgacaagtccatcagcaccgcctacctgc
agtggtacagcctgaaggtcgcggacaccgccaaatattactgtgtgcgccctaggggggc
ttttgatatctggggccaagggaccacggtcaccgtctcctca
027-Heavy chain variable region (amino acid sequence)
[SEQ ID NO: 194]
QVQLVESGAEVKKPGESLKISCKASGYSFSNYWIGWVRQMPGKGLEWMGIIYPDDSDTRYS
PSFQGQVTISADKSISTAYLQWYSLKVADTAKYYCVRPRGAFDIWGQGTTVTVSS
5) Antibody ROR2 clone #084
084-Kappa light chain variable region (DNA sequence)
[SEQ ID NO: 225]
gatgttgtgatgactcagtctccactctccctgcccgtcacccttggacagccggcctcca
tctcctgcaggtctagtcaaagcctcgttcacagtgatggaaacacctacttgaattggtt
tcagcagaggccaggccaatctccaaggcgcctaatttataaagtttctagccgggactct
ggggtcccagatagattcagcggcactgggtcaggcactgatttcacactgaaaatcagca
gggtggaggctgaagatgttggcgtttattactgcatgcaaaccacacactggcctccgac
gttcggccaagggaccaaggtggagatcaaacgt
084-Kappa light chain variable region (amino acid sequence)
[SEQ ID NO: 208]
DVVMTQSPLSLPVTLGQPASISCRSSQSLVHSDGNTYLNWFQQRPGQSPRRLIYKVSSRDS
GVPDRFSGTGSGTDFTLKISRVEAEDVGVYYCMQTTHWPPTFGQGTKVEIKR
084-Heavy chain variable region (DNA sequence)
[SEQ ID NO: 226]
caggtgcagctggtggagtctgggggaggcttggtccagcctggggggtccctgagactct
cctgtgcagcctctggattcacctttagtagctattggatgagctgggtccgccaggctcc
agggaaagggctggagtgggtggccaacataaagcaagatggaagtgagaaatactatgtg
gactctgtgaggggccgattcaccatctccagagacaacgccaagaactcactgtatctgc
aaatgaacagcctgagagccgaggacaccgccatgtattactgtgcgcgcggttctttctc
ttacgacagtgatctgtggggtcaaggtactctggtgaccgtctcctca
084-Heavy chain variable region (amino acid sequence)
[SEQ ID NO: 195]
QVQLVESGGGLVQPGGSLRLSCAASGFTFSSYWMSWVRQAPGKGLEWVANIKQDGSEKYYV
DSVRGRFTISRDNAKNSLYLQMNSLRAEDTAMYYCARGSFSYDSDLWGQGTLVTVSS
6) Antibody ROR2 clone #90
090-Light chain variable region (DNA sequence)
[SEQ ID NO: 227]
cagcctgtgctgactcagccaccctcagcgtctgggacccccgggcagagggtcaccatct
cttgttctggaagcagctccaacatcgggagtgattatgtatcctggtaccaacagctccc
aggaacggcccccaaactcctcatctataggaatgatcagcggccctcaggggtccctgac
cgattctctggctccaagtctggcacctcagcctccctggccatcagtgggctccggtccg
aggatgaggctgattattactgtgtagcatgggatgacagcctgagtggttatgtcttcgg
aagtgggaccaaggtcaccgtcctaggt
090-Light chain variable region (amino acid sequence)
[SEQ ID NO: 209]
QPVLTQPPSASGTPGQRVTISCSGSSSNIGSDYVSWYQQLPGTAPKLLIYRNDQRPSGVPD
RFSGSKSGTSASLAISGLRSEDEADYYCVAWDDSLSGYVFGSGTKVTVLG
090-Heavy chain variable region (DNA sequence)
[SEQ ID NO: 228]
gaggtgcagctggtggagtctggcccaggactggtgaagccttcacagaccctgtccctca
cctgcactgtctctggtggctccatcagcagtggtggttactactggagctggatccgcca
gcacccagggaagggcctggagtggattgggtacatctattacagtgggagcacctactac
aacccgtccctcaagagtcgagttaccatatcagtagacacgtccaagaaccagttctccc
tgaagctgagctctgtgaccgctgcggacaccgccatgtattactgtgcgcgcggtggtct
gtactggacttactctcaggatgtttggggtcaaggtactctggtgaccgtctcctca
090-Heavy chain variable region (Amino acid sequence)
[SEQ ID NO: 196]
EVQLVESGPGLVKPSQTLSLTCTVSGGSISSGGYYWSWIRQHPGKGLEWIGYIYYSGSTYY
NPSLKSRVTISVDTSKNQFSLKLSSVTAADTAMYYCARGGLYWTYSQDVWGQGTLVTVSS
7) Antibody ROR2 clone #093
093-Kappalight chain variable region (DNA sequence)
[SEQ ID NO: 229]
gaaattgtgatgacgcagtctccagccaccctgtctttgtctccaggggaaagagccaccc
tctcctgcggggccagtcagagtgttagcagcagctacttagcctggtaccagcagaaacc
tggcctggcgcccaggctcctcatctatgatacatccagaagggccactggcatcccagac
aggttcagtggcagtgggtctgggacagacttcactctcaccatcagcagactggagccgg
aagattttgcagtgtattactgtcttcactatggtcgctcacctccggtcactttcggcgg
agggaccaaggtggagatcaaacgt
093-Kappalight chain variable region (amino acid sequence)
[SEQ ID NO: 210]
EIVMTQSPATLSLSPGERATLSCGASQSVSSSYLAWYQQKPGLAPRLLIYDTSRRATGIPD
RFSGSGSGTDFTLTISRLEPEDFAVYYCLHYGRSPPVTFGGGTKVEIKR
093-Heavy chain variable region (DNA sequence)
[SEQ ID NO: 230]
cagatgcagctggtgcagtctgggggaggcgtggtccagcctgggaggtccctgagactct
cctgtgcagcctctggattcaccttcagtaactatgacatgcactgggtccgccgggctcc
aggcaaggggctggagtgggtggcagttatatcatatgatggaagtaataattactatgca
gactccgtgaagggccgattcaccatctccagagacaattccaagaacacgctgtatctgc
aaatgaacagcctgagagctgaggacacggccgtgtattactgtgcgcgctcttctgcttg
ggttggtggtggtttcctgtctggtactgatgactggggtcaaggtactctggtgaccgtc
tcctca
093-Heavy chain variable region (amino acid sequence)
[SEQ ID NO: 197]
QMQLVQSGGGVVQPGRSLRLSCAASGFTFSNYDMHWVRRAPGKGLEWVAVISYDGSNNYYAD
SVKGRFTISRDNSKNTLYLQMNSLRAEDTAVYYCARSSAWVGGGFLSGTDDWGQGTLVTVSS
8)Antibody ROR2 clone #096
096-Light chain variable region (DNA sequence)
[SEQ ID NO: 231]
gaaattgtgctgactcagtctccactctccctgcccgtcacccttggacagccggcctcca
tctcctgcaggtctagtcaaagcctcgcatacagtgatggaaacacctacttgaattggtt
tcaccagaggccaggccaatctccaaggcgcctaatctataaggtttctaagcgggactct
ggggtcccagacagattcagcggcagtgggtcaggcactgatttcacactgagaatcagca
gggtggaggctgaggatgttgggatttattactgcatgcaaggtacacactggcctcacac
tttcggccctgggaccaaagtggatatcaaacgt
096-Light chain variable region (amino acid sequence)
[SEQ ID NO: 211]
EIVLTQSPLSLPVTLGQPASISCRSSQSLAYSDGNTYLNWFHQRPGQSPRRLIYKVSKRDS
GVPDRFSGSGSGTDFTLRISRVEAEDVGIYYCMQGTHWPHTFGPGTKVDIKR
096-Heavy chain variable region (DNA sequence)
[SEQ ID NO: 232]
gaagtgcagctggtgcagtctgggggaggcttggtccagcctggagggtccctgagactct
cctgtgcagcctctggattcagcctcaatgactattacatggactgggtccgccaggctcc
aggggaggggctggagtgggttggccgtattagagacaaagctcacggtgacaccacagaa
tacatcgcgtctgtgaaagacagatttatcgtctcaagagatgactccaagaactcactgt
atctgcaaatgaacagcctgaaaaccgaggacaccgccatgtattactgtgcgcgctgggt
tgacgactaccagggttactggatctggtcttaccacgatttctggggtcaaggtactctg
gtgaccgtctcctca
096-Heavy chain variable region (amino acid sequence)
[SEQ ID NO: 198]
EVQLVQSGGGLVQPGGSLRLSCAASGFSLNDYYMDWVRQAPGEGLEWVGRIRDKAHGDTTE
YIASVKDRFIVSRDDSKNSLYLQMNSLKTEDTAMYYCARWVDDYQGYWIWSYHDFWGQGTL
VTVSS
9) Antibody ROR2 clone #121
121-Light chain variable region (DNA sequence)
[SEQ ID NO: 233]
tcctatgtgctgactcagccaccctcagtgtccgtgtccccaggacagacagccagcgtca
cctgttctggatatagattgagagagaagtatgtttcctggtatcaacagaggccaggcca
ctcccctgtcttggtcatctatgaagatactaagaggccttcagggatccctgagcgattc
tctggctccaattctggggacacagccactctgaccatcagagggacccaggctatagatg
aggctgactattactgtcaggcgtgggacagcagcgtgattttcggcggagggaccaagct
gaccgtcctaggt
121-Light chain variable region (amino acid sequence)
[SEQ ID NO: 212]
SYVLTQPPSVSVSPGQTASVTCSGYRLREKYVSWYQQRPGHSPVLVIYEDTKRPSGIPERF
SGSNSGDTATLTIRGTQAIDEADYYCQAWDSSVIFGGGTKLTVLG
121-Heavy chain variable region (DNA sequence)
[SEQ ID NO: 234]
caggtgcagctggtgcagtctgggggaggcttggtacagcctggggggtccctgagactct
cctgtgcagccactggattcacctttagcagctatgccatgagttgggtccgccaggctcc
agggaaggggctggagtgggtctcagttattagtggtagtggtggtagcacatactacgca
gactccgtgaagggccggttcaccatctccagagacaattccaagaacacgttgtatctgc
aaatgaacagcctgagagccgacgacactgccgtgtattactgtgcgcgccattactactc
ttctgattcttggggtcaaggtactctggtgaccgtctcctca
121-Heavy chain variable region (amino acid sequence)
[SEQ ID NO: 199]
QVQLVQSGGGLVQPGGSLRLSCAATGFTFSSYAMSWVRQAPGKGLEWVSVISGSGGSTYYA
DSVKGRFTISRDNSKNTLYLQMNSLRADDTAVYYCARHYYSSDSWGQGTLVTVSS
10) Antibody ROR2 clone #159
159-Light chain variable region (DNA sequence)
[SEQ ID NO: 235]
caatctgccctgactcagcctgcctccgtgtctgggtctcctggacagtcgatcaccatct
cctgcactggaaccagcagtgacgttggtggttataactatgtctcttggtaccaacagca
cccaggcaaagcccccaaattcatgatttatgatgtcagtaagcggccctcaggtgtttct
aatcgcttctctggctccaagtctggcaacacggcctccctgaccatctctgggctccagg
ctgaggacgaggctgattattactgcggctcatttacaagcagcatcacttatgtcttcgg
aactgggaccaaggtcaccgtcctaggt
159-Light chain variable region (amino acid sequence)
[SEQ ID NO: 213]
QSALTQPASVSGSPGQSITISCTGTSSDVGGYNYVSWYQQHPGKAPKFMIYDVSKRPSGVS
NRFSGSKSGNTASLTISGLQAEDEADYYCGSFTSSITYVFGTGTKVTVLG
159-Heavy chain variable region (DNA sequence)
[SEQ ID NO: 236]
cagatgcagctggtgcagtctggggctgaggtgaagaagcctggggcctcagtgaaggttt
cctgcaaggcatctggatacaccttcaccagctactatatgcactgggtgcgacaggcccc
tggacaagggcttgagtggatgggaataatcaaccctagtggtggtagcacaagctacgca
cagaagttccagggcagagtcaccatgaccagggacacgtccacgagcacagtctacatgg
agctgagcagcctgagatctgaggacactgccgtgtattactgtgcgcgcggtggttacac
tggttggtctccgtctgatccgtggggtcaaggtactctggtgaccgtctcctca
159-Heavy chain variable region (amino acid sequence)
[SEQ ID NO: 200]
QMQLVQSGAEVKKPGASVKVSCKASGYTFTSYYMHWVRQAPGQGLEWMGIINPSGGSTSYA
QKFQGRVTMTRDTSTSTVYMELSSLRSEDTAVYYCARGGYTGWSPSDPWGQGTLVTVSS
11) Antibody ROR2 clone #173
173-Lambda light chain variable region (DNA sequence)
[SEQ ID NO: 237]
cagtctgtgttgactcagccaccctcagtgtcagtggccccaggaaagacggccaggatta
cctgtggtggagacaacattggacgtaaaagtgtgcactggtaccagcagaagccaggcca
ggcccctgtgctggtcatctattatgatagcgaccggccctcagggatccctgagcgattc
tctggctccacctctgggaacacggccaccctgaccatcagtagggtcgaagccggggatg
aggccgactattactgtcaggtgtgggatcgtagtagtgacctttatgtcttcggaactgg
gaccaaggtcaccgtcctaggt
173-Lambda light chain variable region (amino acid sequence)
[SEQ ID NO: 214]
QSVLTQPPSVSVAPGKTARITCGGDNIGRKSVHWYQQKPGQAPVLVIYYDSDRPSGIPERF
SGSTSGNTATLTISRVEAGDEADYYCQVWDRSSDLYVFGTGTKVTVLG
173-Heavy chain variable region (DNA acid sequence)
[SEQ ID NO: 238]
caggtgcagctggtgcagtctggggctgaggtgaagaagcctggggcctcagtgaaggtct
cctgcaaggcttctggttacacctttaccagctatggtatcagctgggtgcgacaggcccc
tggacaagggcttgagtggatgggatggatcagcgcttacaatggtaacacaaactatgca
cagaagctccagggcagagtcaccatgaccacagacacatccacgagcacagcctacatgg
agctgaggagcctgagatctgacgacacggctgtgtattactgtgcgcgccatctgggtcc
gatgggtatgtacgactggtctttcgataaatggggtcaaggtactctggtgaccgtctcc
tca
173-Heavy chain variable region (amino acid sequence)
[SEQ ID NO: 201]
QVQLVQSGAEVKKPGASVKVSCKASGYTFTSYGISWVRQAPGQGLEWMGWISAYNGNTNYAQ
KLQGRVTMTTDTSTSTAYMELRSLRSDDTAVYYCARHLGPMGMYDWSFDKWGQGTLVTVSS
12) Antibody ROR2 clone #240
240-Light chain variable region (DNA acid sequence)
[SEQ ID NO: 239]
caatctgccctgactcagcctgcctccgtgtctgggtctcctggacagtcgatcaccatct
cctgcactggaaccagcggtgacgttggcggttataactatgtctcctggtaccaacacca
cccaggcaaagcccccaaactcataatttatgatgtcaataagcggccctcaggtttttct
gatcggttctctggctccaagtctggcaacacggcctccctgacaatctctgggctccagg
ctgaggacgaggctgattattactgcagctcatatacaagcaccagcaccgtcttcggcgg
agggaccaagctgaccgtcctaggt
240-Light chain variable region (amino acid sequence)
[SEQ ID NO: 215]
QSALTQPASVSGSPGQSITISCTGTSGDVGGYNYVSWYQHHPGKAPKLIIYDVNKRPSGFS
DRFSGSKSGNTASLTISGLQAEDEADYYCSSYTSTSTVFGGGTKLTVLG
240-Heavy chain variable region (DNA acids equence)
[SEQ ID NO: 240]
cagatcaccttgaaggagtctggtcctgagctggtgaaacccacacagaccctcacactga
cctgcaccttttctgggttctcactcagcactagtggaatgtctgtgagctggatccgtca
gcccccagggaaggccctggagtggcttgcacgcattgattgggatgatgataaatactac
agcacatctctgaagaccaggctcaccatctccaaggacacctccaaaaaccaggtggtcc
ttacaatgaccaacacggaccctgtggacacagccacgtattactgtgcgcgcggtttcta
cctggcttacggttcttacgattcttggggtcaaggtactctggtgaccgtctcctca
240-Heavy chain variable region (amino acid sequence)
[SEQ ID NO: 202]
QITLKESGPELVKPTQTLTLTCTFSGFSLSTSGMSVSWIRQPPGKALEWLARIDWDDDKYY
STSLKTRLTISKDTSKNQVVLTMTNTDPVDTATYYCARGFYLAYGSYDSWGQGTLVTVSS
13) Antibody ROR2 clone #241
241-Light chain variable region (DNA acid sequence)
[SEQ ID NO: 241]
tcctatgagctgactcagccactctcagtgtcagtggccctgggacagacggccaggatta
cctgtgggggaaacaacattggaagtaaaaatgtgcactggtaccagcagaagccaggcca
ggcccctgtgctggtcatctatagggatagcaaccggccctctgggatccctgagcgattc
tctggctccaactcggggaacacggccaccctgaccatcagcagagcccaagccggggatg
aggctgactattactgtcaggtgtgggacagcagtattgtggtattcggcggagggaccaa
gctgaccgtcctaggt
241-Light chain variable region (amino acid sequence)
[SEQ ID NO: 216]
SYELTQPLSVSVALGQTARITCGGNNIGSKNVHWYQQKPGQAPVLVIYRDSNRPSGIPERF
SGSNSGNTATLTISRAQAGDEADYYCQVWDSSIVVFGGGTKLTVLG
241-Heavy chain variable region (DNA acid sequence)
[SEQ ID NO: 242]
gaagtgcagctggtgcagtctggggctgaggtgaagaagcctggggcctcagtgaaggttt
cctgcaaggcatctggatacaccttcaccaattactatatacactgggtgcgacaggcccc
tggacaagggcttgagtggatgggaataatcaaccctacaagtggtaggacaaggtacgca
cagaggttccagggcagagtcaccatgaccagggacacgtccacgaacacagtctacatgg
acctgagcagcctgagatctgaagacaccgccatgtattactgtgcgcgctctggttacta
ctggggtgttaacggtgatcagtggggtcaaggtactctggtgaccgtctcctca
241-Heavy chain variable region (amino acid sequence)
[SEQ ID NO: 203]
EVQLVQSGAEVKKPGASVKVSCKASGYTFTNYYIHWVRQAPGQGLEWMGIINPTSGRTRYA
QRFQGRVTMTRDTSTNTVYMDLSSLRSEDTAMYYCARSGYYWGVNGDQWGQGTLVTVSS

TABLE 1
Amino acid sequence of the conserved CDR motifs of anti-ROR2 clones

| | HCDR1 | HCDR2 | HCDR3 | LCDR1 | LCDR2 | LCDR3 |
|---|---|---|---|---|---|---|
| Antibody | (SEQ ID | (SEQ ID | (SEQ ID | (SEQ ID | (SEQ ID | (SEQ ID |
| clone # | NO:) | NO:) | NO:) | NO:) | NO:) | NO:) |
| ROR2-16 | GYTFTDYY | MNPNS | ARNSEWHP | SLRSYY | GKN | NSRDSSG |
| | (243) | GNS | WGYYDY | (246) | (247) | NHLV |
| | | (244) | (245) | | | (248) |
| ROR2-23 | GYRFSKY | IYPGDS | ARSFSSFIYD | QSVSSN | GAS | QQYGRSP |
| | W (249) | DT (250) | Y (251) | (252) | (253) | LT (254) |
| ROR2-24 | GYSFSNYW | IYPDDS | VRPRGAFDI | QGVGIN | DAS | QQYYSFP |
| | (255) | DT (256) | (257) | (258) | (259) | WT (260) |
| ROR2-27 | GYSFSNYW | IYPDDS | VRPRGAFDI | SSNIGAG | DNA | GTWDDSP |
| | (261) | DT (262) | (263) | HA (264) | (265) | SAYV |
| | | | | | | (266) |
| ROR2-84 | GFTFSSYW | IKQDGS | ARGSFSYDS | QSLVHS | KVS | MQTTHW |
| | (267) | EK (268) | DL (269) | DGNTY | (271) | PPT (272) |
| | | | | (270) | | |
| ROR2-90 | GGSISSGG | IYYSGS | ARGGLYWT | SSNIGSD | RND | VAWDDS |
| | YY (273) | T (273) | YSQDV (275) | Y (276) | (277) | LSGYV |
| | | | | | | (278) |
| ROR2-93 | GFTFSNYD | ISYDGS | ARSSAWVG | QSVSSSY | DTS | LHYGRSP |
| | (279) | NN (280) | GGFLSGTDD | (282) | (283) | PVT (284) |
| | | | (281) | | | |
| ROR2-96 | GFSLNDYY | IRDKAH | ARWVDDYQ | QSLAYS | KVS | MQGTHW |
| | (285) | GDTT | GYWIWSYH | DGNTY | (289) | PHT (290) |
| | | (286) | DF (287) | (288) | | |
| ROR2-121 | GFTFSSYA | ISGSGG | ARHYYSSDS | RLREKY | EDT | QAWDSS |
| | (291) | ST (292) | (293) | (294) | (295) | VI (296) |
| ROR2-159 | GYTFTSYY | INPSGG | ARGGYTGW | SSDVGG | DVS | GSFTSSIT |
| | (297) | ST (298) | SPSDP (299) | YNY | (301) | YV (302) |
| | | | | (300) | | |
| ROR2-173 | GYTFTSYG | ISAYNG | ARHLGPMG | NIGRKS | YDS | QVWDRS |
| | (303) | NT (304) | MYDWSFDK | (306) | (307) | SDLYV |
| | | | (305) | | | (308) |
| ROR2-240 | GFSLSTSG | IDWDD | ARGFYLAY | SGDVGG | DVN | SSYTSTST |
| | MS (309) | DK (310) | GSYDS (311) | YNY | (313) | V (314) |
| | | | | (312) | | |
| ROR2-241 | GYTFTNYY | INPTSG | ARSGYYWG | NIGSKN | RDS | QVWDSSI |
| | (315) | RT (316) | VNGDQ (317) | (318) | (319) | VV (320) |

Synthesis of CAR T Cells Targeting ROR

A ROR2 scFv sequence is used to generate a second generation CAR targeting ROR2. In some embodiments, the ROR2 scFv sequence comprises any of the LCVRs, HCVRs, LCDRs, and HCDRs described above. The variable heavy and light chains (connected with a (Gly₄Ser)₃ linker) and a detectable tag (e.g., c-myc tag) are added to allow detection of CAR expression by flow cytometry. The CAR is optimized to include a spacer domain upstream of the CD28 transmembrane domain if required. This is cloned into the SFG retroviral vector containing the CD28 and CD3 zeta or 4-1BB or other similar signaling CAR forms that are well known in the art, e.g., Park (2016). Stable 293 viral producing cell lines are generated, and the viral supernatant is used to transduce primary human T cells. A control sample and test sample are transduced. The control sample comprises primary human T cells that are not treated with a FoxP3 targeting agent prior to retroviral transduction. The test sample comprises primary human T cells that are treated with a FoxP3 targeting agent (e.g., anti-FoxP3/anti-CD3 bispecific antibody) prior to retroviral transduction. Retroviral transduction of the control and test samples is performed as described in Rafiq (2017) and Koneru (2015). Following transduction, CAR expression is verified by flow cytometry, staining for the c-myc tag incorporated into the ROR2-CAR. In addition, the number of effector cells (FoxP3 negative cells) and immunosuppressive cells (FoxP3 positive cells) in the control and test samples is determined by flow cytometry.

### Example 5. Synthesis of CAR T Cells Targeting ROR2 Using Selected scFv Fragments

In this example, methods for generating CAR T cells targeting ROR2 using antigen-specific scFv fragments is described. Although the phage display technology allows for the rapid selection and production of antigen-specific scFv fragments, the complete mAbs with Fc domains have a number of advantages over the scFv. First, only Fc carrying antibodies exert immunological functions, such as complement-dependent cytotoxicity (CDC) and antibody-dependent cellular cytotoxicity (ADCC). Second, bivalent monoclonal antibodies (mAbs) offer stronger antigen-binding avidity than monomeric Fab or scFv Abs. Third, plasma half-life and renal clearance is much faster for Fab or scFv compared to full length IgG. Fourth, bivalent mAb can be internalized at a faster rate compared to that of the corresponding univalent Fab or scFv. Although alpha emitters conjugated to the Fc region may not need to be internalized to kill the targets, many drugs and toxins will benefit from internalization of the immune complex.

Based on the affinity ranking result obtained through competitive ELISA and the cell-surface binding against ROR2 positive cancer cell line determined using flow cytometry, five phage display clones with high ROR2 binding affinity that specifically recognize ROR2 are selected for engineering into CAR T cells. The scFv of these selected clones are reconstructed into full-length human IgG1 recombinant antibodies that are incorporated into the engineered receptor (e.g., CAR, caTCR, eTCR).

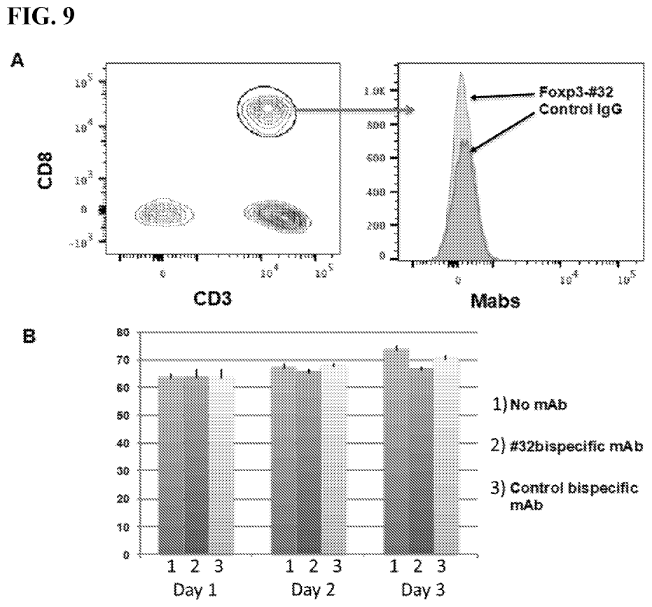

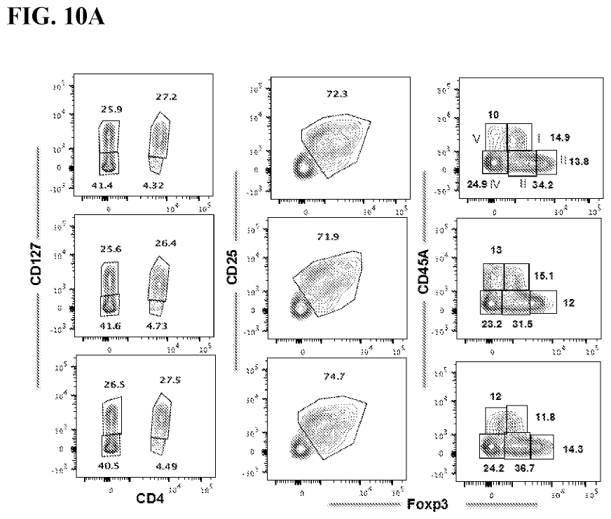

The selected scFv is converted into full length monoclonal IgG using HEK293 cells using the method of Tomimatsu et al. (2009) Biosci Biotechnol Biochem 73 (7) 1465-1469. Antibody variable regions are subcloned into the mammalian expression vectors as disclosed in WO2016142768A1 (see FIGS. 9a and 9b of WO2016142768A1, which is incorporated by reference in its entirety) together with matching Kappa or lambda light chain constant and IgG1 subclass Fc using conventional techniques known in the art.

The polypeptide sequence of one embodiment of the lambda light chain constant region of hIgG1 is provided herein as SEQ ID NO: 322, as follows:

[SEQ ID NO: 322]
QPKANPTVTLFPPSSEELQANKATLVCLISDFYPGAVTVAWKADGSPVKAG
VETTKPSKQSNNKYAASSYLSLTPEQWKSHRSYSCQVTHEGSTVEKTVAPT
ECS

The coding sequence encoding one embodiment of the lambda light chain constant region of hIgG1 is provided herein as SEQ ID NO: 323, as follows:

[SEQ ID NO: 323]
cagcctaaggccaaccctaccgtgaccctgttccccccatcctccgaggaactgcaggcca
acaaggccaccctcgtgtgcctgatctccgacttctaccctggcgccgtgaccgtggcctg
gaaggctgatggatctcctgtgaaggccggcgtggaaaccaccaagccctccaagcagtcc
aacaacaaatacgccgcctcctcctacctgtccctgacccctgagcagtggaagtcccacc
ggtcctacagctgccaagtgacccacgagggctccaccgtggaaaagaccgtggctcctac
cgagtgctcctag

The polypeptide sequence of one embodiment of the kappa light chain constant region of hIgG1 is provided herein as SEO ID NO: 324. as follows:

[SEQ ID NO: 324]
TVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFN
RGEC

The coding sequence encoding one embodiment of the kappa light chain constant region of hIgG1 is provided herein as SEQ ID NO: 325, as follows:

[SEQ ID NO: 325]
accgtggccgctccctccgtgttcatcttcccaccttccgacgagcagctgaagtccggca
ccgcttctgtcgtgtgcctgctgaacaacttctacccccgcgaggccaaggtgcagtggaa
ggtggacaacgccctgcagagcggcaactcccaggaatccgtgaccgagcaggactccaag
gacagcacctactccctgtcctccaccctgaccctgtccaaggccgactacgagaagcaca
aggtgtacgcctgcgaagtgacccaccagggcctgtctagccccgtgaccaagtctttcaa
ccggggcgagtgctag

The polypeptide sequence of one embodiment of the heavy chain constant region of hIgG1 is provided herein as SEQ ID NO: 326, as follows:

[SEQ ID NO: 326]
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

The coding sequence encoding one embodiment of the heavy chain constant region of hIgG1 is provided herein as SEQ ID NO: 327, as follows:

[SEQ ID NO: 327]
gtctcctcagcttccaccaagggcccatcggtcttccccctggcaccctcctccaagagca
cctctgggggcacagcggccctgggctgcctggtcaaggactacttccccgaaccggtgac
ggtgtcgtggaactcaggcgccctgaccagcggcgtgcacaccttcccggccgtcctacag
tcctcaggactctactccctcagcagcgtggtgaccgtgccctccagcagcttgggcaccc
agacctacatctgcaacgtgaatcacaagcccagcaacaccaaggtggacaagaaggttga
gcccaaatcttgtgacaaaactcacacatgcccaccgtgcccagcacctgaactcctgggg
ggaccgtcagtcttcctcttccccccaaaacccaaggacaccctcatgatctcccggaccc
ctgaggtcacatgcgtggtggtggacgtgagccacgaagaccctgaggtcaagttcaactg
gtacgtggacggcgtggaggtgcataatgccaagacaaagccgcgggaggagcagtacaac
agcacgtaccgtgtggtcagcgtcctcaccgtcctgcaccaggactggctgaatggcaagg
agtacaagtgcaaggtctccaacaaagccctcccagcccccatcgagaaaaccatctccaa
agccaaagggcagccccgagaaccacaggtgtacaccctgcccccatcccgggaggagatg
accaagaaccaggtcagcctgacctgcctggtcaaaggcttctatcccagcgacatcgccg
tggagtgggagagcaatgggcagccggagaacaactacaagaccacgcctcccgtgctgga
ctccgacggctccttcttcctctacagcaagctcaccgtggacaagagcaggtggcagcag
gggaacgtcttctcatgctccgtgatgcatgaggctctgcacaaccactacacgcagaaga
gcctctccctgtctccgggtaaatga

The full length anti-ROR2 antibodies are used to generate a CAR targeting ROR2. In some embodiments, the ROR2 scFv sequence comprises any of the light chain or heavy chain constant regions described above. The variable heavy and light chains (connected with a (Gly₄Ser)₃ linker) and a detectable tag (e.g., c-myc tag) are added to allow detection of CAR expression by flow cytometry. The CAR is optimized to include a spacer domain upstream of the CD28 transmembrane domain if required. This is cloned into the SFG retroviral vector containing the CD28 and CD3 zeta or 4-1BB or other similar signaling CAR forms that are well known in the art, e.g., Park (2016). Stable 293 viral producing cell lines are generated, and the viral supernatant is used to transduce primary human T cells. A control and test samples comprising primary human T cells are transduced. Retroviral transduction of the control and test samples is performed as described in Rafiq (2017) and Koneru (2015). Following transduction, the test sample is cultured in culture media supplemented with a FoxP3 targeting agent (e.g., anti-FoxP3/MHC bispecific antibody), whereas the control sample is cultured in culture media alone (e.g., not supplemented with a FoxP3 targeting agent). CAR expression in the test and control samples is subsequently verified by flow cytometry, staining for the c-myc tag incorporated into the ROR2-CAR. In addition, the number of effector cells (FoxP3 negative cells) and immunosuppressive cells (FoxP3 positive cells) in the control and test samples is determined by flow cytometry.

### Example 6. Synthesis of pMSCV-602-90GA-BBz-Ires-EGFP CAR and pMSCV-901scFv-BBz-Ires-EGFP CAR Using a FoxP3 Targeting Agent

In this example, a method of synthesizing pMSCV-602-90GA-BBz-ires-EGFP CAR and pMSCV-901scFv-BBz-ires-EGFP CAR using a FoxP3 targeting agent is described. An anti-ROR2 antibody is engineered into chimeric antibody receptor and expressed on the surface of T cells via a retroviral mammalian expression system. PG13 (GaLV pseudotyped) packaging cell line is used for transfection of the pMSCV plasmids. Human T-cells are used for transduction after 4-day stimulation and expansion with CD3/CD28 beads (Dynabeads®, Invitrogen) in the presence of interleukin-2 at 30 U/ml (control sample), whereas a test sample is additionally treated with a FoxP3 targeting agent (e.g., anti-FoxP3 antibody). Cell free supernatant from the PG13 packaging cell line is filtered and applied on T-cells in Retronectin (Takara) coated 6-well plates at 48 and 72 hours after PG13 virus producer cell line transfection.

Transduction efficiency is assessed by FACS using biotinylated Protein-L (primary) antibody (GeneScript) and PE-conjugated (secondary) antibody (BD Biosciences). In addition, the number of effector cells (FoxP3 negative cells) and immunosuppressive cells (FoxP3 positive cells) in the control and test samples is determined by FACS. Repeat FACS analyses is performed at 72 hours and every 3-4 days thereafter.

### Example 7. Synthesis of CAR T Cells Targeting WT1 Using a FoxP3 Targeting Agent

In this example, a method of producing an engineered immune cell expressing a CAR that targets WT1 is described. An ESK1 scFv sequence is used to generate a second generation CAR targeting WT1. Non-limiting examples of ESK1 scFv amino acid and nucleotide sequences are shown in the tables below. The variable heavy and light chains (connected with a (Gly₄Ser)₃ linker) and a c-myc tag are added to allow detection of CAR expression by flow cytometry. The CAR is optimized to include a spacer domain upstream of the CD28 transmembrane domain if required. This is cloned into the SFG retroviral vector containing the CD28 and CD3 zeta or 4-1BB or other similar signaling CAR forms that are well known in the art, e.g., Park (2016) Blood 127(26):3312-20. Stable 293 viral producing cell lines are generated, and the viral supernatant is used to transduce primary human T cells. A control sample is retrovirally transduced with the viral supernatant, whereas a test sample is retrovirally transduced with the viral supernatant that is supplemented with a FoxP3 targeting agent (e.g., anti-FoxP3 antibody). Retroviral transduction is performed as described in Rafiq et al. (2017) Leukemia 31(8):1788-1797 and Koneru et al. (2015) Oncoimmunology 4(3): e994446. Following transduction, CAR expression is verified by flow cytometry, staining for the c-myc tag incorporated into the WT1-CAR. In addition, the number of effector cells (FoxP3 negative cells) and immunosuppressive cells (FoxP3 positive cells) in the control and test samples is determined by flow cytometry.

TABLE 3
ESK1 scFv Amino Acid Sequences

(SEQ ID NO: 184)
QTVVTQPPSASGTPGQRVTISCSGSSSNIGSNYVYWYQQLPGTAPKLLIYR
SNQRPSGVPDRFSGSKSGTSASLAISGPRSVDEADYYCAAWDDSLNGVVFG
GGTKLTVLGSRGGGGSGGGGSGGGSLEMAQVQLVQSGAEVKKPGSSVKVSC
KASGGTFSSYAISWVRQAPGQGLEWMGGIIPIFGTANYAQKFQGRVTITAD
ESTSTAYMELSSLRSEDTAVYYCARRIPPYYGMDVWGQGTTVTVSS
(SEQ ID NO: 185)
DIQMTQSPSSLSASVGDRVTITCRASQSISSYLNWYQQKPGKAPKLLIYAA
SSLQSGVPSRFSGSGSGTDFTLTISSLQPEDFATYYCQQSYSTPLTFGGGT
KVDIKRSRGGGGSGGGGSGGGGSLEMAQVQLQQSGPGLVKPSQTLSLTCAI
SGDSVSSNSAAWNWIRQSPSRGLEWLGRTYYGSKWYNDYAVSVKSRITINP
DTSKNQFSLQLNSVTPEDTAVYYCARGRLGDAFDIWGQGTMVTVSS
(SEQ ID NO: 186)
QAVVTQPPSASGTPGQRVTISCSGSSSNIGSNTVNWYQQVPGTAPKLLIYS
NNQRPSGVPDRFSGSKSGTSASLAISGLQSEDEADYYCAAWDDSLNGWVFG
GGTKLTVLGSRGGGGSGGGGSGGGGSLEMAQMQLVQSGAEVKEPGESLRIS
CKGSGYSFTNFWISWVRQMPGKGLEWMGRVDPGYSYSTYSPSFQGHVTISA
DKSTSTAYLQWNSLKASDTAMYYCARVQYSGYYDWFDPWGQGTLVTVSS
(SEQ ID NO: 187)
DIQMTQSPSTLSASVGDRVTITCRASQNINKWLAWYQQRPGKAPQLLIYKA
SSLESGVPSRFSGSGSGTEYTLTISSLQPDDFATYYCQQYNSYATFGQGTK
VEIKRSRGGGGSGGGGSGGGGSLEMAQVQLVQSGAEVKKPGESLKISCKGS
GYNFSNKWIGWVRQLPGRGLEWIAIIYPGYSDITYSPSFQGRVTISADTSI
NTAYLHWHSLKASDTAMYYCVRHTALAGFDYWGLGTLVTVSS
(SEQ ID NO: 188)
QSVVTQPPSVSVAPGKTARITCGRNNIGSKSVHWYQQKPGQAPVLVVYDDS
DRPSGIPERFSGSNSGNTATLTISRVEAGDEADYYCQVWDSSSDHVVEGGG
TKLTVLGSRGGGGSGGGGSGGSLEMAEVQLVQSGGGVVRPGGSLRLSCAAS
GFTFDDYGMSWVRQAPGKGLEWVSGINWNGGSTGYADSVRGRFTISRDNAK
NSLYLQMNSLRAEDTALYYCARERGYGYHDPHDYWGQGTLVTVSS
(SEQ ID NO: 189)
QSVLTQPPSVSGAPGQRVTISCTGSSSNIGAGYDVHWYQQLPGTAPKLLIY
GNSNRPSGVPDRFSGSKSGTSASLAISGLQSEDEADYYCAAWDDSLNGYVF
GTGTKLTVLGSRGGGGSGGGGSGGGGSLEMAEVQLVETGGGLLQPGGSLRL
SCAASGFSVSGTYMGWVRQAPGKGLEWVALLYSGGGTYHPASLQGRFIVSR
DSSKNMVYLQMNSLKAEDTAVYYCAKGGAGGGHFDSWGQGTLVTVSS

### Example 8. Depleting T Regulatory Cells Using a TCR-Mimic Monoclonal Antibody Reactive with a Foxp3 Peptide/HLA-A*02 Complex

Depletion of T regulatory cells (Tregs) in the tumor microenvironment is one of the key strategies for successful cancer immunotherapy. However, current approaches for depleting Tregs are limited by the lack of specificity, which results also in concurrent depletion of anti-tumor effector T cells. The transcription factor forkhead box p3 (Foxp3) plays a central role in the development and suppressive function of Tregs and would be an ideal target for eliminating Tregs, but Foxp3 is an intracellular, undruggable protein. A T cell receptor mimic mAb was generated, named Foxp3-#32, reactive with a Foxp3-derived epitope in the context of HLA-A*02:01. The mAb Foxp3-#32 selectively recognizes and depletes CD4+CD25+CD127low and Foxp3+ Tregs and Treg-like T malignant cell lines, expressing both Foxp3 and HLA-A*02:01, via ADCC. A TCRm mAb targeting intracellular Foxp3 epitope could thus be a novel approach to deplete Tregs in the settings of immunotherapy of human cancers.

Materials and Methods

Peptide Synthesis

All peptides used in this study were purchased and synthesized by Genemed Synthesis, Inc. (San Antonio, Tex.). Peptides were sterile and 80% to >90% pure. The peptides were dissolved in DMSO and diluted in saline at 5 mg/mL and stored at −80° C. Control peptides used for HLA-A*02:01 were Ewing sarcoma-derived peptide EW (QLQNPSYDK) and choline transporter-like protein 4-derived peptide CT (KLLVVGGVGV). Biotinylated single chain Foxp3p/HLA-A*02:01 complexes were synthesized by refolding the peptides with recombinant HLA-A*02 and beta2 microglobulin (132M) at Eureka Therapeutics, Inc. (Emeryville, Calif.).

Cytokines, Antibodies and Cells

Human granulocyte-macrophage colony-stimulating factor (GM-CSF), interleukin (IL)-1β, IL-2, IL-4, IL-6, IL-15, tumor necrosis factor (TNF)-α and prostaglandin E2 (PGE2), TGF-β were purchased from R&D Systems (Minneapolis, Minn.). Beta 2-microglobulin (β2-m) and human IFN-γ were purchased from Sigma (St. Louis, Mo.). Cell isolation kits for CD14 and CD3 were purchased from Miltenyi Biotec. (Bergisch Gladbach, Germany). Human Treg isolation kits were purchased from Stem Cell Technology (Canada). Foxp3+ and HLA-A*02:01+ cutaneous T lymphoma cell lines MAC-1 and MAC-2A were kindly provided by Dr. Mads H. Anderson, at University of Denmark. The human T leukemia virus (HTLV) positive cell line C5MJ was kindly provided by Dr. Alexander Rudensky laboratory (MSK, New York) and the cells were transduced with HLA-A*02:01 molecule as described by Latouche et al. (2000) Nat Biotech 18:405-409. The HLA-A*02:01 SFG vector was a gift from Dr. Michelle Sadelain, at MSKCC. MAC-1 and MAC-2A cell lines were engineered to express high level of GFP-luciferase fusion protein, using retroviral vectors containing a plasmid encoding the luc/GFP. The cell lines were cultured in RPMI 1640 supplemented with 10% FCS, penicillin, streptomycin, 2 mmol/1 glutamine, and 2-mercaptoethanol at 37 C/5% CO2. Cells were checked regularly for mycoplasma. Cell identities were confirmed by mAb phenotype or genotype. Peripheral blood mononuclear cells (PBMC) from healthy donors and tumor samples from patients with ovarian cancer undergoing surgery were obtained after informed consent on Memorial Sloan-Kettering Institutional Review Board approved protocols.

The Foxp3-#32 bispecific mAb of mouse IgG1 (for flow cytometry) and their respective controls were produced at Eureka Therapeutics, Inc. (Veomett et al. (2014) Clin Cancer Res 20 (15): 4036-4046; Dao et al. (2015) Nat Biotech 33 (10): 1079-1086). APC conjugation to mouse IgG1 form of Foxp3-#32 and its control was done by using lightening-link APC antibody labeling kit according to the instructions of the manufacturer (Novus Biologicals). Mabs against human HLA A*02 (clone BB7.2), its isotype control mouse IgG2b (clone MPC-11), human CD3 (clone HIT3A and OKT3), CD4 (clone RPA-T4), CD8 (clone RPA-T8), CD25 (clone 2A3), CD33 (clone WM53), mouse anti-His tag mAb (clone F24-796) conjugated to FITC or PE, were purchased from BD Biosciences, (San Diego, Calif.). Mabs specific for human Foxp3 clone PCH101, its isotype control rat IgG2a kappa, clone 236A/E7 and its isotype control mouse IgG1 kappa, CD4 (clone OKT4), CD127 (clone HIL-7R-M21), were purchased from eBioscience. Fixation and permeabilization kit for intracellular staining was also purchased from eBioscience.

Flow Cytometry Analysis

For cell surface staining, cells were incubated with appropriate mAbs for 30 minutes on ice, washed, and incubated with secondary antibody reagents when necessary. For Foxp3-#32-bispecific mAb staining, human T cells or cancer cells were incubated with different concentrations of Foxp3-#32-bispecific mAb or control bispecific mAb for 30 minutes on ice, washed, and incubated with secondary mAb against His-Tag. Flow cytometry data were collected on a Beckman Dickinson Fortesa and analyzed with FlowJo 9.8.1 and FlowJo10 software.

In Vitro Stimulation and Human T-Cell Cultures

PBMCs from HLA-A*02:01 healthy donors were obtained by Ficoll density centrifugation. CD14+ monocytes were isolated by positive selection using mAb to human CD14 coupled with magnetic beads and were used for the first stimulation of T cells. The CD14-fraction of PBMC was used for isolation of CD3, by negative immunomagnetic cell separation using a pan T cell isolation kit. The purity of the cells was always more than 98%. T cells were stimulated for 7 days in the presence of RPMI 1640 supplemented with 5% autologous plasma (AP), 20 μg/mL synthetic peptides, 2 μg/mL β2-m, and 5-10 ng/mL IL-15. Monocyte-derived dendritic cells (DCs) were generated from CD14+ cells, by culturing the cells in RPMI 1640 medium supplemented with 1% AP, 500 units/mL recombinant IL-4, and 1,000 units/mL GM-CSF. On days 2 and 4 of incubation, fresh medium with IL-4 and GM-CSF was either added or replaced half of the culture medium. On day 5, 20 μg/mL class II peptide was added to the immature DCs. On day 6, maturation cytokine cocktail was added (IL-4, GM-CSF, 500 IU/mL IL-1, 1,000 IU/mL IL-6, 10 ng/ml TNF-α, and 1 μg/mL PGE-2). On day 7 or 8, T cells were re-stimulated with mature DCs at a 30:1, T:APC ratio, with IL-15. T cells were stimulated 3 to 5 times in the same manner, using either autologous DCs or CD14+ cells as antigen-presenting cells (APCs). A week after final stimulation, the peptide-specific T cell response was examined by IFN-gamma (γ) enzyme-linked immunospot (ELISPOT) assay (May et al. (2007) Clin Cancer Res 13: 4547-4555; Dao et al. (2009) Plos One 4(8): e6730).

IFN-γ ELISPOT Assay

HA-Multiscreen plates (Millipore) were coated with 100 μL of mouse anti-human IFN-γ antibody (10 Ag/mL; clone 1-D1K; Mabtech) in PBS, incubated overnight at 4 C, washed with PBS to remove unbound antibody, and blocked with RPMI 1640/10% autologous plasma (AP) for 2 h at 37° C. CD3+ T cells were plated with either autologous CD14+(10:1 E:APC ratio) or autologous DCs (30:1 E:APC ratio). Various test peptides were added to the wells at 20 μg/ml. Negative control wells contained APCs and T cells without peptides or with irrelevant peptides. Positive control wells contained T cells plus APCs plus 20 μg/ml phytohemagglutinin (PHA, Sigma). All conditions were done in triplicates. Microtiter plates were incubated for 20 h at 37° C. and then extensively washed with PBS/0.05% Tween and 100 μl/well biotinylated detection antibody against human IFN-γ (2 μg/ml; clone 7-B6-1; Mabtech) was added. Plates were incubated for an additional 2 h at 37° C. and spot development was done as described (7-9). Spot numbers were automatically determined with the use of a computer-assisted video image analyzer with KS ELISPOT 4.0 software (Carl Zeiss Vision) (May et al. (2007) and Dao et al. (2009)).

⁵¹Chromium Release Assay

The presence of specific CTLs was measured in a standard chromium release assay as described (May et al. (2007) and Dao et al. (2009)). Briefly, target cells are labeled with 50 μCi/million cells of Na₂ ⁵¹CrO₄ (NEN Life Science Products, Inc.). After extensive washing, target cells are incubated with T cells at various effector:target (E:T) ratios. All conditions were done in triplicates. Plates were incubated for 4-5 hours at 37° C. in 5% CO₂. Supernatant fluids were harvested and radioactivity was measured in a gamma counter. Percentage specific lysis was determined from the following formula: [(experimental release−spontaneous release)/(maximum release−spontaneous release)]×100%. Maximum release was determined by lysis of radiolabeled targets in 1% SDS.

Phage Screening, Selection of scFv Specific for the Foxp3-Derived Epitopes

A human ScFv antibody phage display library (7×10¹⁰ clones) was used for the selection of mAb clones as described previously (Dao et al. (2013) Sci Transl Med 5(176): 176ra33; Chang et al. (2017)J Clin Invest 127 (7): 2705-2718). In brief, biotinylated irrelevant peptide/HLA-A*02:01 complexes were used to remove any clones that potentially bind to HLA-A*02:01. Remaining clones were screened for the Foxp3p/HLA-A*02:01 complex. The selected clones were enriched by 3-4 rounds of panning. Positive clones were determined by standard ELISA method against biotinylated single chain Foxp3p/HLA-A*02:01 complexes. The positive clones were further tested for their binding to peptide/HLA-A*2 complexes on live cell surfaces by flow cytometry, using a TAP-deficient, HLA-A*02:01+ cell line, T2, which is defective in presentation of endogenous HLA-associated peptides. T2 cells were pulsed with positive and multiple control peptides (50 μg/ml) in the serum-free RPMI1640 medium, in the presence of 20 μg/ml β2M overnight. The cells were washed, and the staining was performed in following steps. The cells were first stained with purified scFv phage clones, and followed by staining with a mouse anti-M13 (bacteriophage) mAb, and finally the goat Fab2 anti-mouse IgG conjugated to FITC or PE. Each step of the staining was done between 30-60 minutes on ice and the cells were washed twice between each step of the staining (Dao et al. (2013) and Chang et al. (2017)).

Engineering Full Length Human IgG1 Using the Selected scFv Fragments

Full-length human IgG1 of the selected phage clones were produced in HEK293 and Chinese hamster ovary (CHO) cell lines, as described (Dao et al. (2009)). In brief, antibody variable regions were subcloned into mammalian expression vectors, with matching Lambda or Kappa light chain constant sequences and IgG1 subclass Fc. Molecular weights of the purified full length IgG antibodies were measured under both reducing and non-reducing conditions by electrophoresis.

Construction, Expression and Purification of Foxp3-#32 Bispecific mAb

The Foxp3-#32 bispecific mAb in the format of a typical bispecific T cell engager was engineered as previously described (Veomett et al. (2014)). N-terminal end of mAb Foxp3-#32 scFv was linked to the C-terminal end of an anti-human CD3ϵ scFv of a mouse monoclonal antibody by a flexible linker. The DNA fragments encoding for the scFv of two mAbs were synthesized by GeneArt (InVitrogen) and subcloned into Eureka's mammalian expression vector pGSN-Hyg using standard DNA technology. A hexhistamine (His) tag was inserted downstream of the Foxp3-#32 bispecific mAb at the C-terminal end for the detection and purification of the bispecific mAb.

Chinese hamster ovary (CHO) cells were transfected with the Foxp3-bispecific mAb expression vector and stable expression was achieved by standard drug selection with methionine sulfoximine (MSX), a glutamine synthetase (GS)-based method. CHO cell supernatants containing secreted Foxp3-#32 bispecific mAb molecules were collected. Foxp3-bispecific mAb was purified using HisTrap HP column (GE healthcare) by FPLC AKTA system. Briefly, CHO cell culture was clarified and loaded onto the column with low imidazole concentration (20 mM), and then an isocratic high imidazole concentration elution buffer (500 mM) was used to elute the bound Foxp3-bispecific mAb protein. A negative control bispecific mAb antibody, was constructed from an irrelevant human IgG1 antibody (Cat #ET901, Eureka Therapeutics) replacing the Foxp3-#32 scFv.

Characterization of the Full-Length Human IgG1 for the Foxp3 Peptide/HLA-A*02:01 Complex

Specificities of the fully human IgG1 mAbs for the Foxp3 peptide/A2 complex were determined by staining T2 cells pulsed with or without Foxp3 peptides or various analogs or control peptides, using direct or indirect staining. The fluorescence intensity was measured by flow cytometry. The same method was used to determine the binding of the mAb to cell lines.

Treg Generation, Phenotypic Analysis and Foxp3-#32 mAb Binding

CD4+ T cells were purified from PBMCs of healthy HLA-A*02:01 positive donors by FACS sorting, and were stimulated with allo-PBMCs (HLA-A*02:01 negative) as stimulator and feeder cells at ratios of effector:stimulator (E:S) 1:5-10, or with tumor cells (E:S: 1:1) in the presence of recombinant human IL-2 (100 unit) and TGF-β (10 ng/ml) for one to two weeks and the same stimulation was repeated to maintain the Treg cells (Levings et al. (2002) J Exp Med 196(10): 1335-1346; Lu et al. (2010) Plos One 5(12): e15150; Godfrey et al. (2004) Blood 104 (2): 453-461). The phenotype of Tregs was determined by surface staining of the cells with mAbs to CD4, CD25+, CD127, CD45RA, mouse Foxp3 mAb-Foxp3-#32 conjugated to APC, for 30 minutes on ice, washed. Foxp3 expression was measured by intracellular protein staining using mAb to human Foxp3 (clone PCH101, or its isotype control rat IgG2a kappa) and Cytofix/CytoPerm kit (eBiosciences), according to the instructions of the manufacture. Analysis was done by flow cytometry on a Beckman Dickinson Fortesa.

Cytotoxicity of Foxp3-#32 Bispecific mAb Specific for Tregs in the Context of HLA-A*02:01

Four methods were used to measure the ADCC against Tregs by Foxp3-#32 bispecific mAb. First, for the natural Tregs, PBMCs from healthy donors who are either HLA-A*02:01 positive or negative were incubated with or without Foxp3-#32 bispecific mAb or control irrelevant bispecific mAb at 1 μg/ml for one to three days. Cells were harvested, washed and stained with mAbs to CD4, CD25, CD127, CD45RA, followed by intracellular staining with mAb to Foxp3 or its isotype control. Treg reduction was accessed on the expression of well-defined Treg markers. In brief, lymphocytes were gated based on forward and side scatter, followed by gating on CD4+CD127 high or CD4+CD127 low population. The CD4+CD127 high or CD4+CD127 low population was further determined by 2 sets of Treg markers: CD25 vs Foxp3; or CD45RA vs Foxp3. Second, natural Tregs only represent a few percent of CD4+ T cells; therefore, in order to obtain sufficient readout on Treg killing, Tregs generated were also used in vitro as targets. The killing of Tregs was determined by reduction of Treg population by flow cytometry. In brief, purified CD3T cells by negative selection from HLA-A*02:01 negative donors used as effectors were incubated with Tregs generated from HLA-A*02:01+ donors at an E:T ratio 5:1, in the presence or absence of Foxp3-#32 bispecific mAb (1 μg/ml) or its control bispecific mAb for over nigh. The cells were washed and stained with mAbs to CD4, CD25, Foxp3 and HLA-A*02. HLA-A*02 positive cells were gated (as Treg targets) and the killing of Tregs was determined by the reduction of percentage of CD4+CD25+Foxp3+ cells in the HLA-A*02:01+ cells, compared to control cultures with effectors alone or with effectors plus control bispecific mAb. Third, Treg-like T lymphoma cell line MAC-2A, or T leukemia cell line C5MJ/A2 (Foxp3+/HLA-A*02:01+) were used as targets in ADCC assay by a standard ⁵¹Cr-release assay. Fourth, since ⁵¹Cr-release assay cannot be used to determine a longer term ADCC, an in vitro bioluminescence imaging (BLI) method was used to test ADCC activity of the Foxp3-#32 bispecific mAb. In brief, PBMCs from HLA-A*02:01 negative donors were incubated with MAC-1, or MAC-2A cells that had been transduced with GFP/luciferase, at an E:T ratio 30:1, in the presence of Foxp3-#32 bispecific mAb or its control bispecific mAb at 1 μg/ml, for 3 days, then 30 μg of luciferin was added to each well, before imaging. Tumor growth was calculated by average of the luminescence signal of triplicate microwell cultures.

In addition, to test if the mAb shows any non-specific or off-target toxicity to normal cells, PBMCs from HLA-A*02:01 positive or negative healthy donors were incubated in the presence or absence of 0.2 or 1 μg/ml Foxp3-#32 bispecific mAb or its control bispecific mAb overnight. Cells were washed and stained with mAbs to human CD3, CD19 and CD33 to determine whether these cell lineages are killed by the bispecific mAbs. Total cell numbers were measured by trypan blue exclusive staining.

Antibody-Dependent Cellular Cytotoxicity (ADCC)

Target cells used for ADCC were T2 cells pulsed with or without Foxp3-TLIp or irrelevant control peptides, or Foxp3+ and HLA-A*02:01+ or negative cell lines MAC2A, C5MJ/A2, C5MJ, Jurkat and HL-60 that were not pulsed with peptides. The Foxp3-#32 bispecific mAb or its isotype control, at various concentrations were incubated with target cells and fresh PBMCs, or activated T cells from HLA-A*02:01− donors, at different E:T ratio for 4-5 hrs. Cytotoxicity was measured by standard ⁵¹Cr-release assay. When activated T cells were used as effectors, CD3 T cells isolated by negative selection were stimulated with Dynabead human T activator CD3/CD28 (Gibco™ 11131D, Gibco) for 5-7 days.

Results

Selection of Foxp3-Derived Epitopes in the Context of HLA-A*02:01

There is little information on the epitopes derived from Foxp3 that could induce T cell responses. Therefore, immunogenic epitopes that could generate cytotoxic CD8 T cells against Foxp3 were identified. The entire human Foxp3 protein sequence was screened using three computer-based predictive algorithms BIMAS (www-bimas.cit.nih.gov/cgi-bin/molbio/ken_parker_comboform), SYFPEITHI (www.syfpeithi.de/) and RANKPEP (bio.dfci.harvard.edu/Tools/rankpep.html) to identify potential high affinity binders to HLA-A*02:01. A number of potential epitopes derived from human Foxp3 for CD8 T cells in the context of HLA-A*02:01 molecule were selected to test if the peptides were able to induce specific CD8 T cell responses (Table 3). Importantly, all the selected HLA-A*02:01-binding peptides were predicted to be cleaved at the C-terminus, suggesting a higher probability of being processed by proteasomes.

TABLE 3
Sequences of Foxp3-derived peptides

| Position | Sequences |
|---|---|
| p344-353 | TLIRWAILEA (SEQ ID NO: 8) |
| p252-260 | KLSAMQAHL (SEQ ID NO: 2) |
| p390-398 | SLHKCFVRV (SEQ ID NO: 3) |
| p304-312 | SLFAVRRHL (SEQ ID NO: 4) |
| p388-396 | NLSLHKCFV (SEQ ID NO: 5) |
| p95-103 | LLQDRPHFM (SEQ ID NO: 6) |
| p69-77 | LQLPTLPLV (SEQ ID NO: 7) |

Peptide-Specific T Cell Response in the Context of HLA-A*02:01 Molecule

As the computer algorithms are not always predictive of in vitro or in vivo activity, immunogenicity of the predicted peptides by HLA-A*02 binding on T2 cells by their ability to stimulate peptide-specific CD8 T cell responses from HLA-HLA-A*02:01+ donors was tested. Initially, 7 peptides were selected to test T cell responses (Table 3). Six out of seven peptides (except for peptide 304-312) consistently induced peptide-specific T cell responses in multiple donors. Because human Foxp3 is a member of a large forkhead family of related proteins, to avoid potential off targets shared within the family proteins, the peptide TLIRWAILEA (SEQ ID NO: 8) (position 344-353; “TLI”) from among other immunogenic epitopes was selected as the epitope on which to focus because the TLI peptide has minimal homology with other Foxp family members, such as Foxp1, 2 and 4. Interestingly, this peptide has also been shown to induce strong peptide-specific CD8+ T cell responses, which recognize Foxp3+/HLA-A*02:01+ cutaneous T lymphoma cells (Larsen et al. (2013) Leukemia 27: 2332-2340).

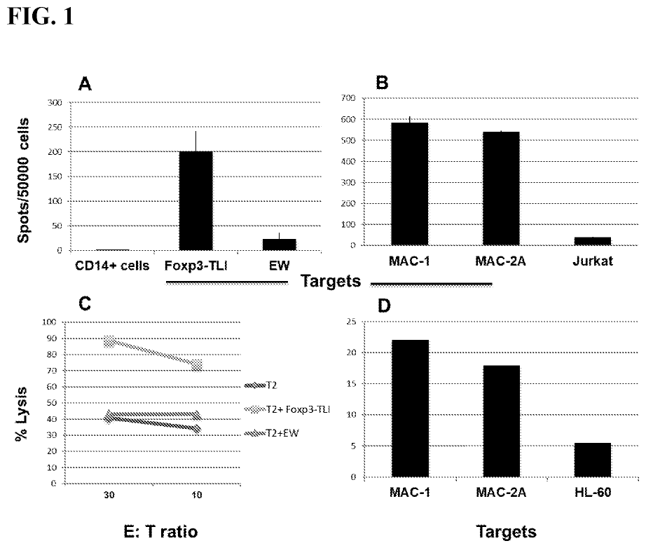

CD3+ T cells from multiple HLA-A*02:01+ donors were stimulated 3 to 5 times with the TLI peptide and the peptide-specific T cell response was measured by IFN-γ ELISPOT and ⁵¹Cr-release assays. After four rounds of stimulation, T cells recognized autologous CD14+ monocytes pulsed with TLI peptide, but not CD14+APC alone or pulsed with an irrelevant HLA-A*02:01-binding peptide EW, by IFN-γ ELISPOT assay (FIG. 1A). Importantly, a T cell response was also observed against HLA-A*02:01+Foxp3+ cutaneous T lymphoma cell lines MAC-1 and MAC-2A, but not the Foxp3 negative/HLA-A* 02:01 negative T leukemia cell line Jurkat, suggesting that TLI-stimulated T cells could recognize a naturally processed Foxp3 epitope presented by HLA-A*02:01 molecule (FIG. 1B). Consistent with the results of IFN-γ secretion, TLI peptide-stimulated T cells killed T2 cells pulsed with the TLI peptide and MAC-1 and MAC-2A cells that had not been pulsed with peptide, but did not kill the HLA-A*02:01 negative, Foxp3+ cell line HL-60 (FIGS. 1C and D.)

Selection of a TCR-Mimic mAb Specific for Foxp3 Peptide TIL in the Context of HLA-A*02:01 Molecule Using Phage Display Technology

By confirming that the Foxp3-TLI peptide is able to induce an epitope-specific T cell response that recognizes tumor cells expressing the Foxp3 protein, a TCRm mAb specific for the TLI/HLA-A*02:01 complex was generated, by using phage display technology as previously described (Dao et al. (2013)). The selected clones were tested for their binding to live T2 cells pulsed with TLI or control peptides. Any clones that showed binding to T2 cells without the TLI peptide or with HLA-A*02:01-binding irrelevant peptides were removed. Based on these data and binding to live cells that express Foxp3 and HLA-A*02:01, eight scFv clones were selected for additional characterization.

Characterization of Bispecific mAbs Specific for Foxp3 TIL/HLA-A*02:01 Complex

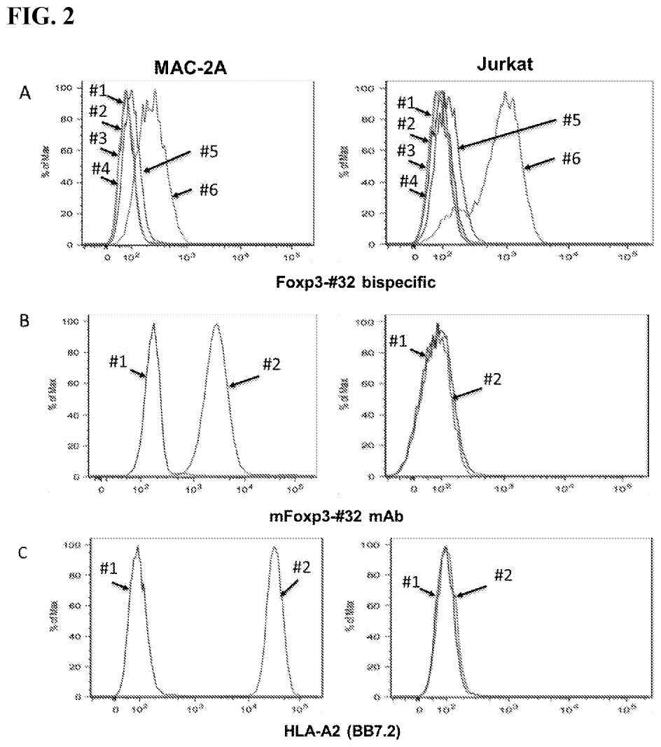

Cell surface epitope density for TCR and TCRm targets is expected to be 50-100 times lower than for typical mAbs recognizing cell surface proteins, which may limit cytolytic activity. Therefore, as a strategy to enhance the TCRm cytotoxicity, bi-specific T cell engager (bispecific mAb) constructs of the eight selected clones reactive with the Foxp3-TLI peptide/HLA-A*02:01 complex were generated (Dao et al. (2015)). The bispecific mAbs were tested against T2 cells pulsed with or without Foxp3-TLI or an irrelevant peptide and also to cell lines MAC-1, MAC-2A and Jurkat that had not been pulsed with peptide. While all the bispecific mAb constructs showed binding to T2 cells pulsed with Foxp3-TLI peptide, none of them bound to T2 cells alone or with control peptide. Further, only bispecific mAb Foxp3-#32 bound to both MAC-1 and MAC-2A cells, suggesting it had a sufficient avidity to recognize naturally processed epitopes (FIG. 2A shows data on MAC-2A). Foxp3-#32 bispecific mAb also bound to CD3+ T cell line Jurkat, demonstrating the binding to CD3 with the anti-CD3 arm of the bispecific mAbs. To exclude non-specific binding to Jurkat cells, mouse IgG1 forms of Foxp3-#32 mAb were used to test the binding to both MAC-2A and Jurkat cells. The mAb-Foxp3-#32 only bound to MAC-2A, but not Jurkat (FIG. 2B), confirming that the binding required HLA-A*02:01 expression; MAC-2A, but not Jurkat is HLA-A2 positive (FIG. 2C).

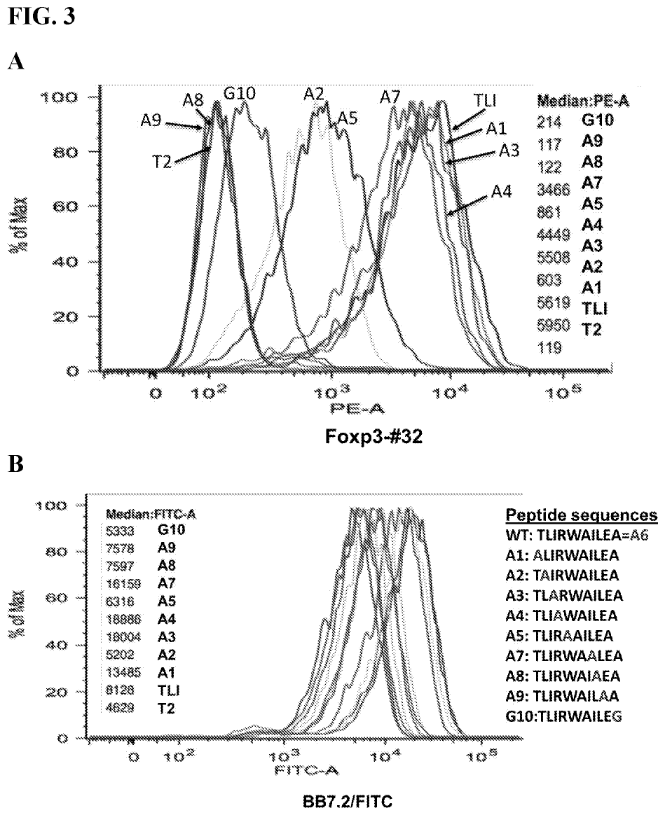

The amino acid specificity of the Foxp3-#32 mAb to the peptide was further analyzed by the binding of Foxp3-#32 bispecific mAb to T2 cells pulsed with analog TLI peptides. TLI peptide was substituted with alanine at position 1, 2, 3, 4, 5, 7, 8 and 9, or with glycine at position 10. Position 6 was already alanine and it was left intact. The mutant peptides were loaded onto T2 cells and tested for Foxp3-bispecific mAb binding. Alanine or glycine substitution at position 2, 5, 8, 9 or 10 strongly reduced the binding of Foxp3-bispecific mAb, and alanine substitution at position 4 and 7 also reduced the Foxp3-#32 mAb binding but in a lesser degree, as compared to the native TLI peptide (FIG. 3A). The loss of the binding at position 2 and to a lesser degree at position 10 could be due to the reduction of the peptide binding to the HLA-A*02 molecule, as both peptides showed reduced binding in T2 stabilization assays, whereas changes at positions 4 and 7 increased binding (FIG. 3B). Overall, mAb Foxp3-#32 showed peptide-wide amino acid requirements for binding. These results further demonstrated the specificity of the Foxp3-bispecific mAb against the TLI peptide/HLA-A*02:01 complex.

Recognition of Human Tregs and Tumor Cells Expressing Foxp3 and HLA-A*02:01 by Foxp3-#32 mAb

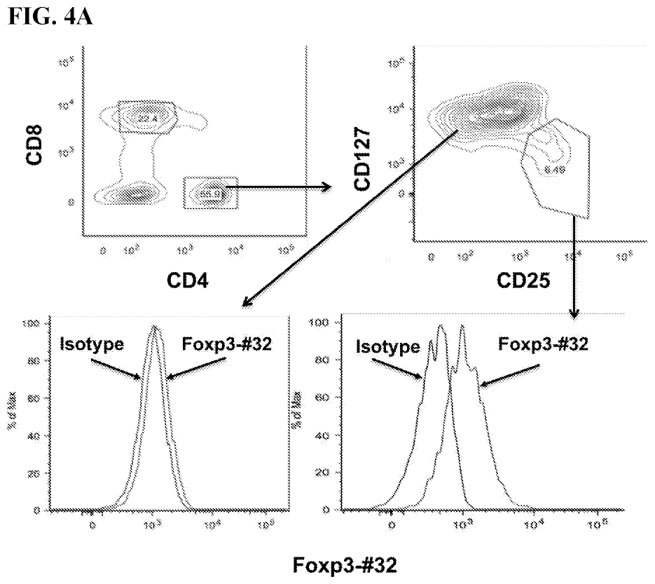

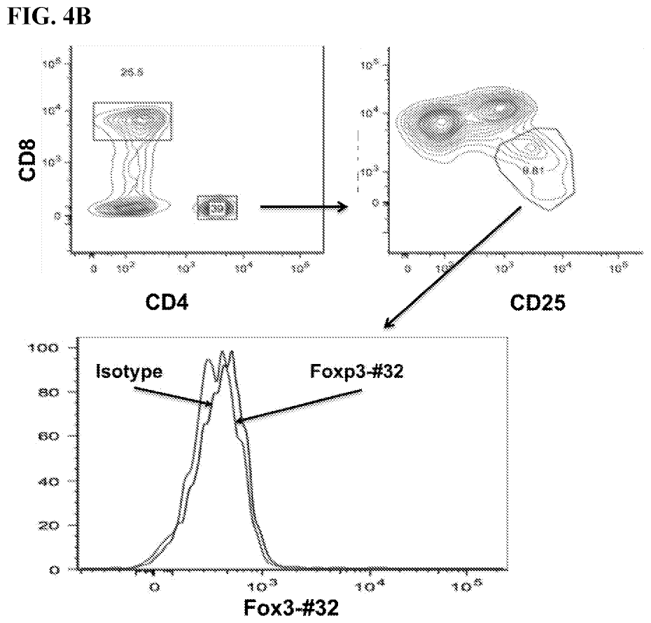

Although the Foxp3-#32 mAb has demonstrated selective binding to T2 cells pulsed with TLI peptide, it was crucial to test if the TLI epitope is processed and presented by HLA-A*02:01 molecule in naturally occurring Tregs and induced Tregs. Foxp3-#32 mAb binding to Tregs from HLA-A*02:01 positive or negative PBMCs from healthy donors were compared. CD4+ T cells were gated on CD25 high/CD127 low population, a characteristic of natural Tregs. The binding by Foxp3-#32 mAb was predominately seen in the CD4+CD25^{hi}CD127^{lo} population compared to its isotype control in HLA-A*02:01+ donor (FIG. 4A, lower right histogram), but not to CD4+CD25^{int/lo}CD127^{hi} cells (FIG. 4A, lower left histogram). The mAb Foxp3-#32 did not bind to the same CD4+CD25^{hi}CD127^{lo} Treg population from a HLA-A*02:01 negative donor (FIG. 4B), nor to the CD3/CD8 double positive T cells from HLA-A*02:01 positive donor (supplementary FIG. 1A).

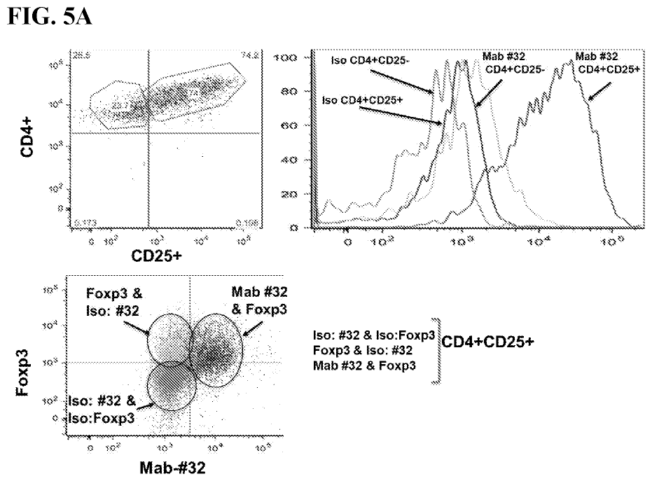

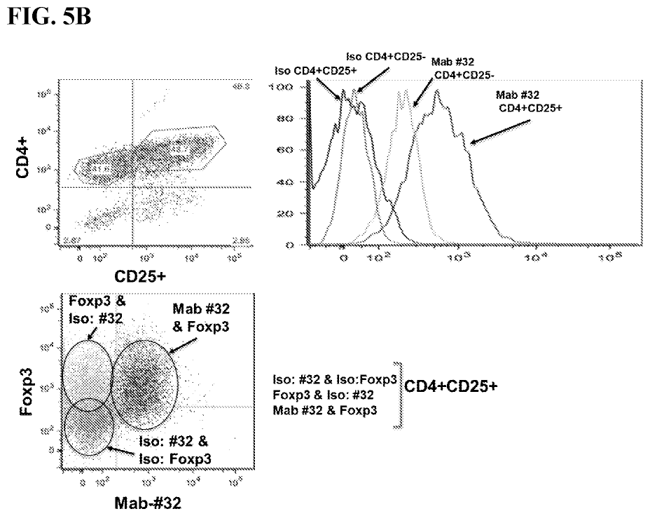

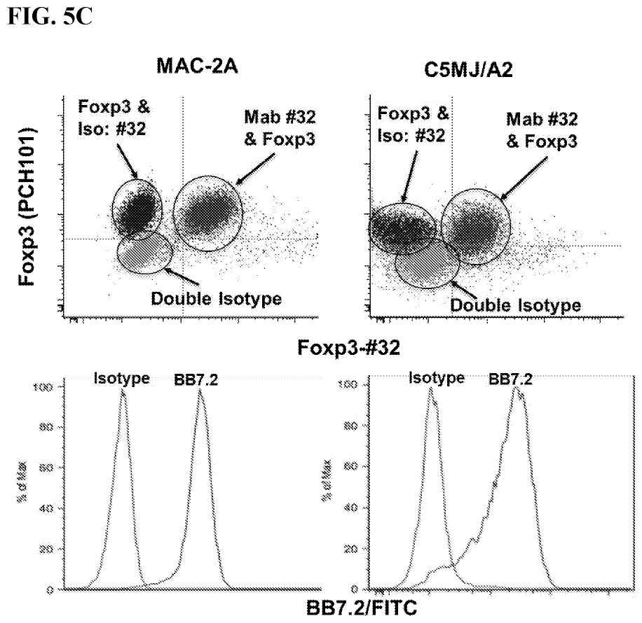

There have been a number of methods to generate Tregs in vitro that would yield substantial numbers of Tregs to study (Levings et al. (2002); Lu et al. (2010); Godfrey et al. (2004)). Therefore, to test if the Foxp3-#32 mAbs could also recognize inducible Tregs, Treg clones by repetitive stimulation of purified CD4+ T cells from HLA-A*02:01+ donors with either allo-PBMCs or tumor cells MAC-2A in the presence of IL-2 and TGF-β were generated, because tumor cells have been shown to induce Tregs (Id.) T cells generated by tumor stimulation resulted in a population of 74% CD4+CD25+ cells (FIG. 5A, upper left panel) that was positive for both intracytoplasmic Foxp3 protein and Foxp3-#32 mAb (FIG. 5A, lower left panel). Dual isotype controls showed no binding to either Foxp3 protein or Foxp3-#32 mAb. When the CD4+CD25+ population was gated, strong binding of mAb Foxp3-#32 was shown as compared to its isotype control (FIG. 5A, upper right panel). There was a weak binding of the mAb-Foxp3-#32 to the CD4+CD25 negative population. Similar results were also seen in Tregs generated by allo-PBMC stimulation using a HLA-A*02:01 negative donor (FIG. 5B). It is possible that Foxp3 may be transiently expressed on activated CD4 T cells, in addition to Tregs bearing the same gated markers, or that an arbitral gates may not precisely reflect Treg population. Nonetheless, the results demonstrated that Foxp3-#32 mAb is able to recognize human Treg cells derived from two different methods of preparation.

Many types of human cancer cells express Foxp3, which is associated with poor prognosis and greater metastatic potential (Karanikas et al. (2008) J Transl Med 6: 19-26; Truiulzi et al. (2013) J Cell Physiol 228: 30-35). Especially, T cell malignancies have been shown to share the characteristics of Tregs, phenotypically and functionally. Therefore, a Foxp3-targeting mAb could also potentially kill tumor cells expressing Foxp3. In addition to MAC-1 and MAC-2A T lymphoma cell lines, T leukemia virus-transduced cell line, CSMJ, also expressed Foxp3. Therefore, CSMJ cell line with HLA-A*02:01 was transduced to test if the Foxp3-#32 mAb could also recognize the epitope in these cells. While dual isotype controls for both mAbs to Foxp3 protein (mouse IgG2k) and Foxp3-#32 (mouse IgG1) was negative for both mAbs, Foxp3-#32 mAb bound only to the cytoplasmic Foxp3+ population in both MAC-2A and C5-MJ/A2 cells (FIG. 5C). In contrast, mouse IgG1 isotype for Foxp3-#32 mAb did not bind to the cytoplasmic Foxp3 protein positive population. The results thus show Foxp3-#32 mAb binding to Foxp3+/HLA-A*02:01 positive cancer cells. However, because a viable A02+/Foxp3 knockout line was not available, the extent the binding to these cancer cell lines is attributable to the TLI peptide expression, as compared to other possible off-target, cross-reactive peptides, was not determined.

Foxp3-#32 Bispecific mAb-Mediated T Cell Cytotoxicity Against Foxp3+ Tregs and Tumor Cells in the Context of HLA-A*02:01

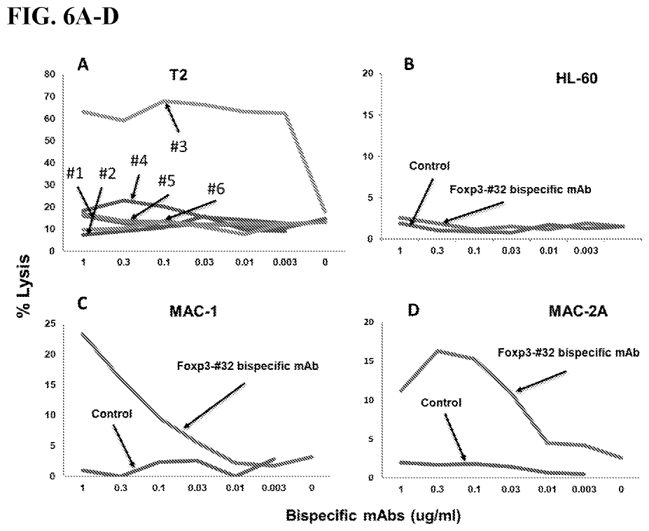

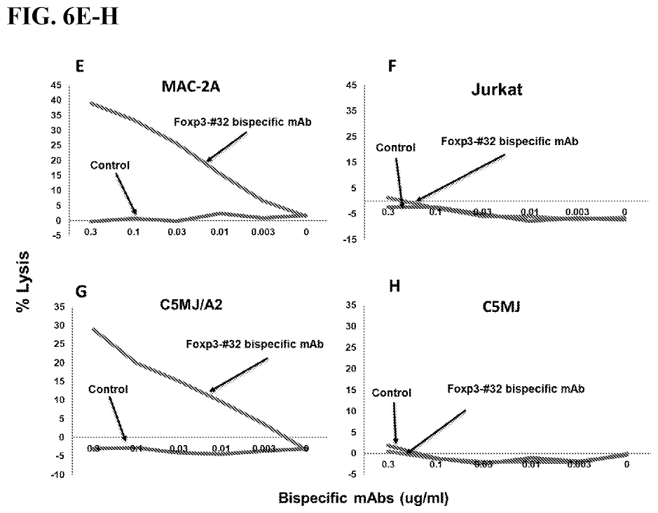

Having demonstrated the binding of the Foxp3-#32 to the Foxp3+HLA-A2+ cells, whether the Foxp3-#32 bispecific mAb mediates cytolytic activity such as ADCC was next tested. First, T2 cells pulsed with TLI or control HLA-A*02:01-binding peptide CT, were incubated with human PBMCs used as effectors, in the presence or absence of the Foxp3-#32-bispecific mAb or its control bispecific mAb. Foxp3-#32 bispecific mAb mediated specific, effective killing activity against T2 cells pulsed with TLI peptide, but not T2 cells alone or pulsed with control peptide (FIG. 6A), nor Foxp3 negative/HLA-A*02:01 negative cell line HL-60 (FIG. 6B-D). Similarly, PBMCs in the presence of Foxp3-#32-bispecific mAb showed dose-dependent killing against Treg-like T lymphoma cell lines MAC-1 and MAC2A cells at the indicated concentrations (FIGS. 6C and D). Neither MAC-1 and MAC-2A cell lines express CD3, and T cell cytotoxicity against these cell lines was not mediated by the scFv arm of the anti-CD3 mAb.

When activated T cells were used as effectors, Foxp3-#32 bispecific mAb-mediated killing was further enhanced against MAC-2A cells. In addition, Foxp3-#32 bispecific mAb mediated T cell killing against another Treg-like T leukemia cell line C5MJ transduced with HLA-A*02:01, but not its parental cells C5MJ, nor Jurkat cells. These results further confirmed that Foxp3-#32 bispecific mAb is able to kill the tumor cells expressing both Foxp3 and HLA-A*02:01 (FIG. 6E-H), with the similar caveat noted above about the role of off-target, cross-reactive peptides that may be contributing to reactivity as well.

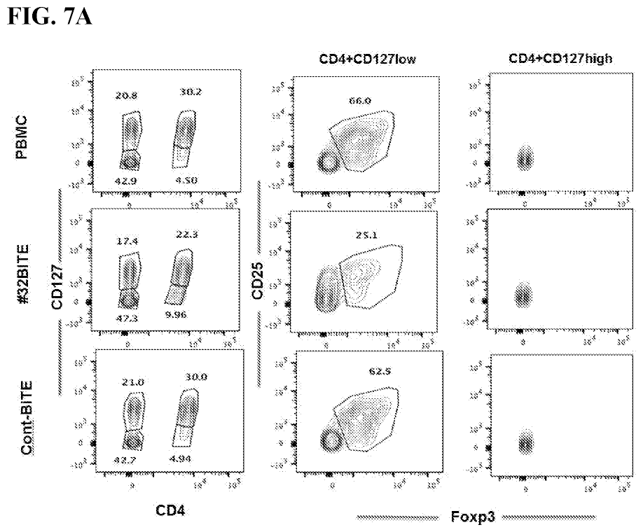

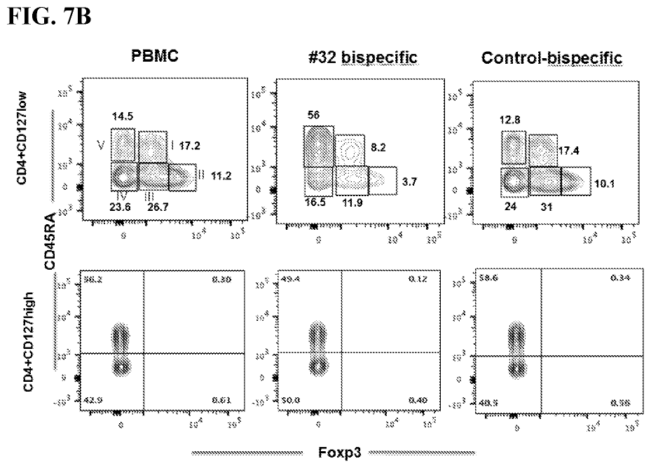

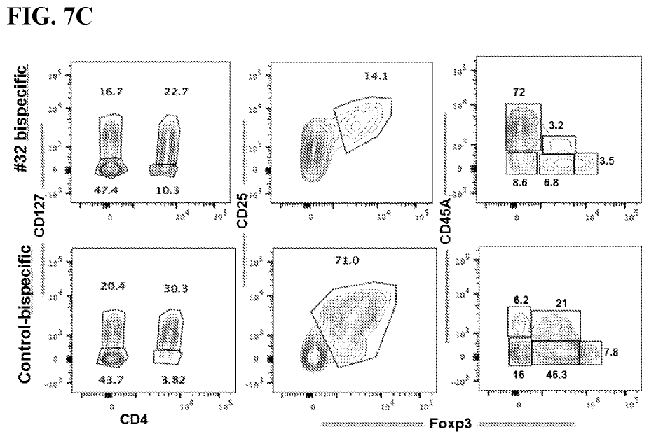

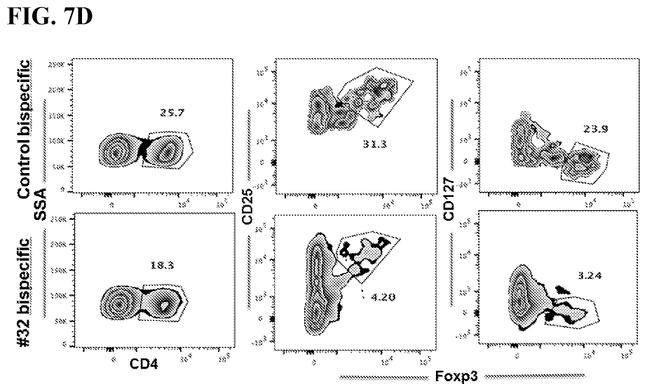

Whether the ADCC function of Foxp3-#32 bispecific mAb is able to selectively deplete natural Tregs from PBMCs by using flow cytometric analyses with a panel of Treg markers was tested. Since the mAb is targeting a Foxp3-derived epitope, the reduction of the Foxp3+ population in the cells that express bona fide Treg markers would provide more direct evidence for depletion of the Foxp3+Tregs. PBMCs from both HLA-A*02:01 positive or negative donors were incubated with Foxp3-#32 bispecific mAb or the control bispecific mAb for one to three days. Several gating strategies were employed: first, gating on the lymphocyte population, then CD4+CD127high (conventional T cells) or CD127low (Tregs) populations, followed by gating with two sets of markers: CD25 vs intracytoplasmic Foxp3 or CD45RA vs intracytoplasmic Foxp3. Representative flow cytometric analysis two days after incubation are shown (FIG. 7A). PBMCs alone and PBMCs treated with the control bispecific mAb (top row and bottom row, respectively) showed similar patterns with approximately 30% CD4+CD127 high and 5% CD4+CD127 low populations. Cells treated with Foxp3-#32 bispecific mAb (middle row) minimally changed the percentage of these two populations (left column panels). Further, CD25+ intracytoplasmic Foxp3+ cells were only detected in CD4+CD127low, but not in the CD4+CD127high population, because resting conventional T cells do not express CD25, nor Foxp3 (See middle panels vs right panels). There was about a 60% reduction in CD25+Foxp3+ cells treated with FoxP3-#32 bispecific mAb, compared to the cells treated with control bispecific mAb or no bispecific mAb (middle column, middle row vs middle column, top or bottom rows). The data are consistent with selective depletion of the Treg population from PBMCs.

Because the CD4+CD127low population increased in the Foxp3-#32 bispecific mAb treated group, to further confirm the cell reduction as an absolute vs relative depletion of Foxp3+Tregs, these two populations were further analyzed using a more detailed set of markers (FIG. 7B) PBMCs and PBMCs treated with control bispecific mAb showed a similar percentage of two Treg subsets. (For clarity these subsets are labeled in the first panel with Roman numerals I to V and the percentage of each cell type within the gate box is indicated by the number.) The upper panels show the CD127 low cells and the lower panels show the CD127 high cells. All populations of FoxP3 positive cells were depleted when cells were treated with Foxp3-#32 bispecific mAb: Fraction I (naïve Tregs) and II (effector and terminally differentiated Tregs) and also fraction III (non-Tregs: CD45RA−, Foxp3 low). Total Tregs from fraction I and II are about 28% in the two control groups. Strikingly, cells treated with #32 bispecific mAb showed a nearly 60% reduction in these cells. Notably, the percentage of Foxp3 low population in fraction III also was reduced, showing a Foxp3-specific depletion, although these fraction III cells are not classic Tregs. In contrast, CD45RA+ T cells increased more than 4 fold in the Foxp3-#32 bispecific mAb treated group compared to the control bispecific mAb group. This suggests that upon engaging with Treg target cells via #32 bispecific mAb, naive T cells are activated to become effector cells. It also suggested that activated conventional T cells are not or minimally depleted by the #32 bispecific mAb treatment. There were no Foxp3+ cells were observed in CD45RA+/CD4+/CD127high populations in all three groups (lower 3 panels).

When cells were analyzed in the same manner after three days of treatment, CD4+/CD127low/CD25+/Foxp3+ Treg populations showed further depletion: 14% remaining in the population in Foxp3-#32 bispecific mAb-treated cells compared to 78% remaining in control bispecific mAb-treated group (an 82% reduction) (FIG. 7C). Furthermore, CD45RA low/Foxp3+ naïve and CD45RA−/Foxp3high effector Tregs were reduced to 7% of the population, compared to 29% remaining with the control bispecific mAb.

The reduction of the CD4+CD25+CD127 low and Foxp3+ cells in the Foxp3-#32 bispecific mAb-treated group was seen as early as the first day after treatment. The CD4+CD127 low population was about 4% in PBMCs, Foxp3-#32 bispecific mAb treated and control bispecific mAb treated groups. However, the CD25+Foxp3+ cells were 62.3%, 42.5% and 57% in these three groups, showing a 30% reduction. CD8+(non-CD4+) CD127 low population showed no CD25+Foxp3+ cells. In addition, total cell numbers did not show any significant change after one to three days of treatment among three groups in two separate experiments. However, the percentage of lymphocytes showed a minimal reduction in the cells treated with Foxp3-#32 bispecific mAb (FIG. 9B).

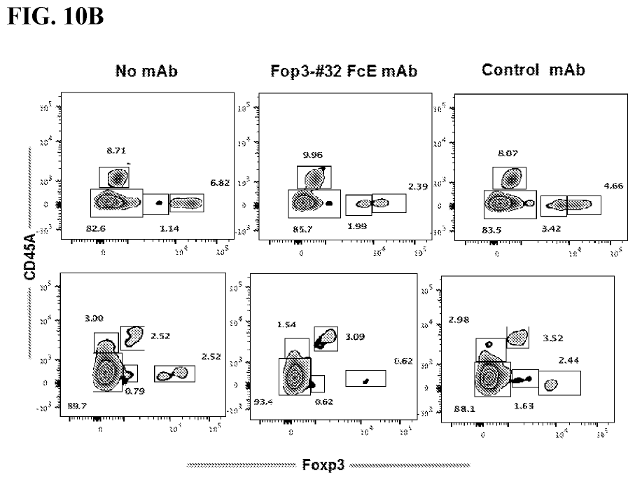

No Foxp3+Tregs were depleted in HLA-A*02:01 negative donor, in the same experiments (FIG. 10A). These results demonstrated that the Foxp3-#32 bispecific mAb selectively depleted Foxp3+ cells, in the context of HLA-A*02:01 molecule.

A similar experiment using ascites from ovarian patients who are HLA-A*02:01 positive was performed. After two days of treatment with Foxp3-#32 bispecific mAb, CD4+CD25high/Foxp3+ Tregs decreased from 32% (control bispecific mAb) to 4% (FIG. 7D). This was confirmed with another set of markers: The CD4+CD127 low/Foxp3+ population decreased from 24% (control) to 3%. The cells were also treated with FoxP3-#32 IgG with an Fc region mutated to improve ADCC (Veomett (2014)), because CD33+CD14+ monocytes/macrophages infiltration was observed in the ascites of the patient. The depletion of effector Tregs (fraction II) was evident on day 2 after treatment with the specific TCRm (FIG. 10B, upper panels) and this population decreased to 0.4%, compared to the un-treated cells (4.8%) and control mAb-treated cells (3.4%) after three days (FIG. 10B, lower panels). There was no typical naïve Treg population (fraction I) on day 2. Similar phenotypes have also been shown in other types of cancer, due to heterogeneity of tumor samples (Tanaka et al. (2017) Cell Res 27: 109-118).

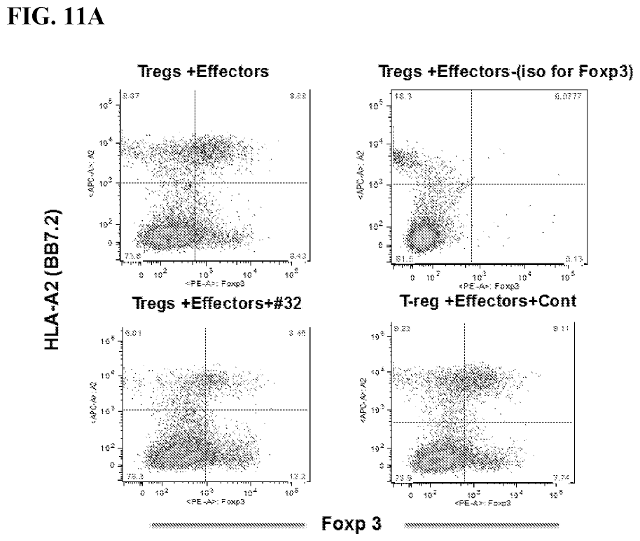

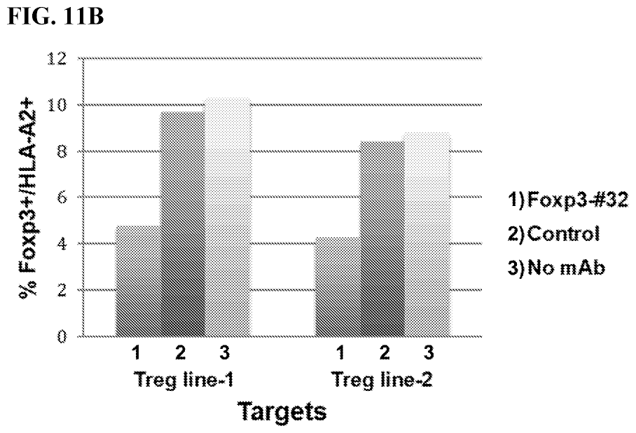

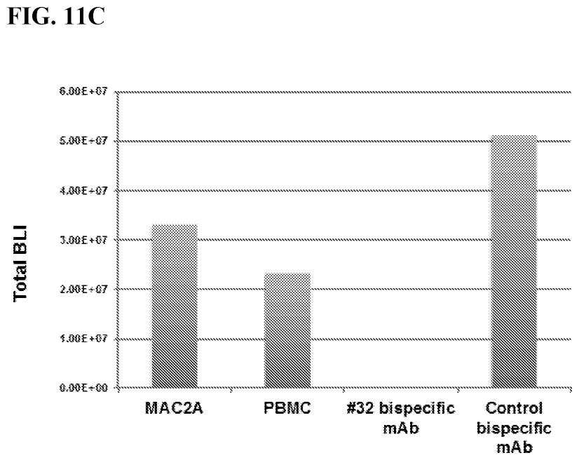

To further confirm these results, Treg lines from HLA-A*02:01+ donors (phenotype shown in FIG. 5B) were generated and used as Treg targets. Treg lines used as targets were incubated overnight with purified T cells from HLA-A*02:01-negative donors, in the presence or absence of Foxp3-#32 bispecific mAb or control bispecific mAb. Following this, the percentage of Foxp3+ cells in HLA-A*02:01+ T cell population was measured by staining the cells with mAbs to HLA-A2 and intracellular Foxp3 protein. Since HLA-A*02:01+ cells are only present in the target Treg lines, reduction of the HLA-A*02:01 and Foxp3 double positive cells indicated Foxp3-#32 bispecific mAb-mediated cytotoxicity against the Tregs (FIG. 11A). While control cell cultures treated with effector PBMCs alone (upper left panel), or effectors with the control bispecific mAb (lower right panel), showed 9-10% HLA-A*02:01/Foxp3 double positive cells in the co-culture, the percentage of HLA-A*02:01+/Foxp3+ T cells decreased more than 60% in the presence of Foxp3-#32-bispecific mAb (lower left panel). Foxp3+/HLA-A*02:01 negative cells (effector T cells, possibly activated by Treg allo-stimulation) were not killed by the Foxp3-#32 mAb, indicating the HLA-A2 restriction for the mAb recognition. Similar results were obtained from a second Treg line #2 (FIG. 11B). These results demonstrated that the Foxp3-#32-bispecific mAb is able to recognize and mediate T cell cytotoxicity against human Tregs in the context of HLA-A*02:01 molecules.

To test a long-term cytotoxic effects of Foxp3-#32-bispecific mAb against Foxp3+/HLA-A*02:01+ cells, GFP/luciferase+ MAC-1 or MAC-2A cells were incubated with effector PBMCs from HLA-A*02:01 negative donors, in the presence of the Foxp3-#32- or control bispecific mAb and measured the total bioluminescent intensity (BLI) after three days. Significant cytotoxicity of the Foxp3-#32 bispecific mAb against MAC-2A was seen as there were little target BLI signal left, indicating that the MAC-2A cells were killed in the presence of the Foxp3-#32-bispecific mAb (FIG. 11C). Similar results were also seen with MAC-1 cell line.

Potential Off Targets for Mab Foxp3-#32 in the Context of HLA-A*02:01

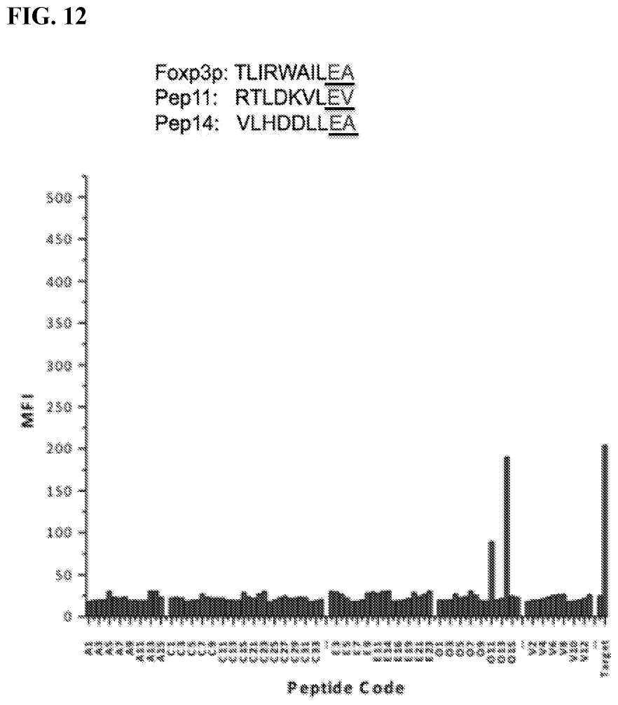

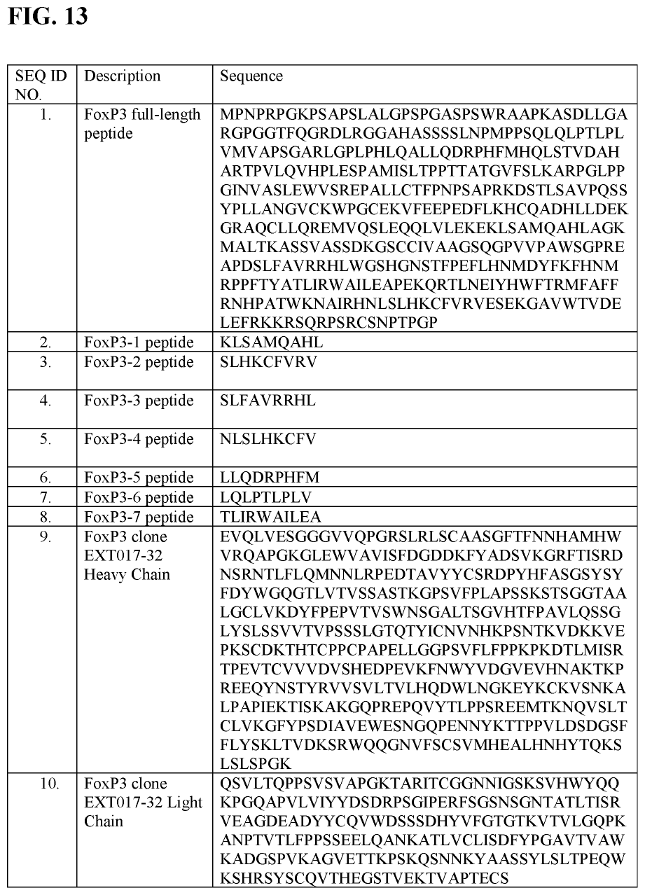

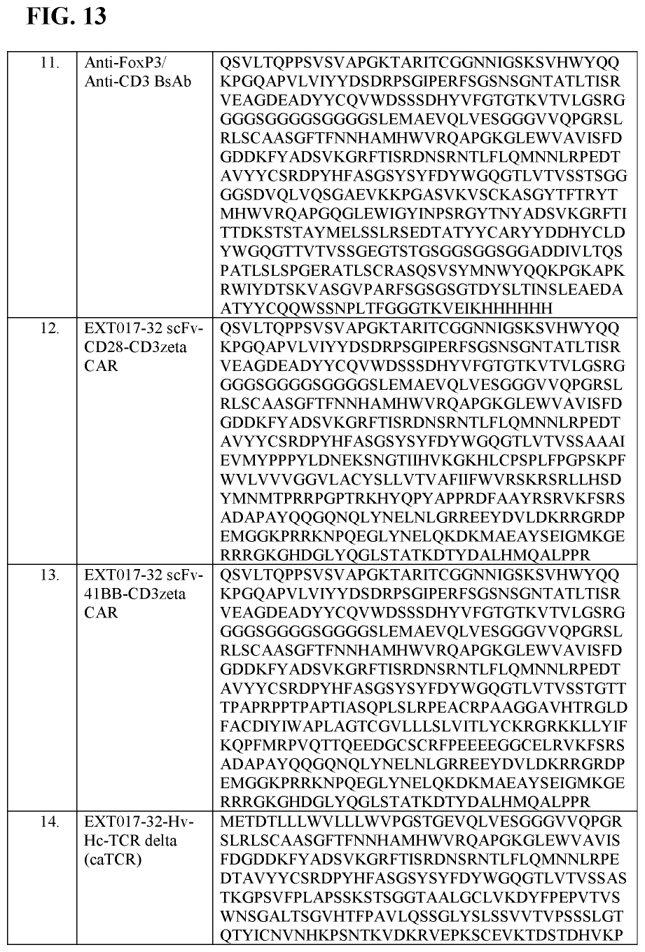

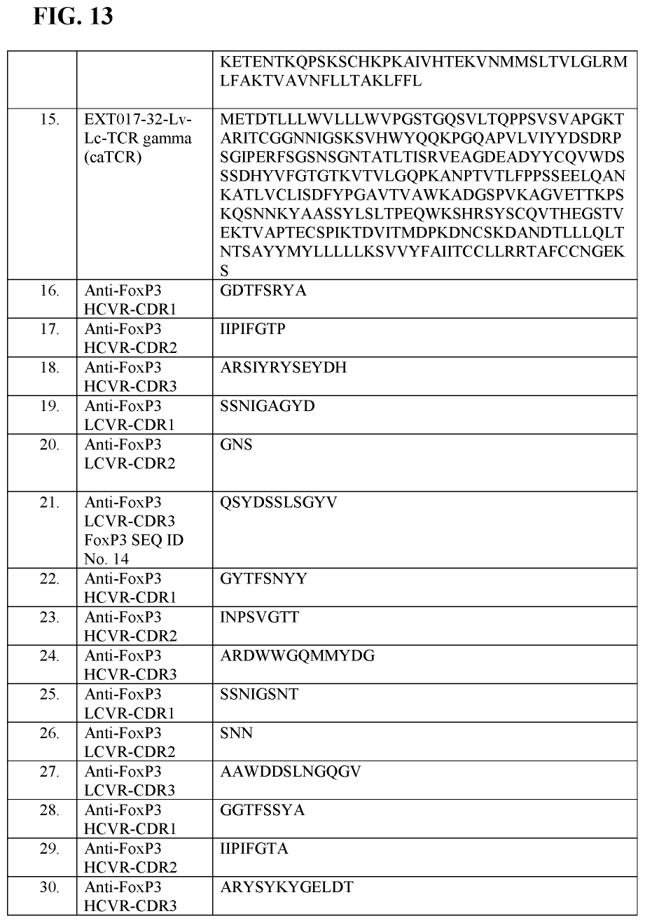

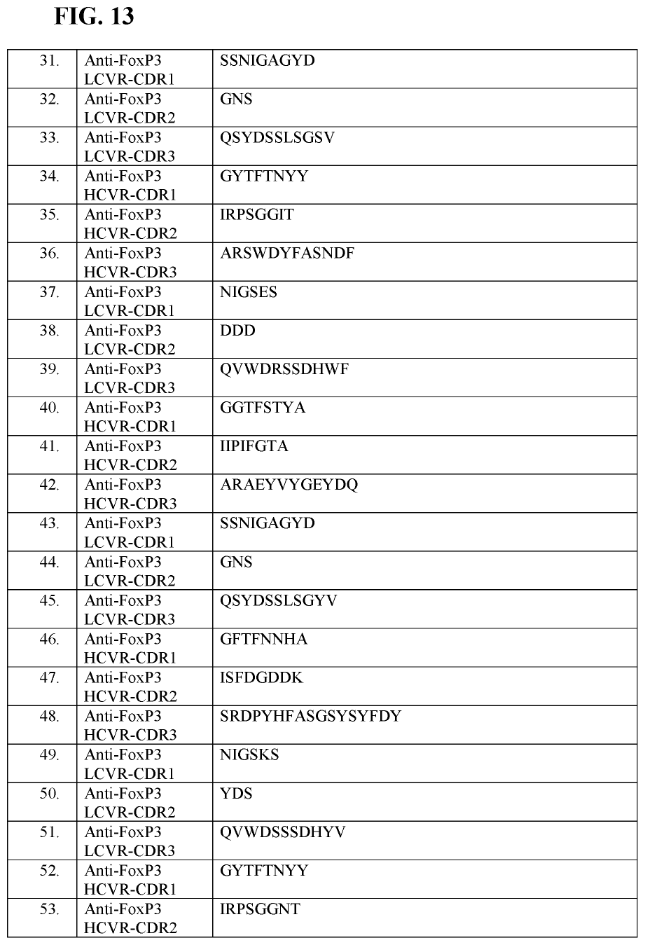

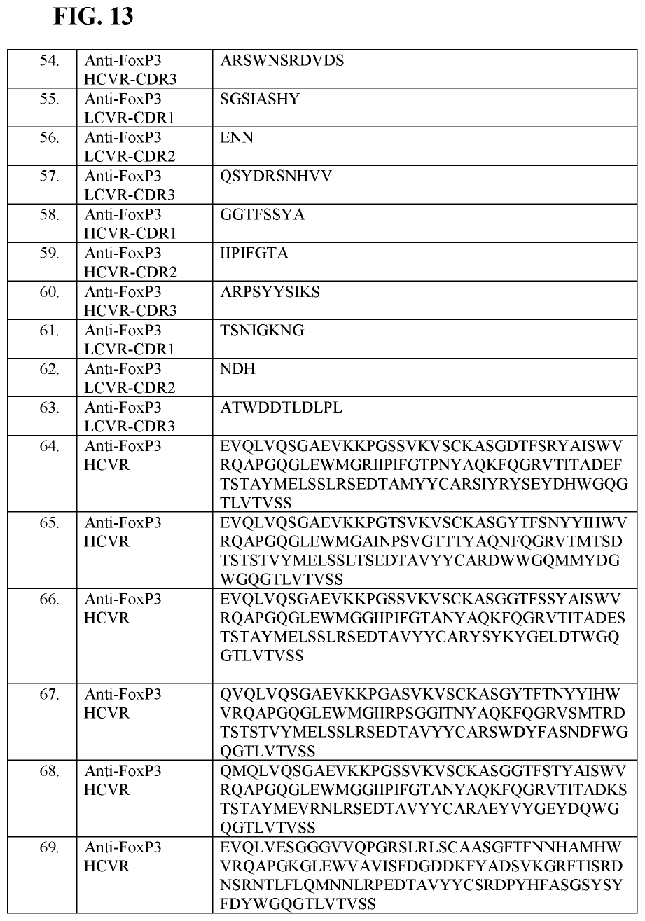

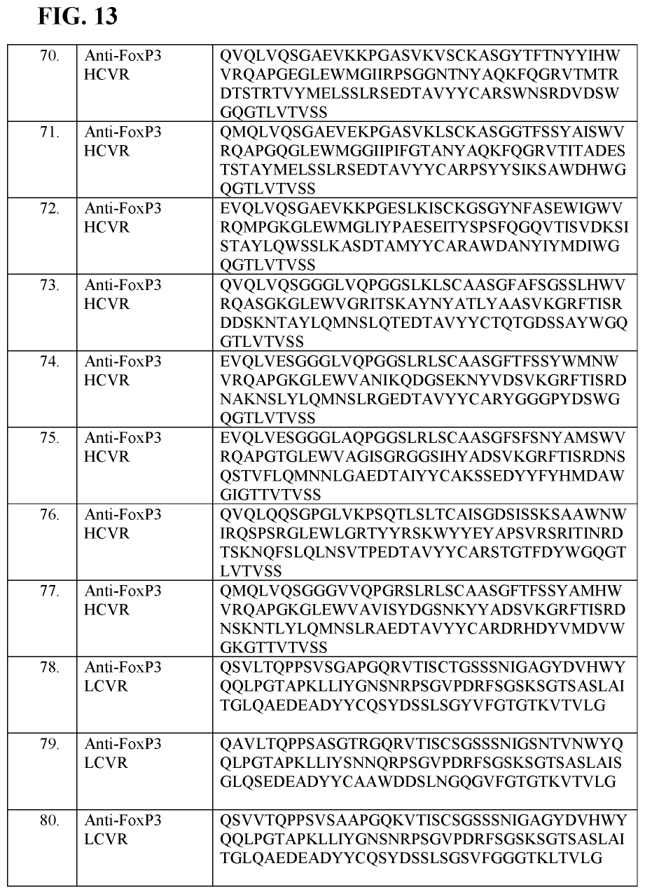

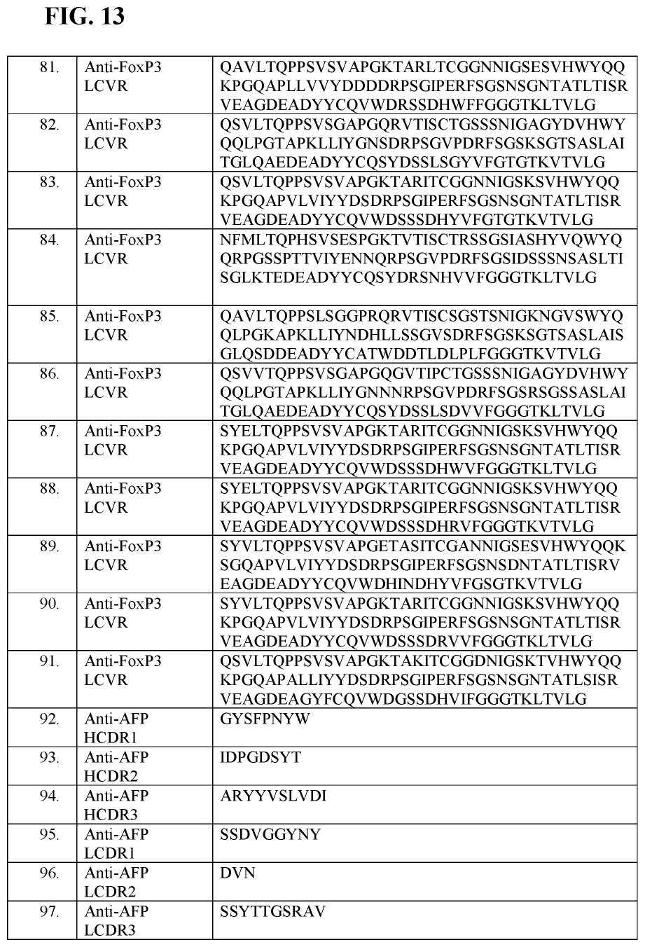

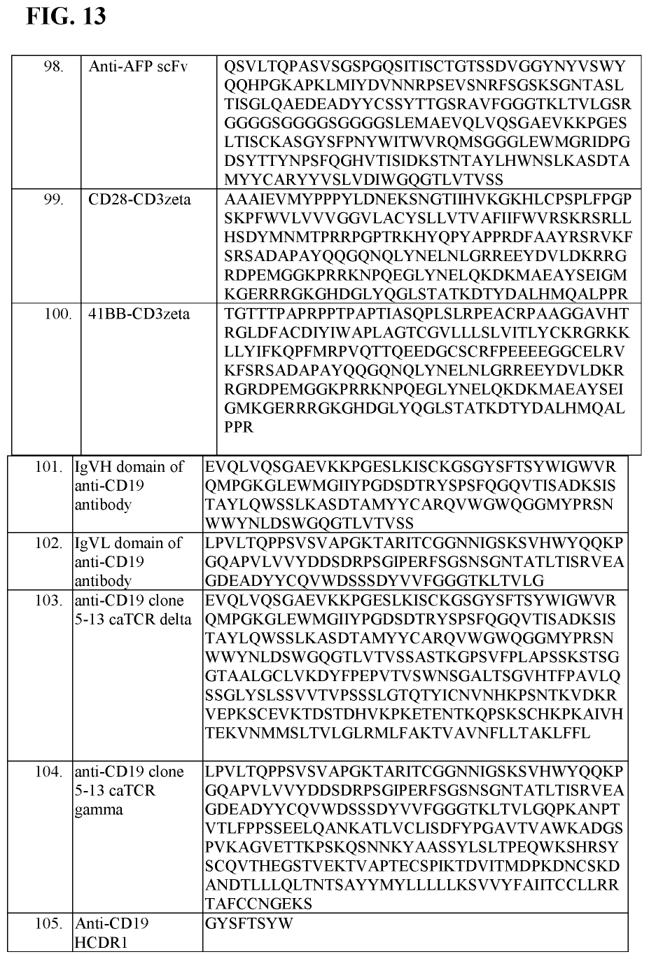

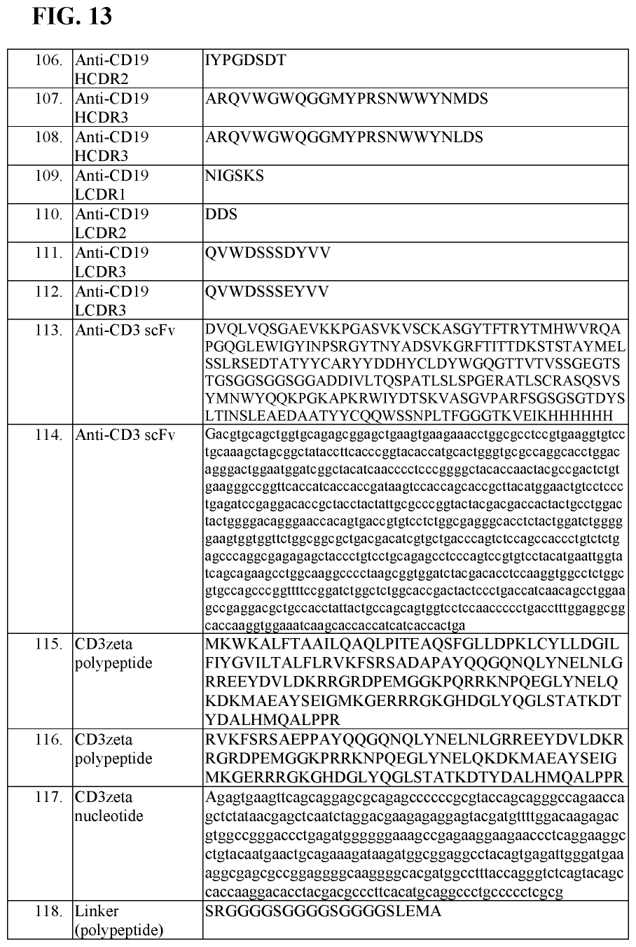

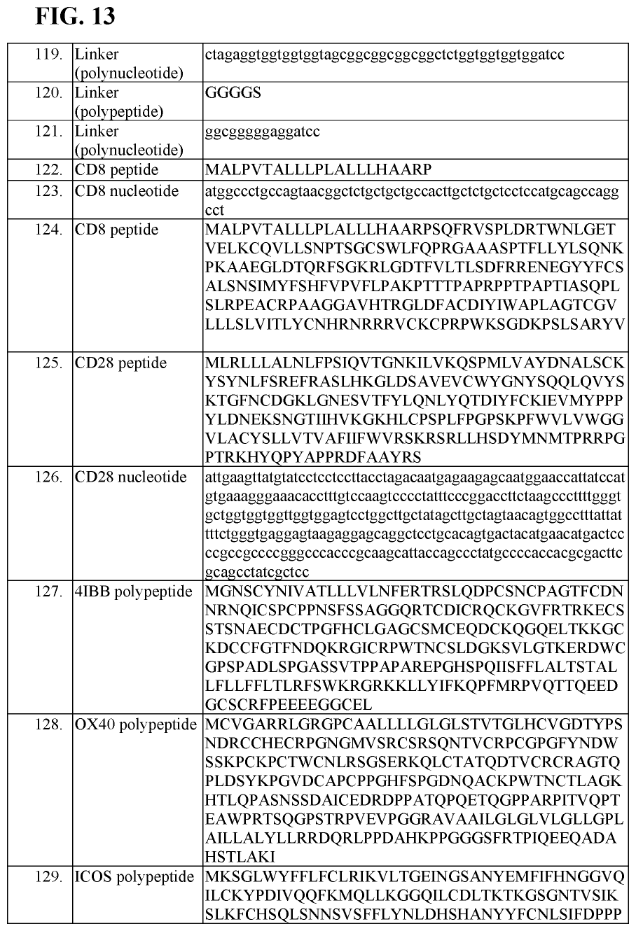

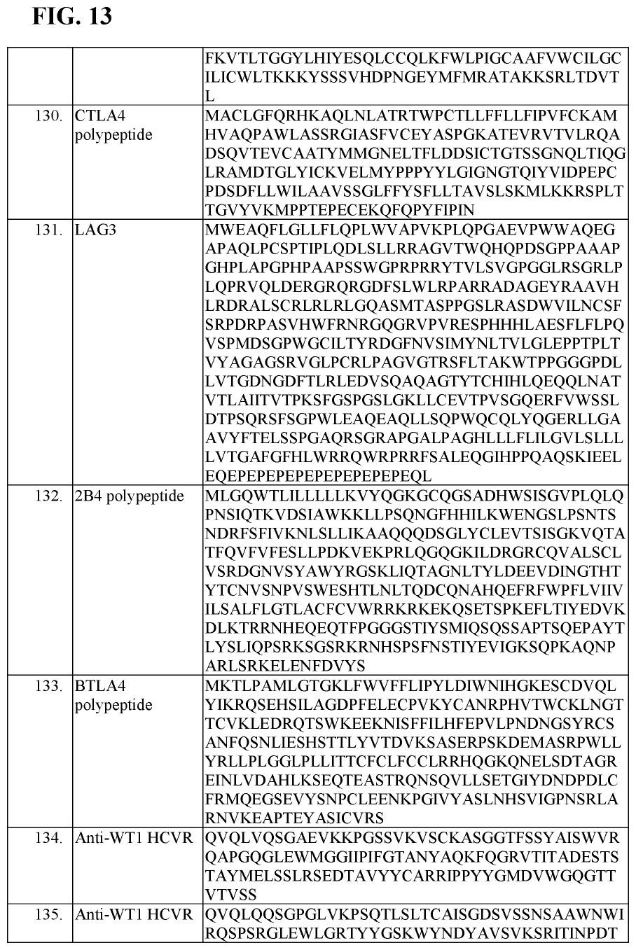

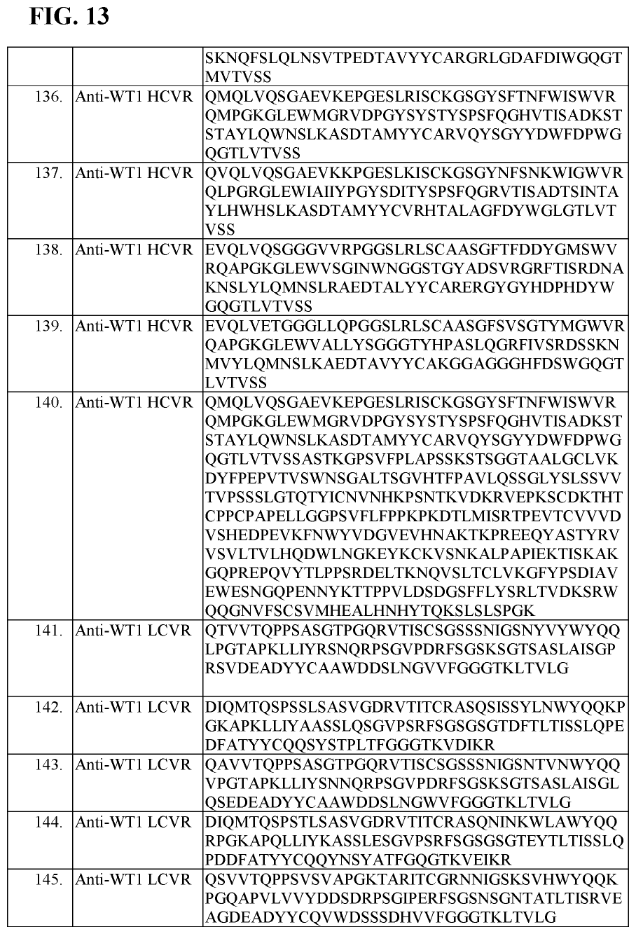

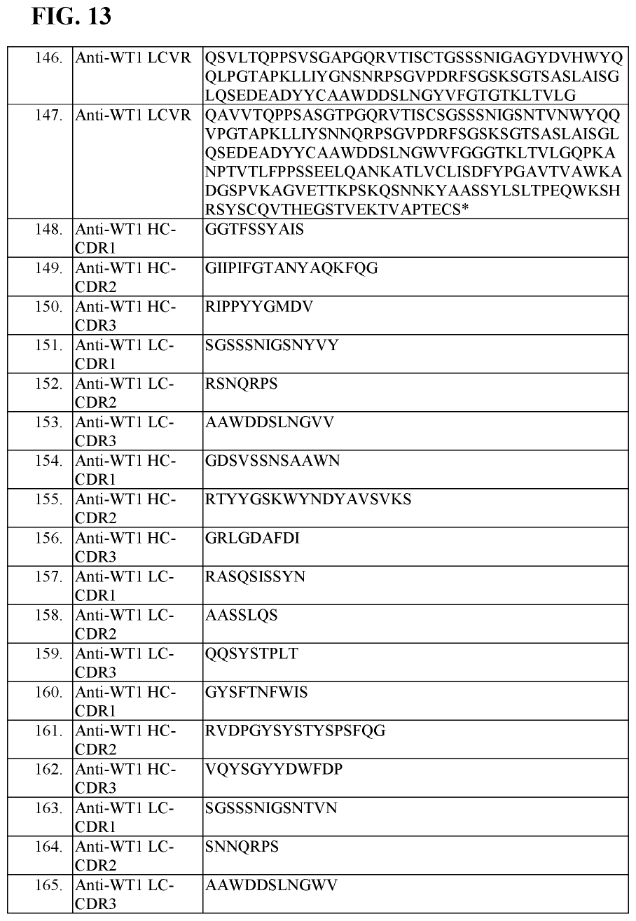

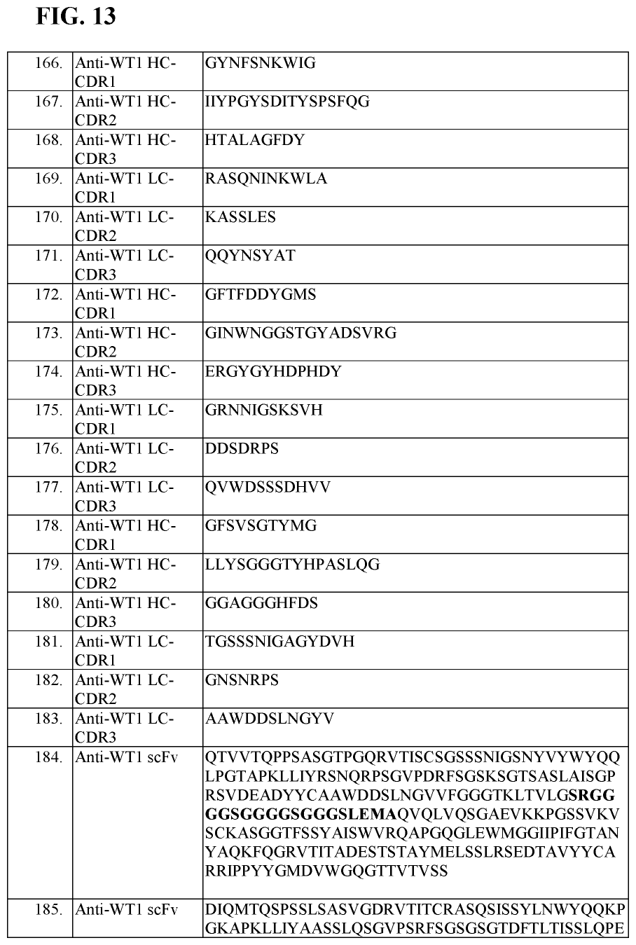

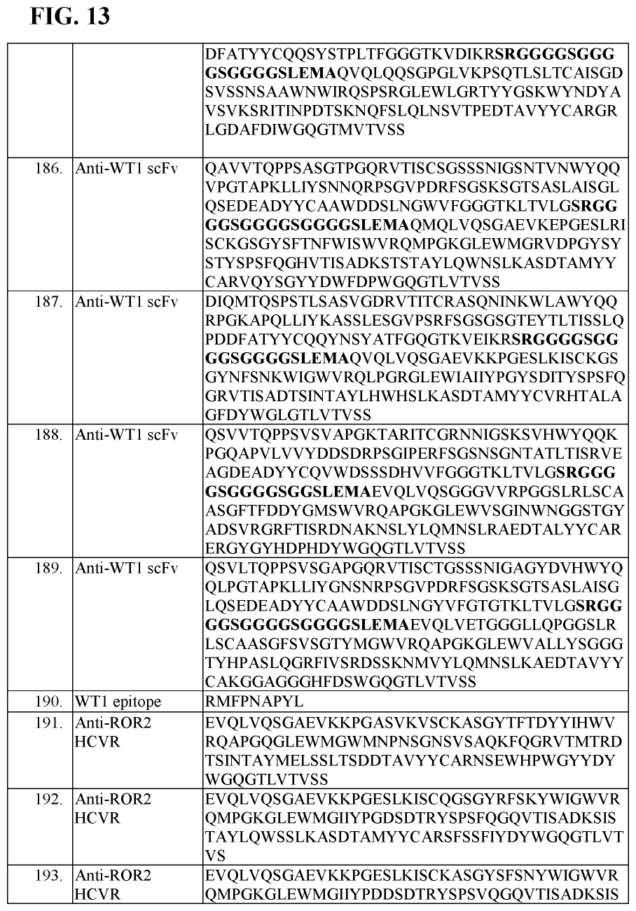

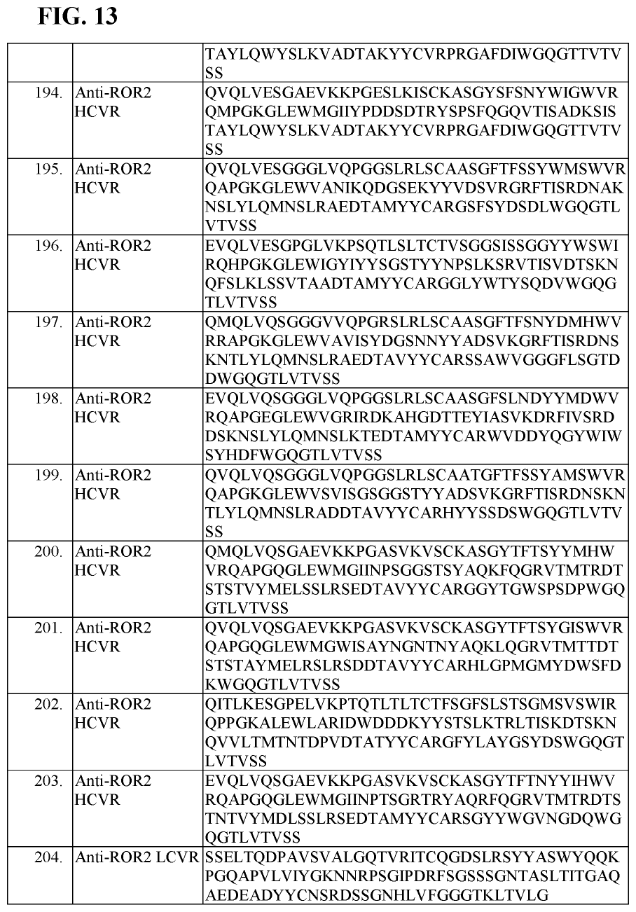

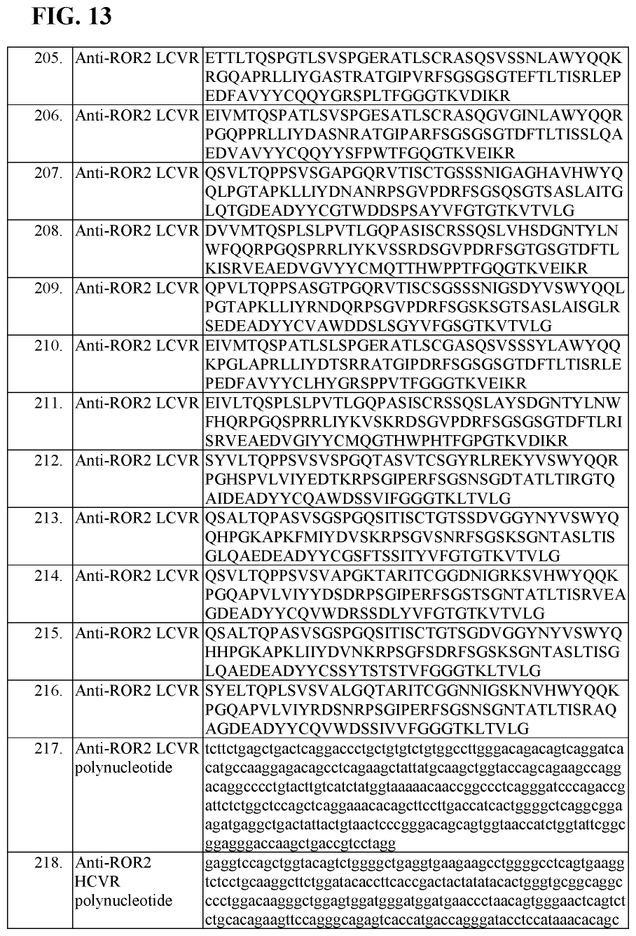

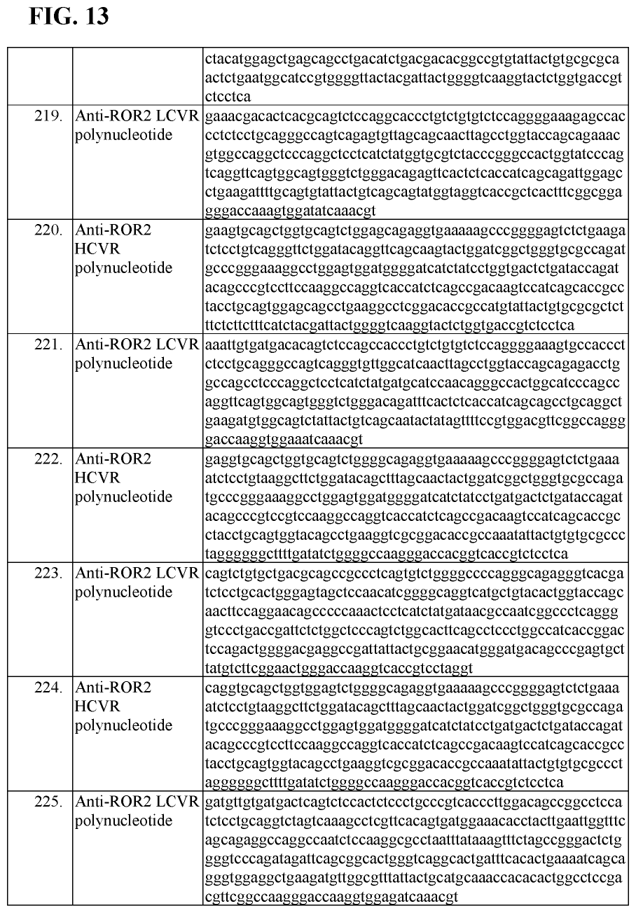

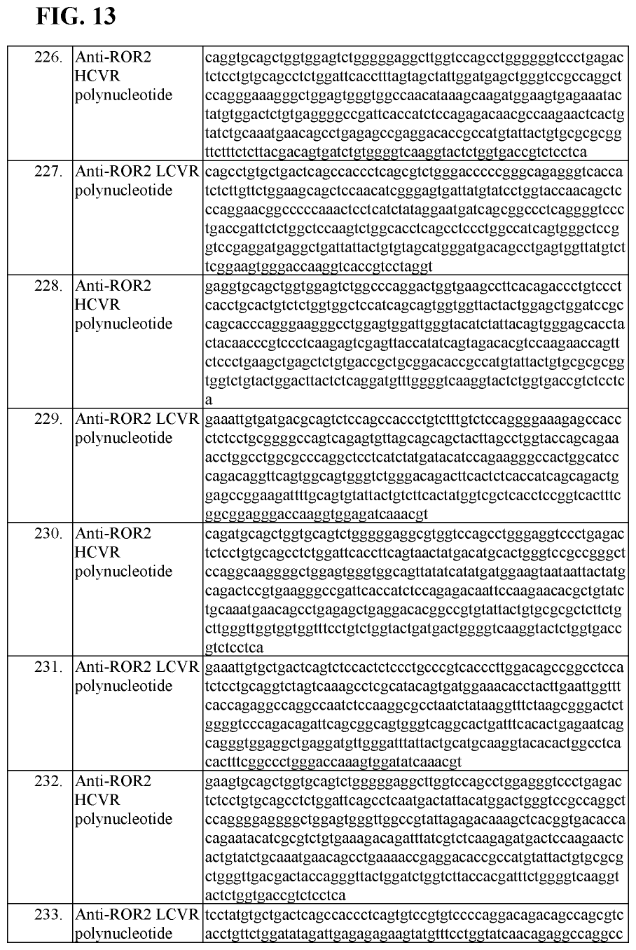

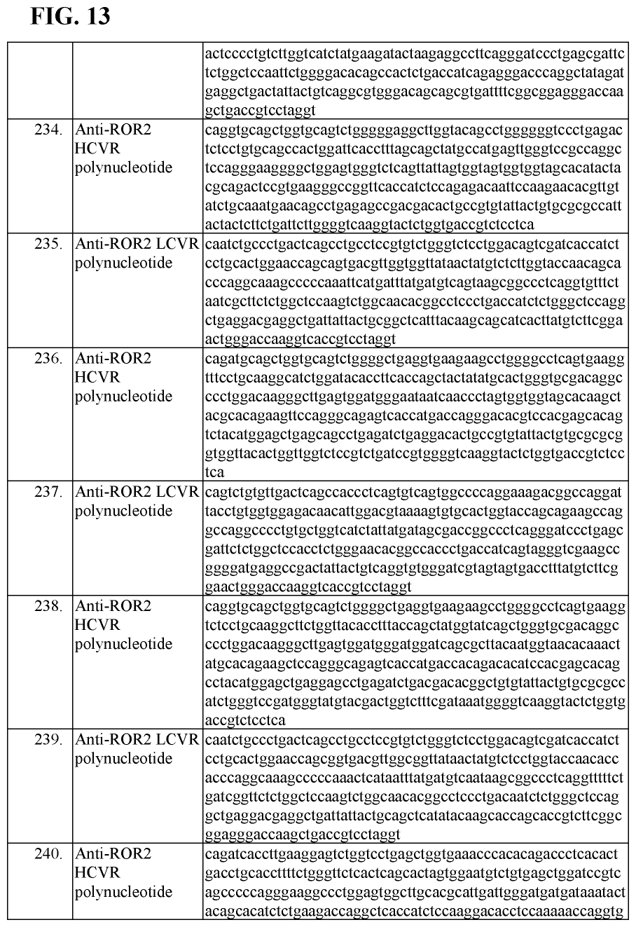

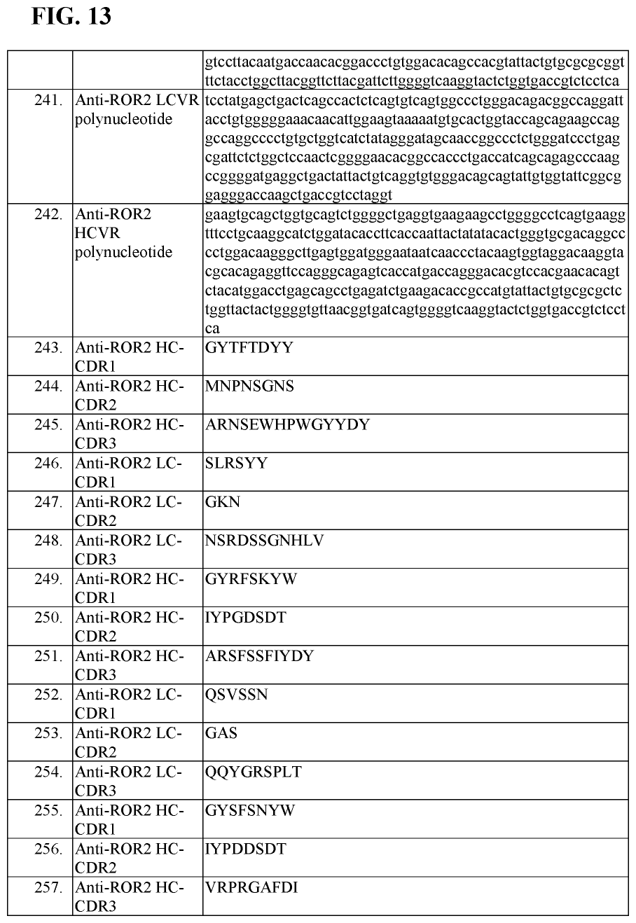

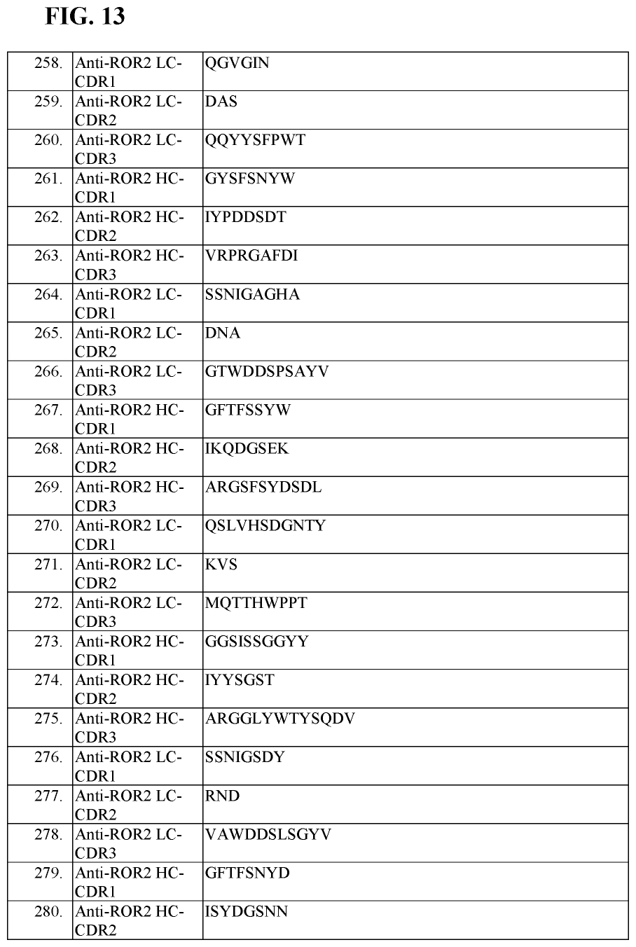

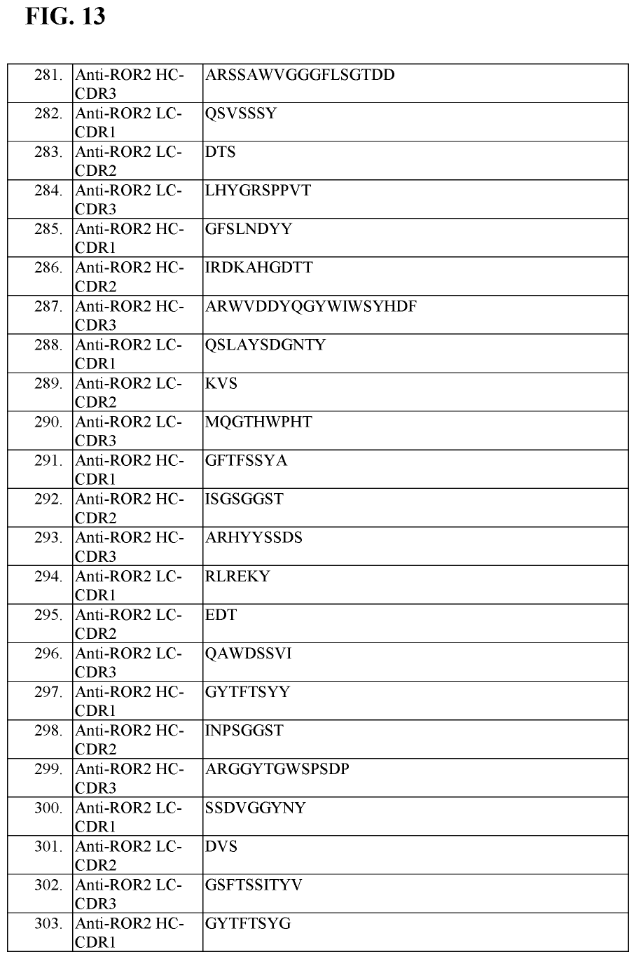

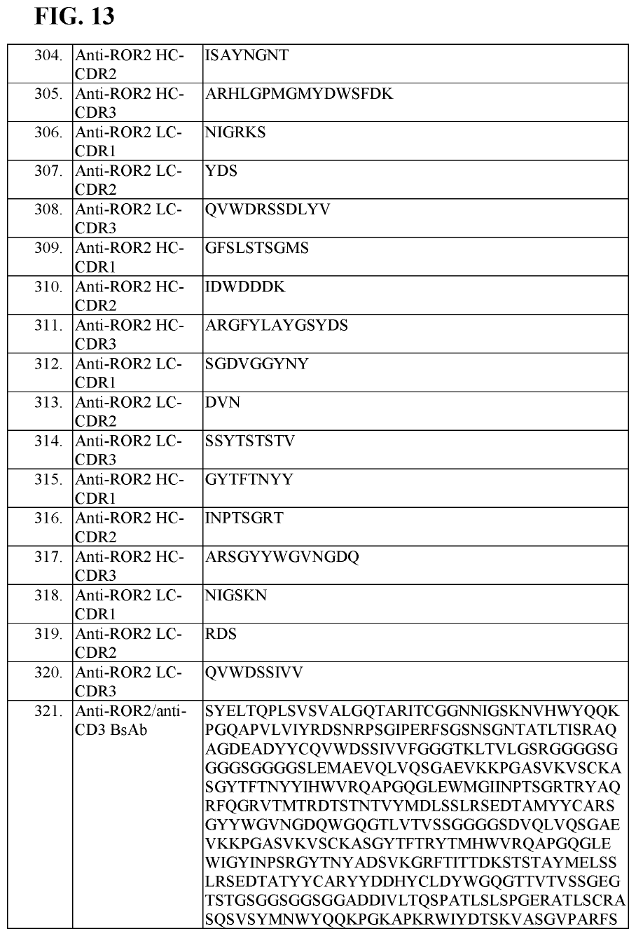

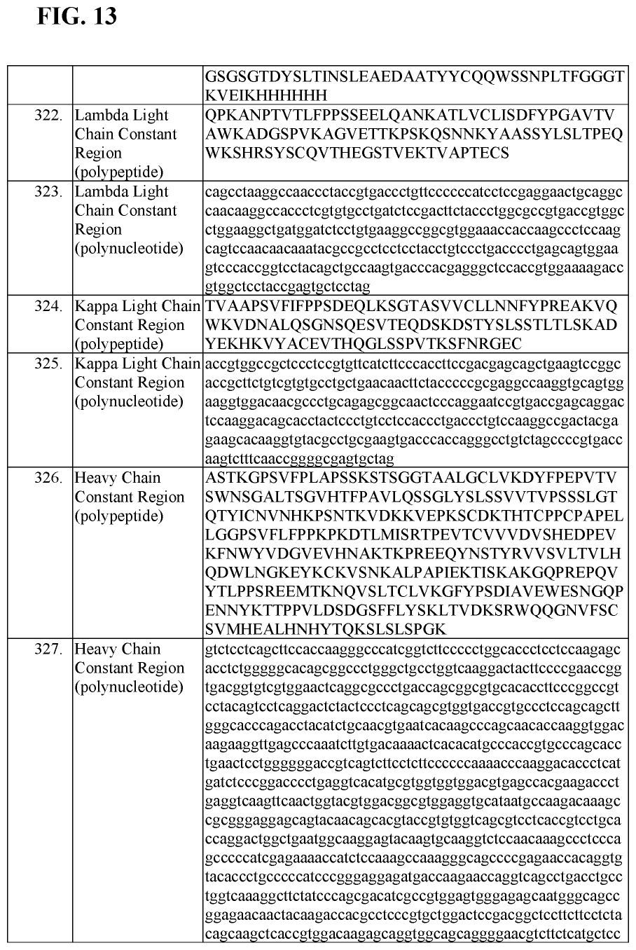

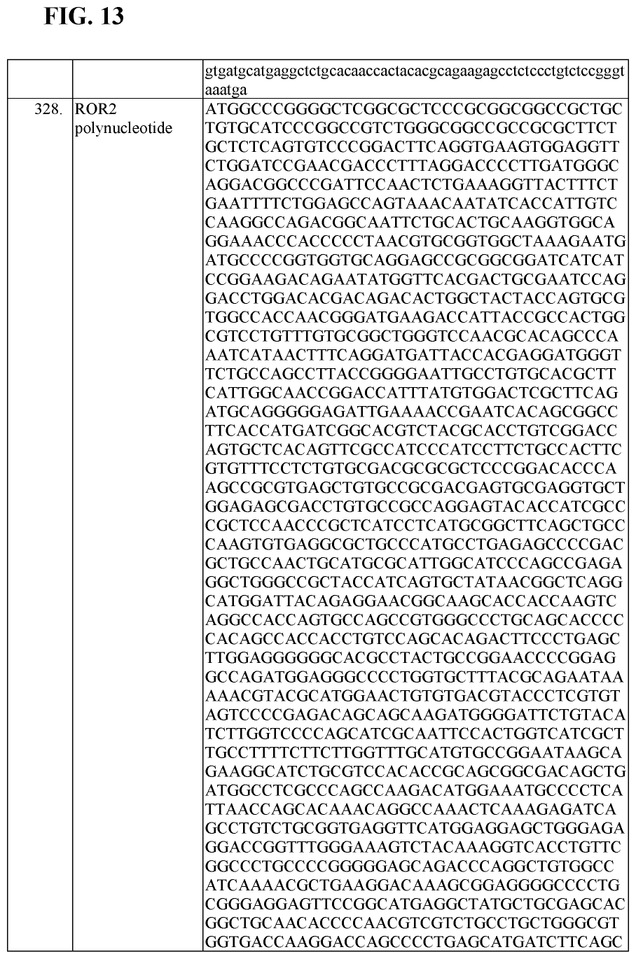

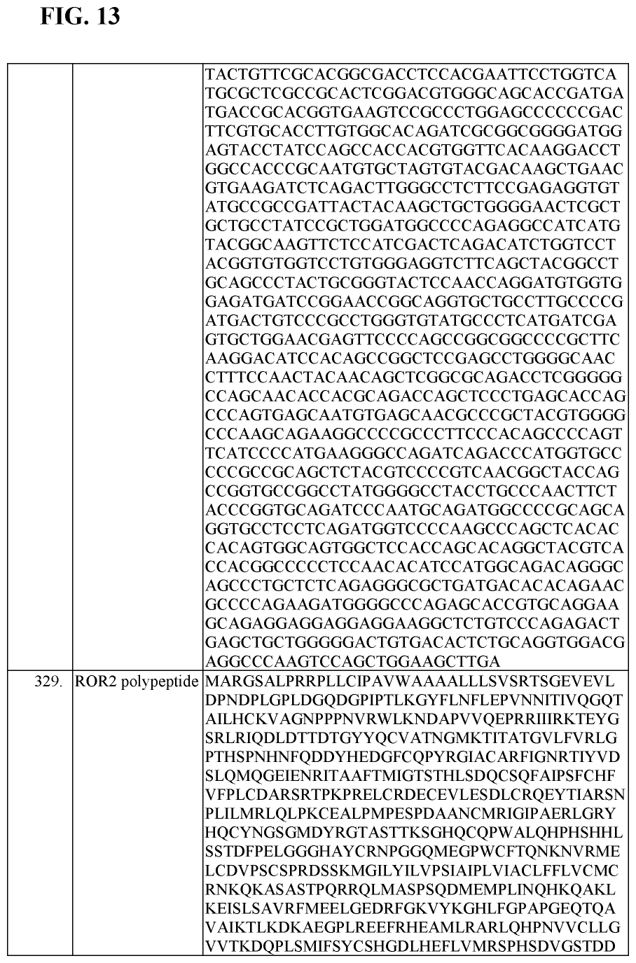

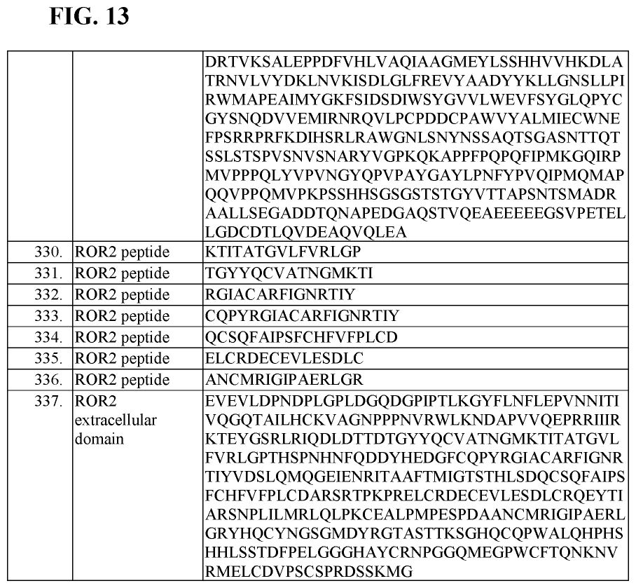

αβ TCRs are known to have significant cross-reactivity to other peptide/MHC complexes (Oates et al. (2015) Mol Immunol 67: 67-74; Attaf et al. (2015) Clin Exp Immunol 181: 1-18). Theoretically, TCRm mAb could have, and do have similar properties, because both TCR and TCRm mAb recognize a short linear peptide epitopes embedded within MHC class I binding groove and other peptides in the exome may share amino acid homologies or physio-chemical features that allow binding. 95 HLA-A2-binding peptides derived from various proteins using T2 cells pulsed with the peptides were screened. The Foxp3-#32 mAb recognized only two peptides derived from two minor antigens HA-1 and HA-8 (FIG. 12); these two peptides share C-terminal leucine and glutamic acids with Foxp3-TLI epitope. As shown above (FIG. 3), position #8 of TLI was one of the key residues recognized by Foxp3-#32 mAb.

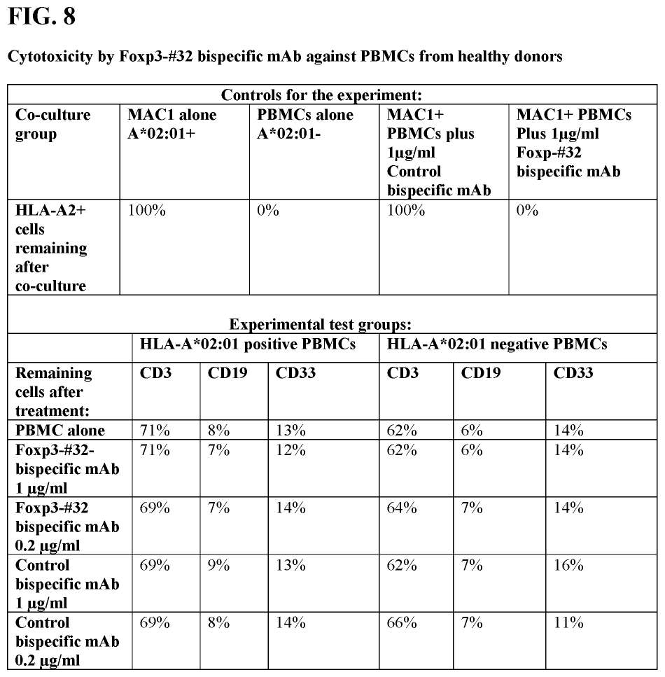

However, to test if the Foxp-#32 mAb was capable of cytotoxicity against normal hematopoietic cells as a result of possible expressed off-target epitopes in these cells, PBMCs from 3 normal healthy donors were incubated that were either HLA-A*02:01positive or negative overnight, in the presence of the Foxp3-#32-bispecific mAb. While control MAC-1 cells were completely killed by Foxp3-#32-bispecific mAb (FIG. 8), no significant reduction of T (CD3+), B (CD19+) and monocytes (CD33+) was detected in either HLA-A*02:01positive or negative donors.

Discussion

The development of therapeutic strategies to deplete or interfere with the function of Tregs, without compromising anti-tumor immunity has been challenging because there is no Treg-specific surface marker, nor a druggable Treg-specific pathway. One of the obstacles for specific depletion of Tregs is that both Tregs and effector T cells may exhibit an activated phenotype, especially in the pattern of expression of key cell surface proteins; both cell types express high levels of CD25, CTLA-4, OX40 and GITR (Scher et al. (2012) Curr Opin Immunol 24 (2): 217-224). Although Tregs express CTLA-4, results from clinical studies suggest that the effects of anti-CTLA-4 treatment is due primarily to increased activation of effector T cells (Colombo et al. (2007) Nat Rev Cancer 7: 880-887). Recent studies have shown that C—C chemokine receptor 4 (CCR4), the cognate receptor for CC chemokines CCL17 and CC122, is predominantly expressed in effector Tregs (eTregs; CD45RA-Foxp3″ CD4+) in TILs in melanoma patients, but also in a variety of other cell types. In vitro depletion of this population using anti-CCR4 mAb enhanced T cell responses when stimulated with NY-ESO-1 peptides (Sugiyama et al. (2013) PNAS 110 (44): 17945-17950) and in a treated patient with T cell leukemia-lymphoma, the Treg fraction is reduced and the NY-ESO-1-specific CD8 T cell response is augmented (Ogura et al. (2014) J Clin Oncol 32 (11): 1157-1163; Ishida et al. (2017) Cancer Sci 108 (10): 2022-2029). Similarly, targeting GITR using cognate ligand or agonistic mAb has been shown to be effective in murine cancer models. However, the clinical efficacy of such a strategy remains to be investigated in human trials.

Foxp3+ Treg cells are recruited by cancer cells and are significantly enriched in the tumor microenvironment, peripheral blood or ascites in cancer patients. In TILs, the ratio of effector T cells to Tregs can predict disease outcome in a variety of cancers, including ovarian, breast, non-small cell lung, hepatocellular, renal cell, pancreatic and gastric carcinomas. Delleuw et al. (2012) Clin Cancer Res 18: 3022-3029; Colombo (2007)), Interestingly, the immunosuppressive function of Foxp3 is not limited to Treg cells, which further supports the important role of Foxp3 in tumor suppressive microenvironment (Karanikas (2008); Truiulzi (2013)). Foxp3 expression was detected in majority of pancreatic cancers (Hinz et al. (2007) Cancer Res 2007; 67 (17): 8344-8350) and these cells induced complete inhibition of T cell proliferation in vitro; this effect was partially abrogated by silencing Foxp3 gene expression using siRNA. Immune suppressive function of Foxp3 has also been suggested in adult T leukemia (ATL) patients which are characterized by constitutive expression of CD4 and CD25 in leukemic cells and marked immune-deficient state (Heid et al. (2009) J Invest Dermatol 129: 2875-2885; Matsubara et al. (2005) Leukemia 19: 482-483).

Foxp3 is an attractive target to identify and selectively kill Tregs and that Foxp3-specific cytotoxic CD8 T cells can be detected in human PBMCs, especially in cancer patients (Larsen (2013)). This prior study demonstrated the possibility of targeting intracellular Foxp3 by an approach of using peptide-specific CTLs. These results here are consistent with this earlier study and formed a premise for making the TCRm mAb to this epitope.

Activated T cells (non Treg) can also transiently express Foxp3 (Wang et al. (2007) EJ Immunol 37: 129-138). However, activated CD4+CD25+ T cells and Tregs, can be distinguished by the expression level of CD127, the alpha chain of IL-7 receptor (Seddiki et al. (2006) J Exp Med 203 (7): 1693-1700; Liu et al. (2006) J Exp Med 203 (7): 1701-1711).

Liu, W. et al. CD127 expression inversely correlates with Foxp3 and suppressive function of human CD4+ Treg cells Tregs express low level of CD127, while conventional T cells express high level of CD127. TCRm mAb Foxp3-#32 only bound to CD127 low/CD25 high/Foxp3 high populations of CD4+ T cells in HLA-A*02:01+ healthy donors was demonstrated (FIG. 4). Remarkably, selective depletion of this small Treg population by the Foxp3-#32 bispecific mAb was detected when the PBMCs from HLA-A*02:01+ donors were treated with the mAb. This selectivity was confirmed using another set of markers, CD45RA vs Foxp3 expression. Both effector Tregs and naïve (resting) Tregs (fraction I and II) along with fraction III (FIG. 7A) were depleted, demonstrating a Foxp3-specific depletion. Importantly, Foxp3-selective depletion in “TILs” in ascites from HLA-A*02:01 positive patients with ovarian cancer by Foxp3-#32 bispecific mAb and Fc-enhanced IgG1 was also detected (FIG. 7D and supplementary FIG. 1B).

Similarly, when Tregs induced in vitro were tested for binding to the mAb Foxp3-#32, only CD4+CD25^{hi} population was bound by the mAb, but not the CD25^{lo}/negative population. The peptide/MHC epitopes are typically found in extremely low density on target cells, making recognition and cytotoxicity difficult. Therefore, a Foxp3 TCRm mAb would only bind to the cells with the highest expression of Foxp3. This opens a possible therapeutic window and approach to designing effective combination therapies by depleting Tregs first using a TCRm mAb directed to Foxp3 epitopes, followed by strategies that activate and expand effector T cells, such as vaccination or check point blockade. In addition, because the goal of a therapeutic anti-Treg antibody is to upset the balance of T cells to favor anti-cancer activity of CD8 and CD4 T cells, complete elimination of the target Treg cells may not be needed, unlike the situation with an antibody directed to the cancer cell itself; furthermore, absolute specificity may not be required.

The properties of TCRm mAb binding to their targets differ from those of typical antibodies in ways that have the potential to limit clinical utility. The peptides must be processed and presented in sufficient amounts to be recognized by the TCRm; the control of these processes are still poorly understood and may be affected by the activation state of the cell (Chang et al. (2016) Expert Opin Biol Ther 16 (8): 979-987). As the epitope is a linear peptide within the constraints of the HLA groove, binding to off target peptides may be possible, if presented by other cells, as has been seen with both TCR and TCRm mAbs (Chang et al. (2016); Ataie et al. (2016) J Mol. Biol 428 (1): 194-205). Binding does not always equate with cytotoxicity, however. While no significant killing was seen against any PBMC by the bispecific mAb format of this TCRm (FIG. 8), nor was binding seen to 93 of 95 other peptides known to bind to HLA-A*02:01 (FIG. 12), the possible off-targets presented on other cells, at both the molecular and cellular level, will need to be defined better before advancing a TCRm such as this forward to systemic clinical use.

The following is a non-limiting list of embodiments of the present invention:

Embodiment 1: A method of manufacturing an engineered immune cell, comprising: contacting a sample comprising a plurality of immune cells with (a) a vector encoding an engineered receptor; and (b) a forkhead box P3 (FoxP3) targeting agent, thereby producing an engineered immune cell that comprises the vector.

Embodiment 2: The method according to embodiment 1, wherein the plurality of immune cells comprises one or more peripheral blood mononuclear cells (PBMCs).

Embodiment 3: The method according to embodiment 2, wherein the one or more PBMCs comprises a leukocyte.

Embodiment 4: The method according to embodiment 3, wherein the leukocyte is a lymphocyte.

Embodiment 5: The method according to embodiment 4, wherein the lymphocyte is a T cell.

Embodiment 6: The method according to embodiment 5, wherein the T cell is an effector T cell.

Embodiment 7: The method according to embodiment 6, wherein the effector T cell is a cytotoxic T cell.

Embodiment 8: The method according to embodiment 7, wherein the cytotoxic T cell is a cluster of differentiation 8 positive (CD8+) T cell.

Embodiment 9: The method according to embodiment 6, wherein the effector cell is a helper T cell.

Embodiment 10: The method according to embodiment 9, wherein the helper T cell is a cluster of differentiation 4 positive (CD4+) T cell.

Embodiment 11: The method according to embodiment 5, wherein the T cell is a regulatory T cell.

Embodiment 12: The method according to any one of embodiments 1 to 11, wherein the plurality of immune cells comprises one or more FoxP3 expressing cells (i.e., FoxP3+ cells).

Embodiment 13: The method according to any one of embodiments 1 to 12, wherein the plurality of immune cells comprises one or more cells that do not express FoxP3.

Embodiment 14: The method according to any one of embodiments 1 to 13, wherein the plurality of immune cells comprises one or more FoxP3 expressing cells and one or more cells that do not express FoxP3.

Embodiment 15: The method according to any one of embodiments 1 to 14, wherein contacting the sample with the FoxP3 targeting agent reduces the number of FoxP3 positive (FoxP3+) cells in the sample.

Embodiment 16: The method according to embodiment 15, wherein contacting the sample with the FoxP3 targeting agent reduces the number of FoxP3+ cells in the sample by at least about 30%, 40%, 50%, 60%, 70%, 80%, 90% or more as compared to the number of FoxP3+ cells in the sample prior to contact with the FoxP3 targeting agent.

Embodiment 17: The method according to embodiment 15, wherein contacting the sample with the FoxP3 targeting agent reduces the number of FoxP3+ cells in the sample by at least about 30%, 40%, 50%, 60%, 70%, 80%, 90% or more as compared to the number of FoxP3+ cells in a control sample that has not been contacted with the FoxP3 targeting agent.

Embodiment 18: The method according to any one of embodiments 12 to 17, wherein at least one of the one or more FoxP3 expressing cells is lysed or killed.

Embodiment 19: The method according to any one of embodiments 12 to 18, wherein at least one of the one or more FoxP3 expressing cells is separated from the cells that do not express FoxP3.

Embodiment 20: The method according to any one of embodiments 12 to 19, wherein at least one of the one or more FoxP3 expressing cells is lysed or killed, and at least one of the one or more FoxP3 expressing cells is separated from the cells that do not express FoxP3.

Embodiment 21: The method according to any one of embodiments 1 to 20, wherein contacting the sample with the FoxP3 targeting agent comprises contacting the sample with two or more different FoxP3 targeting agents.

Embodiment 22: The method according to any one of embodiments 1 to 20, wherein the sample is contacted with the FoxP3 targeting agent prior to being contacted with the vector.

Embodiment 23: The method according to embodiment 22, wherein contacting the sample with the FoxP3 targeting agent occurs at least 4, 6, 8, 10, 12, 16, 20, 24, 36, or 48 hours prior to contacting the sample with the vector.

Embodiment 24: The method according to any one of embodiments 1 to 20, wherein the sample is contacted with the FoxP3 targeting agent and the vector concurrently.

Embodiment 25: The method according to any one of embodiments 1 to 20, wherein the sample is contacted with the FoxP3 targeting agent after being contacted with the vector.

Embodiment 26: The method according to embodiment 25, wherein contacting the sample with the vector occurs at least 12, 24, 36, 48, 60, 72, 84, 96, 108, 120, 132, or 144 hours prior to contacting the sample with the FoxP3 targeting agent.

Embodiment 27: The method according to any one of embodiments 1 to 26, wherein the engineered receptor is selected from the group consisting of a chimeric antigen receptor (CAR), chimeric antibody-T cell receptor (caTCR), and engineered T cell receptor (eTCR).

Embodiment 28: The method according to embodiment 27, wherein the engineered receptor is a CAR.

Embodiment 29: The method according to embodiment 28, wherein the CAR comprises at least one extracellular antigen-binding domain.

Embodiment 30: The method according to embodiment 29, wherein the at least one extracellular antigen-binding domain comprises a single chain variable region fragment (scFv).

Embodiment 31: The method according to any one of embodiments 28 to 30, wherein the CAR comprises at least one intracellular signaling domain.

Embodiment 32: The method according to embodiment 31, wherein the at least one intracellular signaling domain comprises a CD3 polypeptide or fragment thereof.

Embodiment 33: The method according to embodiment 27, wherein the engineered receptor is a caTCR.

Embodiment 34: The method according to embodiment 33, wherein the caTCR comprises: (a) a first polypeptide chain comprising a first antigen-binding domain comprising a V_{H} antibody domain and a first TCR domain (TCRD) comprising a first TCR transmembrane domain (TCR-TM); and (b) a second polypeptide chain comprising a second antigen-binding domain comprising a V_{L} antibody domains and a second TCRD comprising a second TCR-TM, wherein the V_{H} domain of the first antigen-binding domain and the V_{L} domain of the second antigen-binding domain form an antigen-binding module that specifically binds to the target antigen, and wherein the first TCRD and the second TCRD form a TCR module (TCRM) that is capable of recruiting at least one TCR-associated signaling module.

Embodiment 35: The method according to embodiment 34, wherein the first TCR-TM is derived from one of the transmembrane domains of a first naturally occurring TCR and the second TCR-TM is derived from the other transmembrane domain of the first naturally occurring TCR.

Embodiment 36: The method according to embodiment 35, wherein the first naturally occurring TCR is a gamma-delta TCR.

Embodiment 37: The method according to any one of embodiments 34 to 36, wherein the first polypeptide chain further comprises a first peptide linker between the first antigen-binding domain and the first TCRD and the second polypeptide chain further comprises a second peptide linker between the second antigen-binding domain and the second TCRD.

Embodiment 38: The method according to embodiment 37, wherein the first and/or second peptide linkers comprise, individually, a constant domain or fragment thereof from an immunoglobulin or TCR subunit.

Embodiment 39: The method according to embodiment 38, wherein the first and/or second peptide linkers comprise, individually, a CH1, CH2, CH3, CH4, or CL antibody domain, or a fragment thereof.

Embodiment 40: The method according to embodiment 39, wherein the first and/or second peptide linkers comprise, individually, a Cα, Cβ, Cγ, or Cδ TCR domain, or a fragment thereof.

Embodiment 41: The method according to embodiment 27, wherein the engineered receptor is an eTCR.

Embodiment 42: The method according to embodiment 41, wherein the eTCR comprises an antigen/MHC-binding region.

Embodiment 43: The method according to embodiment 42, wherein the antigen/MHC-binding region is derived from an antigen/MHC-binding region of a naturally occurring TCR.

Embodiment 44: The method according to any one of embodiments 1 to 43, wherein the engineered receptor binds to a cell surface antigen.

Embodiment 45: The method according to embodiment 44, wherein the cell surface antigen is selected from the group consisting of a protein, carbohydrate, and lipid.

Embodiment 46: The method according to embodiment 45, wherein the cell surface antigen is selected from the group consisting of cluster of differentiation 19 (CD19), CD20, CD47, glypican 3 (GPC-3), Receptor Tyrosine Kinase-Like Orphan Receptor 1 (ROR1), ROR2, B Cell Maturation Antigen (BCMA), G Protein-Coupled Receptor Class C Group 5 Member D (GPRCSD), and Fc Receptor Like 5 (FCRL5).

Embodiment 47: The method according to embodiment 46, wherein the cell surface antigen is CD19.

Embodiment 48: The method according to any one of embodiments 1 to 43, wherein the engineered receptor binds to a complex comprising a peptide and a major histocompatibility complex (MHC) protein.

Embodiment 49: The method according to embodiment 48, wherein the peptide is derived from a protein selected from the group consisting of Wilms' tumor gene 1 (WT-1), alpha-fetoprotein (AFP), human papilloma virus 16 E7 protein (HPV16-E7), New York Esophageal Squamous Cell Carcinoma 1 (NY-ESO-1), preferentially expressed antigen of melanoma (PRAME), Epstein-Barr virus-latent membrane protein 2 alpha (EBV-LMP2A), human immunodeficiency virus 1 (HIV-1), KRAS, Histone H3.3, and prostate specific antigen (PSA).

Embodiment 50: The method according to embodiment 49, wherein the peptide is derived from AFP.

Embodiment 51: The method according to embodiment 50, wherein the peptide derived from AFP comprises the sequence of FMNKFIYEI (SEQ ID NO: 338).

Embodiment 52: The method according to embodiment 48, wherein the MHC protein is a MHC class I protein.

Embodiment 53: The method according to embodiment 52, wherein the MHC class I protein is the HLA-A*02:01 subtype of the HLA-A02 allele.

Embodiment 54: The method according to any one of embodiments 1 to 53, wherein the engineered receptor is multispecific.

Embodiment 55: The method according to any one of embodiments 1 to 53, wherein the engineered receptor is monospecific.

Embodiment 56: The method according to any one of embodiments 1 to 55, wherein the vector encoding the engineered receptor is a mammalian expression vector.

Embodiment 57: The method according to embodiment 56, wherein the mammalian expression vector is a lentiviral vector or transposon vector.

Embodiment 58: The method according to any one of embodiments 1 to 57, wherein the FoxP3 targeting agent is an antibody, CAR, caTCR, or eTCR, or comprises antigen-binding fragment thereof.

Embodiment 59: The method according to any one of embodiments 1 to 57, wherein the FoxP3 targeting agent is a TCR molecule or comprises an antigen-binding portion of a TCR molecule.

Embodiment 60: The method according to any one of embodiments 1 to 59, wherein the FoxP3 targeting agent comprises an antigen-binding protein that binds to a complex comprising a FoxP3-derived peptide and an MEW protein.

Embodiment 61: The method according to embodiment 60, wherein the MEW protein is a MEW class I protein.

Embodiment 62: The method according to embodiment 61, wherein the MEW class I protein is a human leukocyte antigen (HLA) class I molecule.

Embodiment 63: The method according to embodiment 62, wherein the HLA class I molecule is HLA-A.

Embodiment 64: The method according to embodiment 63, wherein the HLA-A is HLA-A2.

Embodiment 65: The method according to embodiment 64, wherein the HLA-A2 is HLA-A*02:01.

Embodiment 66: The method according to any one of embodiments 60 to 65, wherein the antigen-binding protein is an antibody, a CAR, or a caTCR.

Embodiment 67: The method according to embodiment 66, wherein the antigen-binding protein is monospecific.

Embodiment 68: The method according to embodiment 66, wherein the antigen-binding protein is a full-length antibody.

Embodiment 69: The method according to embodiment 68, wherein the antigen-binding protein is an IgG.

Embodiment 70: The method according to embodiment 68 or 69, wherein the antigen-binding protein is coupled to a solid support.

Embodiment 71: The method according to embodiment 70, wherein the solid support is selected from a group consisting of a bead, microwell, and planar glass surface.

Embodiment 72: The method according to embodiment 71, wherein the bead is selected from a group consisting of a magnetic bead, crosslinked polymer bead, and beaded agarose.

Embodiment 73: The method according to embodiment 66, wherein the antigen-binding protein is multispecific.

Embodiment 74: The method according to embodiment 73, wherein the antigen-binding protein is a bispecific antibody.

Embodiment 75: The method according to embodiment 74, wherein the bispecific antibody comprises: (a) an antigen-binding domain specific for the complex comprising the FoxP3 peptide and the MHC protein, and (b) an antigen-binding domain specific for cluster of differentiation 3 (CD3).

Embodiment 76: The method according to any one of embodiments 66, 67, and 73, wherein the antigen-binding protein is a chimeric antigen receptor (CAR).

Embodiment 77: The method according to embodiment 76, wherein the FoxP3 targeting agent is an anti-FoxP3 CAR-T cell.

Embodiment 78: The method according to any one of embodiments 60 to 77, wherein the FoxP3-derived peptide fragment has a length of 8 to 12 amino acids.

Embodiment 79: The method according to any one of embodiments 60 to 78, wherein the FoxP3-derived peptide fragment is selected from FoxP3-1 having the amino acid sequence set forth in SEQ ID NO: 2 or a portion thereof, FoxP3-2 having the amino acid sequence set forth in SEQ ID NO: 3 or a portion thereof, FoxP3-3 having the amino acid sequence set forth in SEQ ID NO: 4 or a portion thereof, FoxP3-4 having the amino acid sequence set forth in SEQ ID NO: 5 or a portion thereof, FoxP3-5 having the amino acid sequence set forth in SEQ ID NO: 6 or a portion thereof, FoxP3-6 having the amino acid sequence set forth in SEQ ID NO: 7 or a portion thereof; and FoxP3-7 having the amino acid sequence set forth in SEQ ID NO: 8 or a portion thereof.

Embodiment 80: The method according to embodiment 79, wherein the FoxP3-derived peptide fragment is FoxP3-7 having the amino acid sequence set forth in SEQ ID NO: 8 or a portion thereof.

Embodiment 81: The method according to embodiment 79, wherein the antigen-binding protein comprises: (i) a heavy chain variable region CDR1 comprising an amino acid sequence set forth in SEQ ID NO: 16; a heavy chain variable region CDR2 comprising an amino acid sequence set forth in SEQ ID NO: 17; a heavy chain variable region CDR3 comprising an amino acid sequence set forth in SEQ ID NO: 18; a light chain variable region CDR1 comprising an amino acid sequence set forth in SEQ ID NO: 19; a light chain variable region CDR2 comprising an amino acid sequence set forth in SEQ ID NO: 20; and a light chain variable region CDR3 comprising an amino acid sequence set forth in SEQ ID NO: 21; (ii) a heavy chain variable region CDR1 comprising an amino acid sequence set forth in SEQ ID NO: 22; a heavy chain variable region CDR2 comprising an amino acid sequence set forth in SEQ ID NO: 23; a heavy chain variable region CDR3 comprising an amino acid sequence set forth in SEQ ID NO: 24; a light chain variable region CDR1 comprising an amino acid sequence set forth in SEQ ID NO: 25; a light chain variable region CDR2 comprising an amino acid sequence set forth in SEQ ID NO: 26; and a light chain variable region CDR3 comprising an amino acid sequence set forth in SEQ ID NO: 27; (iii) a heavy chain variable region CDR1 comprising an amino acid sequence set forth in SEQ ID NO: 28; a heavy chain variable region CDR2 comprising an amino acid sequence set forth in SEQ ID NO: 29; a heavy chain variable region CDR3 comprising an amino acid sequence set forth in SEQ ID NO: 30; a light chain variable region CDR1 comprising an amino acid sequence set forth in SEQ ID NO: 31; a light chain variable region CDR2 comprising an amino acid sequence set forth in SEQ ID NO: 32; and a light chain variable region CDR3 comprising an amino acid sequence set forth in SEQ ID NO: 33; (iii) a heavy chain variable region CDR1 comprising an amino acid sequence set forth in SEQ ID NO: 34; a heavy chain variable region CDR2 comprising an amino acid sequence set forth in SEQ ID NO: 35; a heavy chain variable region CDR3 comprising an amino acid sequence set forth in SEQ ID NO: 36; a light chain variable region CDR1 comprising an amino acid sequence set forth in SEQ ID NO: 37; a light chain variable region CDR2 comprising an amino acid sequence set forth in SEQ ID NO: 38; and a light chain variable region CDR3 comprising an amino acid sequence set forth in SEQ ID NO: 39; (iv) a heavy chain variable region CDR1 comprising an amino acid sequence set forth in SEQ ID NO: 40; a heavy chain variable region CDR2 comprising an amino acid sequence set forth in SEQ ID NO: 41; a heavy chain variable region CDR3 comprising an amino acid sequence set forth in SEQ ID NO: 42; a light chain variable region CDR1 comprising an amino acid sequence set forth in SEQ ID NO: 43; a light chain variable region CDR2 comprising an amino acid sequence set forth in SEQ ID NO: 44; and a light chain variable region CDR3 comprising an amino acid sequence set forth in SEQ ID NO: 45; (v) a heavy chain variable region CDR1 comprising an amino acid sequence set forth in SEQ ID NO: 46; a heavy chain variable region CDR2 comprising an amino acid sequence set forth in SEQ ID NO: 47; a heavy chain variable region CDR3 comprising an amino acid sequence set forth in SEQ ID NO: 48; a light chain variable region CDR1 comprising an amino acid sequence set forth in SEQ ID NO: 49; a light chain variable region CDR2 comprising an amino acid sequence set forth in SEQ ID NO: 50; and a light chain variable region CDR3 comprising an amino acid sequence set forth in SEQ ID NO: 51; (vi) a heavy chain variable region CDR1 comprising an amino acid sequence set forth in SEQ ID NO: 52; a heavy chain variable region CDR2 comprising an amino acid sequence set forth in SEQ ID NO: 53; a heavy chain variable region CDR3 comprising an amino acid sequence set forth in SEQ ID NO: 54; a light chain variable region CDR1 comprising an amino acid sequence set forth in SEQ ID NO: 55; a light chain variable region CDR2 comprising an amino acid sequence set forth in SEQ ID NO: 56; and a light chain variable region CDR3 comprising an amino acid sequence set forth in SEQ ID NO: 57; or (vii) a heavy chain variable region CDR1 comprising an amino acid sequence set forth in SEQ ID NO: 58; a heavy chain variable region CDR2 comprising an amino acid sequence set forth in SEQ ID NO: 59; a heavy chain variable region CDR3 comprising an amino acid sequence set forth in SEQ ID NO: 60; a light chain variable region CDR1 comprising an amino acid sequence set forth in SEQ ID NO: 61; a light chain variable region CDR2 comprising an amino acid sequence set forth in SEQ ID NO: 62; and a light chain variable region CDR3 comprising an amino acid sequence set forth in SEQ ID NO: 63.

Embodiment 82: The method according to embodiment 81, wherein the antigen-binding protein comprises a heavy chain variable region CDR1 comprising an amino acid sequence set forth in SEQ ID NO: 46; a heavy chain variable region CDR2 comprising an amino acid sequence set forth in SEQ ID NO: 47; a heavy chain variable region CDR3 comprising an amino acid sequence set forth in SEQ ID NO: 48; a light chain variable region CDR1 comprising an amino acid sequence set forth in SEQ ID NO: 49; a light chain variable region CDR2 comprising an amino acid sequence set forth in SEQ ID NO: 50; and a light chain variable region CDR3 comprising an amino acid sequence set forth in SEQ ID NO: 51.

Embodiment 83: The method according to embodiment 29 or embodiment 34, wherein the at least one extracellular antigen binding domain of embodiment 29 or the antigen-binding module of embodiment 34 binds to CD19 and comprises: (i) heavy chain CDR1, CDR2, and CDR3, respectively, comprising amino acid sequences that are at least 80%, at least 85%, at least 90%, or at least 95% identical to SEQ ID NOs: 105, 106, and 107; and/or (ii) light chain CDR1, CDR2, and CDR3, respectively, comprising amino acid sequences that are at least 80%, at least 85%, at least 90%, or at least 95% identical to SEQ ID NOs: 109, 110, or 111; (ii) heavy chain CDR1, CDR2, and CDR3, respectively, comprising amino acid sequences that are at least 80%, at least 85%, at least 90%, or at least 95% identical to SEQ ID NOs: 105, 106, and 108; and/or (ii) light chain CDR1, CDR2, and CDR3, respectively, comprising amino acid sequences that are at least 80%, at least 85%, at least 90%, or at least 95% identical to SEQ ID NOs: 109, 110, or 111; (iii) heavy chain CDR1, CDR2, and CDR3, respectively, comprising amino acid sequences that are at least 80%, at least 85%, at least 90%, or at least 95% identical to SEQ ID NOs: 105, 106, and 107; and/or (ii) light chain CDR1, CDR2, and CDR3, respectively, comprising amino acid sequences that are at least 80%, at least 85%, at least 90%, or at least 95% identical to SEQ ID NOs: 109, 110, or 112; or (iv) heavy chain CDR1, CDR2, and CDR3, respectively, comprising amino acid sequences that are at least 80%, at least 85%, at least 90%, or at least 95% identical to SEQ ID NOs: 105, 106, and 108; and/or (ii) light chain CDR1, CDR2, and CDR3, respectively, comprising amino acid sequences that are at least 80%, at least 85%, at least 90%, or at least 95% identical to SEQ ID NOs: 109, 110, or 112.

Embodiment 84: The method according to any one of embodiments 1 to 58, wherein the FoxP3 targeting agent comprises a FoxP3 targeting CAR and wherein the FoxP3 targeting CAR binds to a complex comprising a FoxP3 peptide and a major histocompatibility complex (MEW) protein.

Embodiment 85: The method according to embodiment 84, wherein the FoxP3 targeting CAR comprises an scFv that binds to complex comprising a FoxP3 peptide and a major histocompatibility complex (MHC) protein.

Embodiment 86: The method according to embodiment 85, wherein the FoxP3 targeting CAR further comprises a CD28-CD3ζ peptide that is fused to the scFv.

Embodiment 87: The method according to embodiment 86, wherein the FoxP3 targeting CAR comprises an scFv-CD28-CD3ζ fusion having an amino acid sequence that is at least 80%, at least 85%, at least 90%, or at least 95% identical to SEQ ID NO: 12.

Embodiment 88: The method according to embodiment 85, wherein the FoxP3 targeting CAR further comprises a 41BB-CD3ζ peptide that is fused to the scFv.

Embodiment 89: The method according to embodiment 88, wherein the FoxP3 targeting CAR comprises an scFv-41BB-CD3ζ fusion having an amino acid sequence that is at least 80%, at least 85%, at least 90%, or at least 95% identical to SEQ ID NO: 13.

Embodiment 90: The method according to any one of embodiments 1 to 58, wherein the FoxP3 targeting agent comprises a FoxP3 targeting caTCR and wherein the FoxP3 targeting caTCR binds to a complex comprising a FoxP3 peptide and a major histocompatibility complex (MHC) protein.

Embodiment 91: The method according to embodiment 90, wherein the FoxP3 targeting caTCR comprises: (a) a first polypeptide chain comprising a first antigen-binding domain comprising a V_{H} antibody domain and a first TCR domain (TCRD) comprising a first TCR transmembrane domain (TCR-TM); and (b) a second polypeptide chain comprising a second antigen-binding domain comprising a V_{L} antibody domains and a second TCRD comprising a second TCR-TM, wherein the V_{H} domain of the first antigen-binding domain and the V_{L} domain of the second antigen-binding domain form an antigen-binding module that specifically binds to the target antigen, and wherein the first TCRD and the second TCRD form a TCR module (TCRM) that is capable of recruiting at least one TCR-associated signaling module.

Embodiment 92: The method according to embodiment 91, wherein the first TCR-TM is derived from one of the transmembrane domains of a first naturally occurring TCR and the second TCR-TM is derived from the other transmembrane domain of the first naturally occurring TCR.

Embodiment 93: The method according to embodiment 92, wherein the first naturally occurring TCR is a gamma-delta TCR.

Embodiment 94: The method according to embodiment 91, wherein the caTCR comprises an anti-FoxP3 light chain/gamma chain fusion having an amino acid sequence that is at least 80%, at least 85%, at least 90%, or at least 95% identical to SEQ ID NO: 15.

Embodiment 95: The method according to embodiment 91, wherein the caTCR comprises an anti-FoxP3 heavy chain/delta chain fusion having an amino acid sequence that is at least 80%, at least 85%, at least 90%, or at least 95% identical to SEQ ID NO: 14.

Embodiment 96: A method for depleting FoxP3 positive cells in a therapeutic composition comprising engineered immune cells expressing engineered receptor, the method comprising contacting the therapeutic composition with a FoxP3 targeting agent.

Embodiment 97: A method for enriching for engineered-receptor-expressing cytotoxic T cells in a sample, comprising contacting the sample with a FoxP3 targeting agent.

Embodiment 98: A composition comprising: (a) an engineered immune cell, wherein the engineered immune cell expresses an engineered receptor; and (b) a FoxP3 targeting agent.

Embodiment 99: A composition comprising: (a) a vector encoding an engineered receptor; and (b) a FoxP3 targeting agent.

1. A method of manufacturing an engineered immune cell, comprising: contacting a sample comprising a plurality of immune cells with (a) a vector encoding an engineered receptor; and (b) a forkhead box P3 (FoxP3) targeting agent, thereby producing an engineered immune cell that comprises the vector, optionally wherein the plurality of immune cells comprises one or more peripheral blood mononuclear cells (PBMCs).

2. (canceled)

3. The method of claim 1, wherein the one or more PBMCs comprise a T cell, optionally wherein the T cell is a cytotoxic T cell, a helper T cell, a cluster of differentiation 8 positive (CD8+) T cell, a cluster of differentiation 4 positive (CD4+) T cell, or a regulatory T cell.

4. (canceled)

5. (canceled)

6. (canceled)

7. The method of claim 1, wherein the plurality of immune cells comprises one or more FoxP3 positive (FoxP3⁺) cells; or comprises one or more FoxP3⁺ cells and one or more cells that do not express FoxP3.

8. The method of claim 1, wherein contacting the sample with the FoxP3 targeting agent reduces the number of FoxP3⁺ cells in the sample, optionally wherein contacting the sample with the FoxP3 targeting agent reduces the number of FoxP3⁺ cells in the sample by at least about 30%, 40%, 50%, 60%, 70%, 80%, 90% or more as compared to the number of FoxP3⁺ cells in the sample prior to contact with the FoxP3 targeting agent or reduces the number of FoxP3⁺ cells in the sample by at least about 30%, 40%, 50%, 60%, 70%, 80%, 90% or more as compared to the number of FoxP3⁺ cells in a control sample that has not been contacted with the FoxP3 targeting agent.

9. (canceled)

10. The method of claim 7, wherein at least one of the one or more FoxP3⁺ cells is separated from the cells that do not express FoxP3.

11. The method of claim 1, wherein contacting the sample with the FoxP3 targeting agent comprises contacting the sample with two or more different FoxP3 targeting agents or wherein the sample is contacted with the FoxP3 targeting agent prior to, concurrently, or after being contacted with the vector.

12. (canceled)

13. The method of claim 1, wherein the engineered receptor is selected from the group consisting of a chimeric antigen receptor (CAR), a chimeric antibody-T cell receptor (caTCR), and an engineered T cell receptor (eTCR).

14. The method of claim 13, wherein the CAR comprises at least one extracellular antigen-binding domain and/or at least one intracellular signaling domain, optionally wherein the at least one extracellular antigen-binding domain comprises a single chain variable region fragment (scFv) and/or the at least one intracellular signaling domain comprises a CD3ξ, polypeptide or fragment thereof.

15. (canceled)

16. The method of claim 1, wherein the engineered receptor binds to a cell surface antigen, optionally wherein the cell surface antigen is selected from the group consisting of cluster of differentiation 19 (CD19), CD20, CD47, glypican 3 (GPC-3), Receptor Tyrosine Kinase-Like Orphan Receptor 1 (ROR1), ROR2, B Cell Maturation Antigen (BCMA), G Protein-Coupled Receptor Class C Group 5 Member D (GPRCSD), and Fc Receptor Like 5 (FCRL5).

17. (canceled)

18. The method of claim 1, wherein the engineered receptor binds to a complex comprising a peptide and a major histocompatibility complex (MHC) protein, optionally wherein the peptide is derived from a protein selected from the group consisting of Wilms' tumor gene 1 (WT-1), alpha-fetoprotein (AFP), human papilloma virus 16 E7 protein (HPV16-E7), New York Esophageal Squamous Cell Carcinoma 1 (NY-ESO-1), preferentially expressed antigen of melanoma (PRAME), Epstein-Barr virus-latent membrane protein 2 alpha (EBV-LMP2A), human immunodeficiency virus 1 (HIV-1), KRAS, Histone H3.3, and prostate specific antigen (PSA).

19. (canceled)

20. The method of claim 1, wherein the vector encoding the engineered receptor is a mammalian expression vector, a lentiviral vector or transposon vector.

21. The method of claim 1, wherein the FoxP3 targeting agent
comprises an antigen-binding protein that is an antibody, CAR, caTCR, or eTCR, or comprises antigen-binding fragment thereof; or
comprises an antigen-binding protein that binds to a complex comprising a FoxP3-derived peptide and an MHC protein; or
is a TCR molecule or comprises an antigen-binding portion of a TCR molecule.

22. (canceled)

23. The method of claim 21, wherein the antigen-binding protein is coupled to a solid support.

24. The method of claim 21, wherein the antigen-binding protein is a bispecific antibody comprising: (a) an antigen-binding domain specific for a complex comprising the FoxP3 peptide and an MHC protein, and (b) an antigen-binding domain specific for cluster of differentiation 3 (CD3).

25. The method of claim 21, wherein the FoxP3 targeting agent is an anti-FoxP3 CAR-T cell.

26. The method of claim 21, wherein the FoxP3-derived peptide fragment is selected from FoxP3-1 having the amino acid sequence set forth in SEQ ID NO: 2 or a portion thereof, FoxP3-2 having the amino acid sequence set forth in SEQ ID NO: 3 or a portion thereof, FoxP3-3 having the amino acid sequence set forth in SEQ ID NO: 4 or a portion thereof, FoxP3-4 having the amino acid sequence set forth in SEQ ID NO: 5 or a portion thereof, FoxP3-5 having the amino acid sequence set forth in SEQ ID NO: 6 or a portion thereof, FoxP3-6 having the amino acid sequence set forth in SEQ ID NO: 7 or a portion thereof; and FoxP3-7 having the amino acid sequence set forth in SEQ ID NO: 8 or a portion thereof.

27. The method of claim 26, wherein the FoxP3 targeting agent comprises an antigen-binding protein comprising:
a. a heavy chain variable region CDR1 comprising an amino acid sequence set forth in SEQ ID NO: 16; a heavy chain variable region CDR2 comprising an amino acid sequence set forth in SEQ ID NO: 17; a heavy chain variable region CDR3 comprising an amino acid sequence set forth in SEQ ID NO: 18; a light chain variable region CDR1 comprising an amino acid sequence set forth in SEQ ID NO: 19; a light chain variable region CDR2 comprising an amino acid sequence set forth in SEQ ID NO: 20; and a light chain variable region CDR3 comprising an amino acid sequence set forth in SEQ ID NO: 21;
b. a heavy chain variable region CDR1 comprising an amino acid sequence set forth in SEQ ID NO: 22; a heavy chain variable region CDR2 comprising an amino acid sequence set forth in SEQ ID NO: 23; a heavy chain variable region CDR3 comprising an amino acid sequence set forth in SEQ ID NO: 24; a light chain variable region CDR1 comprising an amino acid sequence set forth in SEQ ID NO: 25; a light chain variable region CDR2 comprising an amino acid sequence set forth in SEQ ID NO: 26; and a light chain variable region CDR3 comprising an amino acid sequence set forth in SEQ ID NO: 27;
c. a heavy chain variable region CDR1 comprising an amino acid sequence set forth in SEQ ID NO: 28; a heavy chain variable region CDR2 comprising an amino acid sequence set forth in SEQ ID NO: 29; a heavy chain variable region CDR3 comprising an amino acid sequence set forth in SEQ ID NO: 30; a light chain variable region CDR1 comprising an amino acid sequence set forth in SEQ ID NO: 31; a light chain variable region CDR2 comprising an amino acid sequence set forth in SEQ ID NO: 32; and a light chain variable region CDR3 comprising an amino acid sequence set forth in SEQ ID NO: 33;
d. a heavy chain variable region CDR1 comprising an amino acid sequence set forth in SEQ ID NO: 34; a heavy chain variable region CDR2 comprising an amino acid sequence set forth in SEQ ID NO: 35; a heavy chain variable region CDR3 comprising an amino acid sequence set forth in SEQ ID NO: 36; a light chain variable region CDR1 comprising an amino acid sequence set forth in SEQ ID NO: 37; a light chain variable region CDR2 comprising an amino acid sequence set forth in SEQ ID NO: 38; and a light chain variable region CDR3 comprising an amino acid sequence set forth in SEQ ID NO: 39;
e. a heavy chain variable region CDR1 comprising an amino acid sequence set forth in SEQ ID NO: 40; a heavy chain variable region CDR2 comprising an amino acid sequence set forth in SEQ ID NO: 41; a heavy chain variable region CDR3 comprising an amino acid sequence set forth in SEQ ID NO: 42; a light chain variable region CDR1 comprising an amino acid sequence set forth in SEQ ID NO: 43; a light chain variable region CDR2 comprising an amino acid sequence set forth in SEQ ID NO: 44; and a light chain variable region CDR3 comprising an amino acid sequence set forth in SEQ ID NO: 45;
f. a heavy chain variable region CDR1 comprising an amino acid sequence set forth in SEQ ID NO: 46; a heavy chain variable region CDR2 comprising an amino acid sequence set forth in SEQ ID NO: 47; a heavy chain variable region CDR3 comprising an amino acid sequence set forth in SEQ ID NO: 48; a light chain variable region CDR1 comprising an amino acid sequence set forth in SEQ ID NO: 49; a light chain variable region CDR2 comprising an amino acid sequence set forth in SEQ ID NO: 50; and a light chain variable region CDR3 comprising an amino acid sequence set forth in SEQ ID NO: 51;
g. a heavy chain variable region CDR1 comprising an amino acid sequence set forth in SEQ ID NO: 52; a heavy chain variable region CDR2 comprising an amino acid sequence set forth in SEQ ID NO: 53; a heavy chain variable region CDR3 comprising an amino acid sequence set forth in SEQ ID NO: 54; a light chain variable region CDR1 comprising an amino acid sequence set forth in SEQ ID NO: 55; a light chain variable region CDR2 comprising an amino acid sequence set forth in SEQ ID NO: 56; and a light chain variable region CDR3 comprising an amino acid sequence set forth in SEQ ID NO: 57; or
h. a heavy chain variable region CDR1 comprising an amino acid sequence set forth in SEQ ID NO: 58; a heavy chain variable region CDR2 comprising an amino acid sequence set forth in SEQ ID NO: 59; a heavy chain variable region CDR3 comprising an amino acid sequence set forth in SEQ ID NO: 60; a light chain variable region CDR1 comprising an amino acid sequence set forth in SEQ ID NO: 61; a light chain variable region CDR2 comprising an amino acid sequence set forth in SEQ ID NO: 62; and a light chain variable region CDR3 comprising an amino acid sequence set forth in SEQ ID NO: 63.

28. The method of claim 11, wherein contacting the sample with the vector occurs at least 12, 24, 36, 48, 60, 72, 84, 96, 108, 120, 132, or 144 hours prior to contacting the sample with the FoxP3 targeting agent; or contacting the sample with the FoxP3 targeting agent occurs at least 4, 6, 8, 10, 12, 16, 20, 24, 36, or 48 hours prior to contacting the sample with the vector.

29. A composition comprising: (a) an engineered immune cell, wherein the engineered immune cell expresses an engineered receptor; and (b) a FoxP3 targeting agent.

30. A composition comprising: (a) a vector encoding an engineered receptor; and (b) a FoxP3 targeting agent.


SEQUENCE LISTING (354 sequences)

SEQ ID NO: 1 - protein, 431 aa, Artificial Sequence
  Description of Artificial Sequence Synthetic polypeptide
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 2 - protein, 9 aa, Artificial Sequence
  Description of Artificial Sequence Synthetic peptide
KLSAMQAHL

SEQ ID NO: 3 - protein, 9 aa, Artificial Sequence
  Description of Artificial Sequence Synthetic peptide
SLHKCFVRV

SEQ ID NO: 4 - protein, 9 aa, Artificial Sequence
  Description of Artificial Sequence Synthetic peptide
SLFAVRRHL

SEQ ID NO: 5 - protein, 9 aa, Artificial Sequence
  Description of Artificial Sequence Synthetic peptide
NLSLHKCFV

SEQ ID NO: 6 - protein, 9 aa, Artificial Sequence
  Description of Artificial Sequence Synthetic peptide
LLQDRPHFM

SEQ ID NO: 7 - protein, 9 aa, Artificial Sequence
  Description of Artificial Sequence Synthetic peptide
LQLPTLPLV

SEQ ID NO: 8 - protein, 10 aa, Artificial Sequence
  Description of Artificial Sequence Synthetic peptide
TLIRWAILEA

SEQ ID NO: 9 - protein, 454 aa, Artificial Sequence
  Description of Artificial Sequence Synthetic polypeptide
EVQLVESGGGVVQPGRSLRLSCAASGFTFNNHAMHWVRQAPGKGLEWVAVISFDGDDKFYADSVKGRFTISRDNSRNTLFLQMNNLRPEDTAVYYCSRDPYHFASGSYSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  Description of Artificial Sequence Synthetic polypeptide
QSVLTQPPSVSVAPGKTARITCGGNNIGSKSVHWYQQKPGQAPVLVIYYDSDRPSGIPERFSGSNSGNTATLTISRVEAGDEADYYCQVWDSSSDHYVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 11 - protein, 510 aa, Artificial Sequence
  Description of Artificial Sequence Synthetic polypeptide
QSVLTQPPSVSVAPGKTARITCGGNNIGSKSVHWYQQKPGQAPVLVIYYDSDRPSGIPERFSGSNSGNTATLTISRVEAGDEADYYCQVWDSSSDHYVFGTGTKVTVLGSRGGGGSGGGGSGGGGSLEMAEVQLVESGGGVVQPGRSLRLSCAASGFTFNNHAMHWVRQAPGKGLEWVAVISFDGDDKFYADSVKGRFTISRDNSRNTLFLQMNNLRPEDTAVYYCSRDPYHFASGSYSYFDYWGQGTLVTVSSTSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 12 - protein, 476 aa, Artificial Sequence
  Description of Artificial Sequence Synthetic polypeptide
QSVLTQPPSVSVAPGKTARITCGGNNIGSKSVHWYQQKPGQAPVLVIYYDSDRPSGIPERFSGSNSGNTATLTISRVEAGDEADYYCQVWDSSSDHYVFGTGTKVTVLGSRGGGGSGGGGSGGGGSLEMAEVQLVESGGGVVQPGRSLRLSCAASGFTFNNHAMHWVRQAPGKGLEWVAVISFDGDDKFYADSVKGRFTISRDNSRNTLFLQMNNLRPEDTAVYYCSRDPYHFASGSYSYFDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 479 aa, Artificial Sequence
  Description of Artificial Sequence Synthetic polypeptide
QSVLTQPPSVSVAPGKTARITCGGNNIGSKSVHWYQQKPGQAPVLVIYYDSDRPSGIPERFSGSNSGNTATLTISRVEAGDEADYYCQVWDSSSDHYVFGTGTKVTVLGSRGGGGSGGGGSGGGGSLEMAEVQLVESGGGVVQPGRSLRLSCAASGFTFNNHAMHWVRQAPGKGLEWVAVISFDGDDKFYADSVKGRFTISRDNSRNTLFLQMNNLRPEDTAVYYCSRDPYHFASGSYSYFDYWGQGTLVTVSSTGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 315 aa, Artificial Sequence
  Description of Artificial Sequence Synthetic polypeptide
METDTLLLWVLLLWVPGSTGEVQLVESGGGVVQPGRSLRLSCAASGFTFNNHAMHWVRQAPGKGLEWVAVISFDGDDKFYADSVKGRFTISRDNSRNTLFLQMNNLRPEDTAVYYCSRDPYHFASGSYSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCEVKTDSTDHVKPKETENTKQPSKSCHKPKAIVHTEKVNMMSLTVLGLRMLFAKTVAVNFLLTAKLFFL

SEQ ID NO: 15 - protein, 301 aa, Artificial Sequence
  Description of Artificial Sequence Synthetic polypeptide
METDTLLLWVLLLWVPGSTGQSVLTQPPSVSVAPGKTARITCGGNNIGSKSVHWYQQKPGQAPVLVIYYDSDRPSGIPERFSGSNSGNTATLTISRVEAGDEADYYCQVWDSSSDHYVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECSPIKTDVITMDPKDNCSKDANDTLLLQLTNTSAYYMYLLLLLKSVVYFAIITCCLLRRTAFCCNGEKS

SEQ ID NO: 16 - protein, 8 aa, Artificial Sequence
  Description of Artificial Sequence Synthetic peptide
GDTFSRYA

SEQ ID NO: 17 - protein, 8 aa, Artificial Sequence
  Description of Artificial Sequence Synthetic peptide
IIPIFGTP

SEQ ID NO: 18 - protein, 12 aa, Artificial Sequence
  Description of Artificial Sequence Synthetic peptide
ARSIYRYSEYDH

SEQ ID NO: 19 - protein, 9 aa, Artificial Sequence
  Description of Artificial Sequence Synthetic peptide
SSNIGAGYD

SEQ ID NO: 20 - protein, 3 aa, Artificial Sequence
  Description of Artificial Sequence Synthetic peptide
GNS

SEQ ID NO: 21 - protein, 11 aa, Artificial Sequence
  Description of Artificial Sequence Synthetic peptide
QSYDSSLSGYV

SEQ ID NO: 22 - protein, 8 aa, Artificial Sequence
  Description of Artificial Sequence Synthetic peptide
GYTFSNYY

SEQ ID NO: 23 - protein, 8 aa, Artificial Sequence
  Description of Artificial Sequence Synthetic peptide
INPSVGTT

SEQ ID NO: 24 - protein, 12 aa, Artificial Sequence
  Description of Artificial Sequence Synthetic peptide
ARDWWGQMMYDG

SEQ ID NO: 25 - protein, 8 aa, Artificial Sequence
  Description of Artificial Sequence Synthetic peptide
SSNIGSNT

SEQ ID NO: 26 - protein, 3 aa, Artificial Sequence
  Description of Artificial Sequence Synthetic peptide
SNN

SEQ ID NO: 27 - protein, 12 aa, Artificial Sequence
  Description of Artificial Sequence Synthetic peptide
AAWDDSLNGQGV

SEQ ID NO: 28 - protein, 8 aa, Artificial Sequence
  Description of Artificial Sequence Synthetic peptide
GGTFSSYA

SEQ ID NO: 29 - protein, 8 aa, Artificial Sequence
  Description of Artificial Sequence Synthetic peptide
IIPIFGTA

SEQ ID NO: 30 - protein, 12 aa, Artificial Sequence
  Description of Artificial Sequence Synthetic peptide
ARYSYKYGELDT

SEQ ID NO: 31 - protein, 9 aa, Artificial Sequence
  Description of Artificial Sequence Synthetic peptide
SSNIGAGYD

SEQ ID NO: 32 - protein, 3 aa, Artificial Sequence
  Description of Artificial Sequence Synthetic peptide
GNS

SEQ ID NO: 33 - protein, 11 aa, Artificial Sequence
  Description of Artificial Sequence Synthetic peptide
QSYDSSLSGSV

SEQ ID NO: 34 - protein, 8 aa, Artificial Sequence
  Description of Artificial Sequence Synthetic peptide
GYTFTNYY

SEQ ID NO: 35 - protein, 8 aa, Artificial Sequence
  Description of Artificial Sequence Synthetic peptide
IRPSGGIT

SEQ ID NO: 36 - protein, 12 aa, Artificial Sequence
  Description of Artificial Sequence Synthetic peptide
ARSWDYFASNDF

SEQ ID NO: 37 - protein, 6 aa, Artificial Sequence
  Description of Artificial Sequence Synthetic peptide
NIGSES

SEQ ID NO: 38 - protein, 3 aa, Artificial Sequence
  Description of Artificial Sequence Synthetic peptide
DDD

SEQ ID NO: 39 - protein, 11 aa, Artificial Sequence
  Description of Artificial Sequence Synthetic peptide
QVWDRSSDHWF

SEQ ID NO: 40 - protein, 8 aa, Artificial Sequence
  Description of Artificial Sequence Synthetic peptide
GGTFSTYA

SEQ ID NO: 41 - protein, 8 aa, Artificial Sequence
  Description of Artificial Sequence Synthetic peptide
IIPIFGTA

SEQ ID NO: 42 - protein, 12 aa, Artificial Sequence
  Description of Artificial Sequence Synthetic peptide
ARAEYVYGEYDQ

SEQ ID NO: 43 - protein, 9 aa, Artificial Sequence
  Description of Artificial Sequence Synthetic peptide
SSNIGAGYD

SEQ ID NO: 44 - protein, 3 aa, Artificial Sequence
  Description of Artificial Sequence Synthetic peptide
GNS

SEQ ID NO: 45 - protein, 11 aa, Artificial Sequence
  Description of Artificial Sequence Synthetic peptide
QSYDSSLSGYV

SEQ ID NO: 46 - protein, 8 aa, Artificial Sequence
  Description of Artificial Sequence Synthetic peptide
GFTFNNHA

SEQ ID NO: 47 - protein, 8 aa, Artificial Sequence
  Description of Artificial Sequence Synthetic peptide
ISFDGDDK

SEQ ID NO: 48 - protein, 17 aa, Artificial Sequence
  Description of Artificial Sequence Synthetic peptide
SRDPYHFASGSYSYFDY

SEQ ID NO: 49 - protein, 6 aa, Artificial Sequence
  Description of Artificial Sequence Synthetic peptide
NIGSKS

SEQ ID NO: 50 - protein, 3 aa, Artificial Sequence
  Description of Artificial Sequence Synthetic peptide
YDS

SEQ ID NO: 51 - protein, 11 aa, Artificial Sequence
  Description of Artificial Sequence Synthetic peptide
QVWDSSSDHYV

SEQ ID NO: 52 - protein, 8 aa, Artificial Sequence
  Description of Artificial Sequence Synthetic peptide
GYTFTNYY

SEQ ID NO: 53 - protein, 8 aa, Artificial Sequence
  Description of Artificial Sequence Synthetic peptide
IRPSGGNT

SEQ ID NO: 54 - protein, 11 aa, Artificial Sequence
  Description of Artificial Sequence Synthetic peptide
ARSWNSRDVDS

SEQ ID NO: 55 - protein, 8 aa, Artificial Sequence
  Description of Artificial Sequence Synthetic peptide
SGSIASHY

SEQ ID NO: 56 - protein, 3 aa, Artificial Sequence
  Description of Artificial Sequence Synthetic peptide
ENN

SEQ ID NO: 57 - protein, 10 aa, Artificial Sequence
  Description of Artificial Sequence Synthetic peptide
QSYDRSNHVV

SEQ ID NO: 58 - protein, 8 aa, Artificial Sequence
  Description of Artificial Sequence Synthetic peptide
GGTFSSYA

SEQ ID NO: 59 - protein, 8 aa, Artificial Sequence
  Description of Artificial Sequence Synthetic peptide
IIPIFGTA

SEQ ID NO: 60 - protein, 14 aa, Artificial Sequence
  Description of Artificial Sequence Synthetic peptide
ARPSYYSIKSAWDH

SEQ ID NO: 61 - protein, 8 aa, Artificial Sequence
  Description of Artificial Sequence Synthetic peptide
TSNIGKNG

SEQ ID NO: 62 - protein, 3 aa, Artificial Sequence
  Description of Artificial Sequence Synthetic peptide
NDH

SEQ ID NO: 63 - protein, 11 aa, Artificial Sequence
  Description of Artificial Sequence Synthetic peptide
ATWDDTLDLPL

SEQ ID NO: 64 - protein, 119 aa, Artificial Sequence
  Description of Artificial Sequence Synthetic polypeptide
EVQLVQSGAEVKKPGSSVKVSCKASGDTFSRYAISWVRQAPGQGLEWMGRIIPIFGTPNYAQKFQGRVTITADEFTSTAYMELSSLRSEDTAMYYCARSIYRYSEYDHWGQGTLVTVSS

SEQ ID NO: 65 - protein, 119 aa, Artificial Sequence
  Description of Artificial Sequence Synthetic polypeptide
EVQLVQSGAEVKKPGTSVKVSCKASGYTFSNYYIHWVRQAPGQGLEWMGAINPSVGTTTYAQNFQGRVTMTSDTSTSTVYMELSSLTSEDTAVYYCARDWWGQMMYDGWGQGTLVTVSS

SEQ ID NO: 66 - protein, 119 aa, Artificial Sequence
  Description of Artificial Sequence Synthetic polypeptide
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARYSYKYGELDTWGQGTLVTVSS

SEQ ID NO: 67 - protein, 119 aa, Artificial Sequence
  Description of Artificial Sequence Synthetic polypeptide
QVQLVQSGAEVKKPGASVKVSCKASGYTFTNYYIHWVRQAPGQGLEWMGIIRPSGGITNYAQKFQGRVSMTRDTSTSTVYMELSSLRSEDTAVYYCARSWDYFASNDFWGQGTLVTVSS

SEQ ID NO: 68 - protein, 119 aa, Artificial Sequence
  Description of Artificial Sequence Synthetic polypeptide
QMQLVQSGAEVKKPGSSVKVSCKASGGTFSTYAISWVRQAPGQGLEWMGGIIPIFGTANYAQKFQGRVTITADKSTSTAYMEVRNLRSEDTAVYYCARAEYVYGEYDQWGQGTLVTVSS

SEQ ID NO: 69 - protein, 124 aa, Artificial Sequence
  Description of Artificial Sequence Synthetic polypeptide
EVQLVESGGGVVQPGRSLRLSCAASGFTFNNHAMHWVRQAPGKGLEWVAVISFDGDDKFYADSVKGRFTISRDNSRNTLFLQMNNLRPEDTAVYYCSRDPYHFASGSYSYFDYWGQGTLVTVSS

SEQ ID NO: 70 - protein, 118 aa, Artificial Sequence
  Description of Artificial Sequence Synthetic polypeptide
QVQLVQSGAEVKKPGASVKVSCKASGYTFTNYYIHWVRQAPGEGLEWMGIIRPSGGNTNYAQKFQGRVTMTRDTSTRTVYMELSSLRSEDTAVYYCARSWNSRDVDSWGQGTLVTVSS

SEQ ID NO: 71 - protein, 121 aa, Artificial Sequence
  Description of Artificial Sequence Synthetic polypeptide
QMQLVQSGAEVEKPGASVKLSCKASGGTFSSYAISWVRQAPGQGLEWMGGIIPIFGTANYAQKFQGRVTITADESTSTAYMELSSLRSEDTAVYYCARPSYYSIKSAWDHWGQGTLVTVSS

SEQ ID NO: 72 - protein, 120 aa, Artificial Sequence
  Description of Artificial Sequence Synthetic polypeptide
EVQLVQSGAEVKKPGESLKISCKGSGYNFASEWIGWVRQMPGKGLEWMGLIYPAESEITYSPSFQGQVTISVDKSISTAYLQWSSLKASDTAMYYCARAWDANYIYMDIWGQGTLVTVSS

SEQ ID NO: 73 - protein, 118 aa, Artificial Sequence
  Description of Artificial Sequence Synthetic polypeptide
QVQLVQSGGGLVQPGGSLKLSCAASGFAFSGSSLHWVRQASGKGLEWVGRITSKAYNYATLYAASVKGRFTISRDDSKNTAYLQMNSLQTEDTAVYYCTQTGDSSAYWGQGTLVTVSS

SEQ ID NO: 74 - protein, 117 aa, Artificial Sequence
  Description of Artificial Sequence Synthetic polypeptide
EVQLVESGGGLVQPGGSLRLSCAASGFTFSSYWMNWVRQAPGKGLEWVANIKQDGSEKNYVDSVKGRFTISRDNAKNSLYLQMNSLRGEDTAVYYCARYGGGPYDSWGQGTLVTVSS

SEQ ID NO: 75 - protein, 121 aa, Artificial Sequence
  Description of Artificial Sequence Synthetic polypeptide
EVQLVESGGGLAQPGGSLRLSCAASGFSFSNYAMSWVRQAPGTGLEWVAGISGRGGSIHYADSVKGRFTISRDNSQSTVFLQMNNLGAEDTAIYYCAKSSEDYYFYHMDAWGIGTTVTVSS

SEQ ID NO: 76 - protein, 119 aa, Artificial Sequence
  Description of Artificial Sequence Synthetic polypeptide
QVQLQQSGPGLVKPSQTLSLTCAISGDSISSKSAAWNWIRQSPSRGLEWLGRTYYRSKWYYEYAPSVRSRITINRDTSKNQFSLQLNSVTPEDTAVYYCARSTGTFDYWGQGTLVTVSS

SEQ ID NO: 77 - protein, 118 aa, Artificial Sequence
  Description of Artificial Sequence Synthetic polypeptide
QMQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARDRHDYVMDVWGKGTTVTVSS

SEQ ID NO: 78 - protein, 112 aa, Artificial Sequence
  Description of Artificial Sequence Synthetic polypeptide
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGYVFGTGTKVTVLG

SEQ ID NO: 79 - protein, 112 aa, Artificial Sequence
  Description of Artificial Sequence Synthetic polypeptide
QAVLTQPPSASGTRGQRVTISCSGSSSNIGSNTVNWYQQLPGTAPKLLIYSNNQRPSGVPDRFSGSKSGTSASLAISGLQSEDEADYYCAAWDDSLNGQGVFGTGTKVTVLG

SEQ ID NO: 80 - protein, 112 aa, Artificial Sequence
  Description of Artificial Sequence Synthetic polypeptide
QSVVTQPPSVSAAPGQKVTISCSGSSSNIGAGYDVHWYQQLPGTAPKLLIYGNSNRPSGVPDRFSGSKSGTSASLAITGLQAEDEADYYCQSYDSSLSGSVFGGGTKLTVLG

SEQ ID NO: 81 - protein, 109 aa, Artificial Sequence
  Description of Artificial Sequence Synthetic polypeptide
QAVLTQPPSVSVAPGKTARLTCGGNNIGSESVHWYQQKPGQAPLLVVYDDDDRPSGIPERFSGSNSGNTATLTISRVEAGDEADYYCQVWDRSSDHWFFGGGTKLTVLG

SEQ ID NO: 82 - protein, 112 aa, Artificial Sequence
  Description of Artificial Sequence Synthetic polypeptide
QSVLTQPPSVSGAPGQRVTISCTGSSSNIGAGYDVHWYQQLPGTAPKLLIYGNSDRPSGVPDRFSGSKSGTSASLAITGLQAEDEADYYCQSYDSSLSGYVFGTGTKVTVLG

SEQ ID NO: 83 - protein, 109 aa, Artificial Sequence
  Description of Artificial Sequence Synthetic polypeptide
QSVLTQPPSVSVAPGKTARITCGGNNIGSKSVHWYQQKPGQAPVLVIYYDSDRPSGIPERFSGSNSGNTATLTISRVEAGDEADYYCQVWDSSSDHYVFGTGTKVTVLG

SEQ ID NO: 84 - protein, 112 aa, Artificial Sequence
  Description of Artificial Sequence Synthetic polypeptide
NFMLTQPHSVSESPGKTVTISCTRSSGSIASHYVQWYQQRPGSSPTTVIYENNQRPSGVPDRFSGSIDSSSNSASLTISGLKTEDEADYYCQSYDRSNHVVFGGGTKLTVLG

SEQ ID NO: 85 - protein, 111 aa, Artificial Sequence
  Description of Artificial Sequence Synthetic polypeptide
QAVLTQPPSLSGGPRQRVTISCSGSTSNIGKNGVSWYQQLPGKAPKLLIYNDHLLSSGVSDRFSGSKSGTSASLAISGLQSDDEADYYCATWDDTLDLPLFGGGTKVTVLG

SEQ ID NO: 86 - protein, 112 aa, Artificial Sequence
  Description of Artificial Sequence Synthetic polypeptide
QSVVTQPPSVSGAPGQGVTIPCTGSSSNIGAGYDVHWYQQLPGTAPKLLIYGNNNRPSGVPDRFSGSRSGSSASLAITGLQAEDEADYYCQSYDSSLSDVVFGGGTKLTVLG

SEQ ID NO: 87 - protein, 109 aa, Artificial Sequence
  Description of Artificial Sequence Synthetic polypeptide
SYELTQPPSVSVAPGKTARITCGGNNIGSKSVHWYQQKPGQAPVLVIYYDSDRPSGIPERFSGSNSGNTATLTISRVEAGDEADYYCQVWDSSSDHWVFGGGTKLTVLG

SEQ ID NO: 88 - protein, 109 aa, Artificial Sequence
  Description of Artificial Sequence Synthetic polypeptide
SYELTQPPSVSVAPGKTARITCGGNNIGSKSVHWYQQKPGQAPVLVIYYDSDRPSGIPERFSGSNSGNTATLTISRVEAGDEADYYCQVWDSSSDHRVFGGGTKVTVLG

SEQ ID NO: 89 - protein, 109 aa, Artificial Sequence
  Description of Artificial Sequence Synthetic polypeptide
SYVLTQPPSVSVAPGETASITCGANNIGSESVHWYQQKSGQAPVLVIYYDSDRPSGIPERFSGSNSDNTATLTISRVEAGDEADYYCQVWDHINDHYVFGSGTKVTVLG

SEQ ID NO: 90 - protein, 109 aa, Artificial Sequence
  Description of Artificial Sequence Synthetic polypeptide
SYVLTQPPSVSVAPGKTARITCGGNNIGSKSVHWYQQKPGQAPVLVIYYDSDRPSGIPERFSGSNSGNTATLTISRVEAGDEADYYCQVWDSSSDRVVFGGGTKLTVLG

SEQ ID NO: 91 - protein, 109 aa, Artificial Sequence
  Description of Artificial Sequence Synthetic polypeptide
QSVLTQPPSVSVAPGKTAKITCGGDNIGSKTVHWYQQKPGQAPALLIYYDSDRPSGIPERFSGSNSGNTATLSISRVEAGDEAGYFCQVWDGSSDHVIFGGGTKLTVLG

SEQ ID NO: 92 - protein, 8 aa, Artificial Sequence
  Description of Artificial Sequence Synthetic peptide
GYSFPNYW

SEQ ID NO: 93 - protein, 8 aa, Artificial Sequence
  Description of Artificial Sequence Synthetic peptide
IDPGDSYT

SEQ ID NO: 94 - protein, 10 aa, Artificial Sequence
  Description of Artificial Sequence Synthetic peptide
ARYYVSLVDI

SEQ ID NO: 95 - protein, 9 aa, Artificial Sequence
  Description of Artificial Sequence Synthetic peptide
SSDVGGYNY

SEQ ID NO: 96 - protein, 3 aa, Artificial Sequence
  Description of Artificial Sequence Synthetic peptide
DVN

SEQ ID NO: 97 - protein, 10 aa, Artificial Sequence
  Description of Artificial Sequence Synthetic peptide
SSYTTGSRAV

SEQ ID NO: 98 - protein, 249 aa, Artificial Sequence
  Description of Artificial Sequence Synthetic polypeptide
QSVLTQPASVSGSPGQSITISCTGTSSDVGGYNYVSWYQQHPGKAPKLMIYDVNNRPSEVSNRFSGSKSGNTASLTISGLQAEDEADYYCSSYTTGSRAVFGGGTKLTVLGSRGGGGSGGGGSGGGGSLEMAEVQLVQSGAEVKKPGESLTISCKASGYSFPNYWITWVRQMSGGGLEWMGRIDPGDSYTTYNPSFQGHVTISIDKSTNTAYLHWNSLKASDTAMYYCARYYVSLVDIWGQGTLVTVSS

SEQ ID NO: 99 - protein, 222 aa, Artificial Sequence
  Description of Artificial Sequence Synthetic polypeptide
AAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - protein, 225 aa, Artificial Sequence
  Description of Artificial Sequence Synthetic polypeptide
TGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 130 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQVWGWQGGMYPRSNWWYNLDSWGQGTLVTVSS

SEQ ID NO: 102 - protein, 109 aa, Artificial Sequence
  Description of Artificial Sequence Synthetic polypeptide
LPVLTQPPSVSVAPGKTARITCGGNNIGSKSVHWYQQKPGQAPVLVVYDDSDRPSGIPERFSGSNSGNTATLTISRVEAGDEADYYCQVWDSSSDYVVFGGGTKLTVLG

SEQ ID NO: 103 - protein, 301 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQVWGWQGGMYPRSNWWYNLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCEVKTDSTDHVKPKETENTKQPSKSCHKPKAIVHTEKVNMMSLTVLGLRMLFAKTVAVNFLLTAKLFFL

SEQ ID NO: 104 - protein, 281 aa, Artificial Sequence
  Description of Artificial Sequence Synthetic polypeptide
LPVLTQPPSVSVAPGKTARITCGGNNIGSKSVHWYQQKPGQAPVLVVYDDSDRPSGIPERFSGSNSGNTATLTISRVEAGDEADYYCQVWDSSSDYVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECSPIKTDVITMDPKDNCSKDANDTLLLQLTNTSAYYMYLLLLLKSVVYFAIITCCLLRRTAFCCNGEKS

SEQ ID NO: 105 - protein, 8 aa, Artificial Sequence
  Description of Artificial Sequence Synthetic peptide
GYSFTSYW

SEQ ID NO: 106 - protein, 8 aa, Artificial Sequence
  Description of Artificial Sequence Synthetic peptide
IYPGDSDT

SEQ ID NO: 107 - protein, 23 aa, Artificial Sequence
  Description of Artificial Sequence Synthetic peptide
ARQVWGWQGGMYPRSNWWYNMDS

SEQ ID NO: 108 - protein, 23 aa, Artificial Sequence
  Description of Artificial Sequence Synthetic peptide
ARQVWGWQGGMYPRSNWWYNLDS

SEQ ID NO: 109 - protein, 6 aa, Artificial Sequence
  Description of Artificial Sequence Synthetic peptide
NIGSKS

SEQ ID NO: 110 - protein, 3 aa, Artificial Sequence
  Description of Artificial Sequence Synthetic peptide
DDS

SEQ ID NO: 111 - protein, 11 aa, Artificial Sequence
  Description of Artificial Sequence Synthetic peptide
QVWDSSSDYVV

SEQ ID NO: 112 - protein, 11 aa, Artificial Sequence
  Description of Artificial Sequence Synthetic peptide
QVWDSSSEYVV

SEQ ID NO: 113 - protein, 249 aa, Artificial Sequence
  Description of Artificial Sequence Synthetic polypeptide
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 114 - DNA, 750 bp, Artificial Sequence
  Description of Artificial Sequence Synthetic polynucleotide
gacgtgcagctggtgcagagcggagctgaagtgaagaaacctggcgcctccgtgaaggtgtcctgcaaagctagcggctataccttcacccggtacaccatgcactgggtgcgccaggcacctggacagggactggaatggatcggctacatcaacccctcccggggctacaccaactacgccgactctgtgaagggccggttcaccatcaccaccgataagtccaccagcaccgcttacatggaactgtcctccctgagatccgaggacaccgctacctactattgcgcccggtactacgacgaccactactgcctggactactggggacagggaaccacagtgaccgtgtcctctggcgagggcacctctactggatctgggggaagtggtggttctggcggcgctgacgacatcgtgctgacccagtctccagccaccctgtctctgagcccaggcgagagagctaccctgtcctgcagagcctcccagtccgtgtcctacatgaattggtatcagcagaagcctggcaaggcccctaagcggtggatctacgacacctccaaggtggcctctggcgtgccagcccggttttccggatctggctctggcaccgactactccctgaccatcaacagcctggaagccgaggacgctgccacctattactgccagcagtggtcctccaaccccctgacctttggaggcggcaccaaggtggaaatcaagcaccaccatcatcaccactga

SEQ ID NO: 115 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 116 - protein, 112 aa, Unknown
  Description of Unknown CD3-zeta sequence
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - DNA, 337 bp, Homo sapiens
agagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcg

SEQ ID NO: 118 - protein, 21 aa, Artificial Sequence
  Description of Artificial Sequence Synthetic peptide
SRGGGGSGGGGSGGGGSLEMA

SEQ ID NO: 119 - DNA, 50 bp, Artificial Sequence
  Description of Artificial Sequence Synthetic oligonucleotide
ctagaggtggtggtggtagcggcggcggcggctctggtggtggtggatcc

SEQ ID NO: 120 - protein, 15 aa, Artificial Sequence
  Description of Artificial Sequence Synthetic peptide
GGGGSGGGGSGGGGS

SEQ ID NO: 121 - DNA, 15 bp, Artificial Sequence
  Description of Artificial Sequence Synthetic oligonucleotide
ggcgggggaggatcc

SEQ ID NO: 122 - protein, 21 aa, Unknown
  Description of Unknown CD8 signal sequence
MALPVTALLLPLALLLHAARP

SEQ ID NO: 123 - DNA, 63 bp, Unknown
  Description of Unknown CD8 signal sequence
atggccctgccagtaacggctctgctgctgccacttgctctgctcctccatgcagccaggcct

SEQ ID NO: 124 - protein, 234 aa, Unknown
  Description of Unknown CD8 sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPWKSGDKPSLSARYV

SEQ ID NO: 125 - protein, 214 aa, Homo sapiens
MLRLLLALNLFPSIQVTGNKILVKQSPMLVAYDNALSCKYSYNLFSREFRASLHKGLDSAVEVCWYGNYSQQLQVYSKTGFNCDGKLGNESVTFYLQNLYQTDIYFCKIEVMYPPPYLDNEKSNGTIIHVKGKHLCPSPLFPGPSKPFWVLVWGGVLACYSLLVTVAFIIFWVRSKRSRLLHSDYMNMTPRRPGPTRKHYQPYAPPRDFAAYRS

SEQ ID NO: 126 - DNA, 321 bp, Homo sapiens
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 127 - protein, 251 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLGTKERDWCGPSPADLSPGASSVTPPAPAREPGHSPQIISFFLALTSTALLFLLFFLTLRFSWKRGRKKLLYIFKQPFMRPVQTTQEEDGCSCRFPEEEEGGCEL

SEQ ID NO: 128 - protein, 276 aa, Homo sapiens
MCVGARRLGRGPCAALLLLGLGLSTVTGLHCVGDTYPSNDRCCHECRPGNGMVSRCSRSQNTVCRPCGPGFYNDWSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 129 - protein, 196 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVWCILGCILICWLTKKKYSSSVHDPNGEYMFMRATAKKSRLTDVTL

SEQ ID NO: 130 - protein, 220 aa, Homo sapiens
MACLGFQRHKAQLNLATRTWPCTLLFFLLFIPVFCKAMHVAQPAWLASSRGIASFVCEYASPGKATEVRVTVLRQADSQVTEVCAATYMMGNELTFLDDSICTGTSSGNQLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 131 - protein, 524 aa, Homo sapiens
MWEAQFLGLLFLQPLWVAPVKPLQPGAEVPWWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 132 - protein, 368 aa, Homo sapiens
MLGQWTLILLLLLKVYQGKGCQGSADHWSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 133 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 134 - protein, 119 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRIPPYYGMDVWGQGTTVTVSS

SEQ ID NO: 135 - protein, 121 aa, Artificial Sequence
  Description of Artificial Sequence Synthetic polypeptide
QVQLQQSGPGLVKPSQTLSLTCAISGDSVSSNSAAWNWIRQSPSRGLEWLGRTYYGSKWYNDYAVSVKSRITINPDTSKNQFSLQLNSVTPEDTAVYYCARGRLGDAFDIWGQGTMVTVSS

SEQ ID NO: 136 - protein, 121 aa, Artificial Sequence
  Description of Artificial Sequence Synthetic polypeptide
QMQLVQSGAEVKEPGESLRISCKGSGYSFTNFWISWVRQMPGKGLEWMGRVDPGYSYSTYSPSFQGHVTISADKSTSTAYLQWNSLKASDTAMYYCARVQYSGYYDWFDPWGQGTLVTVSS

SEQ ID NO: 137 - protein, 118 aa, Artificial Sequence
  Description of Artificial Sequence Synthetic polypeptide
QVQLVQSGAEVKKPGESLKISCKGSGYNFSNKWIGWVRQLPGRGLEWIAIIYPGYSDITYSPSFQGRVTISADTSINTAYLHWHSLKASDTAMYYCVRHTALAGFDYWGLGTLVTVSS

SEQ ID NO: 138 - protein, 121 aa, Artificial Sequence
  Description of Artificial Sequence Synthetic polypeptide
EVQLVQSGGGVVRPGGSLRLSCAASGFTFDDYGMSWVRQAPGKGLEWVSGINWNGGSTGYADSVRGRFTISRDNAKNSLYLQMNSLRAEDTALYYCARERGYGYHDPHDYWGQGTLVTVSS

SEQ ID NO: 139 - protein, 118 aa, Artificial Sequence
  Description of Artificial Sequence Synthetic polypeptide
EVQLVETGGGLLQPGGSLRLSCAASGFSVSGTYMGWVRQAPGKGLEWVALLYSGGGTYHPASLQGRFIVSRDSSKNMVYLQMNSLKAEDTAVYYCAKGGAGGGHFDSWGQGTLVTVSS

SEQ ID NO: 140 - protein, 451 aa, Artificial Sequence
  Description of Artificial Sequence Synthetic polypeptide
QMQLVQSGAEVKEPGESLRISCKGSGYSFTNFWISWVRQMPGKGLEWMGRVDPGYSYSTYSPSFQGHVTISADKSTSTAYLQWNSLKASDTAMYYCARVQYSGYYDWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 141 - protein, 111 aa, Artificial Sequence
  Description of Artificial Sequence Synthetic polypeptide
QTVVTQPPSASGTPGQRVTISCSGSSSNIGSNYVYWYQQLPGTAPKLLIYRSNQRPSGVPDRFSGSKSGTSASLAISGPRSVDEADYYCAAWDDSLNGVVFGGGTKLTVLG

SEQ ID NO: 142 - protein, 108 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVDIKR

SEQ ID NO: 143 - protein, 111 aa, Artificial Sequence
  Description of Artificial Sequence Synthetic polypeptide
QAVVTQPPSASGTPGQRVTISCSGSSSNIGSNTVNWYQQVPGTAPKLLIYSNNQRPSGVPDRFSGSKSGTSASLAISGLQSEDEADYYCAAWDDSLNGWVFGGGTKLTVLG

SEQ ID NO: 144 - protein, 107 aa, Artificial Sequence
  Description of Artificial Sequence Synthetic polypeptide
DIQMTQSPSTLSASVGDRVTITCRASQNINKWLAWYQQRPGKAPQLLIYKASSLESGVPSRFSGSGSGTEYTLTISSLQPDDFATYYCQQYNSYATFGQGTKVEIKR

SEQ ID NO: 145 - protein, 109 aa, Artificial Sequence
  Description of Artificial Sequence Synthetic polypeptide
QSVVTQPPSVSVAPGKTARITCGRNNIGSKSVHWYQQKPGQAPVLVVYDDSDRPSGIPERFSGSNSGNTATLTISRVEAGDEADYYCQVWDSSSDHVVFGGGTKLTVLG

SEQ ID NO: 146 - protein, 112 aa, Artificial Sequence
  Description of Artificial Sequence Synthetic polypeptide
QSVLTQPPSVSGAPGQRVTISCTGSSSNIGAGYDVHWYQQLPGTAPKLLIYGNSNRPSGVPDRFSGSKSGTSASLAISGLQSEDEADYYCAAWDDSLNGYVFGTGTKLTVLG

SEQ ID NO: 147 - protein, 216 aa, Artificial Sequence
  Description of Artificial Sequence Synthetic polypeptide
QAVVTQPPSASGTPGQRVTISCSGSSSNIGSNTVNWYQQVPGTAPKLLIYSNNQRPSGVPDRFSGSKSGTSASLAISGLQSEDEADYYCAAWDDSLNGWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 148 - protein, 10 aa, Artificial Sequence
  Description of Artificial Sequence Synthetic peptide
GGTFSSYAIS

SEQ ID NO: 149 - protein, 17 aa, Artificial Sequence
  Description of Artificial Sequence Synthetic peptide
GIIPIFGTANYAQKFQG

SEQ ID NO: 150 - protein, 10 aa, Artificial Sequence
  Description of Artificial Sequence Synthetic peptide
RIPPYYGMDV

SEQ ID NO: 151 - protein, 13 aa, Artificial Sequence
  Description of Artificial Sequence Synthetic peptide
SGSSSNIGSNYVY

SEQ ID NO: 152 - protein, 7 aa, Artificial Sequence
  Description of Artificial Sequence Synthetic peptide
RSNQRPS

SEQ ID NO: 153 - protein, 11 aa, Artificial Sequence
  Description of Artificial Sequence Synthetic peptide
AAWDDSLNGVV

SEQ ID NO: 154 - protein, 12 aa, Artificial Sequence
  Description of Artificial Sequence Synthetic peptide
GDSVSSNSAAWN

SEQ ID NO: 155 - protein, 18 aa, Artificial Sequence
  Description of Artificial Sequence Synthetic peptide
RTYYGSKWYNDYAVSVKS

SEQ ID NO: 156 - protein, 9 aa, Artificial Sequence
  Description of Artificial Sequence Synthetic peptide
GRLGDAFDI

SEQ ID NO: 157 - protein, 10 aa, Artificial Sequence
  Description of Artificial Sequence Synthetic peptide
RASQSISSYN

SEQ ID NO: 158 - protein, 7 aa, Artificial Sequence
  Description of Artificial Sequence Synthetic peptide
AASSLQS

SEQ ID NO: 159 - protein, 9 aa, Artificial Sequence
  Description of Artificial Sequence Synthetic peptide
QQSYSTPLT

SEQ ID NO: 160 - protein, 10 aa, Artificial Sequence
  Description of Artificial Sequence Synthetic peptide
GYSFTNFWIS

SEQ ID NO: 161 - protein, 17 aa, Artificial Sequence
  Description of Artificial Sequence Synthetic peptide
RVDPGYSYSTYSPSFQG

SEQ ID NO: 162 - protein, 12 aa, Artificial Sequence
  Description of Artificial Sequence Synthetic peptide
VQYSGYYDWFDP

SEQ ID NO: 163 - protein, 13 aa, Artificial Sequence
  Description of Artificial Sequence Synthetic peptide
SGSSSNIGSNTVN

SEQ ID NO: 164 - protein, 7 aa, Artificial Sequence
  Description of Artificial Sequence Synthetic peptide
SNNQRPS

SEQ ID NO: 165 - protein, 11 aa, Artificial Sequence
  Description of Artificial Sequence Synthetic peptide
AAWDDSLNGWV

SEQ ID NO: 166 - protein, 10 aa, Artificial Sequence
  Description of Artificial Sequence Synthetic peptide
GYNFSNKWIG

SEQ ID NO: 167 - protein, 17 aa, Artificial Sequence
  Description of Artificial Sequence Synthetic peptide
IIYPGYSDITYSPSFQG

SEQ ID NO: 168 - protein, 9 aa, Artificial Sequence
  Description of Artificial Sequence Synthetic peptide
HTALAGFDY

SEQ ID NO: 169 - protein, 11 aa, Artificial Sequence
  Description of Artificial Sequence Synthetic peptide
RASQNINKWLA

SEQ ID NO: 170 - protein, 7 aa, Artificial Sequence
  Description of Artificial Sequence Synthetic peptide
KASSLES

SEQ ID NO: 171 - protein, 8 aa, Artificial Sequence
  Description of Artificial Sequence Synthetic peptide
QQYNSYAT

SEQ ID NO: 172 - protein, 10 aa, Artificial Sequence
  Description of Artificial Sequence Synthetic peptide
GFTFDDYGMS

SEQ ID NO: 173 - protein, 17 aa, Artificial Sequence
  Description of Artificial Sequence Synthetic peptide
GINWNGGSTGYADSVRG

SEQ ID NO: 174 - protein, 12 aa, Artificial Sequence
  Description of Artificial Sequence Synthetic peptide
ERGYGYHDPHDY

SEQ ID NO: 175 - protein, 11 aa, Artificial Sequence
  Description of Artificial Sequence Synthetic peptide
GRNNIGSKSVH

SEQ ID NO: 176 - protein, 7 aa, Artificial Sequence
  Description of Artificial Sequence Synthetic peptide
DDSDRPS

SEQ ID NO: 177 - protein, 11 aa, Artificial Sequence
  Description of Artificial Sequence Synthetic peptide
QVWDSSSDHVV

SEQ ID NO: 178 - protein, 10 aa, Artificial Sequence
  Description of Artificial Sequence Synthetic peptide
GFSVSGTYMG

SEQ ID NO: 179 - protein, 16 aa, Artificial Sequence
  Description of Artificial Sequence Synthetic peptide
LLYSGGGTYHPASLQG

SEQ ID NO: 180 - protein, 10 aa, Artificial Sequence
  Description of Artificial Sequence Synthetic peptide
GGAGGGHFDS

SEQ ID NO: 181 - protein, 14 aa, Artificial Sequence
  Description of Artificial Sequence Synthetic peptide
TGSSSNIGAGYDVH

SEQ ID NO: 182 - protein, 7 aa, Artificial Sequence
  Description of Artificial Sequence Synthetic peptide
GNSNRPS

SEQ ID NO: 183 - protein, 11 aa, Artificial Sequence
  Description of Artificial Sequence Synthetic peptide
AAWDDSLNGYV

SEQ ID NO: 184 - protein, 250 aa, Artificial Sequence
  Description of Artificial Sequence Synthetic polypeptide
QTVVTQPPSASGTPGQRVTISCSGSSSNIGSNYVYWYQQLPGTAPKLLIYRSNQRPSGVPDRFSGSKSGTSASLAISGPRSVDEADYYCAAWDDSLNGVVFGGGTKLTVLGSRGGGGSGGGGSGGGSLEMAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRIPPYYGMDVWGQGTTVTVSS

SEQ ID NO: 185 - protein, 250 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVDIKRSRGGGGSGGGGSGGGGSLEMAQVQLQQSGPGLVKPSQTLSLTCAISGDSVSSNSAAWNWIRQSPSRGLEWLGRTYYGSKWYNDYAVSVKSRITINPDTSKNQFSLQLNSVTPEDTAVYYCARGRLGDAFDIWGQGTMVTVSS

SEQ ID NO: 186 - protein, 253 aa, Artificial Sequence
  Description of Artificial Sequence Synthetic polypeptide
QAVVTQPPSASGTPGQRVTISCSGSSSNIGSNTVNWYQQVPGTAPKLLIYSNNQRPSGVPDRFSGSKSGTSASLAISGLQSEDEADYYCAAWDDSLNGWVFGGGTKLTVLGSRGGGGSGGGGSGGGGSLEMAQMQLVQSGAEVKEPGESLRISCKGSGYSFTNFWISWVRQMPGKGLEWMGRVDPGYSYSTYSPSFQGHVTISADKSTSTAYLQWNSLKASDTAMYYCARVQYSGYYDWFDPWGQGTLVTVSS

SEQ ID NO: 187 - protein, 246 aa, Artificial Sequence
  Description of Artificial Sequence Synthetic polypeptide
DIQMTQSPSTLSASVGDRVTITCRASQNINKWLAWYQQRPGKAPQLLIYKASSLESGVPSRFSGSGSGTEYTLTISSLQPDDFATYYCQQYNSYATFGQGTKVEIKRSRGGGGSGGGGSGGGGSLEMAQVQLVQSGAEVKKPGESLKISCKGSGYNFSNKWIGWVRQLPGRGLEWIAIIYPGYSDITYSPSFQGRVTISADTSINTAYLHWHSLKASDTAMYYCVRHTALAGFDYWGLGTLVTVSS

SEQ ID NO: 188 - protein, 249 aa, Artificial Sequence
  Description of Artificial Sequence Synthetic polypeptide
QSVVTQPPSVSVAPGKTARITCGRNNIGSKSVHWYQQKPGQAPVLVVYDDSDRPSGIPERFSGSNSGNTATLTISRVEAGDEADYYCQVWDSSSDHVVFGGGTKLTVLGSRGGGGSGGGGSGGSLEMAEVQLVQSGGGVVRPGGSLRLSCAASGFTFDDYGMSWVRQAPGKGLEWVSGINWNGGSTGYADSVRGRFTISRDNAKNSLYLQMNSLRAEDTALYYCARERGYGYHDPHDYWGQGTLVTVSS

SEQ ID NO: 189 - protein, 251 aa, Artificial Sequence
  Description of Artificial Sequence Synthetic polypeptide
QSVLTQPPSVSGAPGQRVTISCTGSSSNIGAGYDVHWYQQLPGTAPKLLIYGNSNRPSGVPDRFSGSKSGTSASLAISGLQSEDEADYYCAAWDDSLNGYVFGTGTKLTVLGSRGGGGSGGGGSGGGGSLEMAEVQLVETGGGLLQPGGSLRLSCAASGFSVSGTYMGWVRQAPGKGLEWVALLYSGGGTYHPASLQGRFIVSRDSSKNMVYLQMNSLKAEDTAVYYCAKGGAGGGHFDSWGQGTLVTVSS

SEQ ID NO: 190 - protein, 9 aa, Unknown
  Description of Unknown WT1 epitope
RMFPNAPYL

SEQ ID NO: 191 - protein, 121 aa, Artificial Sequence
  Description of Artificial Sequence Synthetic polypeptide
EVQLVQSGAEVKKPGASVKVSCKASGYTFTDYYIHWVRQAPGQGLEWMGWMNPNSGNSVSAQKFQGRVTMTRDTSINTAYMELSSLTSDDTAVYYCARNSEWHPWGYYDYWGQGTLVTVSS

SEQ ID NO: 192 - protein, 117 aa, Artificial Sequence
  Description of Artificial Sequence Synthetic polypeptide
EVQLVQSGAEVKKPGESLKISCQGSGYRFSKYWIGWVRQMPGKGLEWMGIIYPGDSDTRYSPSFQGQVTISADKSISTAYLQWSSLKASDTAMYYCARSFSSFIYDYWGQGTLVTVS

SEQ ID NO: 193 - protein, 116 aa, Artificial Sequence
  Description of Artificial Sequence Synthetic polypeptide
EVQLVQSGAEVKKPGESLKISCKASGYSFSNYWIGWVRQMPGKGLEWMGIIYPDDSDTRYSPSVQGQVTISADKSISTAYLQWYSLKVADTAKYYCVRPRGAFDIWGQGTTVTVSS

SEQ ID NO: 194 - protein, 116 aa, Artificial Sequence
  Description of Artificial Sequence Synthetic polypeptide
QVQLVESGAEVKKPGESLKISCKASGYSFSNYWIGWVRQMPGKGLEWMGIIYPDDSDTRYSPSFQGQVTISADKSISTAYLQWYSLKVADTAKYYCVRPRGAFDIWGQGTTVTVSS

SEQ ID NO: 195 - protein, 118 aa, Artificial Sequence
  Description of Artificial Sequence Synthetic polypeptide
QVQLVESGGGLVQPGGSLRLSCAASGFTFSSYWMSWVRQAPGKGLEWVANIKQDGSEKYYVDSVRGRFTISRDNAKNSLYLQMNSLRAEDTAMYYCARGSFSYDSDLWGQGTLVTVSS

SEQ ID NO: 196 - protein, 121 aa, Artificial Sequence
  Description of Artificial Sequence Synthetic polypeptide
EVQLVESGPGLVKPSQTLSLTCTVSGGSISSGGYYWSWIRQHPGKGLEWIGYIYYSGSTYYNPSLKSRVTISVDTSKNQFSLKLSSVTAADTAMYYCARGGLYWTYSQDVWGQGTLVTVSS

SEQ ID NO: 197 - protein, 124 aa, Artificial Sequence
  Description of Artificial Sequence Synthetic polypeptide
QMQLVQSGGGVVQPGRSLRLSCAASGFTFSNYDMHWVRRAPGKGLEWVAVISYDGSNNYYADSVKGRFTISRDNSKNTLYLQMNSLRAEDTAVYYCARSSAWVGGGFLSGTDDWGQGTLVTVSS

SEQ ID NO: 198 - protein, 127 aa, Artificial Sequence
  Description of Artificial Sequence Synthetic polypeptide
EVQLVQSGGGLVQPGGSLRLSCAASGFSLNDYYMDWVRQAPGEGLEWVGRIRDKAHGDTTEYIASVKDRFIVSRDDSKNSLYLQMNSLKTEDTAMYYCARWVDDYQGYWIWSYHDFWGQGTLVTVSS

SEQ ID NO: 199 - protein, 116 aa, Artificial Sequence
  Description of Artificial Sequence Synthetic polypeptide
QVQLVQSGGGLVQPGGSLRLSCAATGFTFSSYAMSWVRQAPGKGLEWVSVISGSGGSTYYADSVKGRFTISRDNSKNTLYLQMNSLRADDTAVYYCARHYYSSDSWGQGTLVTVSS

SEQ ID NO: 200 - protein, 120 aa, Artificial Sequence
  Description of Artificial Sequence Synthetic polypeptide
QMQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGGYTGWSPSDPWGQGTLVTVSS

SEQ ID NO: 201 - protein, 123 aa, Artificial Sequence
  Description of Artificial Sequence Synthetic polypeptide
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARHLGPMGMYDWSFDKWGQGTLVTVSS

SEQ ID NO: 202 - protein, 121 aa, Artificial Sequence
  Description of Artificial Sequence Synthetic polypeptide
QITLKESGPELVKPTQTLTLTCTFSGFSLSTSGMSVSWIRQPPGKALEWLARIDWDDDKYYSTSLKTRLTISKDTSKNQVVLTMTNTDPVDTATYYCARGFYLAYGSYDSWGQGTLVTVSS

SEQ ID NO: 203 - protein, 120 aa, Artificial Sequence
  Description of Artificial Sequence Synthetic polypeptide
EVQLVQSGAEVKKPGASVKVSCKASGYTFTNYYIHWVRQAPGQGLEWMGIINPTSGRTRYAQRFQGRVTMTRDTSTNTVYMDLSSLRSEDTAMYYCARSGYYWGVNGDQWGQGTLVTVSS

SEQ ID NO: 204 - protein, 109 aa, Artificial Sequence
  Description of Artificial Sequence Synthetic polypeptide
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHLVFGGGTKLTVLG

SEQ ID NO: 205 - protein, 108 aa, Artificial Sequence
  Description of Artificial Sequence Synthetic polypeptide
ETTLTQSPGTLSVSPGERATLSCRASQSVSSNLAWYQQKRGQAPRLLIYGASTRATGIPVRFSGSGSGTEFTLTISRLEPEDFAVYYCQQYGRSPLTFGGGTKVDIKR

SEQ ID NO: 206 - protein, 108 aa, Artificial Sequence
  Description of Artificial Sequence Synthetic polypeptide
EIVMTQSPATLSVSPGESATLSCRASQGVGINLAWYQQRPGQPPRLLIYDASNRATGIPARFSGSGSGTDFTLTISSLQAEDVAVYYCQQYYSFPWTFGQGTKVEIKR

SEQ ID NO: 207 - protein, 112 aa, Artificial Sequence
  Description of Artificial Sequence Synthetic polypeptide
QSVLTQPPSVSGAPGQRVTISCTGSSSNIGAGHAVHWYQQLPGTAPKLLIYDNANRPSGVPDRFSGSQSGTSASLAITGLQTGDEADYYCGTWDDSPSAYVFGTGTKVTVLG

SEQ ID NO: 208 - protein, 113 aa, Artificial Sequence
  Description of Artificial Sequence Synthetic polypeptide
DVVMTQSPLSLPVTLGQPASISCRSSQSLVHSDGNTYLNWFQQRPGQSPRRLIYKVSSRDSGVPDRFSGTGSGTDFTLKISRVEAEDVGVYYCMQTTHWPPTFGQGTKVEIKR

SEQ ID NO: 209 - protein, 111 aa, Artificial Sequence
  Description of Artificial Sequence Synthetic polypeptide
QPVLTQPPSASGTPGQRVTISCSGSSSNIGSDYVSWYQQLPGTAPKLLIYRNDQRPSGVPDRFSGSKSGTSASLAISGLRSEDEADYYCVAWDDSLSGYVFGSGTKVTVLG

SEQ ID NO: 210 - protein, 110 aa, Artificial Sequence
  Description of Artificial Sequence Synthetic polypeptide
EIVMTQSPATLSLSPGERATLSCGASQSVSSSYLAWYQQKPGLAPRLLIYDTSRRATGIPDRFSGSGSGTDFTLTISRLEPEDFAVYYCLHYGRSPPVTFGGGTKVEIKR

SEQ ID NO: 211 - protein, 113 aa, Artificial Sequence
  Description of Artificial Sequence Synthetic polypeptide
EIVLTQSPLSLPVTLGQPASISCRSSQSLAYSDGNTYLNWFHQRPGQSPRRLIYKVSKRDSGVPDRFSGSGSGTDFTLRISRVEAEDVGIYYCMQGTHWPHTFGPGTKVDIKR

SEQ ID NO: 212 - protein, 106 aa, Artificial Sequence
  Description of Artificial Sequence Synthetic polypeptide
SYVLTQPPSVSVSPGQTASVTCSGYRLREKYVSWYQQRPGHSPVLVIYEDTKRPSGIPERFSGSNSGDTATLTIRGTQAIDEADYYCQAWDSSVIFGGGTKLTVLG

SEQ ID NO: 213 - protein, 111 aa, Artificial Sequence
  Description of Artificial Sequence Synthetic polypeptide
QSALTQPASVSGSPGQSITISCTGTSSDVGGYNYVSWYQQHPGKAPKFMIYDVSKRPSGVSNRFSGSKSGNTASLTISGLQAEDEADYYCGSFTSSITYVFGTGTKVTVLG

SEQ ID NO: 214 - protein, 109 aa, Artificial Sequence
  Description of Artificial Sequence Synthetic polypeptide
QSVLTQPPSVSVAPGKTARITCGGDNIGRKSVHWYQQKPGQAPVLVIYYDSDRPSGIPERFSGSTSGNTATLTISRVEAGDEADYYCQVWDRSSDLYVFGTGTKVTVLG

SEQ ID NO: 215 - protein, 110 aa, Artificial Sequence
  Description of Artificial Sequence Synthetic polypeptide
QSALTQPASVSGSPGQSITISCTGTSGDVGGYNYVSWYQHHPGKAPKLIIYDVNKRPSGFSDRFSGSKSGNTASLTISGLQAEDEADYYCSSYTSTSTVFGGGTKLTVLG

SEQ ID NO: 216 - protein, 107 aa, Artificial Sequence
  Description of Artificial Sequence Synthetic polypeptide
SYELTQPLSVSVALGQTARITCGGNNIGSKNVHWYQQKPGQAPVLVIYRDSNRPSGIPERFSGSNSGNTATLTISRAQAGDEADYYCQVWDSSIVVFGGGTKLTVLG

SEQ ID NO: 217 - DNA, 326 bp, Artificial Sequence
  Description of Artificial Sequence Synthetic polynucleotide
tcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatctggtattcggcggagggaccaagctgaccgtcctagg

SEQ ID NO: 218 - DNA, 363 bp, Artificial Sequence
  Description of Artificial Sequence Synthetic polynucleotide
gaggtccagctggtacagtctggggctgaggtgaagaagcctggggcctcagtgaaggtctcctgcaaggcttctggatacaccttcaccgactactatatacactgggtgcggcaggcccctggacaagggctggagtggatgggatggatgaaccctaacagtgggaactcagtctctgcacagaagttccagggcagagtcaccatgaccagggatacctccataaacacagcctacatggagctgagcagcctgacatctgacgacacggccgtgtattactgtgcgcgcaactctgaatggcatccgtggggttactacgattactggggtcaaggtactctggtgaccgtctcctca

SEQ ID NO: 219 - DNA, 324 bp, Artificial Sequence
  Description of Artificial Sequence Synthetic polynucleotide
gaaacgacactcacgcagtctccaggcaccctgtctgtgtctccaggggaaagagccaccctctcctgcagggccagtcagagtgttagcagcaacttagcctggtaccagcagaaacgtggccaggctcccaggctcctcatctatggtgcgtctacccgggccactggtatcccagtcaggttcagtggcagtgggtctgggacagagttcactctcaccatcagcagattggagcctgaagattttgcagtgtattactgtcagcagtatggtaggtcaccgctcactttcggcggagggaccaaagtggatatcaaacgt

SEQ ID NO: 220 - DNA, 354 bp, Artificial Sequence
  Description of Artificial Sequence Synthetic polynucleotide
gaagtgcagctggtgcagtctggagcagaggtgaaaaagcccggggagtctctgaagatctcctgtcagggttctggatacaggttcagcaagtactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgcgctctttctcttctttcatctacgattactggggtcaaggtactctggtgaccgtctcctca

SEQ ID NO: 221 - DNA, 324 bp, Artificial Sequence
  Description of Artificial Sequence Synthetic polynucleotide
gaaattgtgatgacacagtctccagccaccctgtctgtgtctccaggggaaagtgccaccctctcctgcagggccagtcagggtgttggcatcaacttagcctggtaccagcagagacctggccagcctcccaggctcctcatctatgatgcatccaacagggccactggcatcccagccaggttcagtggcagtgggtctgggacagatttcactctcaccatcagcagcctgcaggctgaagatgtggcagtctattactgtcagcaatactatagttttccgtggacgttcggccaggggaccaaggtggaaatcaaacgt

SEQ ID NO: 222 - DNA, 348 bp, Artificial Sequence
  Description of Artificial Sequence Synthetic polynucleotide
gaggtgcagctggtgcagtctggggcagaggtgaaaaagcccggggagtctctgaaaatctcctgtaaggcttctggatacagctttagcaactactggatcggctgggtgcgccagatgcccgggaaaggcctggagtggatggggatcatctatcctgatgactctgataccagatacagcccgtccgtccaaggccaggtcaccatctcagccgacaagtccatcagcaccgcctacctgcagtggtacagcctgaaggtcgcggacaccgccaaatattactgtgtgcgccctaggggggcttttgatatctggggccaagggaccacggtcaccgtctcctca

SEQ ID NO: 223 - DNA, 336 bp, Artificial Sequence
  Description of Artificial Sequence Synthetic polynucleotide
cagtctgtgctgacgcagccgccctcagtgtctggggccccagggcagagggtcacgatctcctgcactgggagtagctccaacatcggggcaggtcatgctgtacactggtaccagcaacttccaggaacagcccccaaactcctcatctatgataacgccaatcggccctcaggggtccctgaccgattctctggctcccagtctggcacttcagcctccctggccatcaccggactccagactggggacgaggccgattattactgcggaacatgggatgacagcccgagtgcttatgtcttcggaactgggaccaaggtcaccgtcctaggt

SEQ ID NO: 224 - DNA, 348 bp, Artificial Sequence
  Description of Artificial Sequence Synthetic polynucleotide
caggtgcagctggtggagtctggggcagaggtgaaaaagcccggggagtctctgaaaatctcctgtaaggcttctggatacagctttagcaactactggatcggctgggtgcgccagatgcccgggaaaggcctggagtggatggggatcatctatcctgatgactctgataccagatacagcccgtccttccaaggccaggtcaccatctcagccgacaagtccatcagcaccgcctacctgcagtggtacagcctgaaggtcgcggacaccgccaaatattactgtgtgcgccctaggggggcttttgatatctggggccaagggaccacggtcaccgtctcctca

SEQ ID NO: 225 - DNA, 339 bp, Artificial Sequence
  Description of Artificial Sequence Synthetic polynucleotide
gatgttgtgatgactcagtctccactctccctgcccgtcacccttggacagccggcctccatctcctgcaggtctagtcaaagcctcgttcacagtgatggaaacacctacttgaattggtttcagcagaggccaggccaatctccaaggcgcctaatttataaagtttctagccgggactctggggtcccagatagattcagcggcactgggtcaggcactgatttcacactgaaaatcagcagggtggaggctgaagatgttggcgtttattactgcatgcaaaccacacactggcctccgacgttcggccaagggaccaaggtggagatcaaacgt

SEQ ID NO: 226 - DNA, 354 bp, Artificial Sequence
  Description of Artificial Sequence Synthetic polynucleotide
caggtgcagctggtggagtctgggggaggcttggtccagcctggggggtccctgagactctcctgtgcagcctctggattcacctttagtagctattggatgagctgggtccgccaggctccagggaaagggctggagtgggtggccaacataaagcaagatggaagtgagaaatactatgtggactctgtgaggggccgattcaccatctccagagacaacgccaagaactcactgtatctgcaaatgaacagcctgagagccgaggacaccgccatgtattactgtgcgcgcggttctttctcttacgacagtgatctgtggggtcaaggtactctggtgaccgtctcctca

SEQ ID NO: 227 - DNA, 333 bp, Artificial Sequence
  Description of Artificial Sequence Synthetic polynucleotide
cagcctgtgctgactcagccaccctcagcgtctgggacccccgggcagagggtcaccatctcttgttctggaagcagctccaacatcgggagtgattatgtatcctggtaccaacagctcccaggaacggcccccaaactcctcatctataggaatgatcagcggccctcaggggtccctgaccgattctctggctccaagtctggcacctcagcctccctggccatcagtgggctccggtccgaggatgaggctgattattactgtgtagcatgggatgacagcctgagtggttatgtcttcggaagtgggaccaaggtcaccgtcctaggt

SEQ ID NO: 228 - DNA, 363 bp, Artificial Sequence
  Description of Artificial Sequence Synthetic polynucleotide
gaggtgcagctggtggagtctggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtccaagaaccagttctccctgaagctgagctctgtgaccgctgcggacaccgccatgtattactgtgcgcgcggtggtctgtactggacttactctcaggatgtttggggtcaaggtactctggtgaccgtctcctca

SEQ ID NO: 229 - DNA, 330 bp, Artificial Sequence
  Description of Artificial Sequence Synthetic polynucleotide
gaaattgtgatgacgcagtctccagccaccctgtctttgtctccaggggaaagagccaccctctcctgcggggccagtcagagtgttagcagcagctacttagcctggtaccagcagaaacctggcctggcgcccaggctcctcatctatgatacatccagaagggccactggcatcccagacaggttcagtggcagtgggtctgggacagacttcactctcaccatcagcagactggagccggaagattttgcagtgtattactgtcttcactatggtcgctcacctccggtcactttcggcggagggaccaaggtggagatcaaacgt

SEQ ID NO: 230 - DNA, 372 bp, Artificial Sequence
  Description of Artificial Sequence Synthetic polynucleotide
cagatgcagctggtgcagtctgggggaggcgtggtccagcctgggaggtccctgagactctcctgtgcagcctctggattcaccttcagtaactatgacatgcactgggtccgccgggctccaggcaaggggctggagtgggtggcagttatatcatatgatggaagtaataattactatgcagactccgtgaagggccgattcaccatctccagagacaattccaagaacacgctgtatctgcaaatgaacagcctgagagctgaggacacggccgtgtattactgtgcgcgctcttctgcttgggttggtggtggtttcctgtctggtactgatgactggggtcaaggtactctggtgaccgtctcctca

SEQ ID NO: 231 - DNA, 339 bp, Artificial Sequence
  Description of Artificial Sequence Synthetic polynucleotide
gaaattgtgctgactcagtctccactctccctgcccgtcacccttggacagccggcctccatctcctgcaggtctagtcaaagcctcgcatacagtgatggaaacacctacttgaattggtttcaccagaggccaggccaatctccaaggcgcctaatctataaggtttctaagcgggactctggggtcccagacagattcagcggcagtgggtcaggcactgatttcacactgagaatcagcagggtggaggctgaggatgttgggatttattactgcatgcaaggtacacactggcctcacactttcggccctgggaccaaagtggatatcaaacgt

SEQ ID NO: 232 - DNA, 381 bp, Artificial Sequence
  Description of Artificial Sequence Synthetic polynucleotide
gaagtgcagctggtgcagtctgggggaggcttggtccagcctggagggtccctgagactctcctgtgcagcctctggattcagcctcaatgactattacatggactgggtccgccaggctccaggggaggggctggagtgggttggccgtattagagacaaagctcacggtgacaccacagaatacatcgcgtctgtgaaagacagatttatcgtctcaagagatgactccaagaactcactgtatctgcaaatgaacagcctgaaaaccgaggacaccgccatgtattactgtgcgcgctgggttgacgactaccagggttactggatctggtcttaccacgatttctggggtcaaggtactctggtgaccgtctcctca

SEQ ID NO: 233 - DNA, 318 bp, Artificial Sequence
  Description of Artificial Sequence Synthetic polynucleotide
tcctatgtgctgactcagccaccctcagtgtccgtgtccccaggacagacagccagcgtcacctgttctggatatagattgagagagaagtatgtttcctggtatcaacagaggccaggccactcccctgtcttggtcatctatgaagatactaagaggccttcagggatccctgagcgattctctggctccaattctggggacacagccactctgaccatcagagggacccaggctatagatgaggctgactattactgtcaggcgtgggacagcagcgtgattttcggcggagggaccaagctgaccgtcctaggt

SEQ ID NO: 234 - DNA, 348 bp, Artificial Sequence
  Description of Artificial Sequence Synthetic polynucleotide
caggtgcagctggtgcagtctgggggaggcttggtacagcctggggggtccctgagactctcctgtgcagccactggattcacctttagcagctatgccatgagttgggtccgccaggctccagggaaggggctggagtgggtctcagttattagtggtagtggtggtagcacatactacgcagactccgtgaagggccggttcaccatctccagagacaattccaagaacacgttgtatctgcaaatgaacagcctgagagccgacgacactgccgtgtattactgtgcgcgccattactactcttctgattcttggggtcaaggtactctggtgaccgtctcctca

SEQ ID NO: 235 - DNA, 333 bp, Artificial Sequence
  Description of Artificial Sequence Synthetic polynucleotide
caatctgccctgactcagcctgcctccgtgtctgggtctcctggacagtcgatcaccatctcctgcactggaaccagcagtgacgttggtggttataactatgtctcttggtaccaacagcacccaggcaaagcccccaaattcatgatttatgatgtcagtaagcggccctcaggtgtttctaatcgcttctctggctccaagtctggcaacacggcctccctgaccatctctgggctccaggctgaggacgaggctgattattactgcggctcatttacaagcagcatcacttatgtcttcggaactgggaccaaggtcaccgtcctaggt

SEQ ID NO: 236 - DNA, 360 bp, Artificial Sequence
  Description of Artificial Sequence Synthetic polynucleotide
cagatgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacactgccgtgtattactgtgcgcgcggtggttacactggttggtctccgtctgatccgtggggtcaaggtactctggtgaccgtctcctca

SEQ ID NO: 237 - DNA, 327 bp, Artificial Sequence
  Description of Artificial Sequence Synthetic polynucleotide
cagtctgtgttgactcagccaccctcagtgtcagtggccccaggaaagacggccaggattacctgtggtggagacaacattggacgtaaaagtgtgcactggtaccagcagaagccaggccaggcccctgtgctggtcatctattatgatagcgaccggccctcagggatccctgagcgattctctggctccacctctgggaacacggccaccctgaccatcagtagggtcgaagccggggatgaggccgactattactgtcaggtgtgggatcgtagtagtgacctttatgtcttcggaactgggaccaaggtcaccgtcctaggt

SEQ ID NO: 238 - DNA, 369 bp, Artificial Sequence
  Description of Artificial Sequence Synthetic polynucleotide
caggtgcagctggtgcagtctggggctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggctgtgtattactgtgcgcgccatctgggtccgatgggtatgtacgactggtctttcgataaatggggtcaaggtactctggtgaccgtctcctca

SEQ ID NO: 239 - DNA, 330 bp, Artificial Sequence
  Description of Artificial Sequence Synthetic polynucleotide
caatctgccctgactcagcctgcctccgtgtctgggtctcctggacagtcgatcaccatctcctgcactggaaccagcggtgacgttggcggttataactatgtctcctggtaccaacaccacccaggcaaagcccccaaactcataatttatgatgtcaataagcggccctcaggtttttctgatcggttctctggctccaagtctggcaacacggcctccctgacaatctctgggctccaggctgaggacgaggctgattattactgcagctcatatacaagcaccagcaccgtcttcggcggagggaccaagctgaccgtcctaggt

SEQ ID NO: 240 - DNA, 363 bp, Artificial Sequence
  Description of Artificial Sequence Synthetic polynucleotide
cagatcaccttgaaggagtctggtcctgagctggtgaaacccacacagaccctcacactgacctgcaccttttctgggttctcactcagcactagtggaatgtctgtgagctggatccgtcagcccccagggaaggccctggagtggcttgcacgcattgattgggatgatgataaatactacagcacatctctgaagaccaggctcaccatctccaaggacacctccaaaaaccaggtggtccttacaatgaccaacacggaccctgtggacacagccacgtattactgtgcgcgcggtttctacctggcttacggttcttacgattcttggggtcaaggtactctggtgaccgtctcctca

SEQ ID NO: 241 - DNA, 321 bp, Artificial Sequence
  Description of Artificial Sequence Synthetic polynucleotide
tcctatgagctgactcagccactctcagtgtcagtggccctgggacagacggccaggattacctgtgggggaaacaacattggaagtaaaaatgtgcactggtaccagcagaagccaggccaggcccctgtgctggtcatctatagggatagcaaccggccctctgggatccctgagcgattctctggctccaactcggggaacacggccaccctgaccatcagcagagcccaagccggggatgaggctgactattactgtcaggtgtgggacagcagtattgtggtattcggcggagggaccaagctgaccgtcctaggt

SEQ ID NO: 242 - DNA, 360 bp, Artificial Sequence
  Description of Artificial Sequence Synthetic polynucleotide
gaagtgcagctggtgcagtctggggctgaggtgaagaagcctggggcctcagtgaaggtttcctgcaaggcatctggatacaccttcaccaattactatatacactgggtgcgacaggcccctggacaagggcttgagtggatgggaataatcaaccctacaagtggtaggacaaggtacgcacagaggttccagggcagagtcaccatgaccagggacacgtccacgaacacagtctacatggacctgagcagcctgagatctgaagacaccgccatgtattactgtgcgcgctctggttactactggggtgttaacggtgatcagtggggtcaaggtactctggtgaccgtctcctca

SEQ ID NO: 243 - protein, 8 aa, Artificial Sequence
  Description of Artificial Sequence Synthetic peptide
GYTFTDYY

SEQ ID NO: 244 - protein, 8 aa, Artificial Sequence
  Description of Artificial Sequence Synthetic peptide
MNPNSGNS

SEQ ID NO: 245 - protein, 14 aa, Artificial Sequence
  Description of Artificial Sequence Synthetic peptide
ARNSEWHPWGYYDY

SEQ ID NO: 246 - protein, 6 aa, Artificial Sequence
  Description of Artificial Sequence Synthetic peptide
SLRSYY

SEQ ID NO: 247 - protein, 3 aa, Artificial Sequence
  Description of Artificial Sequence Synthetic peptide
GKN

SEQ ID NO: 248 - protein, 11 aa, Artificial Sequence
  Description of Artificial Sequence Synthetic peptide
NSRDSSGNHLV

SEQ ID NO: 249 - protein, 8 aa, Artificial Sequence
  Description of Artificial Sequence Synthetic peptide
GYRFSKYW

SEQ ID NO: 250 - protein, 8 aa, Artificial Sequence
  Description of Artificial Sequence Synthetic peptide
IYPGDSDT

SEQ ID NO: 251 - protein, 11 aa, Artificial Sequence
  Description of Artificial Sequence Synthetic peptide
ARSFSSFIYDY

SEQ ID NO: 252 - protein, 6 aa, Artificial Sequence
  Description of Artificial Sequence Synthetic peptide
QSVSSN

SEQ ID NO: 253 - protein, 3 aa, Artificial Sequence
  Description of Artificial Sequence Synthetic peptide
GAS

SEQ ID NO: 254 - protein, 9 aa, Artificial Sequence
  Description of Artificial Sequence Synthetic peptide
QQYGRSPLT

SEQ ID NO: 255 - protein, 8 aa, Artificial Sequence
  Description of Artificial Sequence Synthetic peptide
GYSFSNYW

SEQ ID NO: 256 - protein, 8 aa, Artificial Sequence
  Description of Artificial Sequence Synthetic peptide
IYPDDSDT

SEQ ID NO: 257 - protein, 9 aa, Artificial Sequence
  Description of Artificial Sequence Synthetic peptide
VRPRGAFDI

SEQ ID NO: 258 - protein, 6 aa, Artificial Sequence
  Description of Artificial Sequence Synthetic peptide
QGVGIN

SEQ ID NO: 259 - protein, 3 aa, Artificial Sequence
  Description of Artificial Sequence Synthetic peptide
DAS

SEQ ID NO: 260 - protein, 9 aa, Artificial Sequence
  Description of Artificial Sequence Synthetic peptide
QQYYSFPWT

SEQ ID NO: 261 - protein, 8 aa, Artificial Sequence
  Description of Artificial Sequence Synthetic peptide
GYSFSNYW

SEQ ID NO: 262 - protein, 8 aa, Artificial Sequence
  Description of Artificial Sequence Synthetic peptide
IYPDDSDT

SEQ ID NO: 263 - protein, 9 aa, Artificial Sequence
  Description of Artificial Sequence Synthetic peptide
VRPRGAFDI

SEQ ID NO: 264 - protein, 9 aa, Artificial Sequence
  Description of Artificial Sequence Synthetic peptide
SSNIGAGHA

SEQ ID NO: 265 - protein, 3 aa, Artificial Sequence
  Description of Artificial Sequence Synthetic peptide
DNA

SEQ ID NO: 266 - protein, 11 aa, Artificial Sequence
  Description of Artificial Sequence Synthetic peptide
GTWDDSPSAYV

SEQ ID NO: 267 - protein, 8 aa, Artificial Sequence
  Description of Artificial Sequence Synthetic peptide
GFTFSSYW

SEQ ID NO: 268 - protein, 8 aa, Artificial Sequence
  Description of Artificial Sequence Synthetic peptide
IKQDGSEK

SEQ ID NO: 269 - protein, 11 aa, Artificial Sequence
  Description of Artificial Sequence Synthetic peptide
ARGSFSYDSDL

SEQ ID NO: 270 - protein, 11 aa, Artificial Sequence
  Description of Artificial Sequence Synthetic peptide
QSLVHSDGNTY

SEQ ID NO: 271 - protein, 3 aa, Artificial Sequence
  Description of Artificial Sequence Synthetic peptide
KVS

SEQ ID NO: 272 - protein, 9 aa, Artificial Sequence
  Description of Artificial Sequence Synthetic peptide
MQTTHWPPT

SEQ ID NO: 273 - protein, 10 aa, Artificial Sequence
  Description of Artificial Sequence Synthetic peptide
GGSISSGGYY

SEQ ID NO: 274 - protein, 7 aa, Artificial Sequence
  Description of Artificial Sequence Synthetic peptide
IYYSGST

SEQ ID NO: 275 - protein, 13 aa, Artificial Sequence
  Description of Artificial Sequence Synthetic peptide
ARGGLYWTYSQDV

SEQ ID NO: 276 - protein, 8 aa, Artificial Sequence
  Description of Artificial Sequence Synthetic peptide
SSNIGSDY

SEQ ID NO: 277 - protein, 3 aa, Artificial Sequence
  Description of Artificial Sequence Synthetic peptide
RND

SEQ ID NO: 278 - protein, 11 aa, Artificial Sequence
  Description of Artificial Sequence Synthetic peptide
VAWDDSLSGYV

SEQ ID NO: 279 - protein, 8 aa, Artificial Sequence
  Description of Artificial Sequence Synthetic peptide
GFTFSNYD

SEQ ID NO: 280 - protein, 8 aa, Artificial Sequence
  Description of Artificial Sequence Synthetic peptide
ISYDGSNN

SEQ ID NO: 281 - protein, 17 aa, Artificial Sequence
  Description of Artificial Sequence Synthetic peptide
ARSSAWVGGGFLSGTDD

SEQ ID NO: 282 - protein, 7 aa, Artificial Sequence
  Description of Artificial Sequence Synthetic peptide
QSVSSSY

SEQ ID NO: 283 - protein, 3 aa, Artificial Sequence
  Description of Artificial Sequence Synthetic peptide
DTS

SEQ ID NO: 284 - protein, 10 aa, Artificial Sequence
  Description of Artificial Sequence Synthetic peptide
LHYGRSPPVT

SEQ ID NO: 285 - protein, 8 aa, Artificial Sequence
  Description of Artificial Sequence Synthetic peptide
GFSLNDYY

SEQ ID NO: 286 - protein, 10 aa, Artificial Sequence
  Description of Artificial Sequence Synthetic peptide
IRDKAHGDTT

SEQ ID NO: 287 - protein, 18 aa, Artificial Sequence
  Description of Artificial Sequence Synthetic peptide
ARWVDDYQGYWIWSYHDF

SEQ ID NO: 288 - protein, 11 aa, Artificial Sequence
  Description of Artificial Sequence Synthetic peptide
QSLAYSDGNTY

SEQ ID NO: 289 - protein, 3 aa, Artificial Sequence
  Description of Artificial Sequence Synthetic peptide
KVS

SEQ ID NO: 290 - protein, 9 aa, Artificial Sequence
  Description of Artificial Sequence Synthetic peptide
MQGTHWPHT

SEQ ID NO: 291 - protein, 8 aa, Artificial Sequence
  Description of Artificial Sequence Synthetic peptide
GFTFSSYA

SEQ ID NO: 292 - protein, 8 aa, Artificial Sequence
  Description of Artificial Sequence Synthetic peptide
ISGSGGST

SEQ ID NO: 293 - protein, 9 aa, Artificial Sequence
  Description of Artificial Sequence Synthetic peptide
ARHYYSSDS

SEQ ID NO: 294 - protein, 6 aa, Artificial Sequence
  Description of Artificial Sequence Synthetic peptide
RLREKY

SEQ ID NO: 295 - protein, 3 aa, Artificial Sequence
  Description of Artificial Sequence Synthetic peptide
EDT

SEQ ID NO: 296 - protein, 8 aa, Artificial Sequence
  Description of Artificial Sequence Synthetic peptide
QAWDSSVI

SEQ ID NO: 297 - protein, 8 aa, Artificial Sequence
  Description of Artificial Sequence Synthetic peptide
GYTFTSYY

SEQ ID NO: 298 - protein, 8 aa, Artificial Sequence
  Description of Artificial Sequence Synthetic peptide
INPSGGST

SEQ ID NO: 299 - protein, 13 aa, Artificial Sequence
  Description of Artificial Sequence Synthetic peptide
ARGGYTGWSPSDP

SEQ ID NO: 300 - protein, 9 aa, Artificial Sequence
  Description of Artificial Sequence Synthetic peptide
SSDVGGYNY

SEQ ID NO: 301 - protein, 3 aa, Artificial Sequence
  Description of Artificial Sequence Synthetic peptide
DVS

SEQ ID NO: 302 - protein, 10 aa, Artificial Sequence
  Description of Artificial Sequence Synthetic peptide
GSFTSSITYV

SEQ ID NO: 303 - protein, 8 aa, Artificial Sequence
  Description of Artificial Sequence Synthetic peptide
GYTFTSYG

SEQ ID NO: 304 - protein, 8 aa, Artificial Sequence
  Description of Artificial Sequence Synthetic peptide
ISAYNGNT

SEQ ID NO: 305 - protein, 16 aa, Artificial Sequence
  Description of Artificial Sequence Synthetic peptide
ARHLGPMGMYDWSFDK

SEQ ID NO: 306 - protein, 6 aa, Artificial Sequence
  Description of Artificial Sequence Synthetic peptide
NIGRKS

SEQ ID NO: 307 - protein, 3 aa, Artificial Sequence
  Description of Artificial Sequence Synthetic peptide
YDS

SEQ ID NO: 308 - protein, 11 aa, Artificial Sequence
  Description of Artificial Sequence Synthetic peptide
QVWDRSSDLYV

SEQ ID NO: 309 - protein, 10 aa, Artificial Sequence
  Description of Artificial Sequence Synthetic peptide
GFSLSTSGMS

SEQ ID NO: 310 - protein, 7 aa, Artificial Sequence
  Description of Artificial Sequence Synthetic peptide
IDWDDDK

SEQ ID NO: 311 - protein, 13 aa, Artificial Sequence
  Description of Artificial Sequence Synthetic peptide
ARGFYLAYGSYDS

SEQ ID NO: 312 - protein, 9 aa, Artificial Sequence
  Description of Artificial Sequence Synthetic peptide
SGDVGGYNY

SEQ ID NO: 313 - protein, 3 aa, Artificial Sequence
  Description of Artificial Sequence Synthetic peptide
DVN

SEQ ID NO: 314 - protein, 9 aa, Artificial Sequence
  Description of Artificial Sequence Synthetic peptide
SSYTSTSTV

SEQ ID NO: 315 - protein, 8 aa, Artificial Sequence
  Description of Artificial Sequence Synthetic peptide
GYTFTNYY

SEQ ID NO: 316 - protein, 8 aa, Artificial Sequence
  Description of Artificial Sequence Synthetic peptide
INPTSGRT

SEQ ID NO: 317 - protein, 13 aa, Artificial Sequence
  Description of Artificial Sequence Synthetic peptide
ARSGYYWGVNGDQ

SEQ ID NO: 318 - protein, 6 aa, Artificial Sequence
  Description of Artificial Sequence Synthetic peptide
NIGSKN

SEQ ID NO: 319 - protein, 3 aa, Artificial Sequence
  Description of Artificial Sequence Synthetic peptide
RDS

SEQ ID NO: 320 - protein, 9 aa, Artificial Sequence
  Description of Artificial Sequence Synthetic peptide
QVWDSSIVV

SEQ ID NO: 321 - protein, 502 aa, Artificial Sequence
  Description of Artificial Sequence Synthetic polypeptide
SYELTQPLSVSVALGQTARITCGGNNIGSKNVHWYQQKPGQAPVLVIYRDSNRPSGIPERFSGSNSGNTATLTISRAQAGDEADYYCQVWDSSIVVFGGGTKLTVLGSRGGGGSGGGGSGGGGSLEMAEVQLVQSGAEVKKPGASVKVSCKASGYTFTNYYIHWVRQAPGQGLEWMGIINPTSGRTRYAQRFQGRVTMTRDTSTNTVYMDLSSLRSEDTAMYYCARSGYYWGVNGDQWGQGTLVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 322 - protein, 105 aa, Homo sapiens
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 323 - DNA, 318 bp, Homo sapiens
cagcctaaggccaaccctaccgtgaccctgttccccccatcctccgaggaactgcaggccaacaaggccaccctcgtgtgcctgatctccgacttctaccctggcgccgtgaccgtggcctggaaggctgatggatctcctgtgaaggccggcgtggaaaccaccaagccctccaagcagtccaacaacaaatacgccgcctcctcctacctgtccctgacccctgagcagtggaagtcccaccggtcctacagctgccaagtgacccacgagggctccaccgtggaaaagaccgtggctcctaccgagtgctcctag

SEQ ID NO: 324 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 325 - DNA, 321 bp, Homo sapiens
accgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagagcggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgctag

SEQ ID NO: 326 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - DNA, 1002 bp, Homo sapiens
gtctcctcagcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 328 - DNA, 2832 bp, Homo sapiens
atggcccggggctcggcgctcccgcggcggccgctgctgtgcatcccggccgtctgggcggccgccgcgcttctgctctcagtgtcccggacttcaggtgaagtggaggttctggatccgaacgaccctttaggaccccttgatgggcaggacggcccgattccaactctgaaaggttactttctgaattttctggagccagtaaacaatatcaccattgtccaaggccagacggcaattctgcactgcaaggtggcaggaaacccaccccctaacgtgcggtggctaaagaatgatgccccggtggtgcaggagccgcggcggatcatcatccggaagacagaatatggttcacgactgcgaatccaggacctggacacgacagacactggctactaccagtgcgtggccaccaacgggatgaagaccattaccgccactggcgtcctgtttgtgcggctgggtccaacgcacagcccaaatcataactttcaggatgattaccacgaggatgggttctgccagccttaccggggaattgcctgtgcacgcttcattggcaaccggaccatttatgtggactcgcttcagatgcagggggagattgaaaaccgaatcacagcggccttcaccatgatcggcacgtctacgcacctgtcggaccagtgctcacagttcgccatcccatccttctgccacttcgtgtttcctctgtgcgacgcgcgctcccggacacccaagccgcgtgagctgtgccgcgacgagtgcgaggtgctggagagcgacctgtgccgccaggagtacaccatcgcccgctccaacccgctcatcctcatgcggcttcagctgcccaagtgtgaggcgctgcccatgcctgagagccccgacgctgccaactgcatgcgcattggcatcccagccgagaggctgggccgctaccatcagtgctataacggctcaggcatggattacagaggaacggcaagcaccaccaagtcaggccaccagtgccagccgtgggccctgcagcacccccacagccaccacctgtccagcacagacttccctgagcttggaggggggcacgcctactgccggaaccccggaggccagatggagggcccctggtgctttacgcagaataaaaacgtacgcatggaactgtgtgacgtaccctcgtgtagtccccgagacagcagcaagatggggattctgtacatcttggtccccagcatcgcaattccactggtcatcgcttgccttttcttcttggtttgcatgtgccggaataagcagaaggcatctgcgtccacaccgcagcggcgacagctgatggcctcgcccagccaagacatggaaatgcccctcattaaccagcacaaacaggccaaactcaaagagatcagcctgtctgcggtgaggttcatggaggagctgggagaggaccggtttgggaaagtctacaaaggtcacctgttcggccctgccccgggggagcagacccaggctgtggccatcaaaacgctgaaggacaaagcggaggggcccctgcgggaggagttccggcatgaggctatgctgcgagcacggctgcaacaccccaacgtcgtctgcctgctgggcgtggtgaccaaggaccagcccctgagcatgatcttcagctactgttcgcacggcgacctccacgaattcctggtcatgcgctcgccgcactcggacgtgggcagcaccgatgatgaccgcacggtgaagtccgccctggagccccccgacttcgtgcaccttgtggcacagatcgcggcggggatggagtacctatccagccaccacgtggttcacaaggacctggccacccgcaatgtgctagtgtacgacaagctgaacgtgaagatctcagacttgggcctcttccgagaggtgtatgccgccgattactacaagctgctggggaactcgctgctgcctatccgctggatggccccagaggccatcatgtacggcaagttctccatcgactcagacatctggtcctacggtgtggtcctgtgggaggtcttcagctacggcctgcagccctactgcgggtactccaaccaggatgtggtggagatgatccggaaccggcaggtgctgccttgccccgatgactgtcccgcctgggtgtatgccctcatgatcgagtgctggaacgagttccccagccggcggccccgcttcaaggacatccacagccggctccgagcctggggcaacctttccaactacaacagctcggcgcagacctcgggggccagcaacaccacgcagaccagctccctgagcaccagcccagtgagcaatgtgagcaacgcccgctacgtggggcccaagcagaaggccccgcccttcccacagccccagttcatccccatgaagggccagatcagacccatggtgcccccgccgcagctctacgtccccgtcaacggctaccagccggtgccggcctatggggcctacctgcccaacttctacccggtgcagatcccaatgcagatggccccgcagcaggtgcctcctcagatggtccccaagcccagctcacaccacagtggcagtggctccaccagcacaggctacgtcaccacggccccctccaacacatccatggcagacagggcagccctgctctcagagggcgctgatgacacacagaacgccccagaagatggggcccagagcaccgtgcaggaagcagaggaggaggaggaaggctctgtcccagagactgagctgctgggggactgtgacactctgcaggtggacgaggcccaagtccagctggaagcttga

SEQ ID NO: 329 - protein, 943 aa, Homo sapiens
MARGSALPRRPLLCIPAVWAAAALLLSVSRTSGEVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRTPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMGILYILVPSIAIPLVIACLFFLVCMCRNKQKASASTPQRRQLMASPSQDMEMPLINQHKQAKLKEISLSAVRFMEELGEDRFGKVYKGHLFGPAPGEQTQAVAIKTLKDKAEGPLREEFRHEAMLRARLQHPNVVCLLGVVTKDQPLSMIFSYCSHGDLHEFLVMRSPHSDVGSTDDDRTVKSALEPPDFVHLVAQIAAGMEYLSSHHVVHKDLATRNVLVYDKLNVKISDLGLFREVYAADYYKLLGNSLLPIRWMAPEAIMYGKFSIDSDIWSYGVVLWEVFSYGLQPYCGYSNQDVVEMIRNRQVLPCPDDCPAWVYALMIECWNEFPSRRPRFKDIHSRLRAWGNLSNYNSSAQTSGASNTTQTSSLSTSPVSNVSNARYVGPKQKAPPFPQPQFIPMKGQIRPMVPPPQLYVPVNGYQPVPAYGAYLPNFYPVQIPMQMAPQQVPPQMVPKPSSHHSGSGSTSTGYVTTAPSNTSMADRAALLSEGADDTQNAPEDGAQSTVQEAEEEEEGSVPETELLGDCDTLQVDEAQVQLEA

SEQ ID NO: 330 - protein, 15 aa, Homo sapiens
KTITATGVLFVRLGP

SEQ ID NO: 331 - protein, 15 aa, Homo sapiens
TGYYQCVATNGMKTI

SEQ ID NO: 332 - protein, 15 aa, Homo sapiens
RGIACARFIGNRTIY

SEQ ID NO: 333 - protein, 19 aa, Homo sapiens
CQPYRGIACARFIGNRTIY

SEQ ID NO: 334 - protein, 19 aa, Homo sapiens
QCSQFAIPSFCHFVFPLCD

SEQ ID NO: 335 - protein, 15 aa, Homo sapiens
ELCRDECEVLESDLC

SEQ ID NO: 336 - protein, 15 aa, Homo sapiens
ANCMRIGIPAERLGR

SEQ ID NO: 337 - protein, 370 aa, Homo sapiens
EVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRTPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMG

SEQ ID NO: 338 - protein, 9 aa, Artificial Sequence
  Description of Artificial Sequence Synthetic peptide
FMNKFIYEI

SEQ ID NO: 339 - protein, 9 aa, Artificial Sequence
  Description of Artificial Sequence Synthetic peptide
QLQNPSYDK

SEQ ID NO: 340 - protein, 10 aa, Artificial Sequence
  Description of Artificial Sequence Synthetic peptide
KLLVVGGVGV

SEQ ID NO: 341 - protein, 10 aa, Artificial Sequence
  Description of Artificial Sequence Synthetic peptide
TLIRWAILEA

SEQ ID NO: 342 - protein, 10 aa, Artificial Sequence
  Description of Artificial Sequence Synthetic peptide
ALIRWAILEA

SEQ ID NO: 343 - protein, 10 aa, Artificial Sequence
  Description of Artificial Sequence Synthetic peptide
TAIRWAILEA

SEQ ID NO: 344 - protein, 10 aa, Artificial Sequence
  Description of Artificial Sequence Synthetic peptide
TLARWAILEA

SEQ ID NO: 345 - protein, 10 aa, Artificial Sequence
  Description of Artificial Sequence Synthetic peptide
TLIAWAILEA

SEQ ID NO: 346 - protein, 10 aa, Artificial Sequence
  Description of Artificial Sequence Synthetic peptide
TLIRAAILEA

SEQ ID NO: 347 - protein, 10 aa, Artificial Sequence
  Description of Artificial Sequence Synthetic peptide
TLIRWAALEA

SEQ ID NO: 348 - protein, 10 aa, Artificial Sequence
  Description of Artificial Sequence Synthetic peptide
TLIRWAIAEA

SEQ ID NO: 349 - protein, 10 aa, Artificial Sequence
  Description of Artificial Sequence Synthetic peptide
TLIRWAILAA

SEQ ID NO: 350 - protein, 10 aa, Artificial Sequence
  Description of Artificial Sequence Synthetic peptide
TLIRWAILEG

SEQ ID NO: 351 - protein, 10 aa, Artificial Sequence
  Description of Artificial Sequence Synthetic peptide
TLIRWAILEA

SEQ ID NO: 352 - protein, 9 aa, Artificial Sequence
  Description of Artificial Sequence Synthetic peptide
RTLDKVLEV

SEQ ID NO: 353 - protein, 9 aa, Artificial Sequence
  Description of Artificial Sequence Synthetic peptide
VLHDDLLEA

SEQ ID NO: 354 - protein, 6 aa, Artificial Sequence
  Description of Artificial Sequence Synthetic 6xHis tag
HHHHHH